(19) United States
(12) Patent Application Publication
(10) Pub. No.: US 2016/0355483 A1
(43) Pub. Date: Dec. 8, 2016

(54) COMPOUND BINDING TO PPARG BUT NOT ACTING AS PROMOTER AND PHARMACEUTICAL COMPOSITION FOR TREATING PPARG-RELATED DISEASES CONTAINING SAME AS ACTIVE INGREDIENT

(71) Applicant: Hyundai Pharm Co., Ltd., Cheonan-si Chungcheongnam-do (KR)

(72) Inventors: In Hee LEE, Hwaseong-si Gyeonggi-do (KR); Hee Il CHAE, Paldal-gu Suwon-si Gyeonggi-do (KR); Se hoan KIM, Paldal-gu Suwon-si Gyeonggi-do (KR); Soon Young MOON, Yeongtong-gu Suwon-si Gyeonggi-do (KR); Tae Young HA, Paldal-gu Suwon-si Gyeonggi-do (KR); Hyo sun CHOI, Paldal-gu Suwon-si Gyeonggi-do (KR); Young Seok KIM, Seoul (KR); Chun hwa KIM, Yongin-si Gyeonggi-do (KR); Jae Keol RHEE, Hwaseong-si Gyeonggi-do (KR)

(73) Assignee: Hyundai Pharm Co., Ltd., Cheonan-si Chungcheongnam-do (KR)

(21) Appl. No.: 15/244,821

(22) Filed: Aug. 23, 2016

Related U.S. Application Data
(63) Continuation of application No. PCT/KR2015/001941, filed on Feb. 27, 2015.

(30) Foreign Application Priority Data
Mar. 20, 2014 (KR) 10-2014-0033041

(51) Int. Cl. C07D 215/48; C07D 401/12; C07D 405/12; C07D 265/36; C07D 413/12

(52) U.S. Cl. CPC C07D 215/48; C07D 265/36; C07D 413/12; C07D 405/12; C07D 401/12

(57) ABSTRACT

The present invention relates to a compound inhibiting CDK5-mediated PPARG phosphorylation and a pharmaceutical composition for treating PPARG-related diseases containing the same as an active ingredient. A compound represented by formula 1 or an optical isomer thereof according to the present invention that binds to PPARG at a high affinity level, but does not act as an agonist, thereby inducing no gene transcription; blocks CDK5, which is a cause of PPARG phosphorylation, thereby causing no side effects of existing anti-diabetic drugs; can be formulated into drugs due to improved pharmacological properties thereof; and can be favorably used as a pharmaceutical composition for treating PPARG-related diseases due to favorable treatment effects on PPARG-related diseases.

### CROSS-REFERENCE TO RELATED APPLICATIONS

This is a continuation of PCT Application No. PCT/KR2015/001941, filed on Feb. 27, 2015, which is incorporated by reference, and which claims priority to Korean Patent Application No. 10-2014-0033041, filed on Mar. 20, 2014.

### FIELD OF THE INVENTION

The present invention relates to a compound binding to PPARG but not acting as a promoter and a pharmaceutical composition for treating PPARG-related diseases comprising the compound as an active ingredient.

### BACKGROUND

According to CDC (Centers for Disease Control and Prevention, USA) data, 24 million people, which are 8% of the total US population, are diabetic patients in US. Over the past decades, the number of diabetic patients has been growing and so has the diabetic drug market. The dominant diabetic-treating drug has been insulin so far, but treatment using insulin injection has a problem of the inconvenience of using a syringe.

Further, it can only supplement the shortage of insulin but cannot be a fundamental solution for diabetes. Therefore, a variety of alternative drugs have been developed and are currently on the market, including sulfonylurea, which increases insulin secretion; metformin, which slowly isolates glucose deposited in the liver; acarbose, which suppresses glycolysis to inhibit sugar adsorption; and rosiglitazone, which increases the sensitivity of insulin receptors. In spite of these drugs, treating diabetes is unsatisfactory and cannot meet the needs of diabetic patients. Recently, understanding of the cause and the mechanism of diabetes has been enhanced, and thereby, the diabetic drug market has become a new land of opportunity.

Thiazolidinedione (TZD) drugs, such as rosiglitazone and pioglitazone, activate the transcription of PPARG (peroxisome proliferator activated receptor-gamma), the nuclear receptor, and thereby increase insulin sensitivity to generate an anti-diabetic effect. It is well-known that this type of drug is effective in regulating PPARG, but recently, it was also confirmed that this type of drug affects another biochemical pathway. The involvement of this drug in the obesity-like insulin resistance mechanism is an example. The known cause of the insulin resistance observed in diabetic patients is phosphorylation of the 273^{rd} amino acid of PPARG, serine, by CDK5 (cyclin-dependent kinase 5), which can be induced by mutation. It has been additionally confirmed that blocking CDK5 from interacting with PPARG is an important approach for developing an efficient anti-diabetic agent.

A patent document (US2012-0309757 A1) introduces compounds that bind to PPARG with high affinity but do not induce transcription of PPARG by acting as an agonist and thereby suppress insulin resistance by blocking CDK5, such that they display anti-diabetic effects (e.g., SR1664 and SR1824). Those compounds were also confirmed in a comparative experiment to have a significantly lower chance of producing side effects, such as weight gain and fluid retention, which frequently accompany conventional drugs, such as rosiglitazone. From a cell culture test, it was confirmed that SR1664 does not induce problems such as fat generation in bone cells, which is a side effect of rosiglitazone. The above results confirm that SR1664 can specifically block CDK5-mediated PPARG phosphorylation.

Even though the said SR1664 has many advantages, there is a problem with developing it as a drug due to poor pharmaco-kinetic (PK) pharmacophysical properties, which render in vivo absorption difficult.

### SUMMARY

It is an object of the present invention to provide a compound that binds PPARG but does not act as a promoter, an optical isomer thereof, or a pharmaceutically acceptable salt of the same.

It is another object of the present invention to provide a method for preparing the compound above.

It is also an object of the present invention to provide a pharmaceutical composition for treating PPARG-related disease which comprises the compound above, the optical isomer thereof, or the pharmaceutically acceptable salt of the same as an active ingredient.

To achieve the above objectives, the present invention provides a compound represented by formula 1 below, an optical isomer thereof, or a pharmaceutically acceptable salt of the same.

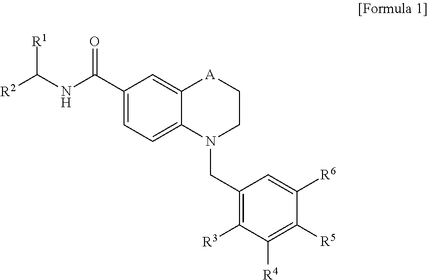

In formula 1,

R¹ is H, unsubstituted or substituted C₁₋₁₀ straight or branched alkyl wherein one or more halogens are substituted, or unsubstituted or substituted C₁₋₁₀ straight or branched alkoxy wherein one or more halogens are substituted;

R² is unsubstituted or substituted C₆₋₁₀ aryl, unsubstituted or substituted C₆₋₁₀ aryl C₁₋₁₀ straight or branched alkyl, or unsubstituted or substituted 5-10 membered heteroaryl containing one or more hetero atoms selected from the group consisting of N, O, and S.

In the said substituted C₆₋₁₀ aryl, the substituted C₆₋₁₀ aryl C₁₋₁₀ straight or branched alkyl, and the substituted 5-10 membered heteroaryl, one or more substituents selected from the group consisting of C₁₋₁₀ straight or branched alkyl unsubstituted or substituted with one or more halogens, C₁₋₁₀ straight or branched alkoxy unsubstituted or substituted with one or more halogens, C₁₋₁₀ straight or branched alkylsulfonyl unsubstituted or substituted with one or more halogens, C₁₋₁₀ straight or branched alkoxycarbonyl, halogen, nitrile, and nitro can be substituted.

In the said unsubstituted or substituted C₆₋₁₀ aryl, phenyl or 5-8 membered heterocycloalkyl containing one or more hetero atoms selected from the group consisting of N, O, and S can be fused.

R¹ and R² can form C₅₋₁₀ cycloalkyl, 5-10 membered heterocycloalkyl containing one or more hetero atoms selected from the group consisting of N, O, and S, or 5-10 membered heteroaryl containing one or more hetero atoms selected from the group consisting of N, O, and S along with the carbon atoms which are conjugated to the same.

In the said C₅₋₁₀ cycloalkyl, the 5-10 membered heterocycloalkyl, or the 5-10 membered heteroaryl, phenyl can be fused.

R³, R⁴, R⁵, and R⁶ are independently H, halogen, unsubstituted or substituted C₁₋₁₀ straight or branched alkyl wherein one or more halogens are substituted, unsubstituted or substituted C₁₋₁₀ straight or branched alkoxy wherein one or more halogens are substituted, or unsubstituted or substituted C₆₋₁₀ aryl.

In the said substituted C₆₋₁₀ aryl, one or more substituents selected from the group consisting of halogen, unsubstituted or substituted C₁₋₁₀ straight or branched alkyl wherein one or more halogens are substituted, hydroxycarbonyl, aminocarbonyl, and sodiumoxycarbonyl can be substituted.

A is —CH₂— or —O—.

The present invention also provides a method for preparing the compound represented by formula 1 comprising the following steps, as shown in reaction formula 1 below:
preparing the compound represented by formula 4 by reacting the compound represented by formula 2 with the compound represented by formula 3 (step 1); preparing the compound represented by formula 5 by substituting methoxycarbonyl group of the compound represented by formula 4 prepared in step 1 with carboxy group (step 2);
preparing the compound represented by formula 7 by reacting the compound represented by formula 5 prepared in step 2 with the compound represented by formula 6 (step 3); and

substituting t-butoxy group of the compound represented by formula 7 prepared in step 3 with —OH group (step 4).

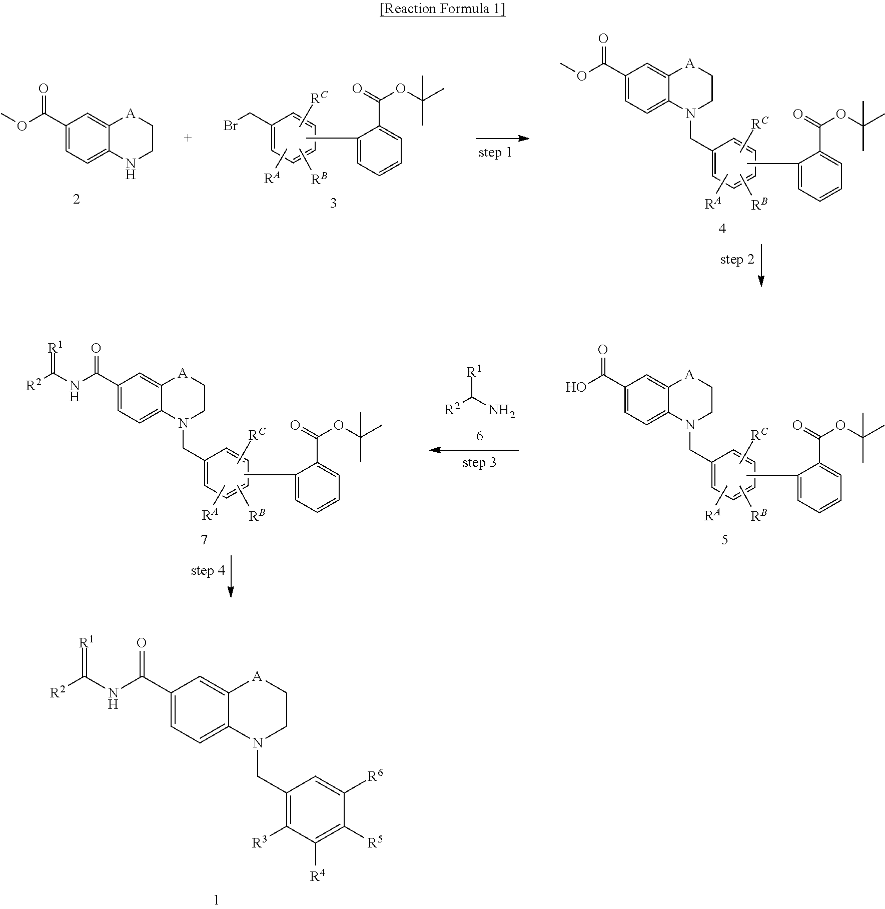

(In the reaction formula 1, R¹-R⁶ and A are as defined in formula 1, and R^{A}, R^{B}, and R^{C} are independently as defined in R³-R⁶).

The present invention also provides a method for preparing the compound represented by formula 1 comprising the following steps, as shown in reaction formula 2 below:

preparing the compound represented by formula 9 by reacting the compound represented by formula 8 with the compound represented by formula 6 (step 1);

preparing the compound represented by formula 7 by reacting the compound represented by formula 9 prepared in step 1 with the compound represented by formula 3 (step 2); and

substituting t-butoxy group of the compound represented by formula 7 prepared in step 2 with —OH group (step 3).

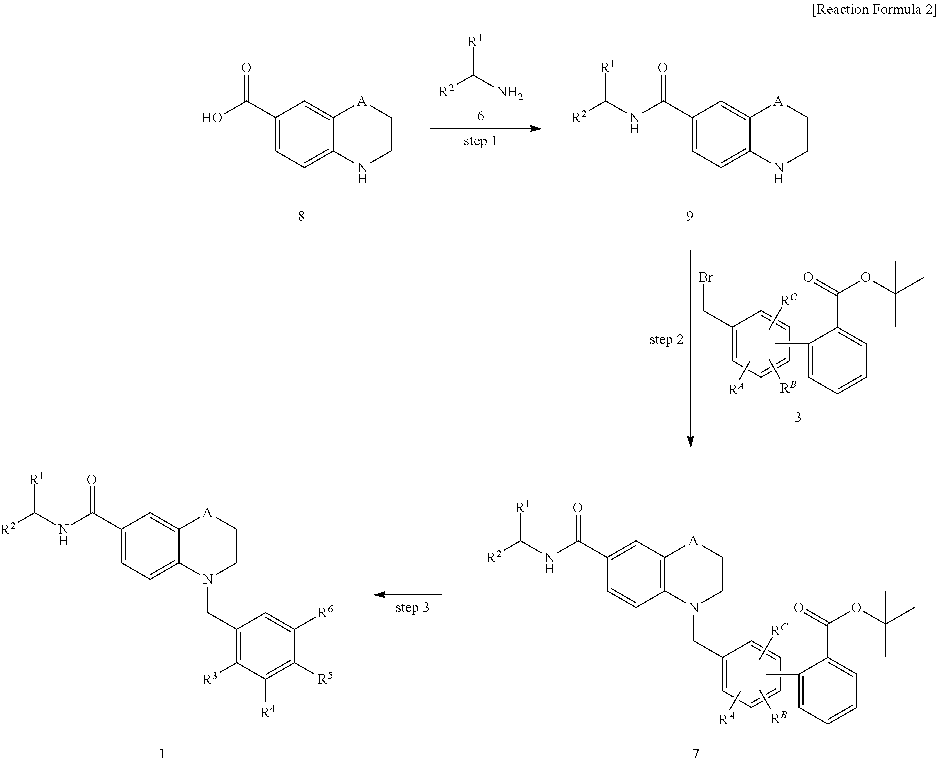

(In the reaction formula 2, R¹-R⁶ and A are as defined in formula 1, and R^{A}, R^{B}, and R^{C} are independently as defined in R³-R⁶).

The present invention further provides a method for preparing the compound represented by formula 1, as shown in reaction formula 3 below, comprising the step of substituting carboxyl group of the compound represented by formula 1A with sodiumoxycarbonyl group or amide group (step 1).

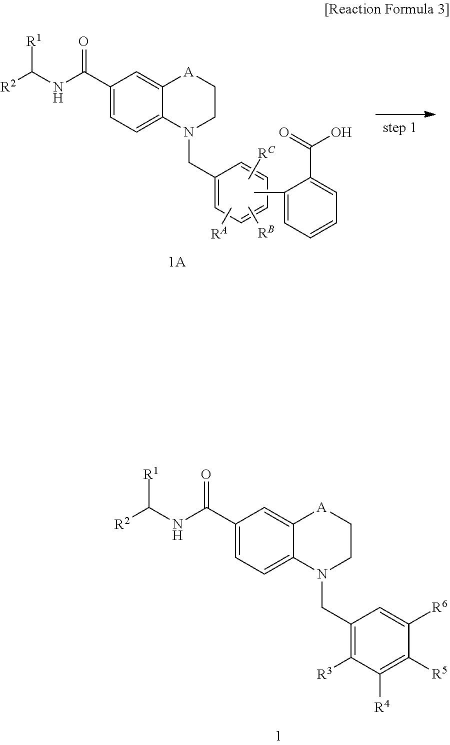

(In the reaction formula 3, R¹-R⁶ and A are as defined in formula 1, and R^{A}, R^{B}, and R^{C} are independently as defined in R³-R⁶).

In addition, the present invention provides a pharmaceutical composition for treating PPARG-related diseases comprising the compound represented by formula 1, the optical isomer thereof, or the pharmaceutically acceptable salt of the same as an active ingredient.

### Advantageous Effect

The compound represented by formula 1 or the optical isomer thereof of the present invention binds to PPARG with a high affinity but does not act as an agonist and thereby does not induce gene transcription of the same and blocks CDK5, which is a cause of PPARG phosphorylation. Therefore, the compound or the optical isomer thereof of the invention does not cause the side effects of the conventional anti-diabetic agents but is easily formulated as a drug with improved pharmacophysical properties and is excellent in treating PPARG-related diseases, indicating that the compound or the optical isomer thereof of the invention can be effectively used as a pharmaceutical composition for PPARG-related diseases.

Thus, the present inventors tried to develop a compound that can be formulated as a drug with improved pharmaco-kinetic pharmacophysical properties. In the course of the study, the present inventors confirmed that a compound with a specific structure that binds to PPARG with high affinity but does not induce transcriptional activity, suggesting that the compound does not act as an agonist but can block CDK5 activity so as to improve insulin resistance, and thereby confirmed that the compound is more excellent in lowering blood sugar and weight than SR1664 and SR1824 and does not significantly inhibit CYP enzyme activity, the drug-drug interaction index. As a result, the inventors confirmed that the compound above can be effectively used for treating PPARG related diseases, leading to the completion of the present invention.

### DETAILED DESCRIPTION

Hereinafter, the present invention is described in detail.

The present invention provides a compound represented by formula 1 below, an optical isomer thereof, or a pharmaceutically acceptable salt of the same.

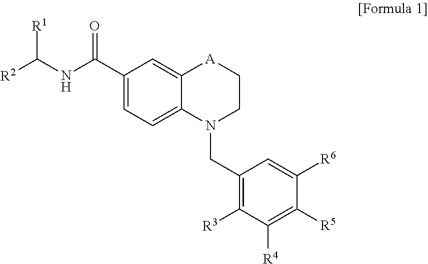

In the formula 1,

R¹ is H, unsubstituted or substituted C₁₋₁₀ straight or branched alkyl wherein one or more halogens are substituted, or unsubstituted or substituted C₁₋₁₀ straight or branched alkoxy wherein one or more halogens are substituted.

R² is unsubstituted or substituted C₆₋₁₀ aryl, unsubstituted or substituted C₆₋₁₀ aryl C₁₋₁₀ straight or branched alkyl, or unsubstituted or substituted 5-10 membered heteroaryl containing one or more hetero atoms selected from the group consisting of N, O, and S.

In the said substituted C₆₋₁₀ aryl, the substituted C₆₋₁₀ aryl C₁₋₁₀ straight or branched alkyl, and the substituted 5-10 membered heteroaryl, one or more substituents selected from the group consisting of C₁₋₁₀ straight or branched alkyl unsubstituted or substituted with one or more halogens, C₁₋₁₀ straight or branched alkoxy unsubstituted or substituted with one or more halogens, C₁₋₁₀ straight or branched alkylsulfonyl unsubstituted or substituted with one or more halogens, C₁₋₁₀ straight or branched alkoxycarbonyl, halogen, nitrile, and nitro can be substituted.

In the said unsubstituted or substituted C₆₋₁₀ aryl, phenyl or 5-8 membered heterocycloalkyl containing one or more hetero atoms selected from the group consisting of N, O, and S can be fused.

R¹ and R² can form C₅₋₁₀ cycloalkyl, 5-10 membered heterocycloalkyl containing one or more hetero atoms selected from the group consisting of N, O, and S, or 5-10 membered heteroaryl containing one or more hetero atoms selected from the group consisting of N, O, and S along with the carbon atoms which are conjugated to the same.

In the said C₅₋₁₀ cycloalkyl, the 5-10 membered heterocycloalkyl, or the 5-10 membered heteroaryl, phenyl can be fused.

R³, R⁴, R⁵, and R⁶ are independently H, halogen, unsubstituted or substituted C₁₋₁₀ straight or branched alkyl wherein one or more halogens are substituted, unsubstituted or substituted C₁₋₁₀ straight or branched alkoxy wherein one or more halogens are substituted, or unsubstituted or substituted C₆₋₁₀ aryl.

In the said substituted C₆₋₁₀ aryl, one or more substituents selected from the group consisting of halogen, unsubstituted or substituted C₁₋₁₀ straight or branched alkyl wherein one or more halogens are substituted, hydroxycarbonyl, aminocarbonyl, and sodiumoxycarbonyl can be substituted.

A is —CH₂— or —O—.

Preferably, R¹ is H, unsubstituted or substituted C₁₋₅ straight or branched alkyl wherein one or more halogens are substituted, or unsubstituted or substituted C₁₋₅ straight or branched alkoxy wherein one or more halogens are substituted.

R² is unsubstituted or substituted C₆₋₈ aryl, unsubstituted or substituted C₆₋₈ aryl C₁₋₅ straight or branched alkyl, or unsubstituted or substituted 5-8 membered heteroaryl containing one or more hetero atoms selected from the group consisting of N, O, and S.

In the said substituted C₆₋₈ aryl, the substituted C₆₋₈ aryl C₁₋₅ straight or branched alkyl, and the substituted 5-8 membered heteroaryl, one or more substituents selected from the group consisting of C₁₋₅ straight or branched alkyl unsubstituted or substituted with one or more halogens, C₁₋₅ straight or branched alkoxy unsubstituted or substituted with one or more halogens, C₁₋₅ straight or branched alkylsulfonyl unsubstituted or substituted with one or more halogens, C₁₋₅ straight or branched alkoxycarbonyl, halogen, nitrile, and nitro can be substituted.

In the said unsubstituted or substituted C₆₋₈ aryl, phenyl or 5-8 membered heterocycloalkyl containing one or more hetero atoms selected from the group consisting of N, O, and S can be fused.

R¹ and R² can form C₅₋₈ cycloalkyl, 5-8 membered heterocycloalkyl containing one or more hetero atoms selected from the group consisting of N, O, and S, or 5-8 membered heteroaryl containing one or more hetero atoms selected from the group consisting of N, O, and S along with the carbon atoms which are conjugated to the same.

In the said C₅₋₈ cycloalkyl, the 5-8 membered heterocycloalkyl, or the 5-8 membered heteroaryl, phenyl can be fused.

R³, R⁴, R⁵, and R⁶ are independently H, halogen, unsubstituted or substituted C₁₋₅ straight or branched alkyl wherein one or more halogens are substituted, unsubstituted or substituted C₁₋₅ straight or branched alkoxy wherein one or more halogens are substituted, or unsubstituted or substituted C₆₋₈ aryl.

In the said substituted C₆₋₈ aryl, one or more substituents selected from the group consisting of halogen, unsubstituted or substituted C₁₋₅ straight or branched alkyl wherein one or more halogens are substituted, hydroxycarbonyl, aminocarbonyl, and sodiumoxycarbonyl can be substituted.

A is —CH₂— or —O—.

More preferably, R¹ is H, methyl, or ethyl.

R² is unsubstituted or substituted phenyl, unsubstituted or substituted benzyl, or unsubstituted or substituted 5-6 membered heteroaryl containing one or more hetero atoms selected from the group consisting of N and O.

In the said substituted phenyl, the substituted benzyl, and the substituted 5-6 membered heteroaryl, one or more substituents selected from the group consisting of C₁₋₅ straight or branched alkyl unsubstituted or substituted with one or more halogens, C₁₋₅ straight or branched alkoxy unsubstituted or substituted with one or more halogens, C₁₋₅ straight or branched alkylsulfonyl unsubstituted or substituted with one or more halogens, C₁₋₅ straight or branched alkoxycarbonyl, halogen, nitrile, and nitro can be substituted.

In the said unsubstituted or substituted phenyl, phenyl or 6 membered heterocycloalkyl containing one or more hetero atoms selected from the group consisting of N and O can be fused.

R¹ and R² can form C₅₋₈ cycloalkyl, 5-8 membered heterocycloalkyl containing one or more hetero atoms selected from the group consisting of N, O, and S, or 5-8 membered heteroaryl containing one or more hetero atoms selected from the group consisting of N, O, and S along with the carbon atoms which are conjugated to the same.

In the said C₅₋₈ cycloalkyl, the 5-8 membered heterocycloalkyl, or the 5-8 membered heteroaryl, phenyl can be fused.

R³, R⁴, R⁵, and R⁶ are independently H, fluoro, or unsubstituted or substituted phenyl.

In the said substituted phenyl, one or more substituents selected from the group consisting of hydroxycarbonyl, aminocarbonyl, and sodiumoxycarbonyl can be substituted.

A is —CH₂— or —O—.

More preferably, R¹ is H, methyl, or ethyl;

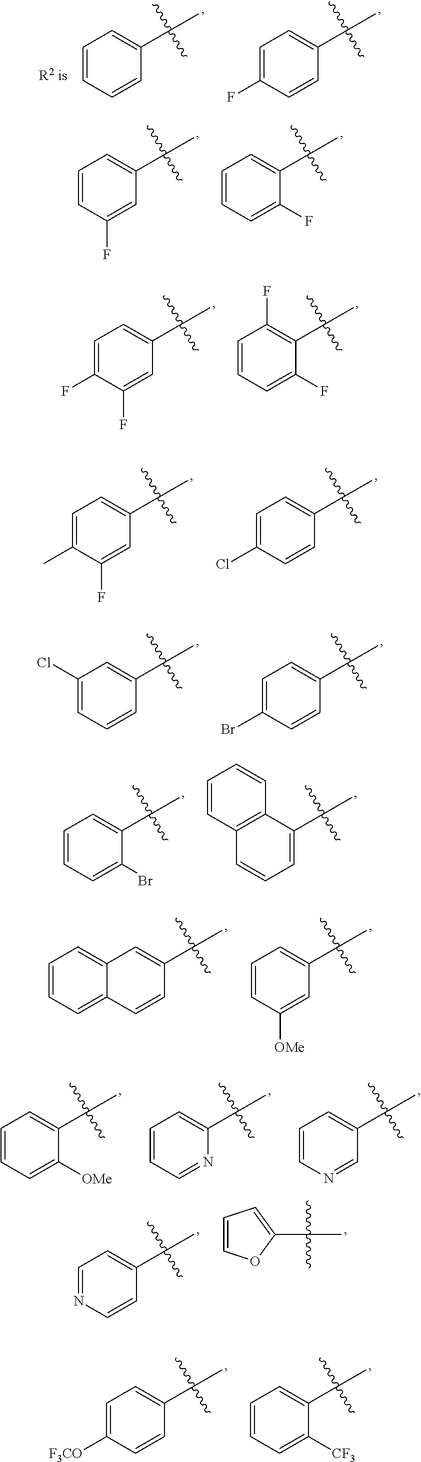

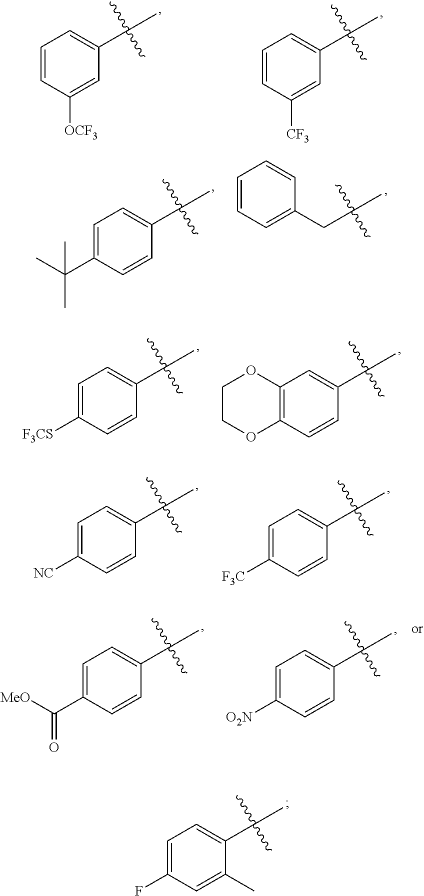

R³ is H, F,

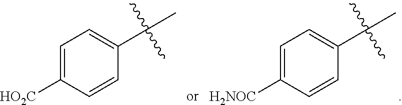 [SMILES: *c1ccc(C(=O)O)cc1 ; *c1ccc(C(N)=O)cc1]

R⁴ is H, F,

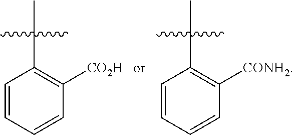 [SMILES: *c1ccccc1C(=O)O ; *c1ccccc1C(N)=O]

R⁵ is H,

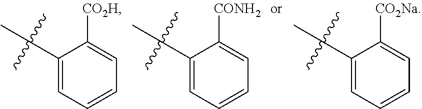

R⁶ is H,

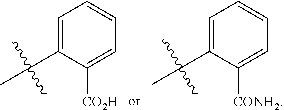 [SMILES: *c1ccccc1C(=O)O ; *c1ccccc1C(N)=O]

A is —CH₂— or —O—.

The compound represented by formula 1 or the optical isomer thereof of the present invention can be exemplified by the following compounds:
(1) (S)-4′-((6-((1-phenylethyl)carbamoyl)-3,4-dihydroquinoline-1(2H)-yl)methyl)-[1,1′-biphenyl]-2-carboxylic acid;
(2) (S)-1-((2′-carbamoyl-[1,1′-biphenyl]-4-yl)methyl)-N-(1-phenylethyl)-1,2,3,4-tetrahydroquinoline-6-carboxamide;
(3) (S)-4′-((6-((1-(4-nitrophenyl)ethyl)carbamoyl)-3,4-dihydroquinoline-1(2H)-yl)methyl)-[1,1′-biphenyl]-2-carboxylic acid;
(4) (S)-4′-((6-((1-(4-fluoro-2-methylphenyl)ethyl)carbamoyl)-3,4-dihydroquinoline-1(2H)-yl)methyl)-[1,1′-biphenyl]-2-carboxylic acid;
(5) (S)-4′-((6-((1-(3-fluoro-4-methylphenyl)ethyl)carbamoyl)-3, 4-dihydroquinoline-1(2H)-yl)methyl)-[1,1′-biphenyl]-2-carboxylic acid;
(6) (S)-4′-((6-((1-(4-fluorophenyl)ethyl)carbamoyl)-3, 4-dihydroquinoline-1(2H)-yl)methyl)-[1,1′-biphenyl]-2-carboxylic acid;
(7) (S)-4′-((6-((1-(4-bromophenyl)ethyl)carbamoyl)-3, 4-dihydroquinoline-1(2H)-yl)methyl)-[1,1′-biphenyl]-2-carboxylic acid;
(8) 4′-((6-((4-nitrobenzyl)carbamoyl)-3,4-dihydroquinoline-1(2H)-yl)methyl)-[1,1′-biphenyl]-2-carboxylic acid;
(9) (S)-4′-((6-((1-(3-chlorophenyl)ethyl)carbamoyl)-3, 4-dihydroquinoline-1(2H)-yl)methyl)-[1,1′-biphenyl]-2-carboxylic acid;
(10) (S)-4′-((6-((1-(naphthalene-1-yl)ethyl)carbamoyl)-3,4-dihydroquinoline-1(2H)-yl)methyl)-[1,1′-biphenyl]-2-carboxylic acid;
(11) (S)-4′-((6-((1-(4-(trifluoromethoxy)phenyl)ethyl)carbamoyl)-3,4-dihydroquinoline-1(2H)-yl)methyl)-[1,1′-biphenyl]-2-carboxylic acid;
(12) (S)-4′-((6-((1-(4-(trifluoromethyl)phenyl)ethyl)carbamoyl)-3,4-dihydroquinoline-1(2H)-yl)methyl)-[1,1′-biphenyl]-2-carboxylic acid;
(13) (S)-4′-((6-((1-(4-((trifluoromethyl)thio)phenyl)ethyl)carbamoyl)-3,4-dihydroquinoline-1 (2H)-yl)methyl)-[1,1′-biphenyl]-2-carboxylic acid;
(14) (S)-4′-((6-((1-(4-cyanophenyl)ethyl)carbamoyl)-3,4-dihydroquinoline-1(2H)-yl)methyl)-[1,1′-biphenyl]-2-carboxylic acid;
(15) 4′-((6-(cromene-3-ylcarbamoyl)-3,4-dihydroquinoline-1(2H)-yl)methyl)-[1,1′-biphenyl]-2-carboxylic acid;
(16) (S)-4′-((6-((1-(2,6- [symbol] fluorophenyl)ethyl)carbamoyl)-3, 4-dihydroquinoline-1(2H)-yl)methyl)-[1,1′-biphenyl]-2-carboxylic acid;
(17) (S)-4′-((6-((1-(3-fluorophenyl)ethyl)carbamoyl)-3,4-dihydroquinoline-1(2H)-yl)methyl)-[1,1′-biphenyl]-2-carboxylic acid;
(18) (S)-4′-((6-((1-(3-(trifluoromethoxy)phenyl)ethyl)carbamoyl)-3,4-dihydroquinoline-1(2H)-yl)methyl)-[1,1′-biphenyl]-2-carboxylic acid;
(19) (S)-4′-((6-((1-(3-(trifluoromethyl)phenyl)ethyl)carbamoyl)-3,4-dihydroquinoline-1(2H)-yl)methyl)-[1,1′-biphenyl]-2-carboxylic acid;
(20) (S)-4′-((6-((1-(naphthalene-2-yl)ethyl)carbamoyl)-3,4-dihydroquinoline-1(2H)-yl)methyl)-[1,1′-biphenyl]-2-carboxylic acid;
(21) (S)-4′-((6-((1-(3,4- [symbol] fluorophenyl)ethyl)carbamoyl)-3,4-dihydroquinoline-1(2H)-yl)methyl)-[1,1′-biphenyl]-2-carboxylic acid;
(22) (S)-4′-((6-((1-(3-methoxyphenyl)ethyl)carbamoyl)-3,4-dihydroquinoline-1(2H)-yl)methyl)-[1,1′-biphenyl]-2-carboxylic acid;
(23) (S)-4′-((6-((1-(2-methoxyphenyl)ethyl)carbamoyl)-3,4-dihydroquinoline-1(2H)-yl)methyl)-[1,1′-biphenyl]-2-carboxylic acid;
(24) (S)-4′-((6-((1-(2-fluorophenyl)ethyl)carbamoyl)-3, 4-dihydroquinoline-1(2H)-yl)methyl)-[1,1′-biphenyl]-2-carboxylic acid;
(25) (S)-4′-((6-((1-(2-(trifluoromethyl)phenyl)ethyl)carbamoyl)-3,4-dihydroquinoline-1(2H)-yl)methyl)-[1,1′-biphenyl]-2-carboxylic acid;
(26) 4′-((6-((pyridine-2-ylmethyl)carbamoyl)-3,4-dihydroquinoline-1(2H)-yl)methyl)-[1,1′-biphenyl]-2-carboxylic acid;
(27) 4′-((6-((pyridine-4-ylmethyl)carbamoyl)-3,4-dihydroquinoline-1(2H)-yl)methyl)-[1,1′-biphenyl]-2-carboxylic acid;
(28) 4′-((6-(benzylcarbamoyl)-3,4-dihydroquinoline-1(2H)-yl)methyl)-[1,1′-biphenyl]-2-carboxylic acid;
(29) 4′-((6-((2-bromobenzyl)carbamoyl)-3,4-dihydroquinoline-1(2H)-yl)methyl)-[1,1′-biphenyl]-2-carboxylic acid;
(30) 4′-((6-(((2,3-dihydrobenzo[b][1,4]dioxin-6-yl)methyl)carbamoyl)-3,4-dihydroquinoline-1(2H)-yl)methyl)-[1,1′-biphenyl]-2-carboxylic acid;
(31) 4′-((6-((4-bromobenzyl)carbamoyl)-3,4-dihydroquinoline-1(2H)-yl)methyl)-[1,1′-biphenyl]-2-carboxylic acid;
(32) 4′-((6-((3-(trifluoromethoxy)benzyl)carbamoyl)-3,4-dihydroquinoline-1(2H)-yl)methyl)-[1,1′-biphenyl]-2-carboxylic acid;
(33) (S)-1-((2′-carbamoyl-[1,1′-biphenyl]-4-yl)methyl)-N-(1-(4-(trifluoromethyl)phenyl)ethyl)-1,2,3,4-tetrahydroquinoline-6-carboxamide
(34) (S)-1-((2′-carbamoyl-[1,1′-biphenyl]-4-yl)methyl)-N-(1-(4-(trifluoromethoxy)phenyl)ethyl)-1,2,3,4-tetrahydroquinoline-6-carboxamide;
(35) (S)-1-((2′-carbamoyl-[1,1′-biphenyl]-4-yl)methyl)-N-(1-(3-chlorophenyl)ethyl)-1,2,3,4-tetrahydroquinoline-6-carboxamide;
(36) (S)-1-((2′-carbamoyl-[1,1′-biphenyl]-4-yl)methyl)-N-(1-(4-fluoro phenyl)ethyl)-1,2,3,4-tetrahydroquinoline-6-carboxamide;
(37) (S)—N-(1-(4-bromophenyl)ethyl)-1-((2′-carbamoyl-[1,1′-biphenyl]-4-yl)methyl)-1,2,3,4-tetrahydroquinoline-6-carboxamide;
(38) (S)-1-((2′-carbamoyl-[1,1′-biphenyl]-4-yl)methyl)-N-(1-(naphthalene-1-yl)ethyl)-1,2,3,4-tetrahydroquinoline-6-carboxamide;
(39) (S)-1-((2′-carbamoyl-[1,1′-biphenyl]-4-yl)methyl)-N-(1-(3,4-difluorophenyl)ethyl)-1,2,3,4-tetrahydroquinoline-6-carboxamide;
(40) (S)-1-((2′-carbamoyl-[1,1′-biphenyl]-4-yl)methyl)-N-(1-(3-methoxyphenyl)ethyl)-1,2,3,4-tetrahydroquinoline-6-carboxamide;
(41) (S)-1-((2′-carbamoyl-[1,1′-biphenyl]-4-yl)methyl)-N-(1-(2-methoxyphenyl)ethyl)-1,2,3,4-tetrahydroquinoline-6-carboxamide;
(42) (S)-1-((2′-carbamoyl-[1,1′-biphenyl]-4-yl)methyl)-N-(1-(2-fluorophenyl)ethyl)-1,2,3,4-tetrahydroquinoline-6-carboxamide;
(43) (S)-1-((2′-carbamoyl-[1,1′-biphenyl]-4-yl)methyl)-N-(1-(2-(trifluoromethyl)phenyl)ethyl)-1,2,3,4-tetrahydroquinoline-6-carboxamide;
(44) (S)-1-((2′-carbamoyl-[1,1′-biphenyl]-4-yl)methyl)-N-(1-(naphthalene-2-yl)ethyl)-1,2,3,4-tetrahydroquinoline-6-carboxamide;
(45) (S)-1-((2′-carbamoyl-[1,1′-biphenyl]-4-yl)methyl)-N-(1-(3-fluorophenyl)ethyl)-1,2,3,4-tetrahydroquinoline-6-carboxamide;
(46) (S)-1-((2′-carbamoyl-[1,1′-biphenyl]-4-yl)methyl)-N-(1-(3-(trifluoromethoxy)phenyl)ethyl)-1,2,3,4-tetrahydroquinoline-6-carboxamide;
(47) (S)-1-((2′-carbamoyl-[1,1′-biphenyl]-4-yl)methyl)-N-(1-(3-(trifluoromethyl)phenyl)ethyl)-1,2,3,4-tetrahydroquinoline-6-carboxamide;
(48) 1-((2′-carbamoyl-[1,1′-biphenyl]-4-yl)methyl)-N-(pyridine-4-ylmethyl)-1,2,3,4-tetrahydroquinoline-6-carboxamide;
(49) (S)-1-((2′-carbamoyl-[1,1′-biphenyl]-4-yl)methyl)-N-(1-(3-fluoro-4-methylphenyl)ethyl)-1,2,3,4-tetrahydroquinoline-6-carboxamide;
(50) N-benzyl-1-((2′-carbamoyl-[1,1′-biphenyl]-4-yl)methyl)-1,2,3,4-tetrahydroquinoline-6-carboxamide;
(51) N-(2-bromobenzyl)-1-((2′-carbamoyl-[1,1′-biphenyl]-4-yl)methyl)-1,2,3,4-tetrahydroquinoline-6-carboxamide;
(52) 1-((2′-carbamoyl-[1,1′-biphenyl]-4-yl)methyl)-N-((2,3-dihydrobenzo[b][1,4]dioxin-6-yl)methyl)-1,2,3,4-tetrahydroquinoline-6-carboxamide;
(53) N-(4-bromobenzyl)-1-((2′-carbamoyl-[1,1′-biphenyl]-4-yl)methyl)-1,2,3,4-tetrahydroquinoline-6-carboxamide;
(54) 1-((2′-carbamoyl-[1,1′-biphenyl]-4-yl)methyl)-N-(3-(trifluoromethoxy)benzyl)-1,2,3,4-tetrahydroquinoline-6-carboxamide;
(55) sodium (S)-4′-((6-((1-phenylethyl)carbamoyl)-3,4-dihydroquinoline-1(2H)-yl)methyl)-[1,1′-biphenyl]-2-carboxylate;
(56) sodium (S)-4′-((6-((1-(3-fluoro-4-methylphenyl)ethyl)carbamoyl)-3,4-dihydroquinoline-1(2H)-yl)methyl)-[1,1′-biphenyl]-2-carboxylate;
(57) sodium (S)-4′-((6-((1-(4-cyanophenyl)ethyl)carbamoyl)-3, 4-dihydroquinoline-1(2H)-yl)methyl)-[1,1′-biphenyl]-2-carboxylate;
(58) (S)-4′-((7-((1-phenylethyl)carbamoyl)-2H-benzo[b][1,4]oxazine-4(3H)-yl)methyl)-[1,1′-biphenyl]2-carboxylic acid;
(59) (S)-4-((2′-carbamoyl-[1,1′-biphenyl]-4-yl)methyl)-N-(1-phenylethyl)-3,4-dihydro-2H-benzo[b][1,4]oxazine-7-carboxamide;
(60) (S)-4′-((7-((1-(4-nitrophenyl)ethyl)carbamoyl)-2H-benzo[b][1,4]oxazine-4(3H)-yl)methyl)-[1,1′-biphenyl]-2-carboxylic acid;
(61) (S)-4′-((7-((1-(4-bromophenyl)ethyl)carbamoyl)-2H-benzo[b][1,4]oxazine-4(3H)-yl)methyl)-[1,1′-biphenyl]-2-carboxylic acid;
(62) (S)-4′-((7-((1-phenylpropyl)carbamoyl)-2H-benzo[b][1,4]oxazine-4(3H)-yl)methyl)-[1,1′-biphenyl]-2-carboxylic acid;
(63) (S)-4′-((7-((1-(4-fluorophenyl)ethyl)carbamoyl)-2H-benzo[b][1,4]oxazine-4(3H)-yl)methyl)-[1,1′-biphenyl]-2-carboxylic acid;
(64) (S)-4′-((7-((1-(4-chlorophenyl)ethyl)carbamoyl)-2H-benzo[b][1,4]oxazine-4(3H)-yl)methyl)-[1,1′-biphenyl]-2-carboxylic acid;
(65) (S)-4′-((7-((1-(3-chlorophenyl)ethyl)carbamoyl)-2H-benzo[b][1,4]oxazine-4(3H)-yl)methyl)-[1,1′-biphenyl]-2-carboxylic acid;
(66) 4′-((7-((4-nitrobenzyl)carbamoyl)-2H-benzo[b][1,4]oxazine-4(3H)-yl)methyl)-[1,1′-biphenyl]-2-carboxylic acid;
(67) 4′-((7-(benzylcarbamoyl)-2H-benzo[b][1,4]oxazine-4(3H)-yl)methyl)-[1,1′-biphenyl]-2-carboxylic acid;
(68) 4′-((7-(phenethylcarbamoyl)-2H-benzo[b][1,4]oxazine-4(3H)-yl)methyl)-[1,1′-biphenyl]-2-carboxylic acid;
(69) 4′-((7-((furan-2-ylmethyl)carbamoyl)-2H-benzo[b][1,4]oxazine-4(3H)-yl)methyl)-[1,1′-biphenyl]-2-carboxylic acid;
(70) (S)-4′-((7-((1-(naphthalene-1-yl)ethyl)carbamoyl)-2H-benzo[b][1,4]oxazine-4(3H)-yl)methyl)-[1,1′-biphenyl]-2-carboxylic acid;
(71) 4′-((7-((4-(methoxycarbonyl)benzyl)carbamoyl)-2H-benzo[b][1,4]oxazine-4(3H)-yl)methyl)-[1,1′-biphenyl]-2-carboxylic acid;
(72) 4′-((7-((3-methoxybenzyl)carbamoyl)-2H-benzo[b][1,4]oxazine-4(3H)-yl)methyl)-[1,1′-biphenyl]-2-carboxylic acid;
(73) 4′-((7-((4-bromobenzyl)carbamoyl)-2H-benzo[b][1, 4]oxazine-4(3H)-yl)methyl)-[1,1′-biphenyl]-2-carboxylic acid;
(74) (S)-4′-((7-((1-(4-cyanophenyl)ethyl)carbamoyl)-2H-benzo[b][1,4]oxazine-4(3H)-yl)methyl)-[1,1′-biphenyl]-2-carboxylic acid;
(75) (S)-4′-((7-((1-(4-(trifluoromethoxy)phenyl)ethyl)carbamoyl)-2H-benzo[b][1,4]oxazine-4(3H)-yl)methyl)-[1,1′-biphenyl]-2-carboxylic acid;
(76) (S)-4′-((7-((1-(4-(trifluoromethyl)phenyl)ethyl)carbamoyl)-2H-benzo[b][1,4]oxazine-4(3H)-yl)methyl)-[1,1′-biphenyl]-2-carboxylic acid;
(77) (S)-4′-((7-((1-(4-((trifluoromethyl)thio)phenyl)ethyl)carbamoyl)-2H-benzo[b][1,4]oxazine-4(3H)-yl)methyl)-[1,1′-biphenyl]-2-carboxylic acid;
(78) (S)-4′-((7-((1-(4-(tert-butyl)phenyl)ethyl)carbamoyl)-2H-benzo[b][1,4]oxazine-4(3H)-yl)methyl)-[1,1′-biphenyl]-2-carboxylic acid;
(79) (S)-4′-((7-((1-(4-fluoro-2-methylphenyl)ethyl)carbamoyl)-2H-benzo[b][1,4]oxazine-4(3H)-yl)methyl)-[1,1′-biphenyl]-2-carboxylic acid;
(80) (S)-4′-((7-((1-(3-fluoro-4-methylphenyl)ethyl)carbamoyl)-2H-benzo[b][1,4]oxazine-4(3H)-yl)methyl)-[1,1′-biphenyl]-2-carboxylic acid;
(81) (R)-4′-((7-((1-(4-cyanophenyl)ethyl)carbamoyl)-2H-benzo[b][1,4]oxazine-4(3H)-yl)methyl)-[1,1′-biphenyl]-2-carboxylic acid;
(82) 4′-((7-(cromene-3-ylcarbamoyl)-2H-benzo[b][1,4]oxazine-4(3H)-yl)methyl)-[1,1′-biphenyl]-2-carboxylic acid;
(83) (S)-4′-((7-((1-(2,6- [symbol] fluorophenyl)ethyl)carbamoyl)-2H-benzo[b][1,4]oxazine-4(3H)-yl)methyl)-[1,1′-biphenyl]-2-carboxylic acid;
(84) (S)-4′-((7-((1-(3-fluorophenyl)ethyl)carbamoyl)-2H-benzo[b][1,4]oxazine-4(3H)-yl)methyl)-[1,1′-biphenyl]-2-carboxylic acid;
(85) (S)-4′-((7-((1-(3-(trifluoromethoxy)phenyl)ethyl)carbamoyl)-2H-benzo[b][1,4]oxazine-4(3H)-yl)methyl)-[1,1′-biphenyl]-2-carboxylic acid;
(86) (S)-4′-((7-((1-(3-(trifluoromethyl)phenyl)ethyl)carbamoyl)-2H-benzo[b][1,4]oxazine-4(3H)-yl)methyl)-[1,1′-biphenyl]-2-carboxylic acid;
(87) (S)-4′-((7-((1-(naphthalene-2-yl)ethyl)carbamoyl)-2H-benzo[b][1,4]oxazine-4(3H)-yl)methyl)-[1,1′-biphenyl]-2-carboxylic acid;
(88) 4′-((7-((pyridine-2-ylmethyl)carbamoyl)-2H-benzo[b][1,4]oxazine-4(3H)-yl)methyl)-[1,1′-biphenyl]-2-carboxylic acid;
(89) 4′-((7-((pyridine-3-ylmethyl)carbamoyl)-2H-benzo[b][1,4]oxazine-4(3H)-yl)methyl)-[1,1′-biphenyl]-2-carboxylic acid;
(90) 4′-((7-((pyridine-4-ylmethyl)carbamoyl)-2H-benzo[b][1,4]oxazine-4(3H)-yl)methyl)-[1,1′-biphenyl]-2-carboxylic acid;
(91) 4′-((7-(benzylcarbamoyl)-2H-benzo[b][1,4]oxazine-4(3H)-yl)methyl)-[1,1′-biphenyl]-2-carboxylic acid;
(92) 4′-((7-((2-bromobenzyl)carbamoyl)-2H-benzo[b][1,4]oxazine-4(3H)-yl)methyl)-[1,1′-biphenyl]-2-carboxylic acid;
(93) 4′-((7-(((2,3-dihydrobenzo[b][1,4]dioxin-6-yl)methyl)carbamoyl)-2H-benzo[b][1,4]oxazine-4(3H)-yl)methyl)-[1,1′-biphenyl]-2-carboxylic acid;
(94) (R)-4-((2′-carbamoyl-[1,1′-biphenyl]-4-yl)methyl)-N-(1-(4-cyanophenyl)ethyl)-3,4-dihydro-2H-benzo[b][1,4]oxazine-7-carboxamide;
(95) (S)-4-((2′-carbamoyl-[1,1′-biphenyl]-4-yl)methyl)-N-(1-(3-fluorophenyl)ethyl)-3,4-dihydro-2H-benzo[b][1,4]oxazine-7-carboxamide;
(96) (S)-4-((2′-carbamoyl-[1,1′-biphenyl]-4-yl)methyl)-N-(1-(naphthalene-2-yl)ethyl)-3,4-dihydro-2H-benzo[b][1,4]oxazine-7-carboxamide;
(97) 4-((2′-carbamoyl-[1,1′-biphenyl]-4-yl)methyl)-N-(pyridine-3-ylmethyl)-3,4-dihydro-2H-benzo[b][1,4]oxazine-7-carboxamide;
(98) 4-((2′-carbamoyl-[1,1′-biphenyl]-4-yl)methyl)-N-(pyridine-4-ylmethyl)-3,4-dihydro-2H-benzo[b][1,4]oxazine-7-carboxamide;
(99) (S)-4-((2′-carbamoyl-[1,1′-biphenyl]-4-yl)methyl)-N-(1-(4-((trifluoromethyl)thio)phenyl)ethyl)-3,4-dihydro-2H-benzo[b][1,4]oxazine-7-carboxamide;
(100) N-benzyl-4-((2′-carbamoyl-[1,1′-biphenyl]-4-yl)methyl)-3,4-dihydro-2H-benzo[b][1,4]oxazine-7-carboxamide;
(101) N-(2-bromobenzyl)-4-((2′-carbamoyl-[1,1′-biphenyl]-4-yl)methyl)-3, 4-dihydro-2H-benzo[b][1,4]oxazine-7-carboxamide;
(102) 4-((2′-carbamoyl-[1,1′-biphenyl]-4-yl)methyl)-N-((2,3-dihydrobenzo[b][1,4]dioxin-6-yl)methyl)-3,4-dihydro-2H-benzo[b][1,4]oxazine-7-carboxamide;
(103) sodium (S)-4′-((7-((1-phenylethyl)carbamoyl)-2H-benzo[b][1,4]oxazine-4(3H)-yl)methyl)-[1,1′-biphenyl]-2-carboxylate;
(104) sodium (S)-4′-((7-((1-(4-cyanophenyl)ethyl)carbamoyl)-2H-benzo[b][1,4]oxazine-4(3H)-yl)methyl)-[1,1′-biphenyl]-2-carboxylate;
(105) sodium (S)-4′-((7-((1-(4-nitrophenyl)ethyl)carbamoyl)-2H-benzo[b][1,4]oxazine-4(3H)-yl)methyl)-[1,1′-biphenyl]-2-carboxylate;
(106) (S)-1-((2′-carbamoyl-3-fluoro-[1,1′-biphenyl]-4-yl)methyl)-N-(1-phenylethyl)-1,2,3,4-tetrahydroquinoline-6-carboxylic acid;
(107) (S)-1-((2′-carbamoyl-3-fluoro-[1,1′-biphenyl]-4-yl)methyl)-N-(1-phenylethyl)-1,2,3,4-tetrahydroquinoline-6-carboxamide;
(108) (S)-3′-fluoro-4′-((6-((1-(naphthalene-2-yl)ethyl)carbamoyl)-3,4-dihydro quinoline-1(2H)-yl)methyl)-[1,1′-biphenyl]-2-carboxylic acid;
(109) (S)-3′-fluoro-4′-((6-((1-(4-nitrophenyl)ethyl)carbamoyl)-3,4-dihydroquinoline-1(2H)-yl)methyl)-[1,1′-biphenyl]-2-carboxylic acid;
(110) (S)-4′-((6-((1-(4-cyanophenyl)ethyl)carbamoyl)-3, 4-dihydroquinoline-1(2H)-yl)methyl)-3′-fluoro-[1,1′-biphenyl]-2-carboxylic acid;
(111) (S)-3′-fluoro-4′-((6-((1-(3-fluoro-4-methylphenyl)ethyl)carbamoyl)-3,4-dihydroquinoline-1(2H)-yl)methyl)-[1,1′-biphenyl]-2-carboxylic acid;
(112) (S)-1-((2′-carbamoyl-3-fluoro-[1,1′-biphenyl]-4-yl)methyl)-N-(1-(4-cyanophenyl)ethyl)-1,2,3,4-tetrahydroquinoline-6-carboxamide;
(113) (S)-1-((2′-carbamoyl-3-fluoro-[1,1′-biphenyl]-4-yl)methyl)-N-(1-(4-nitrophenyl)ethyl)-1,2,3,4-tetrahydroquinoline-6-carboxamide;
(114) (S)-1-((2′-carbamoyl-3-fluoro-[1,1′-biphenyl]-4-yl)methyl)-N-(1-(3-fluoro-4-methylphenyl)ethyl)-1,2,3,4-tetrahydroquinoline-6-carboxamide;
(115) (S)-1-((2′-carbamoyl-3-fluoro-[1,1′-biphenyl]-4-yl)methyl)-N-(1-(naphthalene-2-yl)ethyl)-1,2,3,4-tetrahydroquinoline-6-carboxamide;
(116) (S)-3′-((6-((1-phenylethyl)carbamoyl)-3,4-dihydroquinoline-1(2H)-yl)methyl)-[1,1′-biphenyl]-2-carboxylic acid;
(117) (S)-1-((2′-carbamoyl-[1,1′-biphenyl]-3-yl)methyl)-N-(1-phenylethyl)-1,2,3,4-tetrahydroquinoline-6-carboxamide;
(118) (S)-3′-((6-((1-(4-cyanophenyl)ethyl)carbamoyl)-3,4-dihydroquinoline-1(2H)-yl)methyl)-[1,1′-biphenyl]-2-carboxylic acid;
(119) (S)-3′-((6-((1-(4-nitrophenyl)ethyl)carbamoyl)-3,4-dihydroquinoline-1(2H)-yl)methyl)-[1,1′-biphenyl]-2-carboxylic acid;
(120) (S)-3′-((6-((1-(naphthalene-2-yl)ethyl)carbamoyl)-3,4-dihydroquinoline-1(2H)-yl)methyl)-[1,1′-biphenyl]-2-carboxylic acid;
(121) (S)-3′-((6-((1-(3-fluoro-4-methylphenyl)ethyl)carbamoyl)-3,4-dihydroquinoline-1(2H)-yl)methyl)-[1,1′-biphenyl]-2-carboxylic acid;
(122) (S)-1-((2′-carbamoyl-[1,1′-biphenyl]-3-yl)methyl)-N-(1-(4-cyanophenyl)ethyl)-1,2,3,4-tetrahydroquinoline-6-carboxamide;
(123) (S)-1-((2′-carbamoyl-[1,1′-biphenyl]-3-yl)methyl)-N-(1-(4-nitrophenyl)ethyl)-1,2,3,4-tetrahydroquinoline-6-carboxamide;
(124) (S)-1-((2′-carbamoyl-[1,1′-biphenyl]-3-yl)methyl)-N-(1-(naphthalene-2-yl)ethyl)-1,2,3,4-tetrahydroquinoline-6-carboxamide;
(125) (S)-1-((2′-carbamoyl-[1,1′-biphenyl]-3-yl)methyl)-N-(1-(3-fluoro-4-methylphenyl)ethyl)-1,2,3,4-tetrahydroquinoline-6-carboxamide;
(126) (S)-2′-fluoro-3′-((6-((1-phenylethyl)carbamoyl)-3,4-dihydroquinoline-1(2H)-yl)methyl)-[1,1′-biphenyl]-2-carboxylic acid;
(127) (S)-1-((2′-carbamoyl-2-fluoro-[1,1′-biphenyl]-3-yl)methyl)-N-(1-phenylethyl)-1,2,3,4-tetrahydroquinoline-6-carboxamide compound;
(128) (S)-2′-fluoro-3′-((6-((1-(4-nitrophenyl)ethyl)carbamoyl)-3,4-dihydroquinoline-1(2H)-yl)methyl)-[1,1′-biphenyl]-2-carboxylic acid;
(129) (S)-2′-fluoro-3′-((6-((1-(naphthalene-2-yl)ethyl)carbamoyl)-3,4-dihydroquinoline-1(2H)-yl)methyl)-[1,1′-biphenyl]-2-carboxylic acid;
(130) (S)-2′-fluoro-3′-((6-((1-(3-fluoro-4-methylphenyl)ethyl)carbamoyl)-3,4-dihydroquinoline-1(2H)-yl)methyl)-[1,1′-biphenyl]-2-carboxylic acid;
(131) (S)-1-((2′-carbamoyl-2-fluoro-[1,1′-biphenyl]-3-yl)methyl)-N-(1-(4-nitrophenyl)ethyl)-1,2,3,4-tetrahydroquinoline-6-carboxamide;
(132) (S)-1-((2′-carbamoyl-2-fluoro-[1,1′-biphenyl]-3-yl)methyl)-N-(1-(naphthalene-2-yl)ethyl)-1,2,3,4-tetrahydroquinoline-6-carboxamide;
(133) (S)-1-((2′-carbamoyl-2-fluoro-[1,1′-biphenyl]-3-yl)methyl)-N-(1-(3-fluoro-4-methylphenyl)ethyl)-1,2,3,4-tetrahydroquinoline-6-carboxamide;
(134) (S)-4′-fluoro-3′-((6-((1-phenylethyl)carbamoyl)-3,4-dihydroquinoline-1(2H)-yl)methyl)-[1,1′-biphenyl]-2-carboxylic acid;
(135) (S)-1-((2′-carbamoyl-4-fluoro-[1,1′-biphenyl]-3-yl)methyl)-N-(1-phenylethyl)-1,2,3,4-tetrahydroquinoline-6-carboxamide;
(136) (S)-4′-fluoro-3′-((6-((1-(4-nitrophenyl)ethyl)carbamoyl)-3,4-dihydroquinoline-1(2H)-yl)methyl)-[1,1′-biphenyl]-2-carboxylic acid;
(137) (S)-4′-fluoro-3′-((6-((1-(naphthalene-2-yl)ethyl)carbamoyl)-3,4-dihydroquinoline-1(2H)-yl)methyl)-[1,1′-biphenyl]-2-carboxylic acid;
(138) (S)-4′-fluoro-3′-((6-((1-(3-fluoro-4-methylphenyl)ethyl)carbamoyl)-3,4-dihydroquinoline-1(2H)-yl)methyl)-[1,1′-biphenyl]-2-carboxylic acid;
(139) (S)-1-((2′-carbamoyl-4-fluoro-[1,1′-biphenyl]-3-yl)methyl)-N-(1-(4-nitrophenyl)ethyl)-1,2,3,4-tetrahydroquinoline-6-carboxamide;
(140) (S)-1-((2′-carbamoyl-4-fluoro-[1,1′-biphenyl]-3-yl)methyl)-N-(1-(naphthalene-2-yl)ethyl)-1,2,3,4-tetrahydroquinoline-6-carboxamide;
(141) (S)-1-((2′-carbamoyl-4-fluoro-[1,1′-biphenyl]-3-yl)methyl)-N-(1-(3-fluoro-4-methylphenyl)ethyl)-1,2,3,4-tetrahydroquinoline-6-carboxamide;
(142) (S)-2′-((7-((1-phenylethyl)carbamoyl)-2H-benzo[b][1,4]oxazine-4(3H)-yl)methyl)-[1,1′-biphenyl]-4-carboxylic acid;
(143) (S)-4-((4′-carbamoyl-[1,1′-biphenyl]-2-yl)methyl)-N-(1-phenylethyl)-3,4-dihydro-2H-benzo[b][1,4]oxazine-7-carboxamide;
(144) (S)-2′-((7-((1-(4-cyanophenyl)ethyl)carbamoyl)-2H-benzo[b][1,4]oxazine-4(3H)-yl)methyl)-[1,1′-biphenyl]-4-carboxylic acid;
(145) (S)-2′-((7-((1-(3-fluoro-4-methylphenyl)ethyl)carbamoyl)-2H-benzo[b][1,4]oxazine-4(3H)-yl)methyl)-[1,1′-biphenyl]-4-carboxylic acid;
(146) (S)-2′-((7-((1-(4-nitrophenyl)ethyl)carbamoyl)-2H-benzo[b][1,4]oxazine-4(3H)-yl)methyl)-[1,1′-biphenyl]-4-carboxylic acid;
(147) (S)-2′-((7-((1-(naphthalene-2-yl)ethyl)carbamoyl)-2H-benzo[b][1,4]oxazine-4(3H)-yl)methyl)-[1,1′-biphenyl]-4-carboxylic acid;
(148) (S)-4-((4′-carbamoyl-[1,1′-biphenyl]-2-yl)methyl)-N-(1-(3-fluoro-4-methylphenyl)ethyl)-3,4-dihydro-2H-benzo[b][1,4]oxazine-7-carboxamide;
(149) (S)-4-((4′-carbamoyl-[1,1′-biphenyl]-2-yl)methyl)-N-(1-(4-cyanophenyl)ethyl)-3,4-dihydro-2H-benzo[b][1,4]oxazine-7-carboxamide;
(150) (S)-4-((4′-carbamoyl-[1,1′-biphenyl]-2-yl)methyl)-N-(1-(4-nitrophenyl)ethyl)-3,4-dihydro-2H-benzo[b][1,4]oxazine-7-carboxamide;
(151) (S)-4-((4′-carbamoyl-[1,1′-biphenyl]-2-yl)methyl)-N-(1-(naphthalene-2-yl)ethyl)-3,4-dihydro-2H-benzo[b][1,4]oxazine-7-carboxamide;
(152) (S)-2′-((6-((1-phenylethyl)carbamoyl)-3,4-dihydroquinoline-1(2H)-yl)methyl)-[1,1′-biphenyl]-4-carboxylic acid;
(153) (S)-1-((4′-carbamoyl-[1,1′-biphenyl]-2-yl)methyl)-N-(1-phenylethyl)-1,2,3,4-tetrahydroquinoline-6-carboxamide;
(154) (S)-2′-((6-((1-(3-fluoro-4-methylphenyl)ethyl)carbamoyl)-3,4-dihydroquinoline-1(2H)-yl)methyl)-[1,1′-biphenyl]-4-carboxylic acid;
(155) (S)-2′-((6-((1-(4-nitrophenyl)ethyl)carbamoyl)-3,4-dihydroquinoline-1(2H)-yl)methyl)-[1,1′-biphenyl]-4-carboxylic acid;
(156) (S)-2′-((6-((1-(naphthalene-2-yl)ethyl)carbamoyl)-3,4-dihydroquinoline-1(2H)-yl)methyl)-[1,1′-biphenyl]-4-carboxylic acid;
(157) (S)-2′-((6-((1-(4-cyanophenyl)ethyl)carbamoyl)-3,4-dihydroquinoline-1(2H)-yl)methyl)-[1,1′-biphenyl]-4-carboxylic acid;
(158) (S)-1-((4′-carbamoyl-[1,1′-biphenyl]-2-yl)methyl)-N-(1-(3-fluoro-4-methylphenyl)ethyl)-1,2,3,4-tetrahydroquinoline-6-carboxamide;
(159) (S)-1-((4′-carbamoyl-[1,1′-biphenyl]-2-yl)methyl)-N-(1-(4-nitrophenyl)ethyl)-1,2,3,4-tetrahydroquinoline-6-carboxamide;
(160) (S)-1-((4′-carbamoyl-[1,1′-biphenyl]-2-yl)methyl)-N-(1-(naphthalene-2-yl)ethyl)-1,2,3,4-tetrahydroquinoline-6-carboxamide;
(161) (S)-1-((4′-carbamoyl-[1,1′-biphenyl]-2-yl)methyl)-N-(1-(4-cyanophenyl)ethyl)-1,2,3,4-tetrahydroquinoline-6-carboxamide;
(162) (S)-2′-fluoro-4′-((7-((1-phenylethyl)carbamoyl)-2H-benzo[b][1,4]oxazine-4(3H)-yl)methyl)-[1,1′-biphenyl]-2-carboxylic acid;
(163) (S)-2′-fluoro-4′-((7-((1-(naphthalene-2-yl)ethyl)carbamoyl)-2H-benzo[b][1,4]oxazine-4(3H)-yl)methyl)-[1,1′-biphenyl]-2-carboxylic acid; and
(164) (S)-2′-fluoro-4′-((7-(((3-fluoro-4-methylphenyl)ethyl)carbamoyl)-2H-benzo[b][1,4]oxazine-4(3H)-yl)methyl)-[1,1′-biphenyl]-2-carboxylic acid.

The compound represented by formula 1 of the present invention can be used as a form of a pharmaceutically acceptable salt, in which the salt is preferably an acid addition salt formed by pharmaceutically acceptable free acids. The acid addition salt herein can be obtained from inorganic acids, such as hydrochloric acid, nitric acid, phosphoric acid, sulfuric acid, hydrobromic acid, hydroiodic acid, nitrous acid, and phosphorous acid; non-toxic organic acids, such as aliphatic mono/dicarboxylate, phenyl-substituted alkanoate, hydroxy alkanoate, alkandioate, aromatic acids, and aliphatic/aromatic sulfonic acids; or organic acids, such as acetic acid, benzoic acid, citric acid, lactic acid, maleic acid, gluconic acid, methanesulfonic acid, 4-toluenesulfonic acid, tartaric acid, and fumaric acid. The pharmaceutically non-toxic salts are exemplified by sulfate, pyrosulfate, bisulfate, sulphite, bisulphite, nitrate, phosphate, monohydrogen phosphate, dihydrogen phosphate, metaphosphate, pyrophosphate, chloride, bromide, iodide, fluoride, acetate, propionate, decanoate, caprylate, acrylate, formate, isobutylate, caprate, heptanoate, propiolate, oxalate, malonate, succinate, suberate, cabacate, fumarate, maliate, butyne-1,4-dioate, hexane-1,6-dioate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutylate, citrate, lactate, hydroxybutylate, glycolate, malate, tartrate, methanesulfonate, propanesulfonate, naphthalene-1-sulfonate, naphthalene-2-sulfonate, and mandelate.

The acid addition salt in this invention can be prepared by conventional methods known to those in the art. For example, the compound represented by formula 1 is dissolved in an organic solvent, such as methanol, ethanol, acetone, methylenechloride, or acetonitrile, to which an organic acid or an inorganic acid is added to induce precipitation. Next, the precipitate is filtered and dried to give the salt. Alternatively, the solvent and the excessive acid are distillated under reduced pressure and dried to give the salt. As an additional alternative, the precipitate is crystallized in an organic solvent to give the same.

The present invention includes not only the compound represented by formula 1 but also a pharmaceutically acceptable salt thereof and a solvate, a hydrate, or an isomer possibly produced from the same.

The present invention also provides a method for preparing the compound represented by formula 1 comprising the following steps, as shown in reaction formula 1 below:

preparing the compound represented by formula 4 by reacting the compound represented by formula 2 with the compound represented by formula 3 (step 1);

preparing the compound represented by formula 5 by substituting methoxycarbonyl group of the compound represented by formula 4 prepared in step 1 with carboxy group (step 2);

preparing the compound represented by formula 7 by reacting the compound represented by formula 5 prepared in step 2 with the compound represented by formula 6 (step 3); and

substituting t-butoxy group of the compound represented by formula 7 prepared in step 3 with —OH group (step 4).

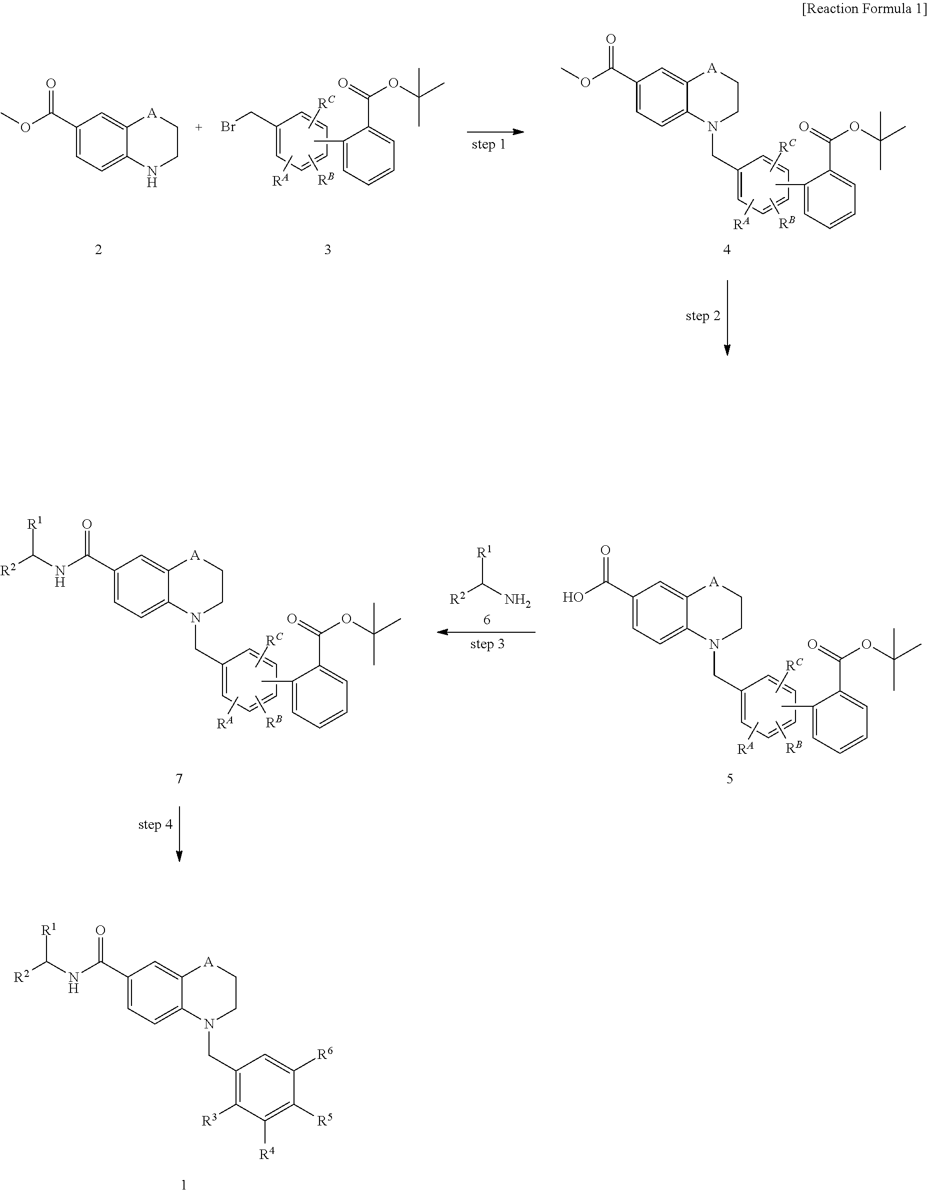

(In the reaction formula 1, R¹-R⁶ and A are as defined in formula 1, and R^{A}, R^{B}, and R^{C} are independently as defined in R³-R⁶).

The method for preparing the compound of the invention is described below in more detail step-by-step.

In the preparation method of the invention, step 1 is to prepare the compound represented by formula 4 by reacting the compound represented by formula 2 with the compound represented by formula 3.

Particularly, the compound represented by formula 2 was reacted with the compound represented by formula 3 via alkylation in an organic solvent in the presence of a base, and as a result, the compound represented by formula 4 was obtained. At this time, the reaction time was 1-30 hours, and the reaction temperature was −20-100° C.

The organic solvent used herein was selected from the group consisting of tetrahydrofuran; dioxan; ether solvents including ethyl ether and 1,2-dimethoxyethane, dimethylformamide (DMF), dimethylsulfoxide (DMSO), dichloromethane (DCM), dichloroethane, acetonitrile, toluene, chlorobenzene, and acetone.

The base used herein was selected from the group consisting of organic bases, such as pyridine, triethylamine, N,N-diisopropylethylamine (DIPEA), and 1,8-diazabicyclo[5.4.0]-7-undecene (DBU), and inorganic bases, such as sodium hydroxide, sodium carbonate, potassium carbonate, cesium carbonate, and sodium hydride, which can be used by equivalent or excessive amounts.

Upon completion of the reaction above, extraction was performed using an organic solvent, followed by drying, filtering, and distillation under reduced pressure. Column chromatography was additionally performed to give the compound represented by formula 4.

In the preparation method of the invention, step 2 is to substitute methoxycarbonyl group of the compound represented by formula 4 with carboxyl group.

Particularly, the compound represented by formula 4 was stirred in an organic solvent in the presence of a base. Upon completion of the reaction, the compound was acidified to give the compound represented by formula 5. At this time, the reaction time was 1-30 hours and the reaction temperature was −20-100° C.

The organic solvent used herein was selected from the group consisting of lower alcohols including ethanol, propanol, and butanol; tetrahydrofuran, and water.

The base used herein was selected from the group consisting of inorganic bases, such as sodium hydroxide and lithium hydroxide, which can be used by equivalent or excessive amounts.

Further, the acid used herein was HCl.

Upon completion of the reaction above, extraction was performed using an organic solvent, followed by drying, filtering, and distillation under reduced pressure. Column chromatography was additionally performed to give the compound represented by formula 5.

In the preparation method of the invention, step 3 is to prepare the compound represented by formula 7 by reacting the compound represented by formula 5 with the compound represented by formula 6.

Particularly, the compound represented by formula 5 was reacted with the compound represented by formula 6 via dehydrating condensation in an organic solvent to form a peptide bond, leading to the preparation of the compound represented by formula 7. At this time, the reaction time was 1-30 hours and the reaction temperature was −20-100° C.

The organic solvent used herein was selected from the group consisting of dimethylformamide (DMF), dimethylsulfoxide (DMSO), dichloromethane (DCM), and dichloroethane.

The base used herein was selected from the group consisting of organic bases, such as DIPEA (N,N′-Diisopropylethylamine), pyridine, and triethylamine, which can be used by equivalent or excessive amounts.

The catalyst used herein was HATU (1-[Bis(dimethylamino)methylene]-1H-1,2,3-triazolo[4,5-b]pyridinium 3-oxid hexafluorophosphate).

Upon completion of the reaction above, extraction was performed using an organic solvent, followed by drying, filtering, and distillation under reduced pressure. Column chromatography was additionally performed to give the compound represented by formula 7.

In the preparation method of the invention, step 4 is to substitute t-butoxy group of the compound represented by formula 7 prepared in step 3 with —OH group.

Particularly, the compound represented by formula 7 was reacted in an organic solvent in the presence of an acid to prepare the compound represented by formula 1. At this time, the reaction time was 1-30 hours and the reaction temperature was −20-100° C.

The organic solvent used herein was selected from the group consisting of dichloromethane (DCM) and dichloroethane.

The acid used herein was selected from the group consisting of TFA (trifluoroacetic acid), HCl, and H₂SO₄.

Upon completion of the reaction above, extraction was performed using an organic solvent, followed by drying, filtering, and distillation under reduced pressure. Column chromatography was additionally performed to give the compound represented by formula 1.

The present invention also provides a method for preparing the compound represented by formula 1 comprising the following steps, as shown in reaction formula 2 below:

preparing the compound represented by formula 9 by reacting the compound represented by formula 8 with the compound represented by formula 6 (step 1);

preparing the compound represented by formula 7 by reacting the compound represented by formula 9 prepared in step 1 with the compound represented by formula 3 (step 2); and

substituting t-butoxy group of the compound represented by formula 7 prepared in step 2 with —OH group (step 3).

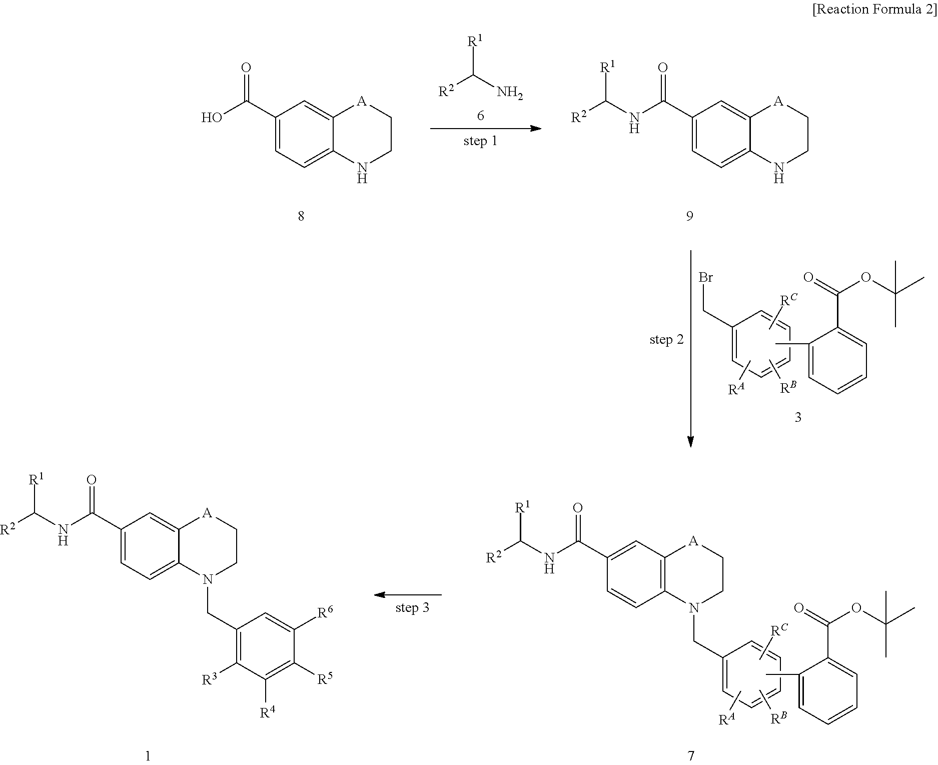

(In the reaction formula 2, R¹-R⁶ and A are as defined in formula 1, and R^{A}, R^{B}, and R^{C} are independently as defined in R³-R⁶).

The method for preparing the compound of the invention is described below in more detail step-by-step.

In the preparation method of the invention, step 1 is to prepare the compound represented by formula 9 by reacting the compound represented by formula 8 with the compound represented by formula 6.

Particularly, the compound represented by formula 8 was reacted with the compound represented by formula 6 via dehydrating condensation in an organic solvent to form a peptide bond, leading to the preparation of the compound represented by formula 9. At this time, the reaction time was 1-30 hours, and the reaction temperature was −20-100° C.

The organic solvent used herein was selected from the group consisting of dimethylformamide (DMF), dimethylsulfoxide (DMSO), dichloromethane (DCM), and dichloroethane.

The base used herein was selected from the group consisting of organic bases, such as DIPEA (N,N′-Diisopropylethylamine), pyridine, and triethylamine, which can be used by equivalent or excessive amounts.

The catalyst used herein was HATU (1-[Bis(dimethylamino)methylene]-1H-1,2,3-triazolo[4,5-b]pyridinium 3-oxid hexafluorophosphate).

Upon completion of the reaction above, extraction was performed using an organic solvent, followed by drying, filtering, and distillation under reduced pressure. Column chromatography was additionally performed to give the compound represented by formula 9.

In the preparation method of the invention, step 2 is to prepare the compound represented by formula 7 by reacting the compound represented by formula 9 with the compound represented by formula 3.

Particularly, the compound represented by formula 9 was reacted with the compound represented by formula 3 via alkylation in an organic solvent in the presence of a base, and as a result, the compound represented by formula 7 was obtained. At this time, the reaction time was 1-30 hours and the reaction temperature was −20-100° C.

The organic solvent used herein was selected from the group consisting of tetrahydrofuran; dioxan; ether solvents including ethyl ether and 1,2-dimethoxyethane, dimethylformamide (DMF), dimethylsulfoxide (DMSO), dichloromethane (DCM), dichloroethane, acetonitrile, toluene, chlorobenzene, and acetone.

The base used herein was selected from the group consisting of organic bases such as pyridine, triethylamine, N,N-diisopropylethylamine (DIPEA), and 1,8-diazabicyclo[5.4.0]-7-undecene (DBU); and inorganic bases, such as sodium hydroxide, sodium carbonate, potassium carbonate, cesium carbonate, and sodium hydride, which can be used by equivalent or excessive amounts.

Upon completion of the reaction above, extraction was performed using an organic solvent, followed by drying, filtering, and distillation under reduced pressure. Column chromatography was additionally performed to give the compound represented by formula 7.

In the preparation method of the invention, step 3 is to substitute t-butoxy group of the compound represented by formula 7 prepared in step 2 with —OH group.

Particularly, the compound represented by formula 7 was reacted in an organic solvent in the presence of an acid to prepare the compound represented by formula 1. At this time, the reaction time was 1-30 hours, and the reaction temperature was −20-100° C.

The organic solvent used herein was selected from the group consisting of dichloromethane (DCM) and dichloroethane.

The acid used herein was selected from the group consisting of TFA (trifluoroacetic acid), HCl, and H₂SO₄.

Upon completion of the reaction above, extraction was performed using an organic solvent, followed by drying, filtering, and distillation under reduced pressure. Column chromatography was additionally performed to give the compound represented by formula 1.

The present invention further provides a method for preparing the compound represented by formula 1, as shown in reaction formula 3A below, comprising the step of substituting carboxyl group of the compound represented by formula 1A with sodiumoxycarbonyl group (step 1).

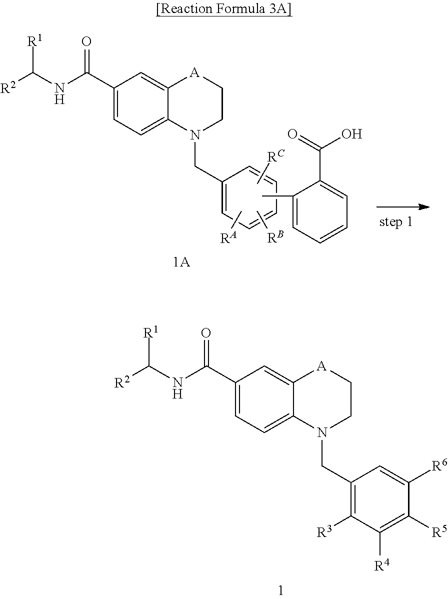

(In the reaction formula 3A, R¹-R⁶ and A are as defined in formula 1, and R^{A}, R^{B}, and R^{C} are independently as defined in R³-R⁶).

Particularly, the compound represented by formula 1A was stirred in an organic solvent in the presence of a base to give the compound represented by formula 1. At this time, the reaction time was 1-30 hours, and the reaction temperature was −20-100° C.

The organic solvent used herein was selected from the group consisting of lower alcohols including methanol, ethanol, propanol, and butanol; dichloromethane (DCM), dichloroethane, and ethylether.

The base used herein was sodium hydroxide, which can be used by equivalent or excessive amounts.

Upon completion of the reaction above, extraction was performed using an organic solvent, followed by drying, filtering, and distillation under reduced pressure. Column chromatography was additionally performed to give the compound represented by formula 1.

The present invention further provides a method for preparing the compound represented by formula 1, as shown in reaction formula 3B below, comprising the step of substituting carboxyl group of the compound represented by formula 1A with amide group (step 1).

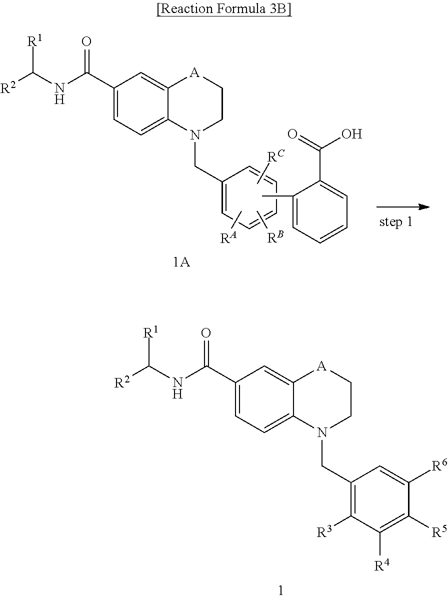

(In the reaction formula 3B, R¹-R⁶ and A are as defined in formula 1, and R^{A}, R^{B}, and R^{C} are independently as defined in R³-R⁶).

Particularly, the compound represented by formula 1A was stirred in an organic solvent in the presence of a catalyst and a base to give the compound represented by formula 1. At this time, the reaction time was 1-30 hours, and the reaction temperature was −20-100° C.

The organic solvent used herein was selected from the group consisting of ammonium hydroxide, dimethylformamide (DMF), dimethylsulfoxide (DMSO), dichloromethane (DCM), dichloroethane, and water.

The catalyst used herein was selected from the group consisting of EDCI (1-Ethyl-3-(3-dimethylaminopropyl)carbodi-imide), DMAP (4-Dimethylamino pyridine), and HOBt (Hydroxybenzotriazole).

Upon completion of the reaction above, extraction was performed using an organic solvent, followed by drying, filtering, and distillation under reduced pressure. Column chromatography was additionally performed to give the compound represented by formula 1.

The present invention also provides a pharmaceutical composition for treating PPARG-related disease which comprises the compound represented by formula 1, the optical isomer thereof, or the pharmaceutically acceptable salt of the same as an active ingredient. Particularly, the PPARG-related disease herein includes diabetes, insulin resistance, impaired glucose tolerance, pre-diabetes, hyperglycemia, hyperinsulinemia, obesity, and inflammation, but not always limited thereto.

The compound represented by formula 1 or the optical isomer thereof binds to PPARG with a high affinity but does not act as an agonist and thereby does not induce gene transcription thereof, so that it can block CDK5, which is a cause of PPARG phosphorylation, to suppress the side effects of the conventional anti-diabetic agents. Particularly, the said side effects are exemplified by weight gain, edema, impairment of bone growth or formation, and cardiac hypertrophy, but not always limited thereto.

The cause of the said side effects is phosphorylation of the 273^{rd} amino acid serine of PPARG of CDK5 kinase induced by various gene mutations. Therefore, it will be an important step for the development of an anti-diabetic agents to suppress PPARG gene transcription or to block CDK5 mediated phosphorylation. In particular, when CDK5 binds to S273 among the sites that are available for the conjugation specifically with PPARG structure (helix, H11, H12, and S273), it induces phosphorylation there, which causes side effects.

The present inventors performed an experiment to investigate whether or not the compounds prepared in the example of the invention could inhibit the phosphorylation of serine, the 273^{rd} amino acid of PPARG. As a result, it was confirmed that the compounds of the examples of the invention bound to PPARG (peroxisome proliferator activated receptor-gamma) but did not act as a CDK5 (cyclin-dependant kinase 5) promoter and thereby exhibited excellent inhibitory effects on the phosphorylation of serine, the 273^{rd} amino acid of PPARG (see Experimental Example 1).

The present inventors also investigated the PPARG (peroxisome proliferator activated receptor-gamma) transcriptional activity of the compounds prepared in the example of the invention. As a result, it was confirmed that the compounds prepared in the example of the invention had excellent activity of binding to PPARG (peroxisome proliferator activated receptor-gamma) but did not induce the transcription or activation of PPARG (peroxisome proliferator activated receptor-gamma) (see Experimental Example 2).

The present inventors also investigated the inhibitory effect of the compounds prepared in the examples of the invention on CYP (cytochrome P450) activity. As a result, it was confirmed that the compounds of examples 14, 36, 44, 56, 57, 58, 60, 74, 96, 110, 113, 115, 118, 123, 131, and 139 inhibited CYP isozymes 1A2, 2C9, 2C19, and 2D6 activity less strongly than the compounds of the Comparative Examples (SR1664 and SR1824).

Particularly, SR1664 inhibited the activity of CYP isozyme 2C9 so that 96% of the substance was not decomposed and instead remained, suggesting that, when SR1664 is administered as a drug, the amount of the drug that reaches a target is much greater than a proper dose, which might induce toxicity.

However, the compounds of the present invention inhibited CYP activity properly, better than SR1664, so the drug was decomposed at least 10 times more than SR1664, suggesting that only the proper amount of the drug reaches the target (see Experimental Example 3).

Further, the present inventors performed another experiment to evaluate the cardiotoxicity of the compounds prepared in the examples of the invention.

As a result, the compounds of the invention displayed a significantly low IC₅₀ (2.7 μM), which was significantly lower than the IC₅₀ that induces cardiotoxicity (less than 10 μM) of the compound of the Comparative Example (SR-1664). The above result indicates that the compound of the Comparative Example has a high chance of causing cardiotoxicity as a drug.

However, the compounds prepared in the examples of the invention displayed very high IC₅₀, which was much higher than IC₅₀ that could cause cardiotoxicity (less than 10 μM), indicating that the compounds had a significantly low chance of causing cardiotoxicity (see Experimental Example 4).

The present inventors also investigated the blood sugar-lowering effect and the weight-reducing effect of the compounds prepared in the examples of the invention. As a result, the compounds of examples 56, 155, 156, and 157 (10 mpk, each) reduced body weight at least 5% even at a comparatively low dose, while the compound of the Comparative Example (SR-1664, 20 mpk) reduced body weight by 1.6%.

The compounds of the examples 56, 156, and 157 (10 mpk respectively) reduced blood sugar at least 24% even at a comparatively low dose, while the compound of the Comparative Example (SR-1664, 20 mpk) reduced blood sugar by 14% (see Experimental Example 5).

The compound represented by formula 1, the optical isomer thereof, or the pharmaceutically acceptable salt thereof of the present invention can be prepared for oral or parenteral administration.

The formulations for oral administration are exemplified by tablets, pills, hard/soft capsules, solutions, suspensions, emulsions, syrups, granules, elixirs, and troches. These formulations can include diluents (for example, lactose, dextrose, sucrose, mannitol, sorbitol, cellulose, and/or glycine) and lubricants (for example, silica, talc, stearate and its magnesium or calcium salt, and/or polyethylene glycol) in addition to the active ingredient. Tablets can include binding agents, such as magnesium aluminum silicate, starch paste, gelatin, methylcellulose, sodium carboxymethylcellulose and/or polyvinylpyrolidone, and if necessary disintegrating agents, such as starch, agarose, alginic acid or its sodium salt or azeotropic mixtures and/or absorbents; coloring agents, flavours, and sweeteners can be additionally included.

The pharmaceutical composition comprising the compound represented by formula 1 as an active ingredient can be administered by parenterally, and the parenteral administration includes subcutaneous injection, intravenous injection, intramuscular injection, or intrathoracic injection.

To prepare the composition as a formulation for parenteral administration, the compound represented by formula 1, the optical isomer thereof, or the pharmaceutically acceptable salt thereof is mixed with a stabilizer or a buffering agent in water to produce a solution or suspension, which is then formulated as ampoules or vials. The composition herein can be sterilized and additionally contains preservatives, stabilizers, wettable powders or emulsifiers, salts and/or buffers for the regulation of osmotic pressure, and other therapeutically useful materials, and the composition can be formulated by the conventional mixing, granulating or coating method.

The effective dosage of the pharmaceutical composition comprising the compound represented by formula 1 as an active ingredient of the present invention can be determined according to age, weight, gender, administration method, health condition, and severity of disease. The dosage is preferably 0.01-1000 mg/kg/day, which can be administered several times a day or preferably 1-3 times a day.

The preparation method of the compound represented by formula 1 of the present invention is described in more detail in preparative examples or examples. The following preparative examples or examples are only examples to describe the method for preparing the compound represented by formula 1 and the present invention is not limited thereto. The preparation methods described in the following preparative examples or examples are performed under the conditions with proper reagents well-known in the field of organic synthesis.

### Preparative Example 1

### 1-((2′-(tert-butoxycarbonyl)-[1,1′-biphenyl]-4-yl)methyl)-1,2,3,4-tetrahydroquinoline-6-carboxylic acid

### Step 1 Methyl 1,2,3,4-tetrahydroquinoline-6-carboxylate

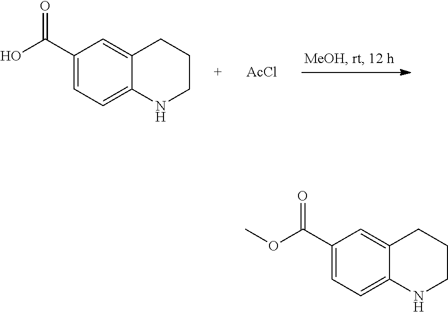

1,2,3,4-tetrahydroquinoline-6-carboxylic acid (50 g, 282 mmol) was dissolved in MeOH (500 mL) in a 2 L flask, to which AcCl (100 mL) was added at 0° C., followed by stirring at room temperature for 12 hours. Upon completion of the reaction, the reaction mixture was concentrated under reduced pressure to give methyl 1,2,3,4-tetrahydroquinoline-6-carboxylate (45 g).

### Step 2 Methyl 1-((2′-(tert-butoxycarbonyl)-[1,1′-biphenyl]-4-yl)methyl)-1,2,3,4-tetrahydroquinoline-6-carboxylate

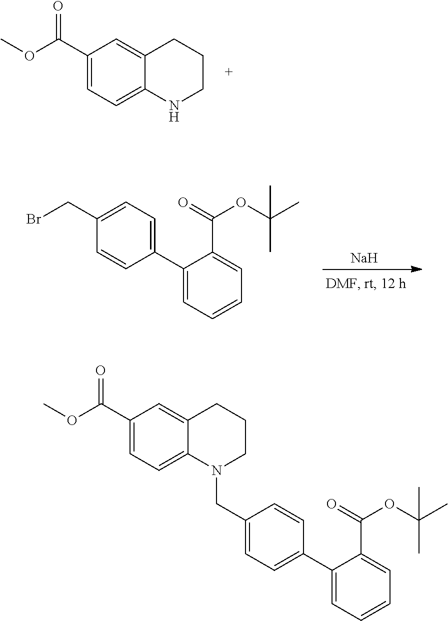

Methyl 1,2,3,4-tetrahydroquinoline-6-carboxylate (2 g, 10.46 mmol) was dissolved in DMF (20 ml) in a 1 L flask, to which NaH (628 mg, 15.69 mmol) and tert-butyl 4′-(bromomethyl)-[1,1′-biphenyl]-2-carboxylate (4.36 g, 12.55 mmol) were added, followed by stirring at room temperature for 12 hours. The organic layer was separated by using ethyl acetate and brine. The organic layer was dried over MgSO₄ and filtered. The solvent was eliminated from the mixture via vacuum distillation, followed by column chromatography to give methyl 1-((2′-(tert-butoxycarbonyl)-[1,1′-biphenyl]-4-yl)methyl)-1,2,3,4-tetrahydroquinoline-6-carboxylate (4 g, 8.55 mmol, 85%).

### Step 3: 1-((2′-(tert-butoxycarbonyl)-[1,1′-biphenyl]-4-yl)methyl)-1,2,3,4-tetrahydroquinoline-6-carboxylic acid

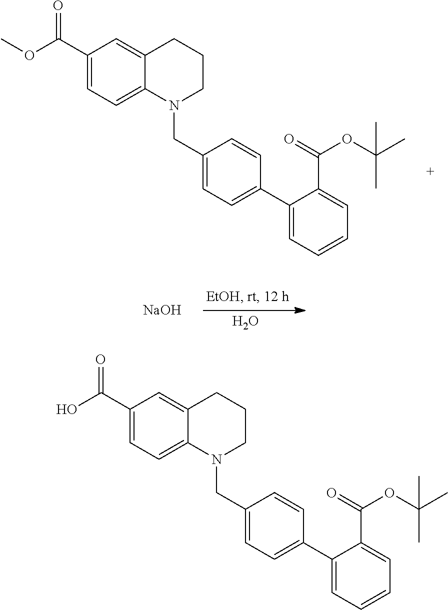 [SMILES: COC(=O)c1ccc2c(c1)CCCN2Cc1ccc(-c2ccccc2C(=O)OC(C)(C)C)cc1 ; "NaOH" ; O ; CC(C)(C)OC(=O)c1ccccc1-c1ccc(CN2CCCc3cc(C(=O)O)ccc32)cc1]

Methyl 1-((2′-(tert-butoxycarbonyl)-[1,1′-biphenyl]-4-yl)methyl)-1,2,3,4-tetrahydroquinoline-6-carboxylate (4 g, 8.55 mmol) was mixed with NaOH (1.48 g, 37 mmol) dissolved in EtOH (50 mL)/H₂O (50 mL) in a 250 mL flask, followed by stirring at room temperature for 12 hours. Upon completion of the reaction, the solvent was concentrated, and the pH was adjusted to 5 with 2N HCl. Extraction was performed with EA, and the organic layer was dried over MgSO₄ and filtered. As a result, 1-((2′-(tert-butoxycarbonyl)-[1,1′-biphenyl]-4-yl)methyl)-1,2,3,4-tetrahydroquinoline-6-carboxylic acid was obtained (3.55 g, 8.0 mmol, 90%).

### Preparative Example 2

### 4-((2′-(tert-butoxycarbonyl)-[1,1′-biphenyl]-4-yl)methyl)-3,4-dihydro-2H-benzo[b][1,4]oxazine-7-carboxylic acid

### Step 1: Methyl 4-amino-3-hydroxybenzoate

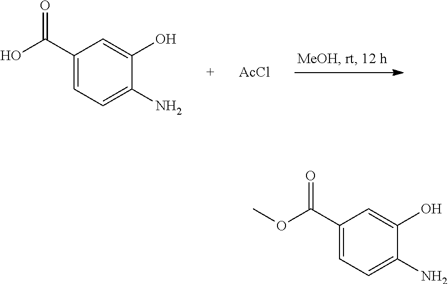

4-amino-3-hydroxybenzoic acid (50 g, 326 mmol) was dissolved in MeOH (500 mL) in a 2 L flask, to which AcCL (100 mL) was added at 0° C., followed by stirring at room temperature for 12 hours. Upon completion of the reaction, the reaction mixture was concentrated under reduced pressure to give 4-amino-3-hydroxybenzoate (50 g).

### Step 2 Methyl 3,4-dihydro-2H-benzo[b][1, 4]oxazine-7-carboxylate

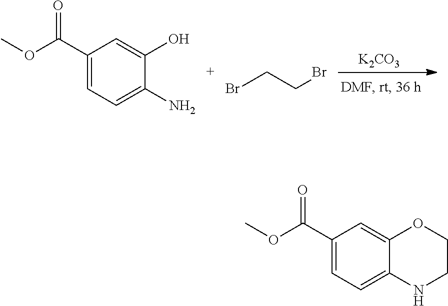

Methyl 4-amino-3-hydroxybenzoate (55 g, 329 mmol) prepared in step 1 and 1,2-dibromoethane (185 g, 986 mmol) were dissolved in DMF (1 L) in a 2 L flask, to which K₂CO₃ (227 g, 1645 mmol) was added, followed by stirring at room temperature for 36 hours. The organic layer was separated by using ethyl acetate and brine. The separated organic layer was dried over MgSO₄ and filtered. The solvent was eliminated from the mixture via vacuum distillation, followed by column chromatography to give methyl 3,4-dihydro-2H-benzo[b][1,4]oxazine-7-carboxylate (41.3 g, 213 mmol, 65%).

### Step 3 Methyl 4-((2′-(tert-butoxycarbonyl)-[1,1′-biphenyl]-4-yl)methyl)-3,4-dihydro-2H-benzo[b][1,4]oxazine-7-carboxylate

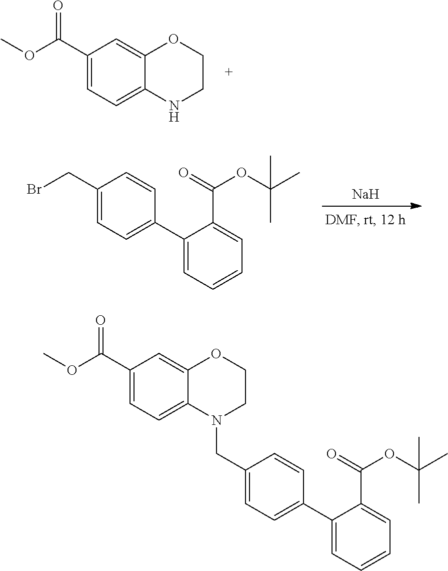

Methyl 3,4-dihydro-2H-benzo[b][1,4]oxazine-7-carboxylate (8.08 g, 41.8 mmol) obtained in step 2 was dissolved in DMF (50 ml) in a 500 mL flask, to which NaH (2 g, 50.2 mmol) and tert-butyl 4′-(bromomethyl)-[1,1′-biphenyl]-2-carboxylate (17.4 g, 50.2 mmol) were added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give methyl 4-((2′-(tert-butoxycarbonyl)-[1,1′-biphenyl]-4-yl)methyl)-3,4-dihydro-2H-benzo[b][1,4]oxazine-7-carboxylate (28.4 g, 68.03 mmol, 68%).

### Step 4: 4-((2′-(tert-butoxycarbonyl)-[1,1′-biphenyl]-4-yl)methyl)-3, 4-dihydro-2H-benzo[b][1,4]oxazine-7-carboxylic acid

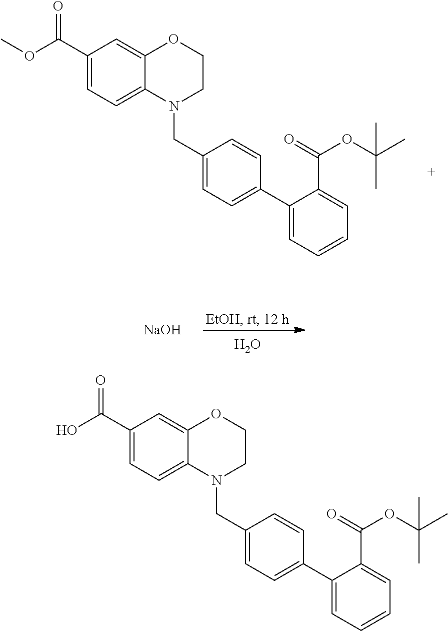 [SMILES: COC(=O)c1ccc2c(c1)OCCN2Cc1ccc(-c2ccccc2C(=O)OC(C)(C)C)cc1 ; "NaOH" ; O ; CC(C)(C)OC(=O)c1ccccc1-c1ccc(CN2CCOc3cc(C(=O)O)ccc32)cc1]

Methyl 4-((2′-(tert-butoxycarbonyl)-[1,1′-biphenyl]-4-yl)methyl)-3,4-dihydro-2H-benzo[b][1,4]oxazine-7-carboxylate (11.8 g, 28.43 mmol) obtained in step 3 was mixed with NaOH (5.68 g, 142 mmol) dissolved in EtOH (50 ml) and H₂O (50 ml) in a 250 mL flask, followed by stirring at room temperature for 12 hours. Upon completion of the reaction, the solvent was concentrated. PH of the reaction mixture was adjusted to 5 by using 2N-HCl.

The mixture was extracted with EA. The organic layer was dried over MgSO₄ and filtered. As a result, 4-((2′-(tert-butoxycarbonyl)-[1,1′-biphenyl]-4-yl)methyl)-3,4-dihydro-2H-benzo[b][1,4]oxazine-7-carboxylic acid was obtained (10.3 g, 25.53 mmol, 90%).

### Preparative Example 3

### 1-((2′-(tert-butoxycarbonyl)-3-fluoro-[1,1′-biphenyl]-4-yl)methyl)-1,2,3,4-tetrahydroquinoline-6-carboxylic acid

### Step 1: tert-butyl 2-bromobenzoate

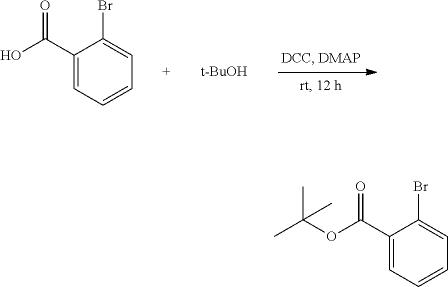

2-bromobenzoic acid (25 g, 124 mmol), DMAP (1.52 g, 12.4 mmol), and t-butanol (9.2 g, 124 mmol) were dissolved in dichloromethane (500 ml) in a 1 L flask, to which DCC (25.7 g, 124 mmol) was added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give tert-butyl 2-bromobenzoate (30 g).

### Step 2 tert-butyl 3′-fluoro-4′-methyl-[1,1′-biphenyl]-2-carboxylate

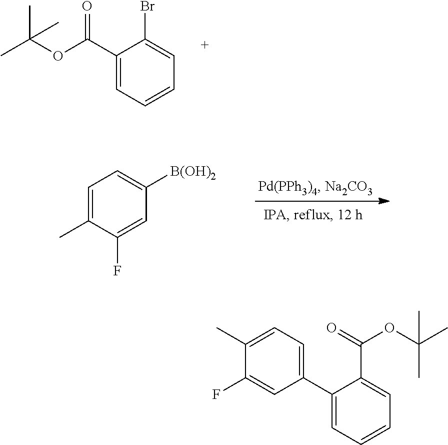 [SMILES: CC(C)(C)OC(=O)c1ccccc1Br ; Cc1ccc(B(O)O)cc1F ; Cc1ccc(-c2ccccc2C(=O)OC(C)(C)C)cc1F]

Tert-butyl 2-bromobenzoate (10 g, 64.8 mmol), (3-fluoro-4-methylphenyl)boronic acid (14 g, 54 mmol), Pd(PPh₃)₄(624 mg, 0.54 mmol), and Na₂CO₃ (11.4 g, 108 mmol) were dissolved in IPA (60 ml) and H₂O (60 ml) in a 1 L flask, followed by reflux-stirring for 12 hours. The reaction mixture was concentrated under reduced pressure. The organic layer was separated by using ethyl acetate and brine. The separated organic layer was dried over MgSO₄ and filtered. The mixture was separated by column chromatography to give tert-butyl 3′-fluoro-4′-methyl-[1,1′-biphenyl]-2-carboxylate (14 g).

### Step 3 tert-butyl 4′-(bromomethyl)-3′-fluoro-[1,1′-biphenyl]-2-carboxylate

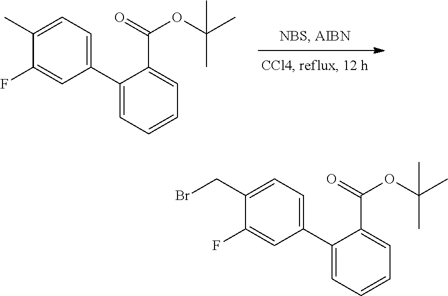 [SMILES: Cc1ccc(-c2ccccc2C(=O)OC(C)(C)C)cc1F ; CC(C)(C)OC(=O)c1ccccc1-c1ccc(CBr)c(F)c1]

Tert-butyl 3′-fluoro-4′-methyl-[1,1′-biphenyl]-2-carboxylate (14 g, 48.9 mmol), NBS (9.57 g, 53.79 mmol), and AIBN (2.4 g, 14.67 mmol) were dissolved in CCl₄(50 ml) in a 1 L flask, followed by reflux-stirring for 12 hours. The reaction mixture was concentrated under reduced pressure. The organic layer was separated by using dichloromethane and brine. The separated organic layer was dried over MgSO₄ and filtered. The mixture was separated by column chromatography to give tert-butyl 4′-(bromomethyl)-3′-fluoro-[1,1′-biphenyl]-2-carboxylate (7.4 g).

### Step 4 methyl 1-((2′-(tert-butoxycarbonyl)-3-fluoro-[1,1′-biphenyl]-4-yl)methyl)-1,2,3,4-tetrahydroquinoline-6-carboxylate

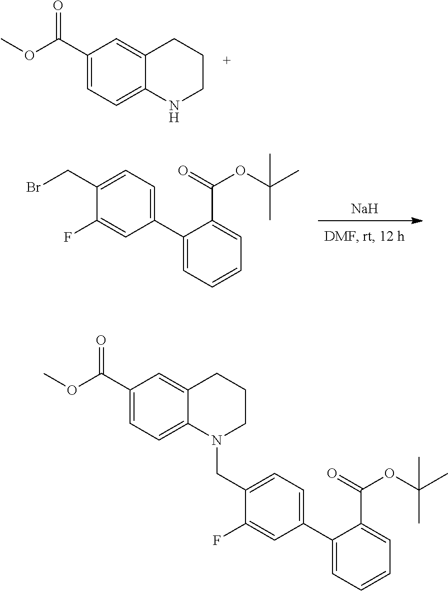

Methyl 1,2,3,4-tetrahydroquinoline-6-carboxylate (3 g, 15.69 mmol) was dissolved in DMF (20 ml) in a 250 mL flask, to which NaH (753 mg, 18.83 mmol) and tert-butyl 4′-(bromomethyl)-3′-fluoro-[1,1′-biphenyl]-2-carboxylate (5.7 g, 15.69 mmol) were added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give methyl 1-((2′-(tert-butoxycarbonyl)-3-fluoro-[1,1′-biphenyl]-4-yl)methyl)-1,2,3,4-tetrahydroquinoline-6-carboxylate (4 g).

### Step 5: 1-((2′-(tert-butoxycarbonyl)-3-fluoro-[1,1′-biphenyl]-4-yl)methyl)-1,2,3,4-tetrahydroquinoline-6-carboxylic acid

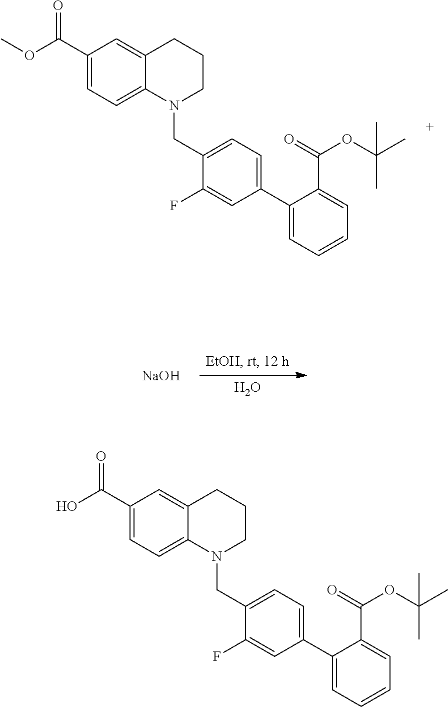

Methyl 1-((2′-(tert-butoxycarbonyl)-3-fluoro-[1,1′-biphenyl]-4-yl)methyl)-1,2,3,4-tetrahydroquinoline-6-carboxylate (4 g, 8.4 mmol) was mixed with NaOH (1.48 g, 37 mmol) dissolved in EtOH (50 ml) and H₂O (50 ml) in a 250 mL flask, followed by stirring at room temperature for 12 hours. Upon completion of the reaction, the solvent was concentrated. PH of the reaction mixture was adjusted to 5 by using 2N-HCl. The mixture was extracted with EA. The organic layer was dried over MgSO₄ and filtered. As a result, 1-((2′-(tert-butoxycarbonyl)-3-fluoro-[1,1′-biphenyl]-4-yl)methyl)-1,2,3,4-tetrahydroquinoline-6-carboxylic acid was obtained (3 g).

### Preparative Example 4

### 1-((2′-(tert-butoxycarbonyl)-[1,1′-biphenyl]-3-yl)methyl)-1,2,3,4-tetrahydroquinoline-6-carboxylic acid

### Step 1: tert-butyl 2-bromobenzoate

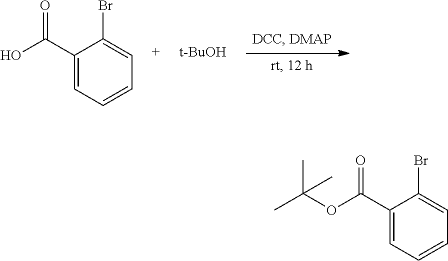

2-bromobenzoic acid (25 g, 124 mmol), DMAP (1.52 g, 12.4 mmol), and t-butanol (9.2 g, 124 mmol) were dissolved in dichloromethane (500 ml) in a 1 L flask, to which DCC (25.7 g, 124 mmol) was added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give tert-butyl 2-bromobenzoate (30 g).

### Step 2 tert-butyl 3′-methyl-[1,1′-biphenyl]-2-carboxylate

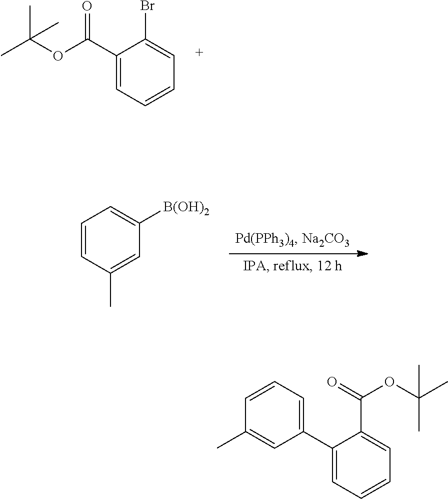

Tert-butyl 2-bromobenzoate (22.7 g, 88.26 mmol), meta-toluoylboronic acid (8 g, 58.84 mmol), Pd(PPh₃)₄ (680 mg, 0.58 mmol), and Na₂CO₃ (12.47 g, 117 mmol) were dissolved in IPA (50 ml) and H₂O (50 ml) in a 1 L flask, followed by reflux-stirring for 12 hours. The reaction mixture was concentrated under reduced pressure. The organic layer was separated by using ethyl acetate and brine. The separated organic layer was dried over MgSO₄ and filtered. The mixture was separated by column chromatography to give tert-butyl 3′-methyl-[1,1′-biphenyl]-2-carboxylate (7.2 g).

### Step 3 tert-butyl 3′-(bromomethyl)-[1,1′-biphenyl]-2-carboxylate

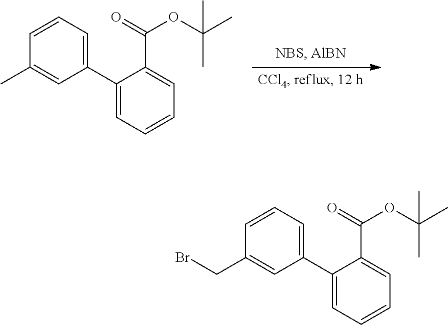

Tert-butyl 3′-methyl-[1,1′-biphenyl]-2-carboxylate (7.2 g, 26.83 mmol), NBS (5.73 g, 32.2 mmol), and AIBN (1.32 g, 8.05 mmol) were dissolved in CCl₄ (100 ml) in a 500 mL flask, followed by reflux-stirring for 12 hours. The reaction mixture was concentrated under reduced pressure. The organic layer was separated by using dichloromethane and brine. The separated organic layer was dried over MgSO₄ and filtered. The mixture was separated by column chromatography to give tert-butyl 3′-(bromomethyl)-[1,1′-biphenyl]-2-carboxylate (5.2 g).

### Step 4 methyl 1-((2′-(tert-butoxycarbonyl)-[1,1′-biphenyl]-3-yl)methyl)-1,2,3,4-tetrahydroquinoline-6-carboxylate

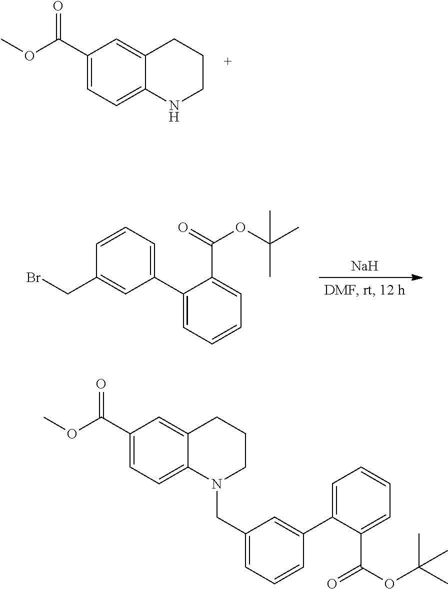

Methyl 1,2,3,4-tetrahydroquinoline-6-carboxylate (3 g, 15.69 mmol) was dissolved in DMF (20 ml) in a 100 mL flask, to which NaH (753 mg, 18.83 mmol) and tert-butyl 3′-(bromomethyl)-[1,1′-biphenyl]-2-carboxylate (5.45 g, 15.69 mmol) were added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give methyl 1-((2′-(tert-butoxycarbonyl)-[1,1′-biphenyl]-3-yl)methyl)-1,2,3,4-tetrahydroquinoline-6-carboxylate (4.76 g).

### Step 5: 1-((2′-(tert-butoxycarbonyl)-[1,1′-biphenyl]-3-yl)methyl)-1,2,3,4-tetrahydroquinoline-6-carboxylic acid

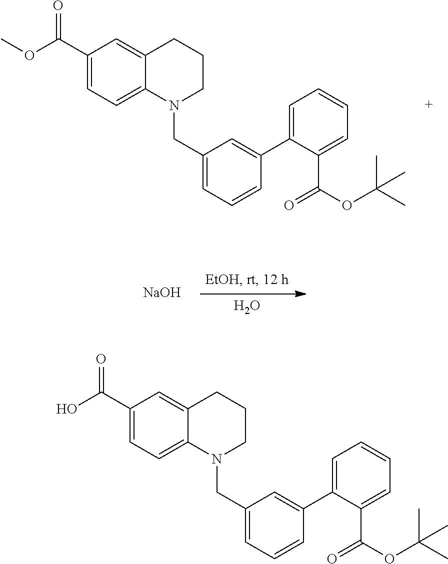

Methyl 1-((2′-(tert-butoxycarbonyl)-[1,1′-biphenyl]-3-yl)methyl)-1,2,3,4-tetrahydroquinoline-6-carboxylate (4.76 g, 10.4 mmol) was mixed with NaOH (2.08 g, 52 mmol) dissolved in EtOH (50 ml) and H₂O (50 ml) in a 250 mL flask, followed by stirring at room temperature for 12 hours. Upon completion of the reaction, the solvent was concentrated. The pH of the reaction mixture was adjusted to 5 by using 2N HCl. The mixture was extracted with EA. The organic layer was dried over MgSO₄ and filtered. As a result, 1-((2′-(tert-butoxycarbonyl)-[1,1′-biphenyl]-3-yl)methyl)-1,2,3,4-tetrahydroquinoline-6-carboxylic acid was obtained (2.7 g).

### Preparative Example 5

### 1-((2′-(tert-butoxycarbonyl)-2-fluoro-[1,1′-biphenyl]-3-yl)methyl)-1,2,3,4-tetrahydroquinoline-6-carboxylic acid

### Step 1: tert-butyl 2-bromobenzoate

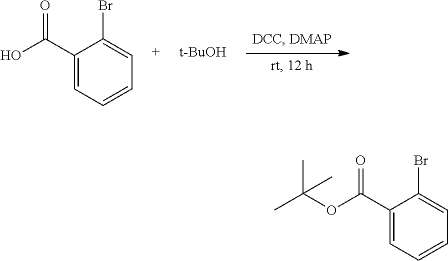

2-bromobenzoic acid (25 g, 124 mmol), DMAP (1.52 g, 12.4 mmol), and t-butanol (9.2 g, 124 mmol) were dissolved in dichloromethane (500 ml) in a 1 L flask, to which DCC (25.7 g, 124 mmol) was added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give tert-butyl 2-bromobenzoate (30 g).

### Step 2 tert-butyl 2′-fluoro-3′-methyl-[1,1′-biphenyl]-2-carboxylate

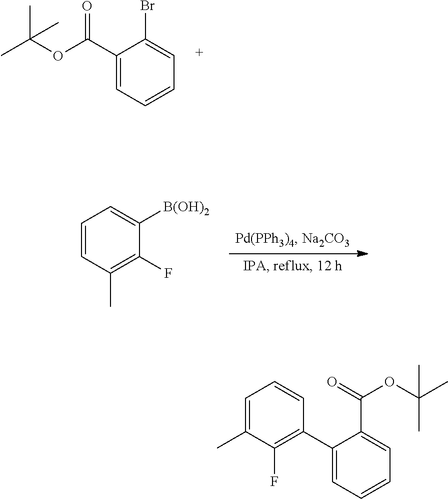

Tert-butyl 2-bromobenzoate (33.38 g, 129.8 mmol), (2-fluoro-3-methylphenyl)boronic acid (10 g, 64.9 mmol), Pd(PPh₃)₄(749 mg, 0.65 mmol), and Na₂CO₃ (13.75 g, 129.8 mmol) were dissolved in IPA (60 ml) and H₂O (60 ml) in a 1 L flask, followed by reflux-stirring for 12 hours. The reaction mixture was concentrated under reduced pressure. The organic layer was separated by using ethyl acetate and brine. The separated organic layer was dried over MgSO₄ and filtered. The mixture was separated by column chromatography to give tert-butyl 2′-fluoro-3′-methyl-[1,1′-biphenyl]-2-carboxylate (15 g).

### Step 3 tert-butyl 3′-(bromomethyl)-2′-fluoro-[1,1′-biphenyl]-2-carboxylate

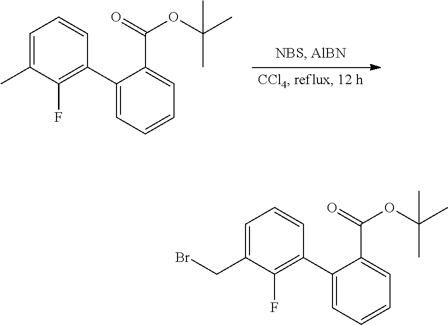

Tert-butyl 2′-fluoro-3′-methyl-[1,1′-biphenyl]-2-carboxylate (18.58 g, 64.89 mmol), NBS (17.3 g, 97.33 mmol), and AIBN (3.2 g, 19.46 mmol) were dissolved in CCl₄ (100 ml) in a 500 mL flask, followed by reflux-stirring for 12 hours. The reaction mixture was concentrated under reduced pressure. The organic layer was separated by using dichloromethane and brine. The separated organic layer was dried over MgSO₄ and filtered. The mixture was separated by column chromatography to give tert-butyl 3′-(bromomethyl)-2′-fluoro-[1,1′-biphenyl]-2-carboxylate (12.5 g).

### Step 4 methyl 1-((2′-(tert-butoxycarbonyl)-2-fluoro-[1,1′-biphenyl]-3-yl)methyl)-1,2,3,4-tetrahydroquinoline-6-carboxylate

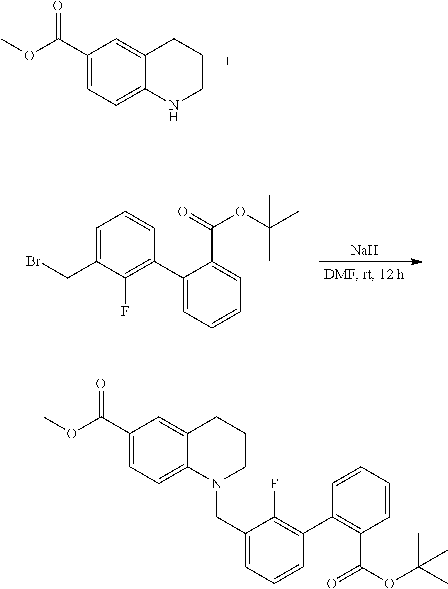

Methyl 1,2,3,4-tetrahydroquinoline-6-carboxylate (4 g, 20.9 mmol) was dissolved in DMF (20 ml) in a 250 mL flask, to which NaH (1 g, 25.08 mmol) and tert-butyl 3′-(bromomethyl)-2′-fluoro-[1,1′-biphenyl]-2-carboxylate (12.5 g, 34.22 mmol) were added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give methyl 1-((2′-(tert-butoxycarbonyl)-2-fluoro-[1,1′-biphenyl]-3-yl)methyl)-1,2,3,4-tetrahydroquinoline-6-carboxylate (6.2 g).

### Step 5: 1-((2′-(tert-butoxycarbonyl)-2-fluoro-[1,1′-biphenyl]-3-yl)methyl)-1,2,3,4-tetrahydroquinoline-6-carboxylic acid

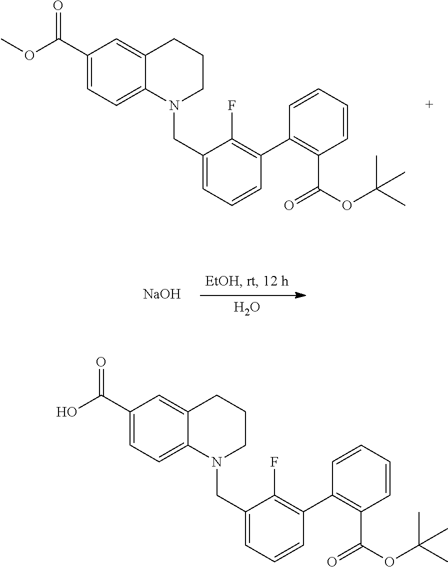

Methyl 1-((2′-(tert-butoxycarbonyl)-2-fluoro-[1,1′-biphenyl]-3-yl)methyl)-1,2,3,4-tetrahydroquinoline-6-carboxylate (6.2 g, 13.04 mmol) was mixed with NaOH (2.6 g, 65.2 mmol) dissolved in EtOH (50 ml) and H₂O (50 ml) in a 250 mL flask, followed by stirring at room temperature for 12 hours. Upon completion of the reaction, the solvent was concentrated. The pH of the reaction mixture was adjusted to 5 by using 2N HCl. The mixture was extracted with EA. The organic layer was dried over MgSO₄ and filtered. As a result, 1-((2′-(tert-butoxycarbonyl)-2-fluoro-[1,1′-biphenyl]-3-yl)methyl)-1,2,3,4-tetrahydroquinoline-6-carboxylic acid was obtained (4 g).

### Preparative Example 6

### 1-((2′-(tert-butoxycarbonyl)-4-fluoro-[1,1′-biphenyl]-3-yl)methyl)-1,2,3,4-tetrahydroquinoline-6-carboxylic acid

### Step 1: tert-butyl 2-bromobenzoate

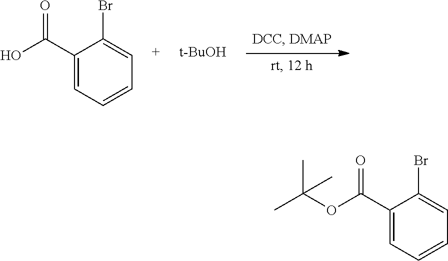

2-bromobenzoic acid (25 g, 124 mmol), DMAP (1.52 g, 12.4 mmol), and t-butanol (9.2 g, 124 mmol) were dissolved in dichloromethane (500 ml) in a 1 L flask, to which DCC (25.7 g, 124 mmol) was added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give tert-butyl 2-bromobenzoate (30 g).

### Step 2 tert-butyl 4′-fluoro-3′-methyl-[1,1′-biphenyl]-2-carboxylate

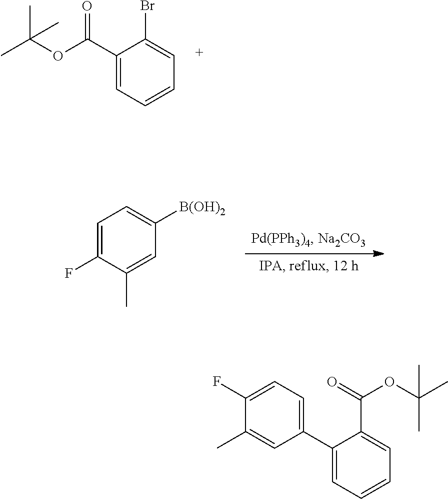

Tert-butyl 2-bromobenzoate (33.38 g, 129.8 mmol), (4-fluoro-3-methylphenyl)boronic acid (10 g, 64.9 mmol), Pd(PPh₃)₄(749 mg, 0.64 mmol), and Na₂CO₃ (13.7 g, 129.8 mmol) were dissolved in IPA (50 ml) and H₂O (50 ml) in a 1 L flask, followed by reflux-stirring for 12 hours. The reaction mixture was concentrated under reduced pressure. The organic layer was separated by using ethyl acetate and brine. The separated organic layer was dried over MgSO₄ and filtered. The mixture was separated by column chromatography to give tert-butyl 4′-fluoro-3′-methyl-[1,1′-biphenyl]-2-carboxylate (13 g).

### Step 3 tert-butyl 3′-(bromomethyl)-4′-fluoro-[1,1′-biphenyl]-2-carboxylate

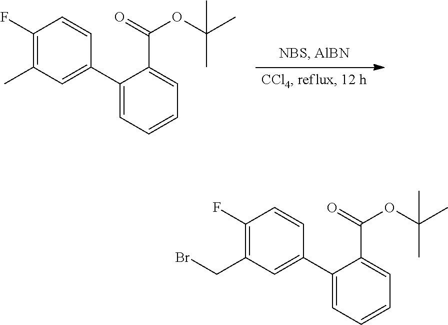

Tert-butyl 4′-fluoro-3′-methyl-[1,1′-biphenyl]-2-carboxylate (13 g, 45.4 mmol), NBS (12.12 g, 68.1 mmol), and AIBN (2.24 g, 13.62 mmol) were dissolved in CCl₄ (100 ml) in a 500 mL flask, followed by reflux-stirring for 12 hours. The reaction mixture was concentrated under reduced pressure. The organic layer was separated by using ethyl acetate and brine. The separated organic layer was dried over MgSO₄ and filtered. The mixture was separated by column chromatography to give tert-butyl 3′-(bromomethyl)-4′-fluoro-[1,1′-biphenyl]-2-carboxylate (11.25 g).

### Step 4: methyl 1-((2′-(tert-butoxycarbonyl)-4-fluoro-[1,1′-biphenyl]-3-yl)methyl)-1,2,3,4-tetrahydroquinoline-6-carboxylate

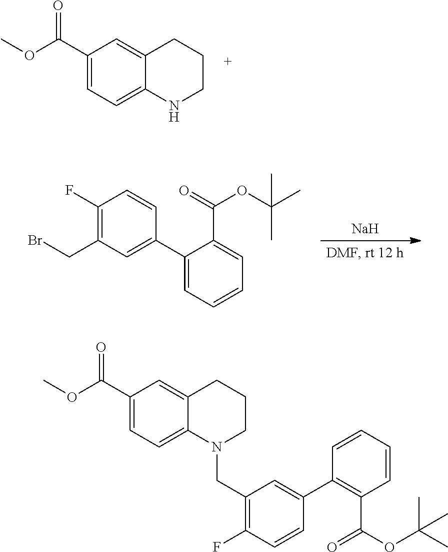

Methyl 1,2,3,4-tetrahydroquinoline-6-carboxylate (4 g, 20.9 mmol) was dissolved in DMF (20 ml) in a 250 mL flask, to which NaH (1 g, 25.08 mmol) and tert-butyl 3′-(bromomethyl)-4′-fluoro-[1,1′-biphenyl]-2-carboxylate (11.25 g, 30.8 mmol) were added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give methyl 1-((2′-(tert-butoxycarbonyl)-4-fluoro-[1,1′-biphenyl]-3-yl)methyl)-1,2,3,4-tetrahydroquinoline-6-carboxylate (6.45 g).

### Step 5: 1-((2′-(tert-butoxycarbonyl)-4-fluoro-[1,1′-biphenyl]-3-yl)methyl)-1,2,3,4-tetrahydroquinoline-6-carboxylic acid

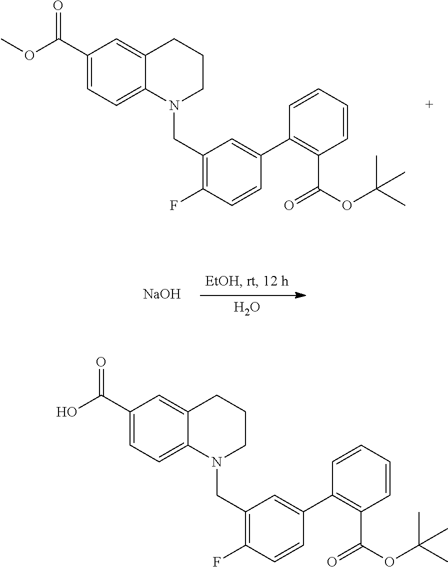

Methyl 1-((2′-(tert-butoxycarbonyl)-4-fluoro-[1,1′-biphenyl]-3-yl)methyl)-1,2,3,4-tetrahydroquinoline-6-carboxylate (6.45 g, 13.56 mmol) was mixed with NaOH (2.71 g, 67.8 mmol) dissolved in EtOH (50 ml) and H₂O (50 ml) in a 250 mL flask, followed by stirring at room temperature for 12 hours. Upon completion of the reaction, the solvent was concentrated. The pH of the reaction mixture was adjusted to 5 by using 2N HCl. The mixture was extracted with EA. The organic layer was dried over MgSO₄ and filtered. As a result, 1-((2′-(tert-butoxycarbonyl)-4-fluoro-[1,1′-biphenyl]-3-yl)methyl)-1,2,3,4-tetrahydroquinoline-6-carboxylic acid was obtained (5.2 g).

### Preparative Example 7

### 3,4-dihydro-2H-benzo[b][1,4]oxazine-7-carboxylic acid

### Step 1 methyl 2′-methyl-[1,1′-biphenyl]-4-carboxylate

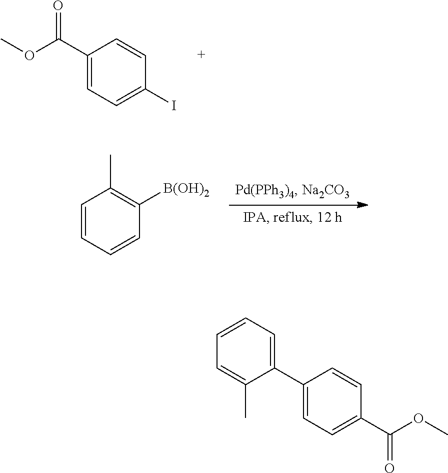

Methyl 4-iodobenzoate (5 g, 19 mmol), ortho-toluylboronic acid (3.1 g, 22.9 mmol), Pd(PPh₃)₄ (1.1 g, 0.95 mmol), and Na₂CO₃ (8.5 g, 38 mmol) were dissolved in IPA (50 ml) and H₂O (50 ml) in a 100 mL flask, followed by reflux-stirring for 12 hours. The reaction mixture was concentrated under reduced pressure. The organic layer was separated by using ethyl acetate and brine. The separated organic layer was dried over MgSO₄ and filtered. The mixture was separated by column chromatography to give methyl 2′-methyl-[1,1′-biphenyl]-4-carboxylate (4.3 g).

### Step 2: methyl 2′-(bromomethyl)-[1,1′-biphenyl]-4-carboxylate

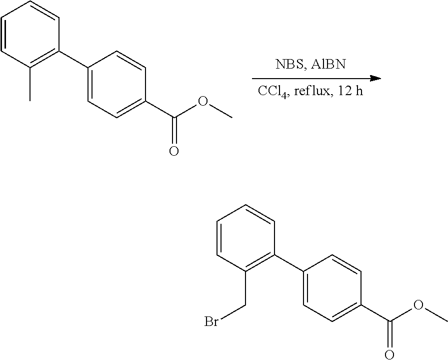

Methyl 2′-methyl-[1,1′-biphenyl]-4-carboxylate (1.1 g, 4.86 mmol), NBS (951 mg, 5.34 mmol), and AIBN (239 mg, 1.45 mmol) were dissolved in CCl₄ (10 ml) in a 50 mL flask, followed by reflux-stirring for 12 hours. The reaction mixture was concentrated under reduced pressure. The organic layer was separated by using dichloromethane and brine. The separated organic layer was dried over MgSO₄ and filtered. The mixture was separated by column chromatography to give methyl 2′-(bromomethyl)-[1,1′-biphenyl]-4-carboxylate (1 g).

### Step 3: 3,4-dihydro-2H-benzo[b][1,4]oxazine-7-carboxylic acid

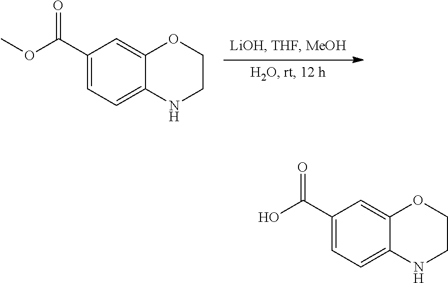

Methyl 3,4-dihydro-2H-benzo[b][1,4]oxazine-7-carboxylate (1 g, 5.2 mmol) was dissolved in MeOH (5 ml) and THF (10 ml) in a 25 mL flask, to which LiOH (2 g) dissolved in H₂O (5 ml) was added, followed by stirring at room temperature for 12 hours. Upon completion of the reaction, the solvent was concentrated. The pH of the reaction mixture was adjusted to 5 by using 2N HCl. The mixture was extracted with EA. The organic layer was dried over MgSO₄ and filtered. As a result, 3,4-dihydro-2H-benzo[b][1,4]oxazine-7-carboxylic acid was obtained (1.2 g).

### Preparative Example 8

### methyl 2′-(bromomethyl)-[1,1′-biphenyl]-4-carboxylate

### Step 1 methyl 2′-methyl-[1,1′-biphenyl]-4-carboxylate

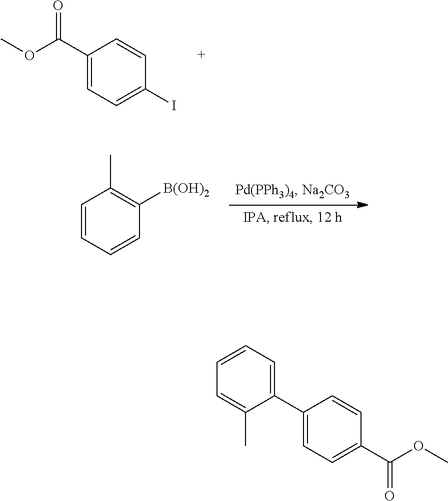

Methyl 4-iodobenzoate (5 g, 19 mmol), ortho-toluylboronic acid (3.1 g, 22.9 mmol), Pd(PPh₃)₄ (1.1 g, 0.95 mmol), and Na₂CO₃ (8.5 g, 38 mmol) were dissolved in IPA (50 ml) and H₂O (50 ml) in a 100 mL flask, followed by reflux-stirring for 12 hours. The reaction mixture was concentrated under reduced pressure. The organic layer was separated by using ethyl acetate and brine. The separated organic layer was dried over MgSO₄ and filtered. The mixture was separated by column chromatography to give methyl 2′-methyl-[1,1′-biphenyl]-4-carboxylate (4.3 g).

### Step 2: methyl 2′-(bromomethyl)-[1,1′-biphenyl]-4-carboxylate

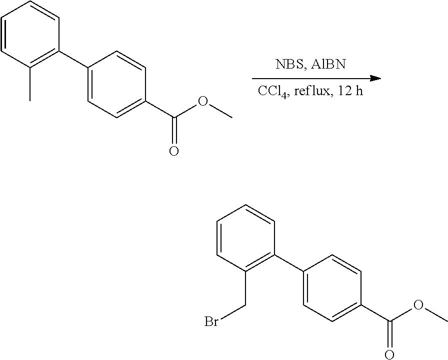

Methyl 2′-methyl-[1,1′-biphenyl]-4-carboxylate (1.1 g, 4.86 mmol), NBS (951 mg, 5.34 mmol), and AIBN (239 mg, 1.45 mmol) were dissolved in CCl₄ (10 ml) in a 50 mL flask, followed by reflux-stirring for 12 hours. The reaction mixture was concentrated under reduced pressure. The organic layer was separated by using dichloromethane and brine. The separated organic layer was dried over MgSO₄ and filtered. The mixture was separated by column chromatography to give methyl 2′-(bromomethyl)-[1,1′-biphenyl]-4-carboxylate (1 g).

### Preparative Example 9

### 4-((2′-(tert-butoxycarbonyl)-2-fluoro-[1,1′-biphenyl]-4-yl)methyl)-3,4-dihydro-2H-benzo[b][1,4]oxazine-7-carboxylic acid

### Step 1: tert-butyl 2-bromobenzoate

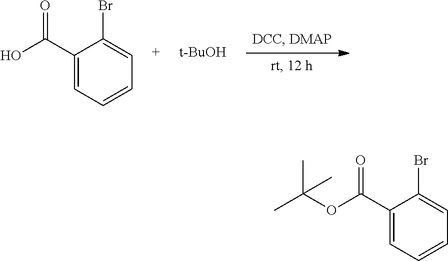

2-bromobenzoic acid (25 g, 124 mmol), DMAP (1.52 g, 12.4 mmol), and t-butanol (9.2 g, 124 mmol) were dissolved in dichloromethane (500 ml) in a 1 L flask, to which DCC (25.7 g, 124 mmol) was added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give tert-butyl 2-bromobenzoate (30 g).

### Step 2 tert-butyl 2′-fluoro-4′-methyl-[1,1′-biphenyl]-2-carboxylate

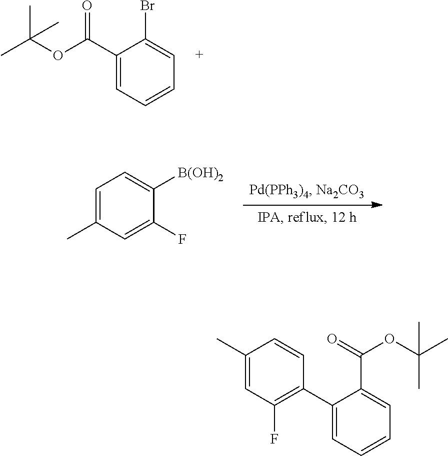

Tert-butyl 2-bromobenzoate (7 g, 27 mmol), (2-fluoro-4-methylphenyl)boronic acid (5 g, 32.5 mmol), Pd(PPh₃)₄(312 mg, 0.27 mmol), and Na₂CO₃ (5.7 g, 54 mmol) were dissolved in IPA (30 ml) and H₂O (30 ml) in a 1 L flask, followed by reflux-stirring for 12 hours. The reaction mixture was concentrated under reduced pressure. The organic layer was separated by using ethyl acetate and brine. The separated organic layer was dried over MgSO₄ and filtered. The mixture was separated by column chromatography to give tert-butyl 2′-fluoro-4′-methyl-[1,1′-biphenyl]-2-carboxylate (5.2 g).

### Step 3 tert-butyl 4′-(bromomethyl)-2′-fluoro-[1,1′-biphenyl]-2-carboxylate

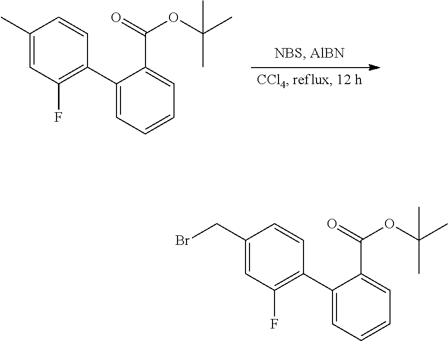

Tert-butyl 2′-fluoro-4′-methyl-[1,1′-biphenyl]-2-carboxylate (3 g, 10.5 mmol), NBS (1.9 g, 10.5 mmol), and AIBN (43 mg, 0.025 mmol) were dissolved in CCl₄ (10 ml) in a 1 L flask, followed by reflux-stirring for 12 hours. The reaction mixture was concentrated under reduced pressure. The organic layer was separated by using ethyl acetate and brine. The separated organic layer was dried over MgSO₄ and filtered. The mixture was separated by column chromatography to give tert-butyl 4′-(bromomethyl)-2′-fluoro-[1,1′-biphenyl]-2-carboxylate (2.75 g).

### Step 4: methyl 4-((2′-(tert-butoxycarbonyl)-2-fluoro-[1,1′-biphenyl]-4-yl)methyl)-3,4-dihydro-2H-benzo[b][1, 4]oxazine-7-carboxylate

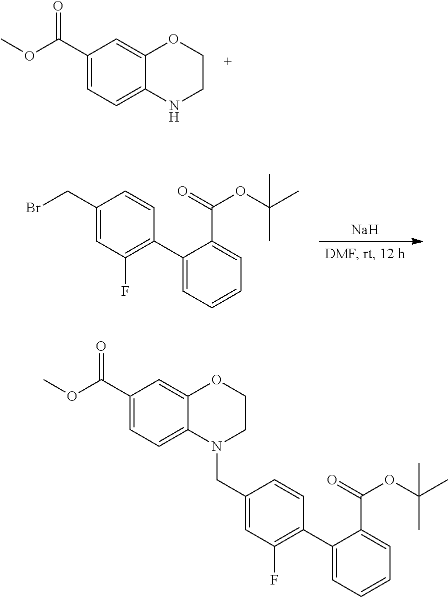

Methyl 3,4-dihydro-2H-benzo[b][1,4]oxazine-7-carboxylate (10 g, 51.7 mmol) was dissolved in DMF (50 ml) in a 250 mL flask, to which NaH (6.2 g, 155.3 mmol) and tert-butyl 4′-(bromomethyl)-2′-fluoro-[1,1′-biphenyl]-2-carboxylate (2.5 g, 67.3 mmol) were added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give methyl 4-((2′-(tert-butoxycarbonyl)-2-fluoro-[1,1′-biphenyl]-4-yl)methyl)-3,4-dihydro-2H-benzo[b][1,4]oxazine-7-carboxylate (15.4 g).

### Step 5: 4-((2′-(tert-butoxycarbonyl)-2-fluoro-[1,1′-biphenyl]-4-yl)methyl)-3,4-dihydro-2H-benzo[b][1,4]oxazine-7-carboxylic acid

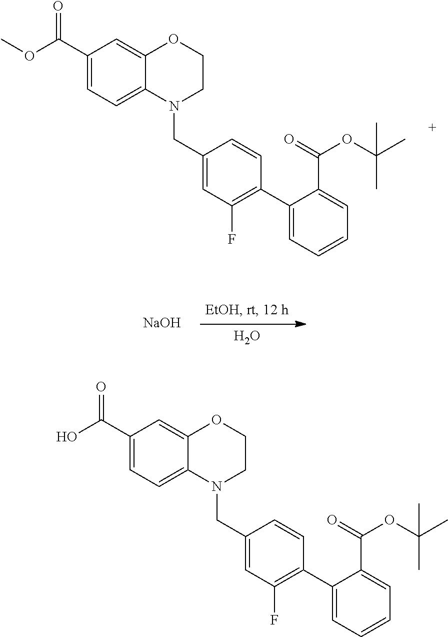

Methyl 4-((2′-(tert-butoxycarbonyl)-2-fluoro-[1,1′-biphenyl]-4-yl)methyl)-3,4-dihydro-2H-benzo[b][1,4]oxazine-7-carboxylate (15 g) was mixed with NaOH (1.48 g, 37 mmol) dissolved in EtOH (50 ml) and H₂O (50 ml) in a 250 mL flask, followed by stirring at room temperature for 12 hours. Upon completion of the reaction, the solvent was concentrated. The pH of the reaction mixture was adjusted to 5 by using 2N-HCl. The mixture was extracted with EA. The organic layer was dried over MgSO₄ and filtered. As a result, 4-((2′-(tert-butoxycarbonyl)-2-fluoro-[1,1′-biphenyl]-4-yl)methyl)-3,4-dihydro-2H-benzo[b][1,4]oxazine-7-carboxylic acid was obtained (13 g).

### Example 1

### (S)-4′-((6-((1-phenylethyl)carbamoyl)-3,4-dihydroquinoline-1(2H)-yl)methyl)-[1,1′-biphenyl]-2-carboxylic acid

### Step 1: (S)-tert-butyl 4′-((6-((1-phenylethyl)carbamoyl)-3,4-dihydroquinoline-1(2H)-yl)methyl)-[1,1′-biphenyl]-2-carboxylate

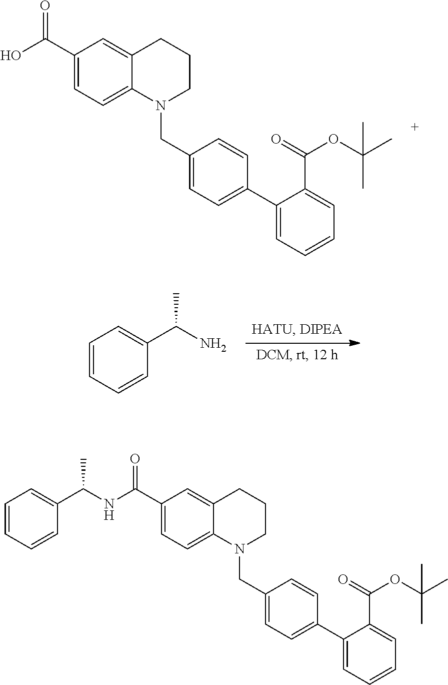

1-((2′-(tert-butoxycarbonyl)-[1,1′-biphenyl]-4-yl)methyl)-1,2,3,4-tetrahydroquinoline-6-carboxylic acid (300 mg, 0.67 mmol) obtained in Preparative Example 1 was dissolved in CH₂Cl₂ (10 ml) in a 100 mL flask, to which HATU (280 mg, 0.74 mmol) and DIPEA (350 ul, 2.01 mmol) were added, followed by stirring at room temperature for 12 hours. Upon completion of the reaction, the organic layer was separated by using CH₂Cl₂ and H₂O. The separated organic layer was dried over MgSO₄ and filtered. The mixture was separated by column chromatography (EA/n-Hex=1:1) to give (S)-tert-butyl 4′-((6-((1-phenylethyl)carbamoyl)-3,4-dihydroquinoline-1(2H)-yl)methyl)-[1,1′-biphenyl]-2-carboxylate (311 mg, 0.56 mmol, 85%).

¹H NMR (400 MHz, CDCl₃) δ 7.79 (dd, J=1.2, 8.0 Hz, 1H), 7.52-7.23 (m, 14H), 6.51 (d, J=8.8 Hz, 1H), 6.14 (d, J=8.0 Hz, 1H), 5.34 (m, 1H), 4.60 (s, 2H), 3.48 (t, J=5.6 Hz, 2H), 2.86 (t, J=6.0 Hz, 2H), 2.05 (m, 2H), 1.59 (d, J=6.8 Hz, 3H), 1.26 (s, 9H).

### Step 2 (S)-4′-((6-((1-phenylethyl)carbamoyl)-3,4-dihydroquinoline-1(2H)-yl)methyl)-[1,1′-biphenyl]-2-carboxylic acid

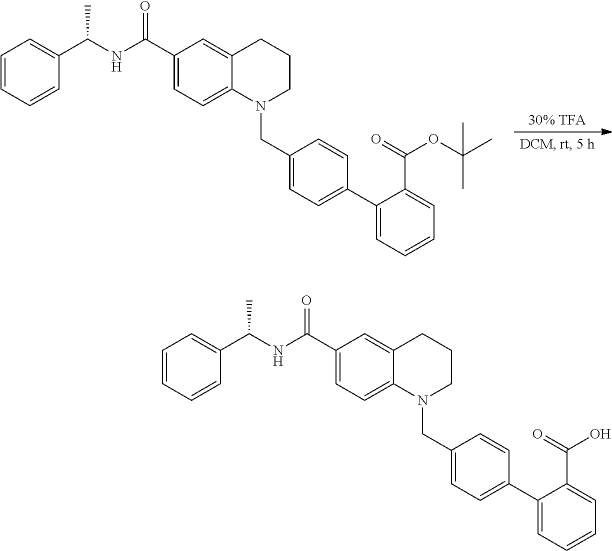 [SMILES: C[C@H](NC(=O)c1ccc2c(c1)CCCN2Cc1ccc(-c2ccccc2C(=O)OC(C)(C)C)cc1)c1ccccc1 ; "30% TFA" ; "DCM, rt, 5 h" ; C[C@H](NC(=O)c1ccc2c(c1)CCCN2Cc1ccc(-c2ccccc2C(=O)O)cc1)c1ccccc1]

(S)-tert-butyl 4′-((6-((1-phenylethyl)carbamoyl)-3,4-dihydroquinoline-1(2H)-yl)methyl)-[1,1′-biphenyl]-2-carboxylate (311 mg, 0.57 mmol) obtained in step 1 was dissolved in CH₂Cl₂ (20 ml) in a 50 mL flask, to which 30% TFA (9 ml) was added, followed by stirring at room temperature for 5 hours. Upon completion of the reaction, the reaction mixture was concentrated under reduced pressure. The mixture was separated by column chromatography (EA) to give (S)-4′-((6-((1-phenylethyl)carbamoyl)-3,4-dihydroquinoline-1(2H)-yl)methyl)-[1,1′-biphenyl]-2-carboxylic acid (176 mg, 0.53 mmol, 63%).

¹H NMR (400 MHz, DMSO-d₆) δ 12.73 (br, OH, 1H), 8.32 (d, J=8.0 Hz, 1H), 7.69 (d, J=8.0 Hz, 1H), 7.60-7.48 (m, 3H), 7.43 (t, J=7.6 Hz, 1H), 7.40-7.25 (m, 9H), 7.19 (t, J=7.2 Hz, 1H), 6.52 (d, J=8.4 Hz, 1H), 5.12 (m, 1H), 4.69 (s, 2H), 3.49 (t, J=4.0 Hz, 2H), 2.81 (t, J=4.0 Hz, 2H), 1.95 (m, 2H), 1.42 (d, J=7.2 Hz, 3H).

### Example 2

### (S)-1-((2′-carbamoyl-[1,1′-biphenyl]-4-yl)methyl)-N-(1-phenylethyl)-1,2,3,4-tetrahydroquinoline-6-carboxamide

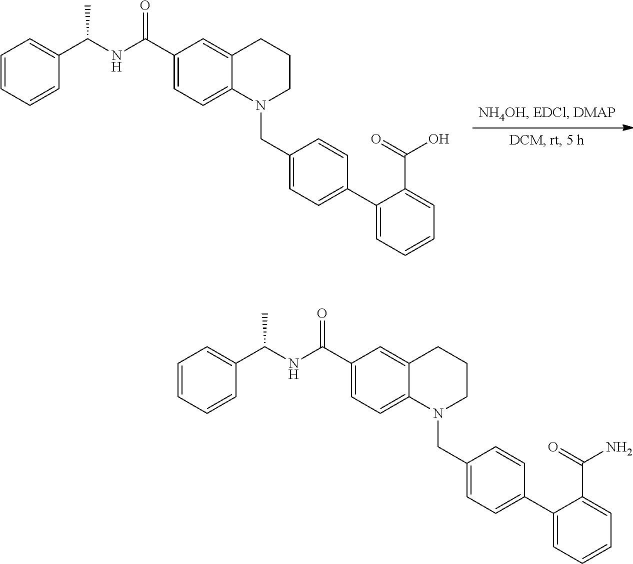 [SMILES: C[C@H](NC(=O)c1ccc2c(c1)CCCN2Cc1ccc(-c2ccccc2C(=O)O)cc1)c1ccccc1 ; "DCM, rt, 5 h" ; "NH4OH, EDCl, DMAP" ; C[C@H](NC(=O)c1ccc2c(c1)CCCN2Cc1ccc(-c2ccccc2C(N)=O)cc1)c1ccccc1]

(S)-4′-((6-((1-phenylethyl)carbamoyl)-3,4-dihydroquinoline-1(2H)-yl)methyl)-[1,1′-biphenyl]-2-carboxylic acid (50 mg, 0.1 mmol) obtained in Example 1 was dissolved in CH₂Cl₂ (2 ml) in a 25 mL flask, to which EDCI (38 mg, 0.2 mmol) and HOBt (38 mg, 0.3 mmol) were added, followed by stirring. Ammonium hydroxide solution (2 ml) was added thereto, followed by stirring at room temperature for 5 hours. Upon completion of the reaction, the organic layer was separated by using CH₂Cl₂ and H₂O. The separated organic layer was dried over MgSO₄ and filtered. The mixture was separated by column chromatography (MeOH/n-Hex/CH₂Cl₂=0.5:0.5:9) to give (S)-1-((2′-carbamoyl-[1,1′-biphenyl]-4-yl)methyl)-N-(1-phenylethyl)-1,2,3,4-tetrahydroquinoline-6-carboxamide (21 mg, 0.04 mmol, 43%).

¹H NMR (400 MHz, DMSO-d₆) δ 8.31-8.29 (m, 1H), 7.66 (m, 1H), 7.52-7.18 (m, 16H), 6.53-6.50 (m, 1H), 5.14-5.10 (m, 1H), 4.61 (s, 2H), 3.48-3.45 (m, 2H), 2.81-2.78 (m, 2H), 1.99-1.95 (m, 2H), 1.44-1.42 (m, 3H).

### Example 3

### (S)-4′-((6-((1-(4-nitrophenyl)ethyl)carbamoyl)-3,4-dihydroquinoline-1(2H)-yl)methyl)-[1,1′-biphenyl]-2-caboxylic acid

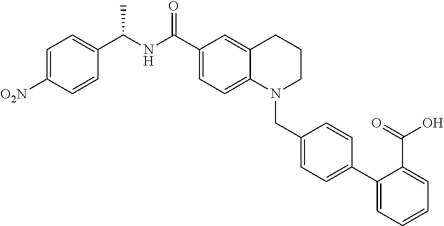 [SMILES: C[C@H](NC(=O)c1ccc2c(c1)CCCN2Cc1ccc(-c2ccccc2C(=O)O)cc1)c1ccc([N+](=O)[O-])cc1]

The target compound was obtained by the same manner as described in Example 1 except that (S)-1-(4-nitrophenyl)ethaneamine was used instead of (S)-1-phenylethylamine.

¹H NMR (400 MHz, DMSO-d₆) δ 12.76 (br, OH, 1H), 8.49 (d, J=7.6 Hz, 1H), 8.19 (d, J=8.8 Hz, 2H), 7.71 (m, 1H), 7.62 (d, J=8.8 Hz, 2H), 7.57-7.25 (m, 9H), 6.52 (d, J=8.0 Hz, 1H), 5.19 (m, 1H), 4.64 (s, 2H), 3.49 (t, J=4.0 Hz, 2H), 2.71 (t, J=4.0 Hz, 2H), 1.95 (m, 2H), 1.47 (d, J=7.2 Hz, 3H).

### Example 4

### (S)-4′-((6-((1-(4-fluoro-2-methylphenyl)ethyl)carbamoyl)-3,4-dihydroquinoline-1(2H)-yl)methyl)-[1,1′-biphenyl]-2-carboxylic acid

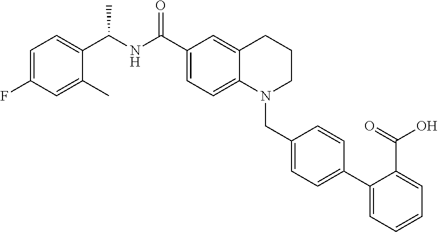 [SMILES: Cc1cc(F)ccc1[C@H](C)NC(=O)c1ccc2c(c1)CCCN2Cc1ccc(-c2ccccc2C(=O)O)cc1]

The target compound was obtained by the same manner as described in Example 1 except that (S)-1-(4-fluoro-2-methylphenyl)ethaneamine was used instead of (S)-1-phenylethylamine.

¹H NMR (400 MHz, DMSO-d₆) δ 12.73 (br, OH, 1H), 8.22 (d, J=8.0 Hz, 1H), 7.79 (d, J=8.0 Hz, 1H), 7.60-7.25 (m, 12H), 6.52 (d, J=8.4 Hz, 1H), 5.12 (m, 1H), 4.69 (s, 2H), 3.49 (t, J=4.0 Hz, 2H), 2.81 (t, J=4.0 Hz, 2H), 2.28 (s, 3H), 1.95 (m, 2H), 1.42 (d, J=7.2 Hz, 3H).

### Example 5

### (S)-4′-((6-((1-(3-fluoro-4-methylphenyl)ethyl)carbamoyl)-3,4-dihydroquinoline-1(2H)-yl)methyl)-[1,1′-biphenyl]-2-caboxylic acid

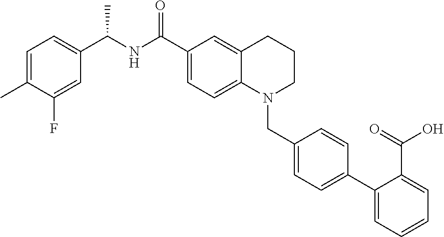 [SMILES: Cc1ccc([C@H](C)NC(=O)c2ccc3c(c2)CCCN3Cc2ccc(-c3ccccc3C(=O)O)cc2)cc1F]

The target compound was obtained by the same manner as described in Example 1 except that (S)-1-(3-fluoro-4-methylphenyl)ethaneamine was used instead of (S)-1-phenylethylamine.

¹H NMR (400 MHz, DMSO-d₆) δ 12.63 (br, OH, 1H), 8.29 (d, J=8.0 Hz, 1H), 7.70 (dd, J=1.2, 7.6 Hz, 1H), 7.57-7.48 (m, 3H), 7.43 (td, J=1.2, 7.6 Hz, 1H), 7.36 (d, J=7.6 Hz, 1H), 7.30 (m, 3H), 7.19 (t, J=7.2 Hz, 1H), 7.11-7.05 (m, 2H), 6.53 (d, J=8.4 Hz, 1H), 5.09 (m, 1H), 4.62 (s, 2H), 3.47 (t, J=5.2 Hz, 2H), 2.80 (t, J=6.0 Hz, 2H), 2.18 (s, 3H), 1.95 (m, 2H), 1.41 (d, J=7.2 Hz, 3H).

### Example 6

### (S)-4′-((6-((1-(4-fluorophenyl)ethyl)carbamoyl)-3,4-dihydroquinoline-1(2H)-yl)methyl)-[1,1′-biphenyl]-2-caboxylic acid

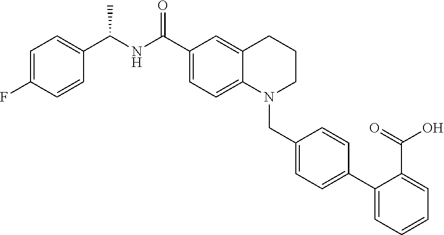 [SMILES: C[C@H](NC(=O)c1ccc2c(c1)CCCN2Cc1ccc(-c2ccccc2C(=O)O)cc1)c1ccc(F)cc1]

The target compound was obtained by the same manner as described in Example 1 except that (S)-1-(4-fluorophenyl)ethaneamine was used instead of (S)-1-phenylethylamine.

¹H NMR (400 MHz, DMSO-d₆) δ 12.63 (br, OH, 1H), 8.34 (d, J=8.0 Hz, 1H), 7.71 (dd, J=1.2, 7.6 Hz, 1H), 7.57-7.35 (m, 7H), 7.30 (m, 4H), 7.13 (m, 2H), 6.52 (d, J=8.8 Hz, 1H), 5.12 (m, 1H), 4.62 (s, 2H), 3.47 (t, J=5.2 Hz, 2H), 2.80 (t, J=6.0 Hz, 2H), 1.95 (m, 2H), 1.41 (d, J=7.2 Hz, 3H).

### Example 7

### (S)-4′-((6-((1-(4-bromophenyl)ethyl)carbamoyl)-3,4-dihydroquinoline-1(2H)-yl)methyl)-[1,1′-biphenyl]-2-caboxylic acid

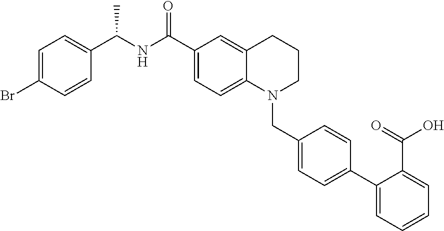 [SMILES: C[C@H](NC(=O)c1ccc2c(c1)CCCN2Cc1ccc(-c2ccccc2C(=O)O)cc1)c1ccc(Br)cc1]

The target compound was obtained by the same manner as described in Example 1 except that (S)-1-(4-bromophenyl)ethaneamine was used instead of (S)-1-phenylethylamine.

¹H NMR (400 MHz, DMSO-d₆) δ 12.71 (br, OH, 1H), 8.34 (d, J=8.0 Hz, 1H), 7.71 (m, 1H), 7.57-7.35 (m, 7H), 7.30 (m, 6H), 6.52 (d, J=8.8 Hz, 1H), 5.12 (m, 1H), 4.62 (s, 2H), 3.47 (t, J=5.2 Hz, 2H), 2.80 (t, J=6.0 Hz, 2H), 1.95 (m, 2H), 1.41 (d, J=7.2 Hz, 3H).

### Example 8

### 4′-((6-((4-nitrobenzyl)carbamoyl)-3,4-dihydroquinoline-1(2H)-yl)methyl)-[1,1′-biphenyl]-2-carboxylic acid

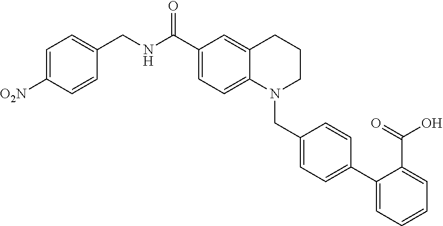 [SMILES: O=C(NCc1ccc([N+](=O)[O-])cc1)c1ccc2c(c1)CCCN2Cc1ccc(-c2ccccc2C(=O)O)cc1]

The target compound was obtained by the same manner as described in Example 1 except that (4-nitrophenyl)methaneamine was used instead of (S)-1-phenylethylamine.

¹H NMR (400 MHz, DMSO-d₆) δ 12.63 (br, OH, 1H), 8.75 (t, J=6.0 Hz, 1H), 8.19 (d, J=7.6 Hz, 2H), 7.70 (dd, J=1.2, 8.0 Hz, 1H), 7.57-7.50 (m, 5H), 7.44 (td, J=1.2, 7.6 Hz, 1H), 7.37 (d, J=7.6 Hz, 1H), 7.30 (m, 4H), 6.54 (d, J=7.2 Hz, 1H), 4.63 (s, 2H), 4.54 (d, J=6.0 Hz, 2H), 3.48 (t, J=5.6 Hz, 2H), 2.80 (t, J=6.0 Hz, 2H), 1.98 (m, 2H).

### Example 9

### (S)-4′-((6-((1-(3-chlorophenyl)ethyl)carbamoyl)-3, 4-dihydroquinoline-1(2H)-yl)methyl)-[1,1′-biphenyl]-2-caboxylic acid

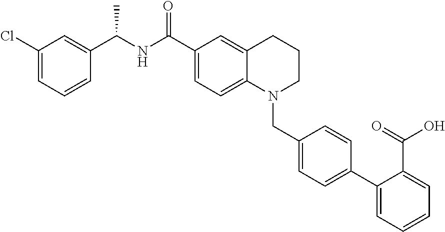 [SMILES: C[C@H](NC(=O)c1ccc2c(c1)CCCN2Cc1ccc(-c2ccccc2C(=O)O)cc1)c1cccc(Cl)c1]

The target compound was obtained by the same manner as described in Example 1 except that (S)-1-(3-chlorophenyl)ethaneamine was used instead of (S)-1-phenylethylamine.

¹H NMR (400 MHz, DMSO-d₆) δ 12.73 (br, OH, 1H), 8.36 (d, J=7.6 Hz, 1H), 7.70 (d, J=6.8 Hz, 1H), 7.60-7.22 (m, 13H), 6.53 (d, J=7.2 Hz, 1H), 5.11 (m, 1H), 4.62 (s, 2H), 3.47 (t, J=5.2 Hz, 2H), 2.80 (t, J=5.6 Hz, 2H), 1.95 (m, 2H), 1.43 (d, J=7.2 Hz, 3H).

### Example 10

### (S)-4′-((6-((1-(naphthalene-1-yl)ethyl)carbamoyl)-3,4-dihydroquinoline-1(2H)-yl)methyl)-[1,1′-biphenyl]-2-carboxylic acid

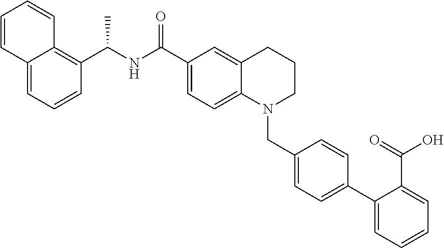 [SMILES: C[C@H](NC(=O)c1ccc2c(c1)CCCN2Cc1ccc(-c2ccccc2C(=O)O)cc1)c1cccc2ccccc12]

The target compound was obtained by the same manner as described in Example 1 except that (S)-1-(naphthalene-1-yl)ethaneamine was used instead of (S)-1-phenylethylamine.

¹H NMR (400 MHz, DMSO-d₆) δ 12.73 (br, OH, 1H), 8.46 (d, J=7.6 Hz, 1H), 8.19 (d, J=7.6 Hz, 1H), 7.81 (d, J=8.0 Hz, 1H), 7.70 (d, J=6.8 Hz, 1H), 7.60-7.35 (m, 10H), 7.28 (m, 4H), 6.52 (d, J=8.8 Hz, 1H), 5.93 (m, 1H), 4.61 (s, 2H), 3.46 (t, J=5.2 Hz, 2H), 2.78 (t, J=5.6 Hz, 2H), 1.95 (m, 2H), 1.57 (d, J=6.8 Hz, 3H).

### Example 11

### (s)-4′-((6-((1-(4-(trifluoromethoxy)phenyl)ethyl)carbamoyl)-3,4-dihydroquinoline-1(2H)-yl)methyl)-[1,1′-biphenyl]-2-carboxylic acid

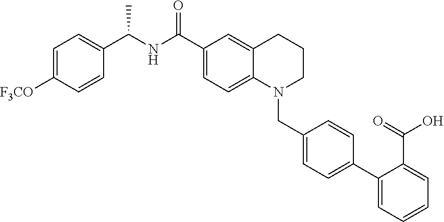 [SMILES: C[C@H](NC(=O)c1ccc2c(c1)CCCN2Cc1ccc(-c2ccccc2C(=O)O)cc1)c1ccc(OC(F)(F)F)cc1]

The target compound was obtained by the same manner as described in Example 1 except that (S)-1-(4-(trifluoromethoxy)phenyl)ethaneamine was used instead of (S)-1-phenylethylamine.

¹H NMR (400 MHz, DMSO-d₆) δ 12.73 (br, OH, 1H), 8.37 (d, J=7.6 Hz, 1H), 7.69 (d, J=8.0 Hz, 1H), 7.60-7.40 (m, 7H), 7.40-7.25 (m, 6H), 6.52 (d, J=8.8 Hz, 1H), 5.14 (m, 1H), 4.62 (s, 2H), 3.58 (t, J=5.2 Hz, 2H), 2.79 (t, J=6.0 Hz, 2H), 1.95 (m, 2H), 1.44 (d, J=6.8 Hz, 3H).

### Example 12

### (S)-4′-((6-((1-(4-(trifluoromethyl)phenyl)ethyl)carbamoyl)-3,4-dihydroquinoline-1(2H)-yl)methyl)-[1,1′-biphenyl]-2-carboxylic acid

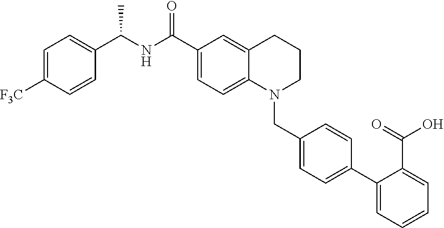 [SMILES: C[C@H](NC(=O)c1ccc2c(c1)CCCN2Cc1ccc(-c2ccccc2C(=O)O)cc1)c1ccc(C(F)(F)F)cc1]

The target compound was obtained by the same manner as described in Example 1 except that (S)-1-(4-(trifluoromethyl)phenyl)ethaneamine was used instead of (S)-1-phenylethylamine.

¹H NMR (400 MHz, DMSO-d₆) δ 12.66 (br, OH, 1H), 8.43 (d, J=8.0 Hz, 1H), 7.75-7.65 (m, 3H), 7.60-7.50 (m, 5H), 7.44 (t, J=7.6 Hz, 1H), 7.37 (d, J=7.2 Hz, 1H), 7.30-7.20 (m, 4H), 6.53 (d, J=8.8 Hz, 1H), 5.16 (m, 1H), 4.62 (s, 2H), 3.47 (t, J=5.2 Hz, 2H), 2.81 (t, J=6.0 Hz, 2H), 1.95 (m, 2H), 1.46 (d, J=6.8 Hz, 3H).

### Example 13

### (S)-4′-((6-((1-(4-((trifluoromethyl)thio)phenyl)ethyl)carbamoyl)-3,4-dihydroquinoline-1 (2H)-yl)methyl)-[1,1′-biphenyl]-2-carboxylic acid

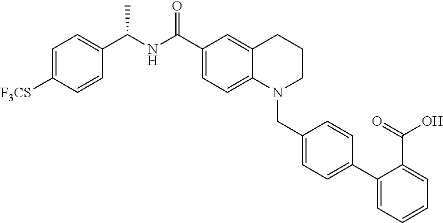

The target compound was obtained by the same manner as described in Example 1 except that (S)-1-(4-((trifluoromethyl)thio)phenyl)ethaneamine was used instead of (S)-1-phenylethylamine.

¹H NMR (400 MHz, DMSO-d₆) δ 12.83 (br, OH, 1H), 8.41 (d, J=7.6 Hz, 1H), 7.94 (d, J=8.0 Hz, 1H), 7.70-7.40 (m, 11H), 7.40-7.25 (m, 6H), 6.53 (d, J=8.4 Hz, 1H), 5.14 (m, 1H), 4.62 (s, 2H), 3.47 (t, J=5.6 Hz, 2H), 2.80 (t, J=5.6 Hz, 2H), 1.95 (m, 2H), 1.45 (d, J=6.8 Hz, 3H).

### Example 14

### (S)-4′-((6-((1-(4-cyanophenyl)ethyl)carbamoyl)-3,4-dihydroquinoline-1(2H)-yl)methyl)-[1,1′-biphenyl]-2-caboxylic acid

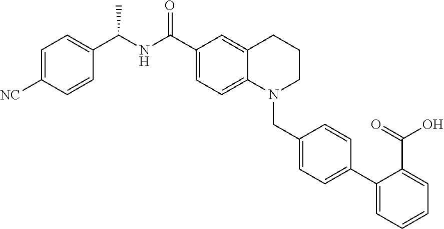 [SMILES: C[C@H](NC(=O)c1ccc2c(c1)CCCN2Cc1ccc(-c2ccccc2C(=O)O)cc1)c1ccc(C#N)cc1]

The target compound was obtained by the same manner as described in Example 1 except that (S)-1-(4-cyanophenyl)ethaneamine was used instead of (S)-1-phenylethylamine.

¹H NMR (400 MHz, DMSO-d₆) δ 12.66 (br, OH, 1H), 8.42 (d, J=7.6 Hz, 1H), 7.87 (d, J=7.0 Hz, 2H), 7.69 (d, J=7.6 Hz, 1H), 7.58-7.49 (m, 5H), 7.43 (t, J=7.6 Hz, 1H), 7.38 (d, J=7.2 Hz, 1H), 7.32-7.25 (m, 4H), 6.53 (d, J=8.4 Hz, 1H), 5.15 (m, 1H), 4.62 (s, 2H), 3.47 (t, J=5.2 Hz, 2H), 2.80 (t, J=6.0 Hz, 2H), 1.96 (m, 2H), 1.44 (d, J=7.2 Hz, 3H).

### Example 15

### 4′-((6-(cromene-3-ylcarbamoyl)-3,4-dihydroquinoline-1(2H)-yl)methyl)-[1,1′-biphenyl]-2-carboxylic acid

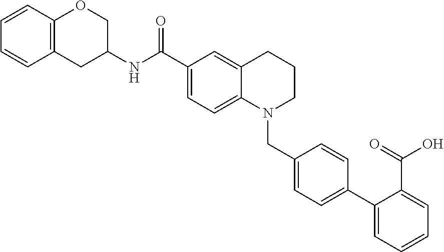 [SMILES: O=C(NC1COc2ccccc2C1)c1ccc2c(c1)CCCN2Cc1ccc(-c2ccccc2C(=O)O)cc1]

The target compound was obtained by the same manner as described in Example 1 except that cromene-3-amine was used instead of (S)-1-phenylethylamine.

¹H NMR (400 MHz, DMSO-d₆) δ 12.62 (br, OH, 1H), 7.97 (m, 1H), 7.71 (m, 1H), 7.60-7.40 (m, 4H), 7.40-7.33 (m, 1H), 7.26 (m, 4H), 7.09 (m, 2H), 6.84 (t, J=8.0 Hz, 1H), 6.79 (d, J=8.0 Hz, 1H), 6.54 (d, J=8.4 Hz, 1H), 4.62 (s, 2H), 4.30 (m, 1H), 4.18 (m, 1H), 3.82 (m, 1H), 3.47 (t, J=5.6 Hz, 2H), 3.05-2.89 (m, 2H), 2.80 (t, J=5.6 Hz, 2H), 1.97 (m, 2H).

### Example 16

### (S)-4′-((6-((1-(2,6-fluorophenyl)ethyl) carbamoyl)-3,4-dihydroquinoline-1 (2H)-yl)methyl)-[1,1′-biphenyl]-2-carboxylic acid

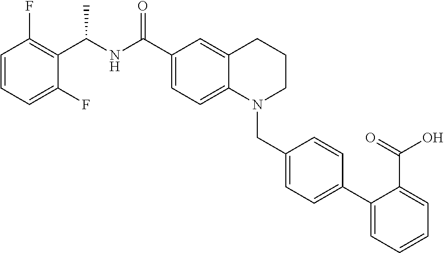 [SMILES: C[C@H](NC(=O)c1ccc2c(c1)CCCN2Cc1ccc(-c2ccccc2C(=O)O)cc1)c1c(F)cccc1F]

The target compound was obtained by the same manner as described in Example 1 except that (S)-1-(2,6-difluorophenyl)ethaneamine was used instead of (S)-1-phenylethylamine.

¹H NMR (400 MHz, DMSO-d₆) δ 12.73 (br, OH, 1H), 8.26 (d, J=6.8 Hz, 1H), 7.69 (d, J=7.6 Hz, 1H), 7.58-7.40 (m, 4H), 7.36 (d, J=7.6 Hz, 1H), 7.31-7.23 (m, 5H), 7.00 (t, J=8.0 Hz, 1H), 6.51 (d, J=8.8 Hz, 1H), 5.35 (m, 1H), 4.61 (s, 2H), 3.47 (t, J=5.2 Hz, 2H), 2.78 (t, J=5.6 Hz, 2H), 1.97 (m, 2H), 1.53 (d, J=6.8 Hz, 3H).

### Example 17

### (S)-4′-((6-((1-(3-fluorophenyl)ethyl)carbamoyl)-3, 4-dihydroquinoline-1(2H)-yl)methyl)-[1,1′-biphenyl]-2-caboxylic acid

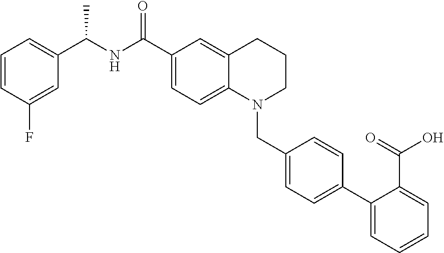 [SMILES: C[C@H](NC(=O)c1ccc2c(c1)CCCN2Cc1ccc(-c2ccccc2C(=O)O)cc1)c1cccc(F)c1]

The target compound was obtained by the same manner as described in Example 1 except that (S)-1-(3-fluorophenyl)ethaneamine was used instead of (S)-1-phenylethylamine.

¹H NMR (400 MHz, DMSO-d₆) δ 12.81 (br, OH, 1H), 8.32 (d, J=7.6 Hz, 1H), 7.70 (d, J=7.6 Hz, 1H), 7.58-7.50 (m, 3H), 7.44 (t, J=7.2 Hz, 1H), 7.38-7.25 (m, 6H), 7.18 (m, 1H), 7.02 (m, 1H), 6.54 (d, J=8.8 Hz, 1H), 5.13 (m, 1H), 4.62 (s, 2H), 3.45 (t, J=5.2 Hz, 2H), 2.80 (t, J=5.6 Hz, 2H), 1.97 (m, 2H), 1.44 (d, J=6.8 Hz, 3H).

### Example 18

### (S)-4′-((6-((1-(3-(trifluoromethoxy)phenyl)ethyl) carbamoyl)-3,4-dihydroquinoline-1(2H)-yl)methyl)-[1,1′-biphenyl]-2-carboxylic acid

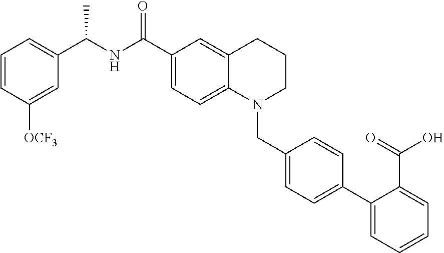 [SMILES: C[C@H](NC(=O)c1ccc2c(c1)CCCN2Cc1ccc(-c2ccccc2C(=O)O)cc1)c1cccc(OC(F)(F)F)c1]

The target compound was obtained by the same manner as described in Example 1 except that (S)-1-(3-(trifluoromethoxy)phenyl)ethaneamine was used instead of (S)-1-phenylethylamine.

¹H NMR (400 MHz, DMSO-d₆) δ 12.73 (br, OH, 1H), 8.37 (d, J=8.0 Hz, 1H), 7.70 (d, J=7.6 Hz, 1H), 7.58-7.46 (m, 3H), 7.44 (t, J=7.2 Hz, 2H), 7.38 (t, J=7.6 Hz, 2H), 7.35-7.25 (m, 5H), 7.19 (d, J=7.6 Hz, 1H), 6.54 (d, J=8.4 Hz, 1H), 5.15 (m, 1H), 4.62 (s, 2H), 3.47 (t, J=5.2 Hz, 2H), 2.80 (t, J=5.6 Hz, 2H), 1.97 (m, 2H), 1.44 (d, J=6.8 Hz, 3H).

### Example 19

### (S)-4′-((6-((1-(3-(trifluoromethyl)phenyl)ethyl) carbamoyl)-3,4-dihydroquinoline-1(2H)-yl)methyl)-[1,1′-biphenyl]-2-carboxylic acid

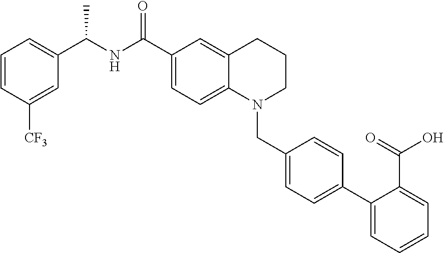 [SMILES: C[C@H](NC(=O)c1ccc2c(c1)CCCN2Cc1ccc(-c2ccccc2C(=O)O)cc1)c1cccc(C(F)(F)F)c1]

The target compound was obtained by the same manner as described in Example 1 except that S)-1-(3-(trifluoromethyl)phenyl)ethaneamine was used instead of (S)-1-phenylethylamine.

¹H NMR (400 MHz, DMSO-d₆) δ 12.81 (br, OH, 1H), 8.42 (d, J=8.0 Hz, 1H), 7.73-7.65 (m, 3H), 7.59-7.50 (m, 5H), 7.4 (td, J=1.2, 7.6 Hz, 1H), 7.39 (d, J=7.2 Hz, 1H), 7.31-7.25 (m, 4H), 6.54 (d, J=7.6 Hz, 1H), 5.19 (m, 1H), 4.62 (s, 2H), 3.47 (t, J=5.2 Hz, 2H), 2.80 (t, J=6.0 Hz, 2H), 1.97 (m, 2H), 1.47 (d, J=6.8 Hz, 3H).

### Example 20

### (S)-4′-((6-((1-(naphthalene-2-yl)ethyl)carbamoyl)-3,4-dihydroquinoline-1(2H)-yl)methyl)-[1,1′-biphenyl]-2-carboxylic acid

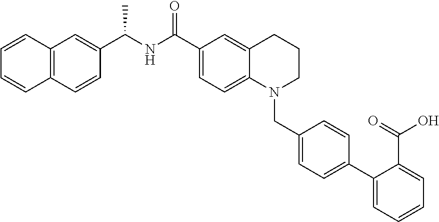 [SMILES: C[C@H](NC(=O)c1ccc2c(c1)CCCN2Cc1ccc(-c2ccccc2C(=O)O)cc1)c1ccc2ccccc2c1]

The target compound was obtained by the same manner as described in Example 1 except that (S)-1-(naphthalene-2-yl)ethaneamine was used instead of (S)-1-phenylethylamine.

¹H NMR (400 MHz, DMSO-d₆) δ 12.73 (br, OH, 1H), 8.41 (d, J=8.0 Hz, 1H), 7.89-7.81 (m, 4H), 7.70 (d, J=7.6 Hz, 1H), 7.60-7.42 (m, 7H), 7.37 (d, J=7.6 Hz, 1H), 7.31-7.25 (m, 4H), 6.54 (d, J=8.8 Hz, 1H), 5.28 (m, 1H), 4.62 (s, 2H), 3.47 (t, J=5.2 Hz, 2H), 2.80 (t, J=5.6 Hz, 2H), 1.97 (m, 2H), 1.54 (d, J=6.8 Hz, 3H).

### Example 21

### (S)-4′-((6-((1-(3,4-fluorophenyl)ethyl)carbamoyl)-3,4-dihydroquinoline-1 (2H)-yl)methyl)-[1,1′-biphenyl]-2-carboxylic acid

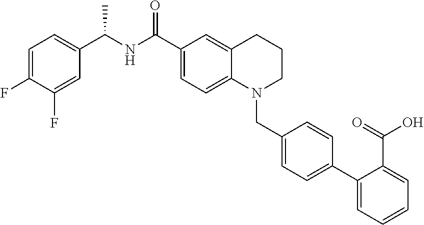 [SMILES: C[C@H](NC(=O)c1ccc2c(c1)CCCN2Cc1ccc(-c2ccccc2C(=O)O)cc1)c1ccc(F)c(F)c1]

The target compound was obtained by the same manner as described in Example 1 except that (S)-1-(3,4-difluorophenyl)ethaneamine was used instead of (S)-1-phenylethylamine.

¹H NMR (400 MHz, DMSO-d₆) δ 12.73 (br, OH, 1H), 8.33 (d, J=8.0 Hz, 1H), 7.70 (d, J=7.6 Hz, 1H), 7.60-7.15 (m, 12H), 6.53 (d, J=8.4 Hz, 1H), 5.10 (m, 1H), 4.62 (s, 2H), 3.47 (t, J=5.2 Hz, 2H), 2.80 (t, J=5.6 Hz, 2H), 1.97 (m, 2H), 1.54 (d, J=6.8 Hz, 3H).

### Example 22

### (S)-4′-((6-((1-(3-methoxyphenyl)ethyl)carbamoyl)-3,4-dihydroquinoline-1(2H)-yl)methyl)-[1,1′-biphenyl]-2-caboxylic acid

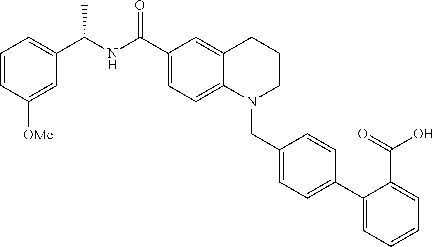 [SMILES: COc1cccc([C@H](C)NC(=O)c2ccc3c(c2)CCCN3Cc2ccc(-c3ccccc3C(=O)O)cc2)c1]

The target compound was obtained by the same manner as described in Example 1 except that (S)-1-(3-methoxyphenyl)ethaneamine was used instead of (S)-1-phenylethylamine.

¹H NMR (400 MHz, DMSO-d₆) δ 12.73 (br, OH, 1H), 8.28 (d, J=8.4 Hz, 1H), 7.70 (dd, J=1.2, 7.6 Hz, 1H), 7.60-7.46 (m, 3H), 7.45 (m, 1H), 7.38 (m, 1H), 7.34-7.19 (m, 5H), 6.95 (m, 2H), 6.78 (m, 1H), 6.52 (d, J=8.4 Hz, 1H), 5.10 (m, 1H), 4.62 (s, 2H), 3.74 (s, 3H), 3.47 (t, J=5.2 Hz, 2H), 2.80 (t, J=5.6 Hz, 2H), 1.97 (m, 2H), 1.42 (d, J=6.8 Hz, 3H).

### Example 23

### (S)-4′-((6-((1-(2-methoxyphenyl)ethyl)carbamoyl)-3,4-dihydroquinoline-1(2H)-yl)methyl)-[1,1′-biphenyl]-2-carboxylic acid

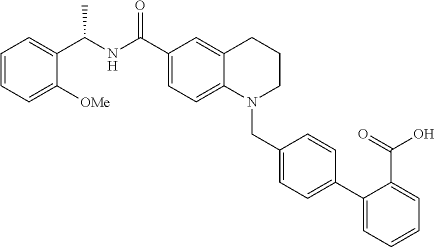 [SMILES: COc1ccccc1[C@H](C)NC(=O)c1ccc2c(c1)CCCN2Cc1ccc(-c2ccccc2C(=O)O)cc1]

The target compound was obtained by the same manner as described in Example 1 except that (S)-1-(2-methoxyphenyl)ethaneamine was used instead of (S)-1-phenylethylamine.

¹H NMR (400 MHz, DMSO-d₆) δ 12.73 (br, OH, 1H), 8.23 (d, J=8.0 Hz, 1H), 7.70 (dd, J=1.2, 7.6 Hz, 1H), 7.60-7.15 (m, 11H), 7.0-6.86 (m, 2H), 6.52 (d, J=8.4 Hz, 1H), 5.39 (m, 1H), 4.62 (s, 2H), 3.82 (s, 3H), 3.47 (t, J=5.2 Hz, 2H), 2.80 (t, J=5.6 Hz, 2H), 1.97 (m, 2H), 1.34 (d, J=7.2 Hz, 3H).

### Example 24

### (S)-4′-((6-((1-(2-fluorophenyl)ethyl)carbamoyl)-3, 4-dihydroquinoline-1(2H)-yl)methyl)-[1,1′-biphenyl]-2-caboxylic acid

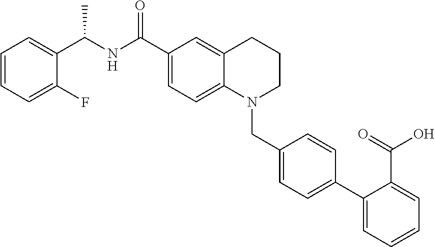 [SMILES: C[C@H](NC(=O)c1ccc2c(c1)CCCN2Cc1ccc(-c2ccccc2C(=O)O)cc1)c1ccccc1F]

The target compound was obtained by the same manner as described in Example 1 except that (S)-1-(2-fluorophenyl)ethaneamine was used instead of (S)-1-phenylethylamine.

¹H NMR (400 MHz, DMSO-d₆) δ 12.73 (br, OH, 1H), 8.31 (d, J=8.0 Hz, 1H), 7.70 (d, J=7.2 Hz, 1H), 7.60-7.10 (m, 13H), 6.53 (d, J=8.8 Hz, 1H), 5.35 (m, 1H), 4.62 (s, 2H), 3.47 (t, J=5.2 Hz, 2H), 2.80 (t, J=5.6 Hz, 2H), 1.97 (m, 2H), 1.42 (d, J=6.8 Hz, 3H).

### Example 25

### (S)-4′-((6-((1-(2-(trifluoromethyl)phenyl)ethyl)carbamoyl)-3,4-dihydroquinoline-1(2)-yl)methyl)-[1,1′-biphenyl]-2-carboxylic acid

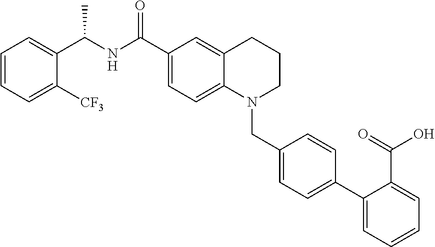 [SMILES: C[C@H](NC(=O)c1ccc2c(c1)CCCN2Cc1ccc(-c2ccccc2C(=O)O)cc1)c1ccccc1C(F)(F)F]

The target compound was obtained by the same manner as described in Example 1 except that (S)-1-(2-(trifluoromethyl)phenyl)ethaneamine was used instead of (S)-1-phenylethylamine.

¹H NMR (400 MHz, DMSO-d₆) δ 12.81 (br, OH, 1H), 8.48 (d, J=7.2 Hz, 1H), 7.92 (d, J=7.6 Hz, 1H), 7.71-7.60 (m, 3H), 7.59-7.35 (m, 6H), 7.32-7.20 (m, 4H), 6.52 (d, J=8.4 Hz, 1H), 5.41 (m, 1H), 4.62 (s, 2H), 3.46 (t, J=5.2 Hz, 2H), 2.80 (t, J=5.6 Hz, 2H), 1.97 (m, 2H), 1.42 (d, J=6.8 Hz, 3H).

### Example 26

### 4′-((6-((pyridine-2-ylmethyl)carbamoyl)-3,4-dihydroquinoline-1(2H)-yl)methyl)-[1,1′-biphenyl]-2-carboxylic acid

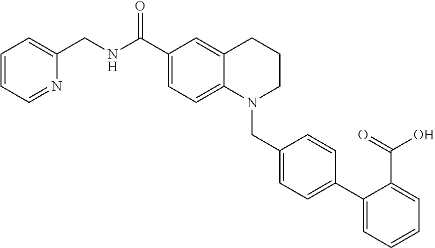 [SMILES: O=C(NCc1ccccn1)c1ccc2c(c1)CCCN2Cc1ccc(-c2ccccc2C(=O)O)cc1]

The target compound was obtained by the same manner as described in Example 1 except that pyridine-2-ylmethaneamine was used instead of (S)-1-phenylethylamine.

¹H NMR (400 MHz, DMSO-d₆) δ 12.75 (br, OH, 1H), 8.68-8.67 (m, 1H), 8.52-8.51 (m, 1H), 7.81-7.77 (m, 1H), 7.70-7.69 (m, 1H), 7.57-7.54 (m, 3H), 7.46-7.42 (m, 1H), 7.38-7.36 (m, 1H), 7.31-7.26 (m, 6H), 4.63 (s, 2H), 4.53-4.52 (m, 2H), 3.34 (m, 2H), 2.80 (m, 2H), 1.97 (m, 2H).

### Example 27

### 4′-((6-((pyridine-4-ylmethyl)carbamoyl)-3,4-dihydroquinoline-1(2H)-yl)methyl)-[1,1′-biphenyl]-2-carboxylic acid

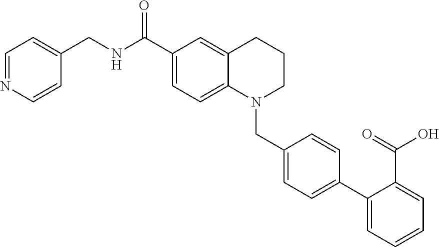 [SMILES: O=C(NCc1ccncc1)c1ccc2c(c1)CCCN2Cc1ccc(-c2ccccc2C(=O)O)cc1]

The target compound was obtained by the same manner as described in Example 1 except that pyridine-4-ylmethaneamine was used instead of (S)-1-phenylethylamine.

¹H NMR (400 MHz, DMSO-d₆) δ 12.75 (br, OH, 1H), 8.74-8.72 (m, 1H), 8.58-8.56 (m, 2H), 7.71-7.69 (m, 1H), 7.57-7.51 (m, 3H), 7.46-7.42 (m, 3H), 7.38-7.36 (m, 1H), 7.31-7.26 (m, 4H), 6.56-6.53 (m, 1H), 4.63 (s, 2H), 4.50-4.48 (m, 2H), 3.49-3.46 (m, 2H), 2.81-2.79 (m, 2H), 1.98-1.95 (m, 2H).

### Example 28

### 4′-((6-(benzylcarbamoyl)-3,4-dihydroquinoline-1(2H)-yl)methyl)-[1,1′-biphenyl]-2-carboxylic acid

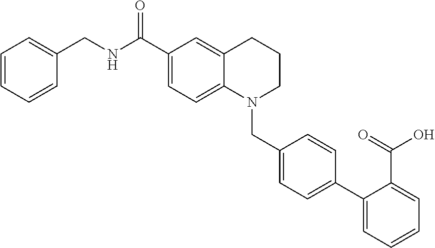 [SMILES: O=C(NCc1ccccc1)c1ccc2c(c1)CCCN2Cc1ccc(-c2ccccc2C(=O)O)cc1]

The target compound was obtained by the same manner as described in Example 1 except that phenylmethaneamine was used instead of (S)-1-phenylethylamine.

¹H NMR (400 MHz, DMSO-d₆) δ 12.75 (br, OH, 1H), 7.71-7.68 (m, 1H), 7.57-7.49 (m, 3H), 7.46-7.42 (m, 1H), 7.38-7.36 (m, 1H), 7.32-7.25 (m, 8H), 7.23-7.19 (m, 1H), 6.53-6.51 (m, 1H), 4.62 (s, 2H), 4.43-4.41 (m, 2H), 3.48-3.45 (m, 2H), 2.81-2.78 (m, 2H), 1.99-1.91 (m, 2H).

### Example 29

### 4′-((6-((2-bromobenzyl) carbamoyl)-3,4-dihydroquinoline-1(2H)-yl)methyl)-[1,1′-biphenyl]-2-carboxylic acid

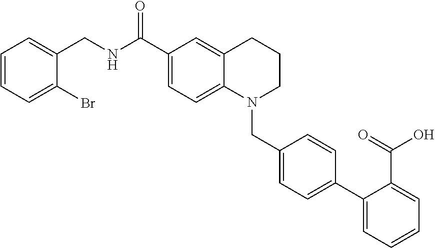 [SMILES: O=C(NCc1ccccc1Br)c1ccc2c(c1)CCCN2Cc1ccc(-c2ccccc2C(=O)O)cc1]

The target compound was obtained by the same manner as described in Example 1 except that (2-bromophenyl)methaneamine was used instead of (S)-1-phenylethylamine.

¹H NMR (400 MHz, DMSO-d₆) δ 8.63-8.60 (m, 1H), 7.65-7.59 (m, 2H), 7.56-7.53 (m, 2H), 7.50-7.47 (m, 1H), 7.41-7.32 (m, 5H), 7.26-7.24 (m, 3H), 7.21-7.18 (m, 1H), 6.56-6.53 (m, 1H), 4.62 (s, 2H), 4.44-4.42 (m, 2H), 3.49-3.47 (m, 2H), 2.82-2.79 (m, 2H), 1.97 (m, 2H).

### Example 30

### 4′-((6-(((2,3-dihydrobenzo[b][1,4]dioxin-6-yl)methyl) carbamoyl)-3,4-dihydroquinoline-1(2H)-yl)methyl)-[1,1′-biphenyl]-2-carboxylic acid

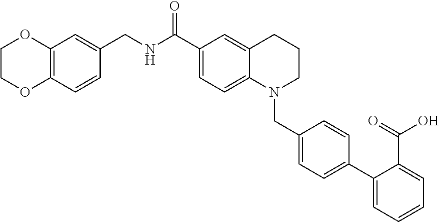 [SMILES: O=C(NCc1ccc2c(c1)OCCO2)c1ccc2c(c1)CCCN2Cc1ccc(-c2ccccc2C(=O)O)cc1]

The target compound was obtained by the same manner as described in Example 1 except that (2,3-dihydrobenzo[b][1,4]dioxin-6-yl)methaneamine was used instead of (S)-1-phenylethylamine.

¹H NMR (400 MHz, DMSO-d₆) δ 12.75 (br, OH, 1H), 8.50-8.47 (m, 1H), 7.56-7.41 (m, 4H), 7.38-7.36 (m, 1H), 7.31-7.25 (m, 4H), 6.83-6.71 (m, 3H), 6.53-6.51 (m, 1H), 4.61 (s, 2H), 4.29-4.28 (m, 2H), 4.19 (s, 4H), 3.47-3.45 (m, 2H), 2.80-2.77 (m, 2H), 1.97-1.94 (m, 2H).

### Example 31

### 4′-((6-((4-bromobenzyl) carbamoyl)-3,4-dihydroquinoline-1 (2H)-yl)methyl)-[1,1′-biphenyl]-2-carboxylic acid

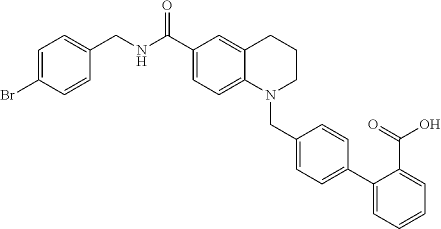 [SMILES: O=C(NCc1ccc(Br)cc1)c1ccc2c(c1)CCCN2Cc1ccc(-c2ccccc2C(=O)O)cc1]

The target compound was obtained by the same manner as described in Example 1 except that (4-bromophenyl)methaneamine was used instead of (S)-1-phenylethylamine.

¹H NMR (400 MHz, CDCl₃) δ 7.94 (d, J=7.2 Hz, 1H), 7.95-7.20 (m, 13H), 6.49 (d, J=8.8 Hz, 1H), 6.25 (s, 1H), 4.59-4.55 (m, 4H), 3.50 (t, J=5.6 Hz, 2H), 2.86 (t, J=6.0 Hz, 2H), 2.07-1.98 (m, 2H).

### Example 32

### 4′-((6-((3-(trifluoromethoxy)benzyl) carbamoyl)-3,4-dihydroquinoline-1(2H)-yl)methyl)-[1,1′-biphenyl]-2-carboxylic acid

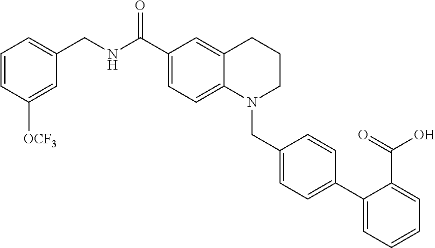 [SMILES: O=C(NCc1cccc(OC(F)(F)F)c1)c1ccc2c(c1)CCCN2Cc1ccc(-c2ccccc2C(=O)O)cc1]

The target compound was obtained by the same manner as described in Example 1 except that (3-(trifluoromethoxy)phenyl)methaneamine was used instead of (S)-1-phenylethylamine.

¹H NMR (400 MHz, CDCl₃) δ 7.91 (s, 1H), 7.60-7.02 (m, 13H), 6.47 (d, J=8.4 Hz, 1H), 6.37 (s, 1H), 4.60-4.55 (m, 4H), 3.51-3.45 (m, 2H), 2.84 (m, 2H), 1.94-1.90 (m, 2H).

### Example 33

### (S)-1-((2′-carbamoyl-[1,1′-biphenyl]-4-yl)methyl)-N-(1-(4-(trifluoromethyl)phenyl)ethyl)-1,2,3,4-tetrahydroquinoline-6-carboxamide

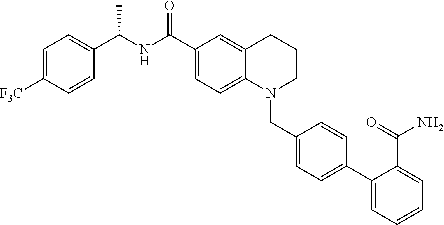 [SMILES: C[C@H](NC(=O)c1ccc2c(c1)CCCN2Cc1ccc(-c2ccccc2C(N)=O)cc1)c1ccc(C(F)(F)F)cc1]

The target compound was obtained by the same manner as described in Example 2 except that (S)-4′-((6-((1-(4-(trifluoromethyl)phenyl)ethyl)carbamoyl)-3,4-dihydroquinoline-1(2H)-yl)methyl)-[1,1′-biphenyl]-2-carboxylic acid obtained in Example 12 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.42 (d, J=7.6 Hz, 1H), 7.68-7.73 (m, 3H), 7.60-7.35 (m, 10H), 7.28-2.22 (m, 3H), 6.53 (d, J=8.8 Hz, 1H), 5.16 (m, 1H), 4.61 (s, 2H), 3.47 (t, J=5.2 Hz, 2H), 2.80 (t, J=5.6 Hz, 2H), 1.97 (m, 2H), 1.46 (d, J=7.2 Hz, 3H).

### Example 34

### (S)-1-((2′-carbamoyl-[1,1′-biphenyl]-4-yl)methyl)-N-(1-(4-(trifluoromethoxy)phenyl)ethyl)-1,2,3,4-tetrahydroquinoline-6-carboxamide

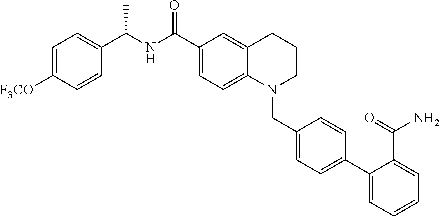 [SMILES: C[C@H](NC(=O)c1ccc2c(c1)CCCN2Cc1ccc(-c2ccccc2C(N)=O)cc1)c1ccc(OC(F)(F)F)cc1]

The target compound was obtained by the same manner as described in Example 2 except that (S)-4′-((6-((1-(4-(trifluoromethoxy)phenyl)ethyl)carbamoyl)-3,4-dihydroquinoline-1(2H)-yl)methyl)-[1,1′-biphenyl]-2-carboxylic acid obtained in Example 11 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.36 (d, J=8.0 Hz, 1H), 7.65 (s, 1H), 7.58-7.23 (m, 15H), 6.52 (d, J=8.4 Hz, 1H), 5.14 (m, 1H), 4.61 (s, 2H), 3.47 (t, J=5.2 Hz, 2H), 2.80 (t, J=5.6 Hz, 2H), 1.97 (m, 2H), 1.44 (d, J=7.2 Hz, 3H).

### Example 35

### (S)-1-((2′-carbamoyl-[1,1′-biphenyl]-4-yl)methyl)-N-(1-(3-chlorophenyl)ethyl)-1,2,3,4-tetrahydroquinoline-6-carboxamide

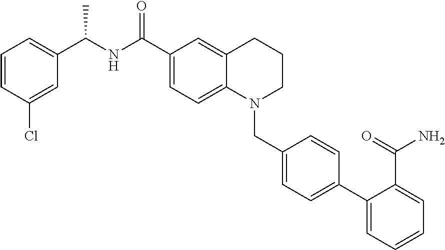 [SMILES: C[C@H](NC(=O)c1ccc2c(c1)CCCN2Cc1ccc(-c2ccccc2C(N)=O)cc1)c1cccc(Cl)c1]

The target compound was obtained by the same manner as described in Example 2 except that (S)-4′-((6-((1-(3-chlorophenyl)ethyl)carbamoyl)-3,4-dihydroquinoline-1(2H)-yl)methyl)-[1,1′-biphenyl]-2-carboxylic acid obtained in Example 9 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.33 (d, J=7.6 Hz, 1H), 7.63 (m, 1H), 7.55-7.25 (m, 15H), 6.54 (d, J=8.4 Hz, 1H), 5.10 (m, 1H), 4.61 (s, 2H), 3.47 (t, J=5.2 Hz, 2H), 2.80 (t, J=5.6 Hz, 2H), 1.97 (m, 2H), 1.43 (d, J=6.8 Hz, 3H).

### Example 36

### (S)-1-((2′-carbamoyl-[1,1′-biphenyl]-4-yl)methyl)-N-(1-(4-fluorophenyl)ethyl)-1,2,3,4-tetrahydroquinoline-6-carboxamide

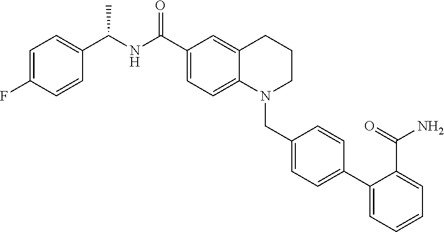 [SMILES: C[C@H](NC(=O)c1ccc2c(c1)CCCN2Cc1ccc(-c2ccccc2C(N)=O)cc1)c1ccc(F)cc1]

The target compound was obtained by the same manner as described in Example 2 except that (S)-4′-((6-((1-(4-fluorophenyl)ethyl)carbamoyl)-3,4-dihydroquinoline-1(2H)-yl)methyl)-[1,1′-biphenyl]-2-carboxylic acid obtained in Example 6 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.39 (d, J=7.6 Hz, 1H), 7.64 (m, 1H), 7.56-7.35 (m, 10H), 7.28-2.22 (m, 3H), 7.15-1.08 (m, 2H), 6.52 (d, J=8.8 Hz, 1H), 5.12 (m, 1H), 4.60 (s, 2H), 3.47 (t, J=5.2 Hz, 2H), 2.79 (t, J=5.6 Hz, 2H), 1.97 (m, 2H), 1.44 (d, J=7.2 Hz, 3H).

### Example 37

### (S)—N-(1-(4-bromophenyl)ethyl)-1-((2′-carbamoyl-[1,1′-biphenyl]-4-yl)methyl)-1,2,3,4-tetrahydroquinoline-6-carboxamide

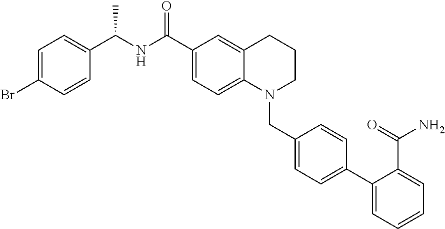 [SMILES: C[C@H](NC(=O)c1ccc2c(c1)CCCN2Cc1ccc(-c2ccccc2C(N)=O)cc1)c1ccc(Br)cc1]

The target compound was obtained by the same manner as described in Example 2 except that (S)-4′-((6-((1-(4-bromophenyl)ethyl)carbamoyl)-3,4-dihydroquinoline-1(2H)-yl)methyl)-[1,1′-biphenyl]-2-carboxylic acid obtained in Example 7 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.31 (d, J=8.0 Hz, 1H), 7.63 (m, 1H), 7.55-7.20 (m, 15H), 6.52 (d, J=8.4 Hz, 1H), 5.08 (m, 1H), 4.61 (s, 2H), 3.47 (t, J=5.2 Hz, 2H), 2.79 (t, J=6.0 Hz, 2H), 1.97 (m, 2H), 1.42 (d, J=7.2 Hz, 3H).

### Example 38

### (S)-1-((2′-carbamoyl-[1,1′-biphenyl]-4-yl)methyl)-N-(1-(naphthalene-1-yl)ethyl)-1,2,3,4-tetrahydroquinoline-6-carboxamide

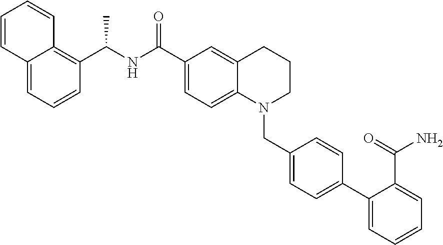 [SMILES: C[C@H](NC(=O)c1ccc2c(c1)CCCN2Cc1ccc(-c2ccccc2C(N)=O)cc1)c1cccc2ccccc12]

The target compound was obtained by the same manner as described in Example 2 except that (S)-4′-((6-((1-(naphthalene-1-yl)ethyl)carbamoyl)-3,4-dihydroquinoline-1(2H)-yl)methyl)-[1,1′-biphenyl]-2-carboxylic acid obtained in Example 10 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.44 (d, J=8.0 Hz, 1H), 8.19 (d, J=8.0 Hz, 1H), 7.93 (m, 1H), 7.68-7.32 (m, 12H), 7.28-7.22 (m, 3H), 6.53 (d, J=8.8 Hz, 1H), 5.92 (m, 1H), 4.60 (s, 2H), 3.46 (t, J=5.2 Hz, 2H), 2.79 (t, J=5.6 Hz, 2H), 1.95 (m, 2H), 1.59 (d, J=6.8 Hz, 3H).

### Example 39

### (S)-1-((2′-carbamoyl-[1,1′-biphenyl]-4-yl)methyl)-N-(1-(3,4-difluorophenyl)ethyl)-1,2,3,4-tetrahydroquinoline-6-carboxamide

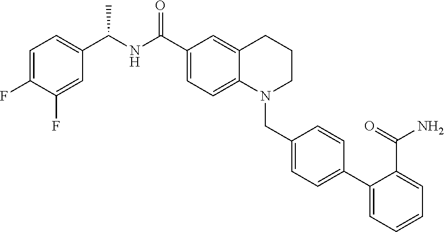 [SMILES: C[C@H](NC(=O)c1ccc2c(c1)CCCN2Cc1ccc(-c2ccccc2C(N)=O)cc1)c1ccc(F)c(F)c1]

The target compound was obtained by the same manner as described in Example 2 except that (S)-4′-((6-((1-(3,4- [symbol] fluorophenyl)ethyl)carbamoyl)-3,4-dihydroquinoline-1(2H)-yl)methyl)-[1,1′-biphenyl]-2-carboxylic acid obtained in Example 21 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.33 (d, J=8.0 Hz, 1H), 7.66 (m, 1H), 7.55-7.15 (m, 13H), 6.52 (d, J=8.8 Hz, 1H), 5.08 (m, 1H), 4.61 (s, 2H), 3.47 (t, J=5.2 Hz, 2H), 2.80 (t, J=6.0 Hz, 2H), 1.97 (m, 2H), 1.42 (d, J=7.2 Hz, 3H).

### Example 40

### (S)-1-((2′-carbamoyl-[1,1′-biphenyl]-4-yl)methyl)-N-(1-(3-methoxyphenyl)ethyl)-1,2,3,4-tetrahydroquinoline-6-carboxamide

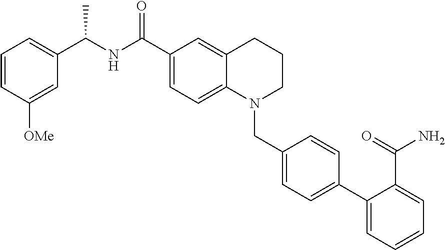 [SMILES: COc1cccc([C@H](C)NC(=O)c2ccc3c(c2)CCCN3Cc2ccc(-c3ccccc3C(N)=O)cc2)c1]

The target compound was obtained by the same manner as described in Example 2 except that (S)-4′-((6-((1-(3-methoxyphenyl)ethyl)carbamoyl)-3,4-dihydroquinoline-1(2H)-yl)methyl)-[1,1′-biphenyl]-2-carboxylic acid obtained in Example 22 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.28 (d, J=7.6 Hz, 1H), 7.65 (m, 1H), 7.55-7.20 (m, 12H), 6.93 (m, 1H), 6.77 (d, J=6.8 Hz, 1H), 6.52 (d, J=8.4 Hz, 1H), 5.09 (m, 1H), 4.61 (s, 2H), 3.73 (s, 3H), 3.47 (t, J=5.2 Hz, 2H), 2.80 (t, J=6.0 Hz, 2H), 1.97 (m, 2H), 1.42 (d, J=6.0 Hz, 3H).

### Example 41

### (S)-1-((2′-carbamoyl-[1,1′-biphenyl]-4-yl)methyl)-N-(1-(2-methoxyphenyl)ethyl)-1,2,3,4-tetrahydroquinoline-6-carboxamide

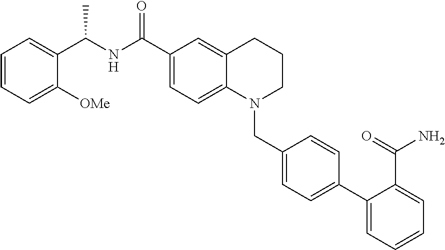 [SMILES: COc1ccccc1[C@H](C)NC(=O)c1ccc2c(c1)CCCN2Cc1ccc(-c2ccccc2C(N)=O)cc1]

The target compound was obtained by the same manner as described in Example 2 except that (S)-4′-((6-((1-(2-methoxyphenyl)ethyl)carbamoyl)-3,4-dihydroquinoline-1(2H)-yl)methyl)-[1,1′-biphenyl]-2-carboxylic acid obtained in Example 23 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.22 (d, J=8.0 Hz, 1H), 7.60 (m, 1H), 7.55-7.25 (m, 13H), 6.95 (d, J=7.6 Hz, 1H), 6.88 (t, J=7.6 Hz, 1H), 6.52 (d, J=8.4 Hz, 1H), 5.39 (m, 1H), 4.61 (s, 2H), 3.82 (s, 3H), 3.47 (t, J=5.2 Hz, 2H), 2.80 (t, J=6.0 Hz, 2H), 1.97 (m, 2H), 1.34 (d, J=7.2 Hz, 3H).

### Example 42

### (S)-1-((2′-carbamoyl-[1,1′-biphenyl]-4-yl)methyl)-N-(1-(2-fluorophenyl)ethyl)-1,2,3,4-tetrahydroquinoline-6-carboxamide

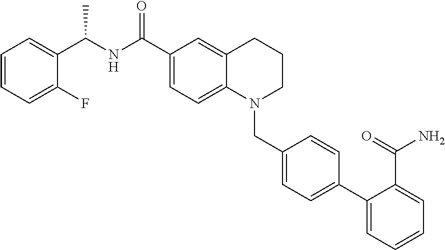 [SMILES: C[C@H](NC(=O)c1ccc2c(c1)CCCN2Cc1ccc(-c2ccccc2C(N)=O)cc1)c1ccccc1F]

The target compound was obtained by the same manner as described in Example 2 except that (S)-4′-((6-((1-(2-fluorophenyl)ethyl)carbamoyl)-3,4-dihydroquinoline-1(2H)-yl)methyl)-[1,1′-biphenyl]-2-carboxylic acid obtained in Example 24 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.37 (d, J=8.0 Hz, 1H), 7.63 (s, 1H), 7.55-7.10 (m, 15H), 6.52 (d, J=8.8 Hz, 1H), 5.35 (m, 1H), 4.61 (s, 2H), 3.47 (t, J=5.2 Hz, 2H), 2.79 (t, J=6.0 Hz, 2H), 1.97 (m, 2H), 1.42 (d, J=7.2 Hz, 3H).

### Example 43

### (S)-1-((2′-carbamoyl-[1,1′-biphenyl]-4-yl)methyl)-N-(1-(2-(trifluoromethyl)phenyl)ethyl)-1,2,3,4-tetrahydroquinoline-6-carboxamide

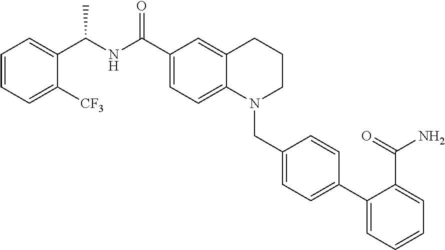 [SMILES: C[C@H](NC(=O)c1ccc2c(c1)CCCN2Cc1ccc(-c2ccccc2C(N)=O)cc1)c1ccccc1C(F)(F)F]

The target compound was obtained by the same manner as described in Example 2 except that (S)-4′-((6-((1-(2-(trifluoromethyl)phenyl)ethyl)carbamoyl)-3,4-dihydroquinoline-1(2H)-yl)methyl)-[1,1′-biphenyl]-2-carboxylic acid obtained in Example 25 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.47 (d, J=7.2 Hz, 1H), 7.81 (d, J=8.0 Hz, 1H), 7.68-7.60 (m, 3H), 7.55-7.35 (m, 9H), 7.30-7.20 (m, 3H), 6.52 (d, J=8.4 Hz, 1H), 5.41 (m, 1H), 4.61 (s, 2H), 3.47 (t, J=5.2 Hz, 2H), 2.80 (t, J=6.4 Hz, 2H), 1.97 (m, 2H), 1.42 (d, J=6.8 Hz, 3H).

### Example 44

### (S)-1-((2′-carbamoyl-[1,1′-biphenyl]-4-yl)methyl)-N-(1-(naphthalene-2-yl)ethyl)-1,2,3,4-tetrahydroquinoline-6-carboxamide

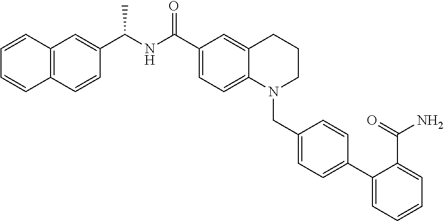 [SMILES: C[C@H](NC(=O)c1ccc2c(c1)CCCN2Cc1ccc(-c2ccccc2C(N)=O)cc1)c1ccc2ccccc2c1]

The target compound was obtained by the same manner as described in Example 2 except that (S)-4′-((6-((1-(naphthalene-2-yl)ethyl)carbamoyl)-3,4-dihydroquinoline-1(2H)-yl)methyl)-[1,1′-biphenyl]-2-carboxylic acid obtained in Example 20 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.84-7.82 (m, 4H), 7.78-7.76 (m, 1H), 7.53-7.40 (m, 9H), 7.37-7.35 (m, 1H), 7.30-7.28 (m, 3H), 6.50-6.48 (m, 1H), 6.22-6.20 (m, NH, 1H), 5.53-5.48 (m, 1H), 5.44 (br, NH, 1H), 5.30 (br, NH, 1H), 4.59 (s, 2H), 3.51-3.47 (m, 2H), 2.89-2.86 (m, 2H), 2.09-2.04 (m, 2H), 1.74-1.68 (m, 3H).

### Example 45

### (S)-1-((2′-carbamoyl-[1,1′-biphenyl]-4-yl)methyl)-N-(1-(3-fluorophenyl)ethyl)-1,2,3,4-tetrahydroquinoline-6-carboxamide

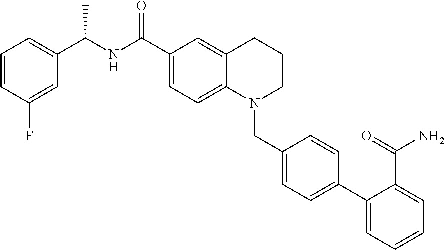 [SMILES: C[C@H](NC(=O)c1ccc2c(c1)CCCN2Cc1ccc(-c2ccccc2C(N)=O)cc1)c1cccc(F)c1]

The target compound was obtained by the same manner as described in Example 2 except that (S)-4′-((6-((1-(3-fluorophenyl)ethyl)carbamoyl)-3,4-dihydroquinoline-1(2H)-yl)methyl)-[1,1′-biphenyl]-2-carboxylic acid obtained in Example 17 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.34-8.32 (m, 1H), 7.66 (m, 1H), 7.52-7.31 (m, 9H), 7.27-7.24 (m, 3H), 7.19-7.15 (m, 2H), 7.05-7.00 (m, 1H), 6.54-6.51 (m, 1H), 5.14-5.11 (m, 1H), 4.61 (s, 2H), 3.47-3.43 (m, 2H), 2.80-2.79 (m, 2H), 1.99-1.96 (m, 2H), 1.44-1.42 (m, 3H).

### Example 46

### (S)-1-((2′-carbamoyl-[1,1′-biphenyl]-4-yl)methyl)-N-(1-(3-(trifluoromethoxy)phenyl)ethyl)-1,2,3,4-tetrahydroquinoline-6-carboxamide

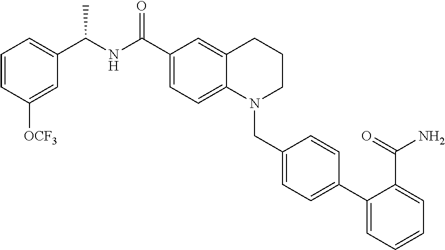 [SMILES: C[C@H](NC(=O)c1ccc2c(c1)CCCN2Cc1ccc(-c2ccccc2C(N)=O)cc1)c1cccc(OC(F)(F)F)c1]

The target compound was obtained by the same manner as described in Example 2 except that (S)-4′-((6-((1-(3-(trifluoromethoxy)phenyl)ethyl)carbamoyl)-3,4-dihydroquinoline-1(2H)-yl)methyl)-[1,1′-biphenyl]-2-carboxylic acid obtained in Example 18 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.40 (d, J=8.0 Hz, 1H), 7.67 (s, 1H), 7.55-7.19 (m, 15H), 6.53 (d, J=8.4 Hz, 1H), 5.15 (m, 1H), 4.61 (s, 2H), 3.47 (t, J=5.2 Hz, 2H), 2.80 (t, J=5.6 Hz, 2H), 1.96 (m, 2H), 1.46 (d, J=6.8 Hz, 3H).

### Example 47

### (S)-1-((2′-carbamoyl-[1,1′-biphenyl]-4-yl)methyl)-N-(1-(3-(trifluoromethyl)phenyl)ethyl)-1,2,3,4-tetrahydroquinoline-6-carboxamide

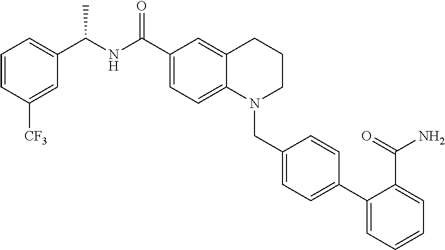 [SMILES: C[C@H](NC(=O)c1ccc2c(c1)CCCN2Cc1ccc(-c2ccccc2C(N)=O)cc1)c1cccc(C(F)(F)F)c1]

The target compound was obtained by the same manner as described in Example 2 except that (S)-4′-((6-((1-(3-(trifluoromethyl)phenyl)ethyl)carbamoyl)-3,4-dihydroquinoline-1(2H)-yl)methyl)-[1,1′-biphenyl]-2-carboxylic acid obtained in Example 19 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.44 (d, J=8.0 Hz, 1H), 7.75-7.24 (m, 16H), 6.53 (d, J=8.8 Hz, 1H), 5.18 (m, 1H), 4.61 (s, 2H), 3.47 (t, J=5.2 Hz, 2H), 2.79 (t, J=5.6 Hz, 2H), 1.95 (m, 2H), 1.46 (d, J=7.2 Hz, 3H).

### Example 48

### 1-((2′-carbamoyl-[1,1′-biphenyl]-4-yl)methyl)-N-(pyridine-4-ylmethyl)-1,2,3,4-tetrahydroquinoline-6-carboxamide

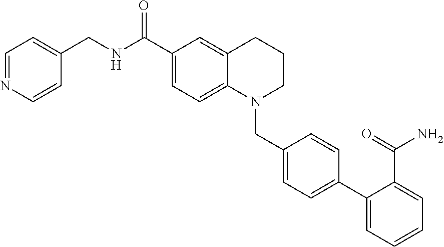 [SMILES: NC(=O)c1ccccc1-c1ccc(CN2CCCc3cc(C(=O)NCc4ccncc4)ccc32)cc1]

The target compound was obtained by the same manner as described in Example 2 except that 4′-((6-((pyridine-4-ylmethyl)carbamoyl)-3,4-dihydroquinoline-1(2H)-yl)methyl)-[1,1′-biphenyl]-2-carboxylic acid obtained in Example 27 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.67-8.64 (m, 1H), 8.48-8.47 (m, 2H), 7.65 (m, 1H), 7.53-7.35 (m, 8H), 7.27-7.25 (m, 5H), 6.55-6.53 (m, 1H), 4.62 (s, 2H), 4.44-4.42 (m, 2H), 3.49-3.46 (m, 2H), 2.82-2.79 (m, 2H), 1.98-1.97 (m, 2H).

### Example 49

### (S)-1-((2′-carbamoyl-[1,1′-biphenyl]-4-yl)methyl)-N-(1-(3-fluoro-4-methylphenyl)ethyl)-1,2,3,4-tetrahydroquinoline-6-carboxamide

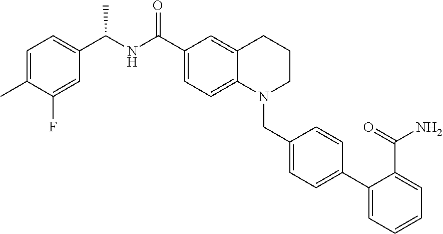 [SMILES: Cc1ccc([C@H](C)NC(=O)c2ccc3c(c2)CCCN3Cc2ccc(-c3ccccc3C(N)=O)cc2)cc1F]

The target compound was obtained by the same manner as described in Example 2 except that (S)-4′-((6-((1-(3-fluoro-4-methyl)phenyl)ethyl)carbamoyl)-3,4-dihydroquinoline-1(2H)-yl)methyl)-[1,1′-biphenyl]-2-carboxylic acid obtained in Example 5 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.74 (d, J=8.0 Hz, 1H), 7.59-7.25 (m, 9H), 7.13 (d, J=8.0 Hz, 1H), 7.03 (t, J=8.0 Hz, 1H), 6.46 (d, J=7.6 Hz, 1H), 6.19 (d, J=7.6 Hz, 1H), 5.66 (s, 1H), 5.39 (s, 1H), 5.26 (m, 1H), 4.58 (s, 2H), 3.49 (t, J=5.2 Hz, 2H), 2.86 (t, J=6.0 Hz, 2H), 2.24 (s, 3H), 2.05 (m, 2H), 1.54 (d, J=6.8 Hz, 3H).

### Example 50

### N-benzyl-1-((2′-carbamoyl-[1,1′-biphenyl]-4-yl)methyl)-1,2,3,4-tetrahydroquinoline-6-carboxamide

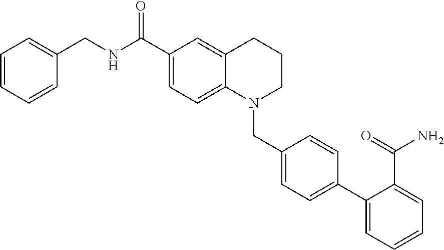 [SMILES: NC(=O)c1ccccc1-c1ccc(CN2CCCc3cc(C(=O)NCc4ccccc4)ccc32)cc1]

The target compound was obtained by the same manner as described in Example 2 except that 4′-((6-(benzylcarbamoyl)-3,4-dihydroquinoline-1(2H)-yl)methyl)-[1,1′-biphenyl]-2-carboxylic acid obtained in Example 28 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.53-7.49 (m, 2H), 7.45-7.41 (m, 4H), 7.37-7.35 (m, 5H), 7.33-7.28 (m, 3H), 6.49-6.47 (m, 1H), 6.23-6.21 (m, NH, 1H), 5.49 (br, NH, 1H), 5.32 (br, NH, 1H), 4.64-4.63 (m, 2H), 4.59 (s, 2H), 3.59-3.47 (m, 2H), 2.89-2.85 (m, 2H), 2.09-2.03 (m, 2H).

### Example 51

### N-(2-bromobenzyl)-1-((2′-carbamoyl-[1,1′-biphenyl]-4-yl)methyl)-1,2,3,4-tetrahydroquinoline-6-carboxamide

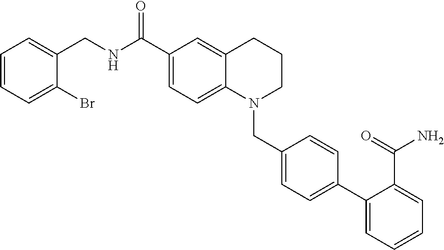 [SMILES: NC(=O)c1ccccc1-c1ccc(CN2CCCc3cc(C(=O)NCc4ccccc4Br)ccc32)cc1]

The target compound was obtained by the same manner as described in Example 2 except that 4′-((7-((2-bromobenzyl)carbamoyl)-2H-benzo[b][1,4]oxazine-4(3H)-yl)methyl)-[1,1′-biphenyl]-2-carboxylic acid obtained in Example 29 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.78-7.76 (m, 1H), 7.57-7.55 (m, 1H), 7.54-7.35 (m, 8H), 7.31-7.29 (m, 3H), 7.17-7.13 (m, 1H), 6.49-6.47 (m, 1H), 6.47-6.46 (m, NH, 1H), 5.50 (br, NH, 1H), 5.32 (br, NH, 1H), 4.70-4.69 (m, 2H), 4.60 (s, 2H), 3.50-3.48 (m, 2H), 2.89-2.86 (m, 2H), 2.09-2.03 (m, 2H).

### Example 52

### 1-((2′-carbamoyl-[1,1′-biphenyl]-4-yl)methyl)-N-((2,3-dihydrobenzo[b][1,4]dioxin-6-yl)methyl)-1,2,3,4-tetrahydroquinoline-6-carboxamide

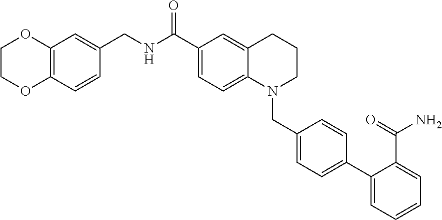 [SMILES: NC(=O)c1ccccc1-c1ccc(CN2CCCc3cc(C(=O)NCc4ccc5c(c4)OCCO5)ccc32)cc1]

The target compound was obtained by the same manner as described in Example 2 except that 4′-((6-(((2,3-dihydrobenzo[b][1,4]dioxin-6-yl)methyl)carbamoyl)-3,4-dihydroquinoline-1(2H)-yl)methyl)-[1,1′-biphenyl]-2-carboxylic acid obtained in Example 30 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.78-7.76 (m, 1H), 7.53-7.48 (m, 2H), 7.46-7.41 (m, 4H), 7.37-7.35 (m, 1H), 7.30-7.28 (m, 2H), 6.86 (m, 1H), 6.83 (m, 2H), 6.14-6.12 (m, NH, 1H), 5.46 (br, NH, 1H), 5.31 (br, NH, 1H), 4.59 (s, 2H), 4.52-4.51 (m, 2H), 4.26 (s, 4H), 3.50-3.47 (m, 2H), 2.88-2.85 (m, 2H), 2.09-2.03 (m, 2H).

### Example 53

### N-(4-bromobenzyl)-1-((2′-carbamoyl-[1,1′-biphenyl]-4-yl)methyl)-1,2,3,4-tetrahydroquinoline-6-carboxamide

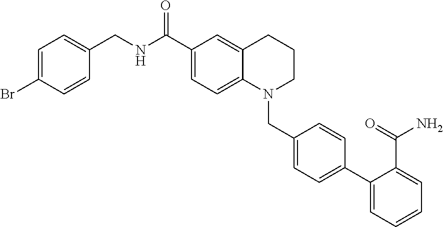 [SMILES: NC(=O)c1ccccc1-c1ccc(CN2CCCc3cc(C(=O)NCc4ccc(Br)cc4)ccc32)cc1]

The target compound was obtained by the same manner as described in Example 2 except that 4′-((6-((4-bromobenzyl)carbamoyl)-3,4-dihydroquinoline-1(2H)-yl)methyl)-[1,1′-biphenyl]-2-carboxylic acid obtained in Example 31 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.78-7.76 (m, 1H), 7.54-7.36 (m, 10H), 7.30-7.22 (m, 3H), 6.49 (d, J=8.8 Hz, 1H), 6.23 (t, J=5.6 Hz, 1H), 5.44 (s, 1H), 5.32 (s, 1H), 4.60-4.58 (m, 4H) 4.50 (t, J=5.6 Hz, 2H), 2.88 (t, J=6.0 Hz, 2H), 2.10-2.04 (m, 2H).

### Example 54

### 1-((2′-carbamoyl-[1,1′-biphenyl]-4-yl)methyl)-N-(3-(trifluoromethoxy)benzyl)-1,2,3,4-tetrahydroquinoline-6-carboxamide

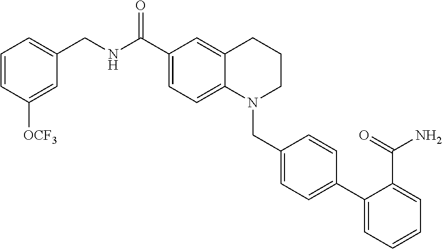 [SMILES: NC(=O)c1ccccc1-c1ccc(CN2CCCc3cc(C(=O)NCc4cccc(OC(F)(F)F)c4)ccc32)cc1]

The target compound was obtained by the same manner as described in Example 2 except that 4′-((6-((3-(trifluoromethoxy)benzyl)carbamoyl)-3,4-dihydroquinoline-1(2H)-yl)methyl)-[1,1′-biphenyl]-2-carboxylic acid obtained in Example 32 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.78-7.76 (m, 1H), 7.54-7.30 (m, 11H), 7.20 (s, 1H), 7.15-7.13 (m, 1H), 6.50 (d, J=8.4 Hz, 1H), 6.28 (t, J=5.6 Hz, 1H), 5.42 (s, 1H), 5.31 (s, 1H), 4.66 (d, J=5.6 Hz, 1H), 4.61 (s, 2H), 3.50 (t, J=5.6 Hz, 2H), 2.89 (t, J=6.0 Hz, 2H), 2.11-2.06 (m, 2H).

### Example 55

### sodium (S)-4′-((6-((1-phenylethyl)carbamoyl)-3,4-dihydroquinoline-1(2H)-yl)methyl)-[1,1′-biphenyl]-2-carboxylate

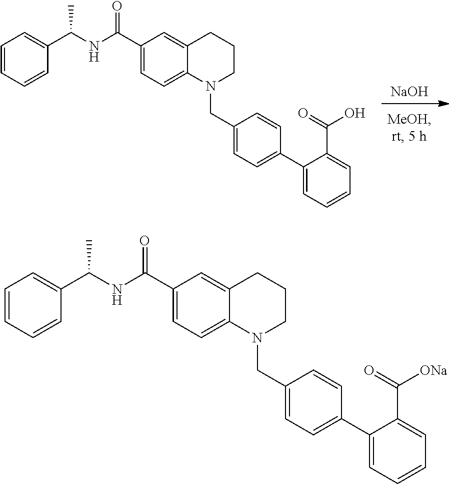

(S)-4′-((6-((1-phenylethyl)carbamoyl)-3,4-dihydroquinoline-1(2H)-yl)methyl)-[1,1′-biphenyl]-2-carboxylic acid (300 mg, 0.61 mmol) obtained in Example 2 was dissolved in MeOH (10 ml) in a 25 mL flask, to which NaOH (24.4 mg, 0.61 mmol) was added, followed by stirring at room temperature for 5 hours. Upon completion of the reaction, the reaction mixture was concentrated under reduced pressure. As a result, sodium (S)-4′-((6-((1-phenylethyl)carbamoyl)-3,4-dihydroquinoline-1(2H)-yl)methyl)-[1,1′-biphenyl]-2-carboxylate was obtained (300 mg).

¹H NMR (400 MHz, DMSO-d₆) δ 8.33 (d, J=8.0 Hz, 1H), 7.68 (d, J=8.0 Hz, 1H), 7.60-7.46 (m, 3H), 7.43 (m, 1H), 7.42-7.25 (m, 9H), 7.19 (m, 1H), 6.52 (m, 1H), 5.12 (m, 1H), 4.69 (s, 2H), 3.49 (t, J=4.0 Hz, 2H), 2.81 (t, J=4.0 Hz, 2H), 1.95 (m, 2H), 1.42 (d, J=7.2 Hz, 3H).

### Example 56

### sodium (S)-4′-((6-((1-(3-fluoro-4-methylphenyl)ethyl) carbamoyl)-3, 4-dihydroquinoline-1(2H)-yl)methyl)-[1,1′-biphenyl]-2-carboxylate

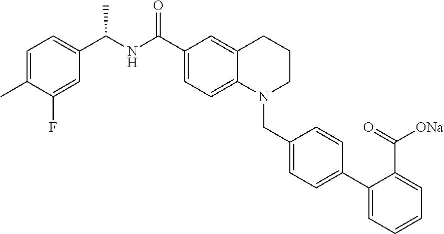

The target compound was obtained by the same manner as described in Example 55 except that (S)-4′-((6-((1-(3-fluoro-4-methylphenyl)ethyl)carbamoyl)-3,4-dihydroquinoline-1(2H)-yl)methyl)-[1,1′-biphenyl]-2-carboxylic acid obtained in Example 5 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.33 (d, J=8.0 Hz, 1H), 7.50-7.42 (m, 4H), 7.30 (m, 1H) 7.35-7.05 (m, 8H), 6.53 (d, J=8.8 Hz, 1H), 5.08 (m, 1H), 4.57 (s, 2H), 3.46 (t, J=5.2 Hz, 2H), 2.79 (t, J=6.0 Hz, 2H), 2.18 (s, 3H), 1.95 (m, 2H), 1.40 (d, J=7.2 Hz, 3H).

### Example 57

### sodium (S)-4′-((6-((1-(4-cyanophenyl)ethyl)carbamoyl)-3,4-dihydroquinoline-1(2H)-yl)methyl)-[1,1′-biphenyl]-2-carboxylate

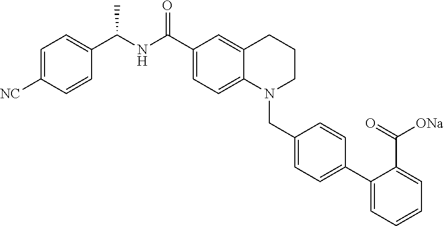

The target compound was obtained by the same manner as described in Example 55 except that (S)-4′-((6-((1-(4-cyanophenyl)ethyl)carbamoyl)-3,4-dihydroquinoline-1(2H)-yl)methyl)-[1,1′-biphenyl]-2-carboxylic acid obtained in Example 14 was used instead of (S)-4′-((6-((1-phenylethyl)carbamoyl)-3,4-dihydroquinoline-1(2H)-yl)methyl)-[1,1′-biphenyl]-2-carboxylic acid.

¹H NMR (400 MHz, DMSO-d₆) δ 8.47 (d, J=6.8 Hz, 1H), 7.77 (d, J=8.0 Hz, 2H), 7.60-7.42 (m, 6H), 7.30-7.13 (m, 6H), 6.53 (d, J=9.2 Hz, 1H), 5.14 (m, 1H), 4.58 (s, 2H), 3.47 (t, J=5.2 Hz, 2H), 2.79 (t, J=5.6 Hz, 2H), 1.95 (m, 2H), 1.43 (d, J=6.8 Hz, 3H).

### Example 58

### (S)-4′-((7-((1-phenylethyl)carbamoyl)-2H-benzo[b][1,4]oxazine-4(3H)-yl)methyl)-[1,1′-biphenyl]-2-carboxylic acid

### Step 1 (S)-tert-butyl 4′-((7-((1-phenylethyl)carbamoyl)-2H-benzo[b][1,4]oxazine-4(3H)-yl)methyl)-[1,1′-biphenyl]-2-carboxylate

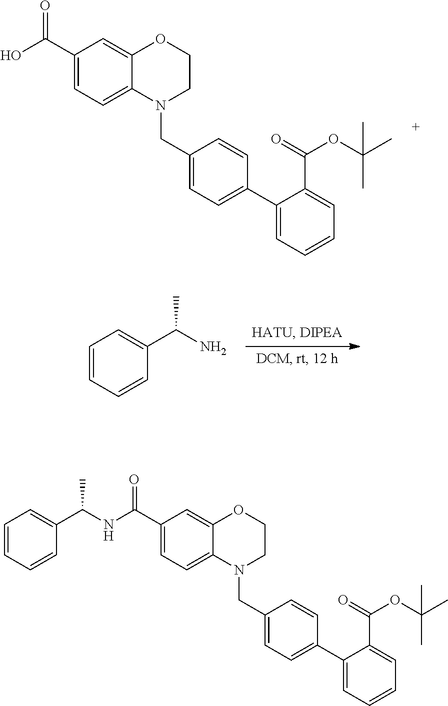

4-((2′-(tert-butoxycarbonyl)-[1,1′-biphenyl]-4-yl)methyl)-3,4-dihydro-2H-benzo[b][1,4]oxazine-7-carboxylic acid (300 mg, 0.67 mmol) obtained in Preparative Example 2 was dissolved in CH₂Cl₂ (10 ml) in a 100 mL flask, to which HATU (280 mg, 0.74 mmol) and DIPEA (350 ul, 2.01 mmol) were added, followed by stirring. (S)-1-phenylethylamine (90 mg, 0.74 mmol) was added thereto, followed by stirring at room temperature for 12 hours. Upon completion of the reaction, the organic layer was separated by using CH₂Cl₂ and H₂O. The separated organic layer was dried over MgSO₄ and filtered. The mixture was separated by column chromatography (EA/n-Hex=1:1) to give(S)-tert-butyl 4′-((7-((1-phenylethyl)carbamoyl)-2H-benzo[b][1,4]oxazine-4(3H)-yl)methyl)-[1,1′-biphenyl]-2-carboxylate (341 mg, 0.62 mmol, 93%).

¹H NMR (400 MHz, CDCl₃) δ 7.80 (d, J=8.0 Hz, 1H), 7.61-7.23 (m, 15H), 6.68 (d, J=8.4 Hz, 1H), 6.14 (d, J=7.6 Hz, 1H), 5.34 (m, 1H), 4.59 (s, 2H), 4.30 (t, J=4.4 Hz, 2H), 3.52 (t, J=4.4 Hz, 2H), 1.54 (d, J=7.2 Hz, 3H).

### Step 2 (S)-4′-((7-((1-phenylethyl)carbamoyl)-2H-benzo[b][1,4]oxazine-4(3H)-yl)methyl)-[1,1′-biphenyl]-2-carboxylic acid

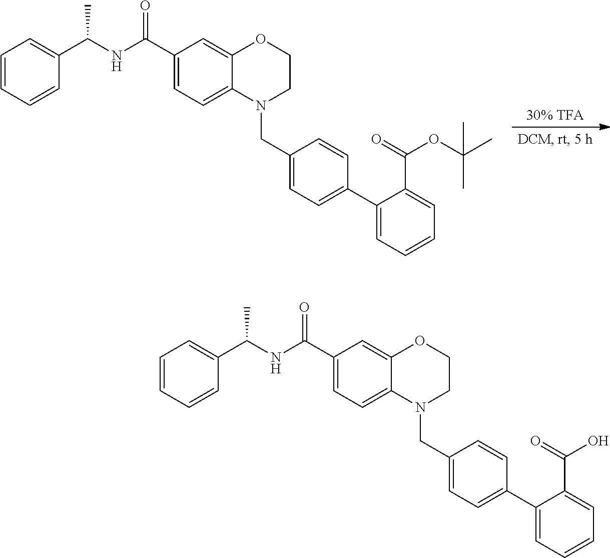 [SMILES: C[C@H](NC(=O)c1ccc2c(c1)OCCN2Cc1ccc(-c2ccccc2C(=O)OC(C)(C)C)cc1)c1ccccc1 ; "30% TFA" ; "DCM, rt, 5 h" ; C[C@H](NC(=O)c1ccc2c(c1)OCCN2Cc1ccc(-c2ccccc2C(=O)O)cc1)c1ccccc1]

(S)-tert-butyl 4′-((7-((1-phenylethyl)carbamoyl)-2H-benzo[b][1,4]oxazine-4(3H)-yl)methyl)-[1,1′-biphenyl]-2-carboxylate (341 mg, 0.62 mmol) obtained in step 1 was dissolved in CH₂Cl₂ (20 ml) in a 50 mL flask, to which 30% TFA (9 ml) was added, followed by stirring at room temperature for 5 hours. Upon completion of the reaction, the reaction mixture was concentrated under reduced pressure. The mixture was separated by column chromatography (EA) to give (S)-4′-((7-((1-phenylethyl)carbamoyl)-2H-benzo[b][1,4]oxazine-4(3H)-yl)methyl)-[1,1′-biphenyl]-2-carboxylic acid (156 mg, 0.31 mmol, 51%).

¹H NMR (400 MHz, DMSO-d₆) δ 12.66 (br, OH, 1H), 8.39 (d, J=8.0 Hz, 1H), 7.70 (d, J=7.2 Hz, 1H), 7.61-7.18 (m, 14H), 6.73 (d, J=8.4 Hz, 1H), 5.11 (m, 1H), 4.63 (s, 2H), 4.25 (t, J=4.0 Hz, 2H), 3.51 (t, J=4.0 Hz, 2H), 1.43 (d, J=7.2 Hz, 3H).

### Example 59

### (S)-4-((2′-carbamoyl-[1,1′-biphenyl]-4-yl)methyl)-N-(1-phenylethyl)-3,4-dihydro-2H-benzo[b][1, 4]oxazine-7-carboxamide

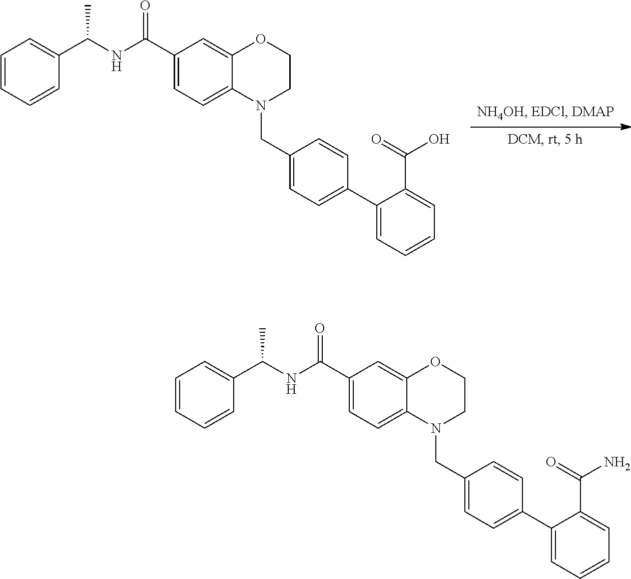 [SMILES: C[C@H](NC(=O)c1ccc2c(c1)OCCN2Cc1ccc(-c2ccccc2C(=O)O)cc1)c1ccccc1 ; "DCM, rt, 5 h" ; "NH4OH, EDCl, DMAP" ; C[C@H](NC(=O)c1ccc2c(c1)OCCN2Cc1ccc(-c2ccccc2C(N)=O)cc1)c1ccccc1]

1-((2′-(tert-butoxycarbonyl)-[1,1′-biphenyl]-4-yl)methyl)-1,2,3,4-tetrahydroquinoline-6-carboxylic acid (300 mg, 0.67 mmol) obtained in Example 57 was dissolved in CH₂Cl₂ (2 ml) in a 25 mL flask, to which EDCI (38 mg, 0.2 mmol) and HOBt (38 mg, 0.3 mmol) were added, followed by stirring. Ammonium hydroxide solution (2 ml) was added thereto, followed by stirring at room temperature for 5 hours. Upon completion of the reaction, the organic layer was separated by using CH₂Cl₂ and H₂O. The separated organic layer was dried over MgSO₄ and filtered. The mixture was separated by column chromatography (MeOH/n-Hex/CH₂Cl₂=0.5:0.5:9) to give (S)-4-((2′-carbamoyl-[1,1′-biphenyl]-4-yl)methyl)-N-(1-phenylethyl)-3,4-dihydro-2H-benzo[b][1,4]oxazine-7-carboxamide (23 mg, 0.04 mmol, 44%).

1H NMR (400 MHz, CDCl₃) δ 7.74 (d, J=7.6 Hz, 1H), 7.51 (td, J=1.2, 7.6 Hz, 1H), 7.47-7.24 (m, 13H), 6.63 (d, J=9.2 Hz, 1H), 6.20 (d, J=8.0 Hz, 1H), 5.61 (s, 1H), 5.39 (s, 1H), 5.32 (m, 1H), 4.57 (s, 2H), 4.30 (t, J=4.4 Hz, 2H), 3.50 (t, J=4.4 Hz, 2H), 1.48 (d, J=6.8 Hz, 3H).

### Example 60

### (S)-4′-((7-((1-(4-nitrophenyl)ethyl)carbamoyl)-2H-benzo[b][1,4]oxazine-4(3H)-yl)methyl)-[1,1′-biphenyl]-2-carboxylic acid

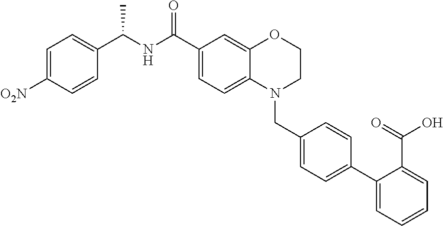 [SMILES: C[C@H](NC(=O)c1ccc2c(c1)OCCN2Cc1ccc(-c2ccccc2C(=O)O)cc1)c1ccc([N+](=O)[O-])cc1]

The target compound was obtained by the same manner as described in Example 58 except that (S)-1-(4-nitrophenyl)ethaneamine was used instead of (S)-1-phenylethylamine.

1H NMR (400 MHz, DMSO-d₆) δ 12.76 (br, OH, 1H), 8.57 (d, J=7.6 Hz, 1H), 8.16 (d, J=8.8 Hz, 2H), 7.70 (m, 1H), 7.69-7.52 (m, 3H), 7.47-7.28 (m, 8H), 6.74 (d, J=8.0 Hz, 1H), 5.19 (m, 1H), 4.64 (s, 2H), 4.26 (t, J=4.0 Hz, 2H), 3.52 (t, J=4.0 Hz, 2H), 1.47 (d, J=7.2 Hz, 3H).

### Example 61

### (S)-4′-((7-((1-(4-bromophenyl)ethyl)carbamoyl)-2H-benzo[b][1,4]oxazine-4(3H)-yl)methyl)-[1,1′-biphenyl]-2-carboxylic acid

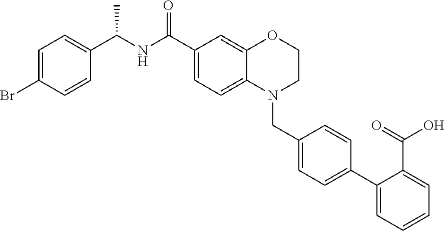 [SMILES: C[C@H](NC(=O)c1ccc2c(c1)OCCN2Cc1ccc(-c2ccccc2C(=O)O)cc1)c1ccc(Br)cc1]

The target compound was obtained by the same manner as described in Example 58 except that (S)-1-(4-bromophenyl)ethaneamine was used instead of (S)-1-phenylethylamine.

¹H NMR (400 MHz, DMSO-d₆) δ 12.76 (br, OH, 1H), 8.43 (d, J=7.6 Hz, 1H), 7.70 (d, J=7.6 Hz, 1H), 7.58-7.41 (m, 4H), 7.39-7.28 (m, 9H), 6.73 (d, J=8.4 Hz, 1H), 5.08 (m, 1H), 4.63 (s, 2H), 4.25 (t, J=4.0 Hz, 2H), 3.52 (t, J=4.0 Hz, 2H), 1.42 (d, J=7.2 Hz, 3H).

### Example 62

### (S)-4′-((7-((1-phenylpropyl)carbamoyl)-2H-benzo[b][1,4]oxazine-4(3H)-yl)methyl)-[1,1′-biphenyl]-2-carboxylic acid

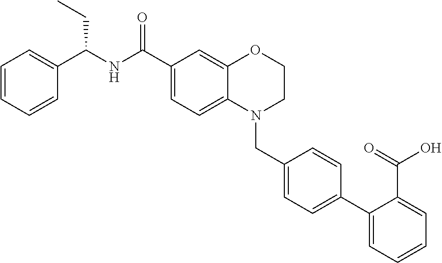 [SMILES: CC[C@H](NC(=O)c1ccc2c(c1)OCCN2Cc1ccc(-c2ccccc2C(=O)O)cc1)c1ccccc1]

The target compound was obtained by the same manner as described in Example 58 except that (S)-1-phenylpropane-1-amine was used instead of (S)-1-phenylethylamine.

1H NMR (400 MHz, DMSO-d₆) δ 12.76 (br, OH, 1H), 8.34 (d, J=8.4 Hz, 1H), 7.70 (dd, J=1.2, 8.0 Hz, 1H), 7.54 (m, 1H), 7.47-7.19 (m, 12H), 6.53 (d, J=8.4 Hz, 1H), 4.85 (q, J=8.4 Hz, 1H), 4.63 (s, 2H), 4.25 (t, J=4.0 Hz, 2H), 3.53 (t, J=4.0 Hz, 2H), 1.78 (m, 2H), 0.87 (t, J=7.2 Hz, 3H).

### Example 63

### (S)-4′-((7-((1-(4-fluorophenyl)ethyl)carbamoyl)-2H-benzo[b][1,4]oxazine-4(3H)-yl)methyl)-[1,1′-biphenyl]-2-carboxylic acid

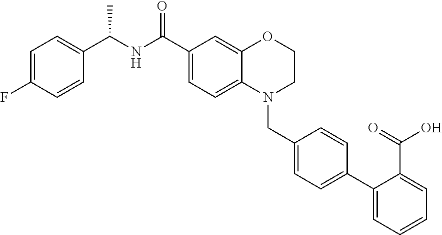 [SMILES: C[C@H](NC(=O)c1ccc2c(c1)OCCN2Cc1ccc(-c2ccccc2C(=O)O)cc1)c1ccc(F)cc1]

The target compound was obtained by the same manner as described in Example 58 except that (S)-1-(4-fluorophenyl)ethaneamine was used instead of (S)-1-phenylethylamine.

¹H NMR (400 MHz, DMSO-d₆) δ 12.86 (br, OH, 1H), 8.42 (d, J=8.0 Hz, 1H), 7.72 (dd, J=1.2, 8.0 Hz, 1H), 7.65-7.28 (m, 13H), 6.74 (d, J=8.4 Hz, 1H), 5.13 (m, 1H), 4.63 (s, 2H), 4.28 (t, J=4.0 Hz, 2H), 3.58 (t, J=4.0 Hz, 2H), 1.42 (d, J=7.2 Hz, 3H).

### Example 64

### (S)-4′-((7-((1-(4-chlorophenyl)ethyl)carbamoyl)-2H-benzo[b][1,4]oxazine-4(3H)-yl)methyl)-[1,1′-biphenyl]-2-carboxylic acid

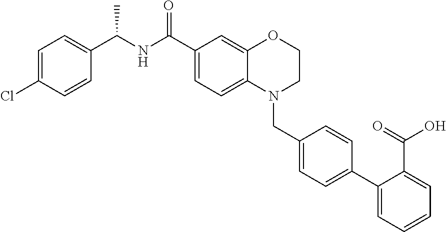 [SMILES: C[C@H](NC(=O)c1ccc2c(c1)OCCN2Cc1ccc(-c2ccccc2C(=O)O)cc1)c1ccc(Cl)cc1]

The target compound was obtained by the same manner as described in Example 58 except that (S)-1-(4-chlorophenyl)ethaneamine was used instead of (S)-1-phenylethylamine.

¹H NMR (400 MHz, DMSO-d₆) δ 12.76 (br, OH, 1H), 8.46 (d, J=7.6 Hz, 1H), 7.72 (m, 1H), 7.55 (m, 1H), 7.47-7.28 (m, 12H), 6.74 (d, J=8.0 Hz, 1H), 5.09 (m, 1H), 4.63 (s, 2H), 4.26 (t, J=4.0 Hz, 2H), 3.52 (t, J=4.0 Hz, 2H), 1.42 (d, J=7.2 Hz, 3H).

### Example 65

### (S)-4′-((7-((1-(3-chlorophenyl)ethyl)carbamoyl)-2H-benzo[b][1,4]oxazine-4(3H)-yl)methyl)-[1,1′-biphenyl]-2-carboxylic acid

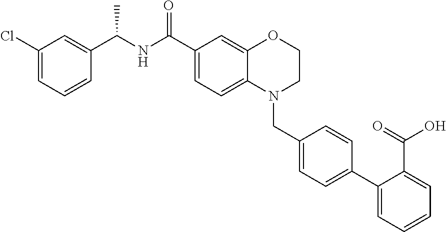 [SMILES: C[C@H](NC(=O)c1ccc2c(c1)OCCN2Cc1ccc(-c2ccccc2C(=O)O)cc1)c1cccc(Cl)c1]

The target compound was obtained by the same manner as described in Example 58 except that (S)-1-(3-chlorophenyl)ethaneamine was used instead of (S)-1-phenylethylamine.

¹H NMR (400 MHz, DMSO-d₆) δ 12.86 (br, OH, 1H), 8.45 (d, J=8.0 Hz, 1H), 7.72 (m, 1H), 7.56 (m, 1H), 7.47-7.28 (m, 12H), 6.74 (d, J=8.0 Hz, 1H), 5.09 (m, 1H), 4.63 (s, 2H), 4.26 (t, J=4.0 Hz, 2H), 3.52 (t, J=4.0 Hz, 2H), 1.42 (d, J=7.2 Hz, 3H).

### Example 66

### 4′-((7-((4-nitrobenzyl)carbamoyl)-2H-benzo[b][1,4]oxazine-4(3H)-yl)methyl)-[1,1′-biphenyl]-2-carboxylic acid

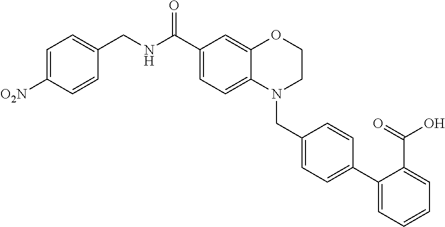 [SMILES: O=C(NCc1ccc([N+](=O)[O-])cc1)c1ccc2c(c1)OCCN2Cc1ccc(-c2ccccc2C(=O)O)cc1]

The target compound was obtained by the same manner as described in Example 58 except that (4-nitrophenyl)methaneamine was used instead of (S)-1-phenylethylamine.

¹H NMR (400 MHz, DMSO-d₆) δ 12.83 (br, OH, 1H), 8.75 (m, 1H), 8.19 (dd, J=1.2, 8.8 Hz, 1H), 7.71-7.19 (m, 12H), 6.73 (d, J=8.4 Hz, 1H), 4.63 (s, 2H), 4.52 (d, J=6.0 Hz, 1H), 4.25 (t, J=4.0 Hz, 2H), 3.53 (t, J=4.0 Hz, 2H).

### Example 67

### 4′-((7-(benzylcarbamoyl)-2H-benzo[b][1,4]oxazine-4(38)-yl)methyl)-[1,1′-biphenyl]-2-carboxylic acid

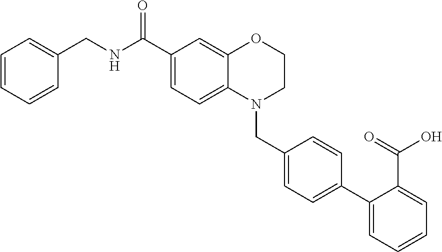 [SMILES: O=C(NCc1ccccc1)c1ccc2c(c1)OCCN2Cc1ccc(-c2ccccc2C(=O)O)cc1]

The target compound was obtained by the same manner as described in Example 58 except that phenylmethaneamine was used instead of (S)-1-phenylethylamine.

¹H NMR (400 MHz, DMSO-d₆) δ 12.63 (br, OH, 1H), 8.75 (t, J=6.0 Hz, 1H), 7.71 (dd, J=1.2, 8.8 Hz, 1H), 7.58-7.19 (m, 14H), 6.76 (d, J=8.8 Hz, 1H), 4.67 (s, 2H), 4.42 (d, J=6.0 Hz, 1H), 4.25 (t, J=4.0 Hz, 2H), 3.53 (t, J=4.0 Hz, 2H).

### Example 68

### 4′-((7-(phenethylcarbamoyl)-2H-benzo[b][1,4]oxazine-4(3H)-yl)methyl)-[1,1′-biphenyl]-2-carboxylic acid

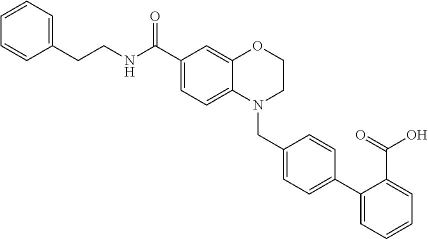 [SMILES: O=C(NCCc1ccccc1)c1ccc2c(c1)OCCN2Cc1ccc(-c2ccccc2C(=O)O)cc1]

The target compound was obtained by the same manner as described in Example 58 except that 2-phenylethaneamine was used instead of (S)-1-phenylethylamine.

¹H NMR (400 MHz, DMSO-d₆) δ 12.63 (br, OH, 1H), 8.17 (t, J=5.6 Hz, 1H), 7.71 (dd, J=1.2, 8.8 Hz, 1H), 7.58-7.19 (m, 14H), 6.72 (d, J=8.4 Hz, 1H), 4.62 (s, 2H), 4.25 (t, J=4.0 Hz, 1H), 4.52 (t, J=4.0 Hz, 2H), 3.40 (m, 2H), 2.74 (t, J=7.6 Hz, 2H).

### Example 69

### 4′-((7-((furan-2-ylmethyl)carbamoyl)-2H-benzo[b][1,4]oxazine-4(3H)-yl)methyl)-[1,1′-biphenyl]-2-carboxylic acid-A

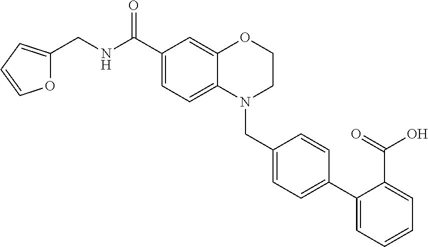 [SMILES: O=C(NCc1ccco1)c1ccc2c(c1)OCCN2Cc1ccc(-c2ccccc2C(=O)O)cc1]

The target compound was obtained by the same manner as described in Example 58 except that furan-2-ylmethaneamine was used instead of (S)-1-phenylethylamine.

¹H NMR (400 MHz, DMSO-d₆) δ 12.88 (br, OH, 1H), 8.56 (t, J=5.6 Hz, 1H), 7.73 (dd, J=1.2, 8.8 Hz, 1H), 7.58-7.19 (m, 10H), 6.72 (d, J=8.8 Hz, 1H), 6.37 (m, 1H), 6.21 (m, 1H), 4.62 (s, 2H), 4.41 (d, J=6.0 Hz, 1H), 4.25 (t, J=4.0 Hz, 2H), 3.51 (t, J=4.0 Hz, 2H).

### Example 70

### (S)-4′-((7-((1-(naphthalene-1-yl)ethyl)carbamoyl)-2H-benzo[b][1,4]oxazine-4(3H)-yl)methyl)-[1,1′-biphenyl]-2-carboxylic acid

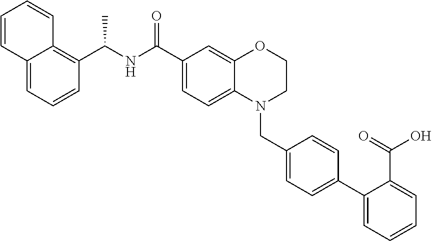 [SMILES: C[C@H](NC(=O)c1ccc2c(c1)OCCN2Cc1ccc(-c2ccccc2C(=O)O)cc1)c1cccc2ccccc12]

The target compound was obtained by the same manner as described in Example 58 except that (S)-1-(naphthalene-1-yl)ethaneamine was used instead of (S)-1-phenylethylamine.

¹H NMR (400 MHz, DMSO-d₆) δ 12.76 (br, OH, 1H), 8.57 (d, J=8.4 Hz, 1H), 8.18 (d, J=8.4 Hz, 1H), 7.93 (m, 1H), 7.81 (d, J=8.4 Hz, 1H), 7.70 (dd, J=1.2, 7.6 Hz, 1H), 7.62-7.25 (m, 13H), 6.70 (d, J=8.8 Hz, 1H), 5.91 (m, 1H), 4.62 (s, 2H), 4.25 (t, J=4.0 Hz, 2H), 3.51 (t, J=4.0 Hz, 2H), 1.57 (d, J=6.8 Hz, 1H).

### Example 71

### 4′-((7-((4-(methoxycarbonyl)benzyl)carbamoyl)-2H-benzo[b][1,4]oxazine-4(3H)-yl)methyl)-[1,1′-biphenyl]-2-carboxylic acid

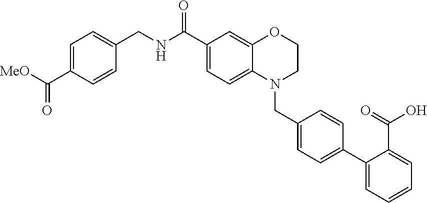 [SMILES: COC(=O)c1ccc(CNC(=O)c2ccc3c(c2)OCCN3Cc2ccc(-c3ccccc3C(=O)O)cc2)cc1]

The target compound was obtained by the same manner as described in Example 58 except that methyl 4-(aminomethyl)benzoate was used instead of (S)-1-phenylethylamine.

¹H NMR (400 MHz, DMSO-d₆) δ 12.86 (br, OH, 1H), 8.75 (t, J=6.0 Hz, 1H), 7.91 (d, J=8.0 Hz, 1H), 7.91 (m, 3H), 7.70 (d, J=7.6 Hz, 1H), 7.56 (t, J=7.6 Hz, 1H), 7.47-7.19 (m, 11H), 6.74 (d, J=8.8 Hz, 1H), 4.63 (s, 2H), 4.49 (d, J=5.2 Hz, 1H), 4.25 (t, J=4.0 Hz, 2H), 3.83 (s, 3H), 3.51 (t, J=4.0 Hz, 2H).

### Example 72

### 4′-((7-((3-methoxybenzyl) carbamoyl)-2H-benzo[b][1,4]oxazine-4(3H)-yl)methyl)-[1,1′-biphenyl]-2-carboxylic acid

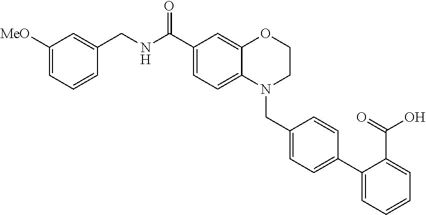 [SMILES: COc1cccc(CNC(=O)c2ccc3c(c2)OCCN3Cc2ccc(-c3ccccc3C(=O)O)cc2)c1]

The target compound was obtained by the same manner as described in Example 58 except that (3-methoxyphenyl)methaneamine was used instead of (S)-1-phenylethylamine.

¹H NMR (400 MHz, DMSO-d₆) δ 12.76 (br, OH, 1H), 7.70 (dd, J=1.2, 8.0 Hz, 1H), 7.56 (m, 1H), 7.46 (m, 1H), 7.40-7.19 (m, 8H), 6.85-6.82 (m, 2H), 6.79-6.74 (m, 1H), 6.73 (d, J=8.8 Hz, 1H), 4.63 (s, 2H), 4.39 (d, J=6.0 Hz, 1H), 4.25 (t, J=4.0 Hz, 2H), 3.72 (s, 3H), 3.51 (t, J=4.0 Hz, 2H).

### Example 73

### 4′-((7-((4-bromobenzyl) carbamoyl)-2H-benzo[b][1,4]oxazine-4(3H)-yl)methyl)-[1,1′-biphenyl]-2-carboxylic acid

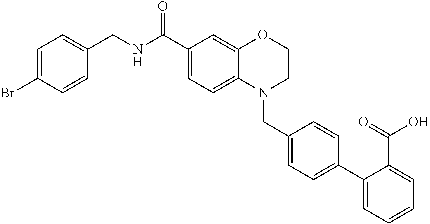 [SMILES: O=C(NCc1ccc(Br)cc1)c1ccc2c(c1)OCCN2Cc1ccc(-c2ccccc2C(=O)O)cc1]

The target compound was obtained by the same manner as described in Example 58 except that (4-bromophenyl)methaneamine was used instead of (S)-1-phenylethylamine.

¹H NMR (400 MHz, DMSO-d₆) δ 12.76 (br, OH, 1H), 8.69 (t, J=6.0 Hz, 1H), 7.70 (dd, J=1.2, 8.4 Hz, 1H), 7.62-7.20 (m, 13H), 6.73 (d, J=8.4 Hz, 1H), 4.63 (s, 2H), 4.42 (d, J=8.0 Hz, 1H), 4.25 (t, J=4.0 Hz, 2H), 3.51 (t, J=4.0 Hz, 2H).

### Example 74

### (S)-4′-((7-((1-(4-cyanophenyl)ethyl)carbamoyl)-2H-benzo[b][1,4]oxazine-4(3H)-yl)methyl)-[1,1′-biphenyl]-2-carboxylic acid

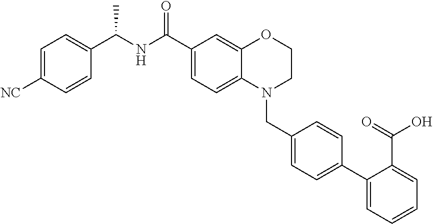 [SMILES: C[C@H](NC(=O)c1ccc2c(c1)OCCN2Cc1ccc(-c2ccccc2C(=O)O)cc1)c1ccc(C#N)cc1]

The target compound was obtained by the same manner as described in Example 58 except that (S)-1-(4-cyanophenyl)ethaneamine was used instead of (S)-1-phenylethylamine.

¹H NMR (400 MHz, DMSO-d₆) δ 12.74 (br, OH, 1H), 8.51 (d, J=7.6 Hz, 1H), 7.80 (m, 2H), 7.61-7.18 (m, 10H), 6.74 (d, J=8.0 Hz, 1H), 5.12 (m, 1H), 4.63 (s, 2H), 4.25 (t, J=4.0 Hz, 2H), 3.51 (t, J=4.0 Hz, 2H), 1.44 (d, J=7.2 Hz, 3H).

### Example 75

### (S)-4′-((7-((1-(4-(trifluoromethoxy)phenyl)ethyl)carbamoyl)-2H-benzo[b][1,4]oxazine-4(3H)-yl)methyl)-[1,1′-biphenyl]-2-carboxylic acid

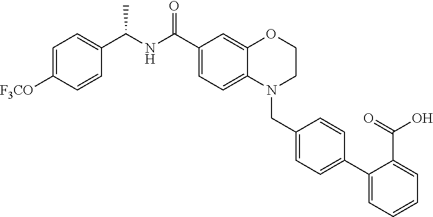 [SMILES: C[C@H](NC(=O)c1ccc2c(c1)OCCN2Cc1ccc(-c2ccccc2C(=O)O)cc1)c1ccc(OC(F)(F)F)cc1]

The target compound was obtained by the same manner as described in Example 58 except that (S)-1-(4-(trifluoromethoxy)phenyl)ethaneamine was used instead of (S)-1-phenylethylamine.

¹H NMR (400 MHz, DMSO-d₆) δ 12.90 (br, OH, 1H), 8.44 (d, J=8.0 Hz, 1H), 7.95 (d, J=8.0 Hz, 1H), 7.71-7.18 (m, 13H), 6.74 (d, J=8.4 Hz, 1H), 5.13 (m, 1H), 4.63 (s, 2H), 4.25 (t, J=4.0 Hz, 2H), 3.51 (t, J=4.0 Hz, 2H), 1.44 (d, J=7.2 Hz, 3H).

### Example 76

### (S)-4′-((7-((1-(4-(trifluoromethyl)phenyl)ethyl)carbamoyl)-2H-benzo[b][1,4]oxazine-4(3H)-yl)methyl)-[1,1′-biphenyl]-2-carboxylic acid

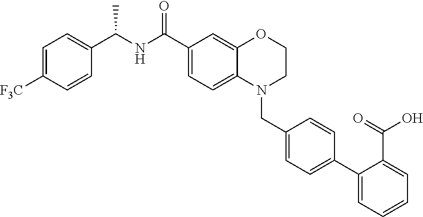 [SMILES: C[C@H](NC(=O)c1ccc2c(c1)OCCN2Cc1ccc(-c2ccccc2C(=O)O)cc1)c1ccc(C(F)(F)F)cc1]

The target compound was obtained by the same manner as described in Example 58 except that (S)-1-(4-(trifluoromethyl)phenyl)ethaneamine used instead of (S)-1-phenylethylamine.

¹H NMR (400 MHz, DMSO-d₆) δ 12.73 (br, OH, 1H), 8.50 (d, J=8.0 Hz, 1H), 7.94 (d, J=8.4 Hz, 1H), 7.75-7.43 (m, 6H), 7.48-7.40 (m, 1H), 7.38-7.25 (m, 6H), 6.74 (d, J=8.4 Hz, 1H), 5.16 (m, 1H), 4.63 (s, 2H), 4.25 (t, J=4.0 Hz, 2H), 3.52 (t, J=4.0 Hz, 2H), 1.46 (d, J=7.2 Hz, 3H).

### Example 77

### (S)-4′-((7-((1-(4-((trifluoromethyl)thio)phenyl)ethyl)carbamoyl)-2H-benzo[b][1,4]oxazine-4(3H)-yl)methyl)-[1,1′-biphenyl]-2-carboxylic acid

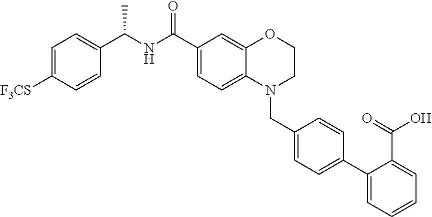

The target compound was obtained by the same manner as described in Example 58 except that (S)-1-(4-((trifluoromethyl)thio)phenyl)ethaneamine was used instead of (S)-1-phenylethylamine.

¹H NMR (400 MHz, DMSO-d₆) δ 12.63 (br, OH, 1H), 8.49 (d, J=8.0 Hz, 1H), 7.74 (d, J=8.0 Hz, 1H), 7.65 (m, 2H), 7.60-7.50 (m, 3H), 7.45 (m, 1H), 7.40-7.15 (m, 5H), 6.74 (d, J=8.4 Hz, 1H), 5.15 (m, 1H), 4.63 (s, 2H), 4.25 (t, J=4.0 Hz, 2H), 3.52 (t, J=4.0 Hz, 2H), 1.44 (d, J=6.8 Hz, 3H).

### Example 78

### (S)-4′-((7-((1-(4-(tert-butyl)phenyl)ethyl)carbamoyl)-2H-benzo[b][1,4]oxazine-4(3H)-yl)methyl)-[1,1′-biphenyl]-2-carboxylic acid

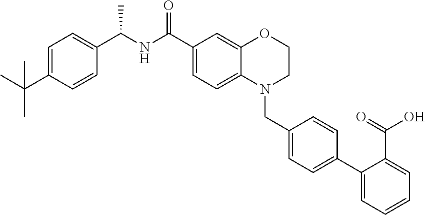 [SMILES: C[C@H](NC(=O)c1ccc2c(c1)OCCN2Cc1ccc(-c2ccccc2C(=O)O)cc1)c1ccc(C(C)(C)C)cc1]

The target compound was obtained by the same manner as described in Example 58 except that (S)-1-(4-(tert-butyl)phenyl)ethaneamine was used instead of (S)-1-phenylethylamine.

¹H NMR (400 MHz, DMSO-d₆) δ 12.63 (br, OH, 1H), 8.35 (d, J=8.0 Hz, 1H), 8.00 (d, J=8.0 Hz, 1H), 7.65-7.15 (m, 13H), 6.73 (d, J=8.4 Hz, 1H), 5.10 (m, 1H), 4.63 (s, 2H), 4.25 (t, J=4.0 Hz, 2H), 3.51 (t, J=4.0 Hz, 2H), 1.42 (d, J=7.2 Hz, 3H), 1.25 (s, 9H).

### Example 79

### (S)-4′-((7-((1-(4-fluoro-2-methylphenyl)ethyl)carbamoyl)-2H-benzo[b][1,4]oxazine-4(3H)-yl)methyl)-[1,1′-biphenyl]-2-carboxylic acid

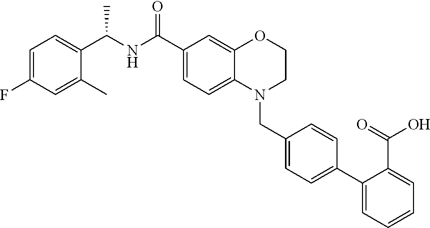 [SMILES: Cc1cc(F)ccc1[C@H](C)NC(=O)c1ccc2c(c1)OCCN2Cc1ccc(-c2ccccc2C(=O)O)cc1]

The target compound was obtained by the same manner as described in Example 58 except that (S)-1-(4-fluoro-2-methylphenyl)ethaneamine was used instead of (S)-1-phenylethylamine.

¹H NMR (400 MHz, DMSO-d₆) δ 12.63 (br, OH, 1H), 8.41 (d, J=8.0 Hz, 1H), 7.71 (d, J=8.0 Hz, 1H), 7.60-7.52 (m, 1H), 7.47-7.10 (m, 6H), 6.98 (m, 2H), 6.72 (d, J=8.0 Hz, 1H), 5.23 (m, 1H), 4.62 (s, 2H), 4.25 (t, J=4.0 Hz, 2H), 3.52 (t, J=4.0 Hz, 2H), 2.36 (s, 3H), 1.38 (d, J=6.8 Hz, 3H).

### Example 80

### (S)-4′-((7-((1-(3-fluoro-4-methylphenyl)ethyl)carbamoyl)-2H-benzo[b][1,4]oxazine-4(3H)-yl)methyl)-[1,1′-biphenyl]-2-carboxylic acid

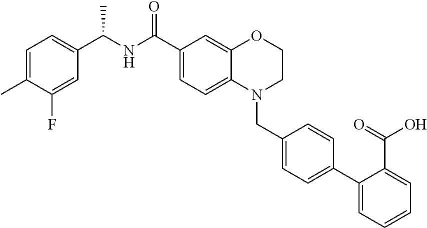 [SMILES: Cc1ccc([C@H](C)NC(=O)c2ccc3c(c2)OCCN3Cc2ccc(-c3ccccc3C(=O)O)cc2)cc1F]

The target compound was obtained by the same manner as described in Example 58 except that (S)-1-(3-fluoro-4-methylphenyl)ethaneamine was used instead of (S)-1-phenylethylamine.

¹H NMR (400 MHz, DMSO-d₆) δ 12.71 (br, OH, 1H), 8.39 (d, J=8.0 Hz, 1H), 7.71 (d, J=8.0 Hz, 1H), 7.60-7.00 (m, 12H), 6.74 (d, J=8.4 Hz, 1H), 5.08 (m, 1H), 4.63 (s, 2H), 4.25 (t, J=4.0 Hz, 2H), 3.52 (t, J=4.0 Hz, 2H), 2.18 (s, 3H), 1.41 (d, J=7.2 Hz, 3H).

### Example 81

### (R)-4′-((7-((1-(4-cyanophenyl)ethyl)carbamoyl)-2H-benzo[b][1,4]oxazine-4(3H)-yl)methyl)-[1,1′-biphenyl]-2-carboxylic acid

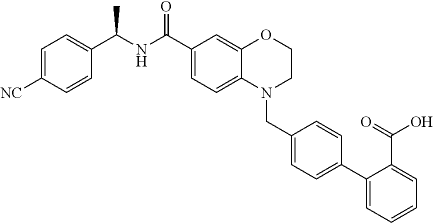 [SMILES: C[C@@H](NC(=O)c1ccc2c(c1)OCCN2Cc1ccc(-c2ccccc2C(=O)O)cc1)c1ccc(C#N)cc1]

The target compound was obtained by the same manner as described in Example 58 except that (R)-1-(4-cyanophenyl)ethaneamine was used instead of (S)-1-phenylethylamine.

¹H NMR (400 MHz, DMSO-d₆) δ 8.51 (d, J=8.0 Hz, 1H), 7.78 (d, J=8.0 Hz, 2H), 7.66 (s, 1H), 7.54 (d, J=8.0 Hz, 2H), 7.50-7.20 (m, 11H), 6.74 (d, J=8.4 Hz, 1H), 5.14 (m, 1H), 4.62 (s, 2H), 4.26 (t, J=4.0 Hz, 2H), 3.52 (t, J=4.0 Hz, 2H), 1.42 (d, J=7.2 Hz, 3H).

### Example 82

### 4′-((7-(cromene-3-ylcarbamoyl)-2H-benzo[b][1,4]oxazine-4(3H)-yl)methyl)-[1,1′-biphenyl]-2-carboxylic acid

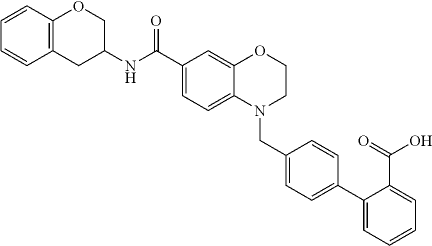 [SMILES: O=C(NC1COc2ccccc2C1)c1ccc2c(c1)OCCN2Cc1ccc(-c2ccccc2C(=O)O)cc1]

The target compound was obtained by the same manner as described in Example 58 except that cromene-3-amine was used instead of (S)-1-phenylethylamine.

¹H NMR (400 MHz, DMSO-d₆) δ 12.74 (br, OH, 1H), 8.08 (m, 1H), 7.95 (m, 1H), 7.72 (m, 1H), 7.45 (m, 1H), 7.40-7.28 (m, 6H), 7.13-7.05 (m, 2H), 6.88 (m, 1H), 6.80-6.71 (m, 2H), 4.63 (s, 2H), 4.31-4.13 (m, 4H), 3.83 (m, 1H), 3.51 (t, J=4.0 Hz, 2H), 3.03-2.86 (m, 2H).

### Example 83

### (S)-4′-((7-((1-(2,6-fluorophenyl)ethyl)carbamoyl)-2H-benzo[b][1,4]oxazine-4(3H)-yl)methyl)-[1,1′-biphenyl]-2-carboxylic acid

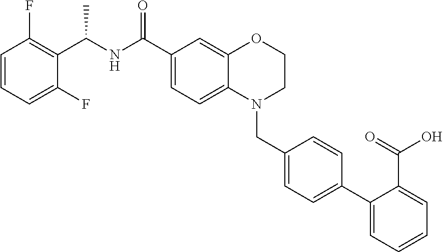 [SMILES: C[C@H](NC(=O)c1ccc2c(c1)OCCN2Cc1ccc(-c2ccccc2C(=O)O)cc1)c1c(F)cccc1F]

The target compound was obtained by the same manner as described in Example 58 except that (S)-1-(2,6-difluorophenyl)ethaneamine was used instead of (S)-1-phenylethylamine.

¹H NMR (400 MHz, DMSO-d₆) δ 12.78 (br, OH, 1H), 8.40 (d, J=6.8 Hz, 1H), 7.70 (dd, J=0.8, 7.6 Hz, 1H), 7.56 (td, J=1.2, 7.2 Hz, 1H), 7.44 (td, J=0.8, 7.2 Hz, 1H), 7.38-7.25 (m, 8H), 7.01 (t, J=8.0 Hz, 1H), 6.72 (d, J=8.8 Hz, 1H), 5.34 (m, 1H), 4.62 (s, 2H), 4.24 (t, J=4.0 Hz, 2H), 3.51 (t, J=4.0 Hz, 2H), 1.53 (d, J=7.2 Hz, 3H).

### Example 84

### (S)-4′-((7-((1-(3-fluorophenyl)ethyl)carbamoyl)-2H-benzo[b][1,4]oxazine-4(3H)-yl)methyl)-[1,1′-biphenyl]-2-carboxylic acid

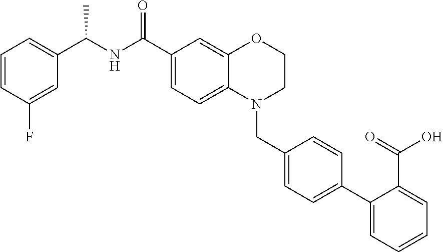 [SMILES: C[C@H](NC(=O)c1ccc2c(c1)OCCN2Cc1ccc(-c2ccccc2C(=O)O)cc1)c1cccc(F)c1]

The target compound was obtained by the same manner as described in Example 58 except that (S)-1-(3-fluorophenyl)ethaneamine was used instead of (S)-1-phenylethylamine.

¹H NMR (400 MHz, DMSO-d₆) δ 12.68 (br, OH, 1H), 8.42 (d, J=8.0 Hz, 1H), 7.71 (d, J=6.8 Hz, 1H), 7.56 (td, J=1.2, 7.6 Hz, 1H), 7.45 (m, 1H), 7.40-7.28 (m, 8H), 7.23-7.15 (m, 2H), 7.02 (td, J=2.0, 8.4 Hz, 1H), 6.74 (d, J=8.4 Hz, 1H), 5.12 (m, 1H), 4.63 (s, 2H), 4.25 (t, J=4.0 Hz, 2H), 3.51 (t, J=4.0 Hz, 2H), 1.44 (d, J=6.8 Hz, 3H).

### Example 85

### (S)-4′-((7-((1-(3-(trifluoromethoxy)phenyl)ethyl)carbamoyl)-2H-benzo[b][1,4]oxazine-4(3H)-yl)methyl)-[1,1′-biphenyl]-2-carboxylic acid

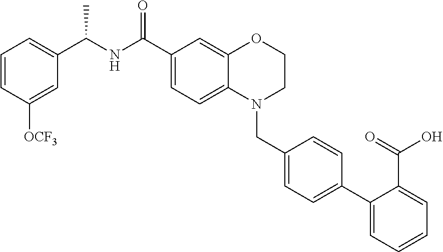 [SMILES: C[C@H](NC(=O)c1ccc2c(c1)OCCN2Cc1ccc(-c2ccccc2C(=O)O)cc1)c1cccc(OC(F)(F)F)c1]

The target compound was obtained by the same manner as described in Example 58 except that (S)-1-(3-(trifluoromethoxy)phenyl)ethaneamine was used instead of (S)-1-phenylethylamine.

¹H NMR (400 MHz, DMSO-d₆) δ 12.72 (br, OH, 1H), 8.47 (d, J=8.0 Hz, 1H), 7.71 (dd, J=0.8, 7.6 Hz, 1H), 7.55 (td, J=1.2, 7.6 Hz, 1H), 7.49-7.28 (m, 11H), 7.20 (m, 1H), 6.75 (d, J=7.6 Hz, 1H), 5.15 (m, 1H), 4.63 (s, 2H), 4.26 (t, J=4.0 Hz, 2H), 3.52 (t, J=4.0 Hz, 2H), 1.46 (d, J=6.8 Hz, 3H).

### Example 86

### (S)-4′-((7-((1-(3-(trifluoromethyl)phenyl)ethyl)carbamoyl)-2H-benzo[b][1,4]oxazine-4(3H)-yl)methyl)-[1,1′-biphenyl]-2-carboxylic acid

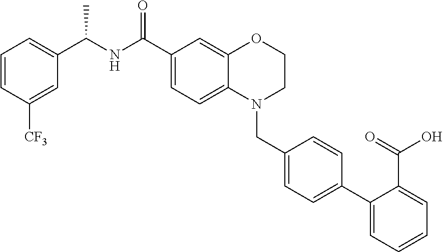 [SMILES: C[C@H](NC(=O)c1ccc2c(c1)OCCN2Cc1ccc(-c2ccccc2C(=O)O)cc1)c1cccc(C(F)(F)F)c1]

The target compound was obtained by the same manner as described in Example 58 except that (S)-1-(3-(trifluoromethyl)phenyl)ethaneamine was used instead of (S)-1-phenylethylamine.

¹H NMR (400 MHz, DMSO-d₆) δ 12.74 (br, OH, 1H), 8.51 (d, J=7.6 Hz, 1H), 7.80-7.20 (m, 14H), 6.75 (d, J=8.4 Hz, 1H), 5.19 (m, 1H), 4.63 (s, 2H), 4.25 (t, J=4.0 Hz, 2H), 3.51 (t, J=4.0 Hz, 2H), 1.46 (d, J 10=7.2 Hz, 3H).

### Example 87

### (S)-4′-((7-((1-(naphthalene-2-yl)ethyl)carbamoyl)-2H-benzo[b][1,4]oxazine-4(3H)-yl)methyl)-[1,1′-biphenyl]-2-carboxylic acid

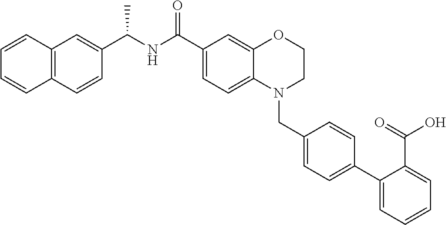 [SMILES: C[C@H](NC(=O)c1ccc2c(c1)OCCN2Cc1ccc(-c2ccccc2C(=O)O)cc1)c1ccc2ccccc2c1]

The target compound was obtained by the same manner as described in Example 58 except that (S)-1-(naphthalene-2-yl)ethaneamine was used instead of (S)-1-phenylethylamine.

¹H NMR (400 MHz, DMSO-d₆) δ 12.72 (br, OH, 1H), 8.50 (d, J=8.0 Hz, 1H), 7.90-7.80 (m, 4H), 7.71 (d, J=7.6 Hz, 1H), 7.58-7.28 (m, 12H), 6.74 (d, J=8.4 Hz, 1H), 5.29 (m, 1H), 4.63 (s, 2H), 4.25 (t, J=4.0 Hz, 2H), 3.51 (t, J=4.0 Hz, 2H), 1.54 (d, J=6.8 Hz, 3H).

### Example 88

### 4′-((7-((pyridine-2-ylmethyl)carbamoyl)-2H-benzo[b][1,4]oxazine-4(3H)-yl)methyl)-[1,1′-biphenyl]-2-carboxylic acid

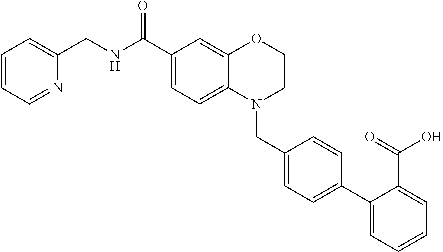 [SMILES: O=C(NCc1ccccn1)c1ccc2c(c1)OCCN2Cc1ccc(-c2ccccc2C(=O)O)cc1]

The target compound was obtained by the same manner as described in Example 58 except that pyridine-2-ylmethaneamine was used instead of (S)-1-phenylethylamine.

¹H NMR (400 MHz, DMSO-d₆) δ 12.75 (br, OH, 1H), 8.76-8.73 (m, 1H), 8.53-8.52 (m, 1H), 7.83-7.80 (m, 1H), 7.71-7.69 (m, 1H), 7.57-7.54 (m, 1H), 7.46-7.42 (m, 1H), 7.38-7.30 (m, 9H), 6.76-6.74 (m, 1H), 4.64 (s, 2H), 4.54-4.52 (m, 2H), 4.26-4.25 (m, 2H), 3.52-3.38 (m, 2H).

### Example 89

### 4′-((7-((pyridine-3-ylmethyl)carbamoyl)-2H-benzo[b][1,4]oxazine-4(3H)-yl)methyl)-[1,1′-biphenyl]-2-carboxylic acid

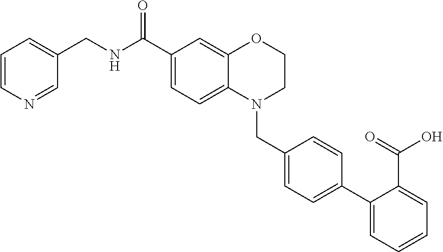 [SMILES: O=C(NCc1cccnc1)c1ccc2c(c1)OCCN2Cc1ccc(-c2ccccc2C(=O)O)cc1]

The target compound was obtained by the same manner as described in Example 58 except that pyridine-3-ylmethaneamine was used instead of (S)-1-phenylethylamine.

¹H NMR (400 MHz, DMSO-d₆) δ 12.75 (br, OH, 1H), 8.82-8.76 (m, 1H), 8.60 (m, 1H), 8.54-8.53 (m, 1H), 7.89-7.87 (m, 1H), 7.71-7.69 (m, 1H), 7.57-7.50 (m, 2H), 7.46-7.42 (m, 1H), 7.38-7.32 (m. 7H), 6.75-6.73 (m, 1H), 4.64 (s, 2H), 4.48-4.46 (m, 2H), 4.25-4.24 (m, 2H), 3.52 (m, 2H).

### Example 90

### 4′-((7-((pyridine-4-ylmethyl)carbamoyl)-2H-benzo[b][1,4]oxazine-4(3H)-yl)methyl)-[1,1′-biphenyl]-2-carboxylic acid

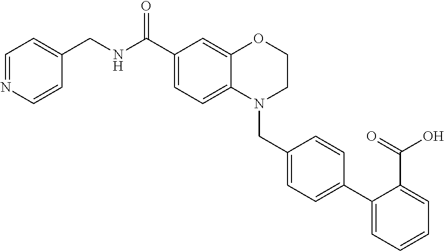 [SMILES: O=C(NCc1ccncc1)c1ccc2c(c1)OCCN2Cc1ccc(-c2ccccc2C(=O)O)cc1]

The target compound was obtained by the same manner as described in Example 58 except that pyridine-4-ylmethaneamine was used instead of (S)-1-phenylethylamine.

¹H NMR (400 MHz, DMSO-d₆) δ 12.75 (br, OH, 1H), 8.84-8.81 (m, 1H), 8.60 (m, 2H), 7.71-7.69 (m, 1H), 7.58-7.54 (m, 1H), 7.49-7.42 (m, 3H), 7.38-7.35 (m, 2H), 7.33-7.31 (m, 5H), 6.77-6.75 (m, 1H), 4.65 (s, 2H), 4.52-4.50 (m, 2H), 4.26-4.25 (m, 2H), 3.54-3.53 (m, 2H).

### Example 91

### 4′-((7-(benzylcarbamoyl)-2H-benzo[b][1,4]oxazine-4(3H)-yl)methyl)-[1,1′-biphenyl]-2-carboxylic acid

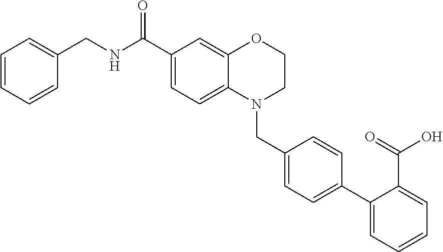 [SMILES: O=C(NCc1ccccc1)c1ccc2c(c1)OCCN2Cc1ccc(-c2ccccc2C(=O)O)cc1]

The target compound was obtained by the same manner as described in Example 58 except that phenylmethaneamine was used instead of (S)-1-phenylethylamine.

¹H NMR (400 MHz, DMSO-d₆) δ 12.75 (br, OH, 1H), 8.69-8.66 (m, 1H), 7.71-7.69 (m, 1H), 7.58-7.54 (m, 1H), 7.46-7.42 (m, 1H), 7.38-7.26 (m, 1H), 7.23-7.20 (m, 1H), 6.75-6.72 (m, 1H), 4.63 (s, 2H), 4.43-4.41 (m, 2H), 4.26-4.24 (m, 2H), 3.52-3.50 (m, 2H).

### Example 92

### 4′-((7-((2-bromobenzyl) carbamoyl)-2H-benzo[b][1,4]oxazine-4(3H)-yl)methyl)-[1,1′-biphenyl]-2-carboxylic acid

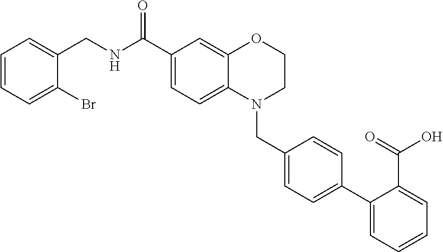 [SMILES: O=C(NCc1ccccc1Br)c1ccc2c(c1)OCCN2Cc1ccc(-c2ccccc2C(=O)O)cc1]

The target compound was obtained by the same manner as described in Example 58 except that (2-bromophenyl)methaneamine was used instead of (S)-1-phenylethylamine.

¹H NMR (400 MHz, DMSO-d₆) δ 12.75 (br, OH, 1H), 8.71-8.64 (m, 1H), 7.71-7.69 (m, 1H), 7.62-7.59 (m, 1H), 7.58-7.54 (m, 1H), 7.46-7.42 (m, 1H), 7.39-7.33 (m, 8H), 7.28-7.24 (m, 1H), 7.22-7.18 (m, 1H), 6.77-6.75 (m, 1H), 4.65 (s, 2H), 4.44-4.43 (m, 2H), 4.28-4.25 (m, 2H), 3.54-3.52 (m, 2H).

### Example 93

### 4′-((7-(((2,3-dihydrobenzo[b][1,4]dioxin-6-yl)methyl)carbamoyl)-2H-benzo[b][1,4]oxazine-4(3H)-yl)methyl)-[1,1′-biphenyl]-2-carboxylic acid

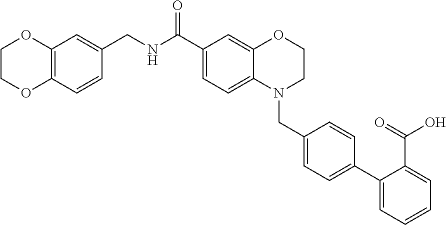 [SMILES: O=C(NCc1ccc2c(c1)OCCO2)c1ccc2c(c1)OCCN2Cc1ccc(-c2ccccc2C(=O)O)cc1]

The target compound was obtained by the same manner as described in Example 58 except that (2,3-dihydrobenzo[b][1,4]dioxin-6-yl)methylamine was used instead of (S)-1-phenylethylamine.

¹H NMR (400 MHz, DMSO-d₆) δ 8.62-8.59 (m, 1H), 7.56-7.54 (m, 1H), 7.41-7.39 (m, 2H), 7.35-7.30 (m, 4H), 7.25-7.23 (m, 3H), 6.78-6.71 (m, 4H), 4.60 (s, 2H), 4.29-4.27 (m, 2H), 4.26-4.25 (m, 2H), 4.19 (s, 4H), 3.51 (m, 2H).

### Example 94

### (R)-4-((2′-carbamoyl-[1,1′-biphenyl]-4-yl)methyl)-N-(1-(4-cyanophenyl)ethyl)-3,4-dihydro-2H-benzo[b][1,4]oxazine-7-carboxamide

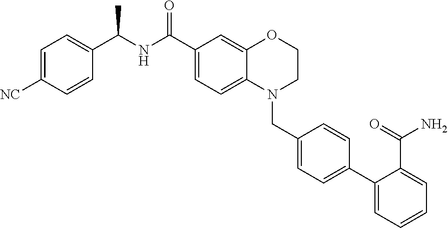 [SMILES: C[C@@H](NC(=O)c1ccc2c(c1)OCCN2Cc1ccc(-c2ccccc2C(N)=O)cc1)c1ccc(C#N)cc1]

The target compound was obtained by the same manner as described in Example 59 except that (R)-4′-((7-((1-(4-cyanophenyl)ethyl)carbamoyl)-2H-benzo[b][1,4]oxazine-4(3H)-yl)methyl)-[1,1′-biphenyl]-2-carboxylic acid obtained in Example 81 was used instead of (S)-4′-((7-((1-phenylethyl)carbamoyl)-2H-benzo[b][1,4]oxazine-4(3H)-yl)methyl)-[1,1′-biphenyl]-2-carboxylic acid (50 mg, 0.1 mmol).

¹H NMR (400 MHz, DMSO-d₆) δ 8.51 (d, J=8.0 Hz, 1H), 7.78 (d, J=8.0 Hz, 2H), 7.66 (s, 1H), 7.54 (d, J=8.0 Hz, 2H), 7.50-7.20 (m, 11H), 6.74 (d, J=8.4 Hz, 1H), 5.14 (m, 1H), 4.62 (s, 2H), 4.26 (t, J=4.0 Hz, 2H), 3.52 (t, J=4.0 Hz, 2H), 1.42 (d, J=7.2 Hz, 3H).

### Example 95

### (S)-4-((2′-carbamoyl-[1,1′-biphenyl]-4-yl)methyl)-N-(1-(3-fluorophenyl)ethyl)-3,4-dihydro-2H-benzo[b][1,4]oxazine-7-carboxamide

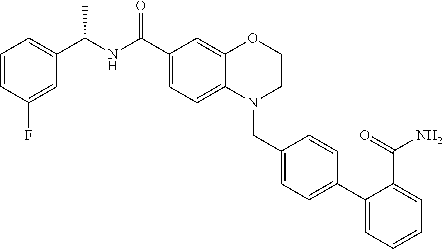 [SMILES: C[C@H](NC(=O)c1ccc2c(c1)OCCN2Cc1ccc(-c2ccccc2C(N)=O)cc1)c1cccc(F)c1]

The target compound was obtained by the same manner as described in Example 59 except that (S)-4′-((7-((1-(3-fluorophenyl)ethyl)carbamoyl)-2H-benzo[b][1,4]oxazine-4(3H)-yl)methyl)-[1,1′-biphenyl]-2-carboxylic acid obtained in Example 84 was used instead of (S)-4′-((7-((1-phenylethyl)carbamoyl)-2H-benzo[b][1,4]oxazine-4(3H)-yl)methyl)-[1,1′-biphenyl]-2-carboxylic acid (50 mg, 0.1 mmol).

¹H NMR (400 MHz, CDCl₃) δ 7.77-7.75 (m, 1H), 7.54-7.50 (m, 1H), 7.46-7.43 (m, 3H), 7.38-7.28 (m, 6H), 7.17-7.15 (m, 1H), 7.09-7.06 (m, 1H), 6.98-6.94 (m, 1H), 6.67-6.65 (m, 1H), 6.15-6.13 (m, NH, 1H), 5.46 (br, NH, 1H), 5.34-5.27 (m, 2H), 4.59 (s, 2H), 4.33-4.31 (m, 2H), 3.54-3.47 (m, 2H), 1.58-1.56 (m, 3H).

### Example 96

### (S)-4-((2′-carbamoyl-[1,1′-biphenyl]-4-yl)methyl)-N-(1-(naphthalene-2-yl)ethyl)-3,4-dihydro-2H-benzo[b][1,4]oxazine-7-carboxamide

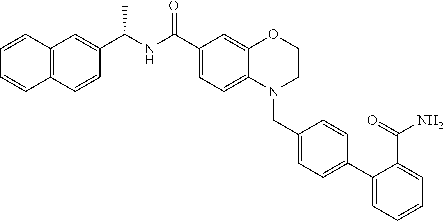 [SMILES: C[C@H](NC(=O)c1ccc2c(c1)OCCN2Cc1ccc(-c2ccccc2C(N)=O)cc1)c1ccc2ccccc2c1]

The target compound was obtained by the same manner as described in Example 59 except that (S)-4′-((7-((1-(naphthalene-2-yl)ethyl)carbamoyl)-2H-benzo[b][1,4]oxazine-4(3H)-yl)methyl)-[1,1′-biphenyl]-2-carboxylic acid obtained in Example 87 was used instead of (S)-4′-((7-((1-phenylethyl)carbamoyl)-2H-benzo[b][1,4]oxazine-4(3H)-yl)methyl)-[1,1′-biphenyl]-2-carboxylic acid (50 mg, 0.1 mmol).

¹H NMR (400 MHz, CDCl₃) δ 7.85-7.82 (m, 4H), 7.77-7.74 (m, 1H), 7.53-7.42 (m, 8H), 7.37-7.35 (m, 1H), 7.33-7.29 (m, 3H), 6.66-6.64 (m, 1H), 6.25-6.23 (m, NH, 1H), 5.51-5.46 (m, 1H), 5.46 (br, NH, 1H), 5.32 (br, NH, 1H), 4.58 (s, 2H), 4.32-4.30 (m, 2H), 3.53-3.49 (m, 2H), 1.67-1.67 (m, 3H).

### Example 97

### 4-((2′-carbamoyl-[1,1′-biphenyl]-4-yl)methyl)-N-(pyridine-3-ylmethyl)-3,4-dihydro-2H-benzo[b][1,4]oxazine-7-carboxamide

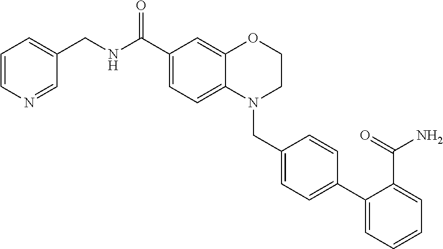 [SMILES: NC(=O)c1ccccc1-c1ccc(CN2CCOc3cc(C(=O)NCc4cccnc4)ccc32)cc1]

The target compound was obtained by the same manner as described in Example 59 except that 4′-((7-((pyridine-3-ylmethyl)carbamoyl)-2H-benzo[b][1,4]oxazine-4(3H)-yl)methyl)-[1,1′-biphenyl]-2-carboxylic acid obtained in Example 89 was used instead of (S)-4′-((7-((1-phenylethyl)carbamoyl)-2H-benzo[b][1,4]oxazine-4(3H)-yl)methyl)-[1,1′-biphenyl]-2-carboxylic acid (50 mg, 0.1 mmol).

¹H NMR (400 MHz, DMSO-d₆) δ 8.72-8.70 (m, 1H), 8.51 (m, H), 8.44-8.43 (m, 1H), 7.68-7.64 (m, 2H), 7.48-7.26 (m, 12H), 6.75-6.72 (m, 1H), 4.62 (s, 2H), 4.44-4.42 (m, 2H), 4.26-4.24 (m, 2H), 3.53-3.50 (m, 2H).

### Example 98

### 4-((2′-carbamoyl-[1,1′-biphonyl]-4-yl)methyl)-N-(pyridine-4-ylmethyl)-3,4-dihydro-2H-benzo[b][1,4]oxazine-7-carboxamide

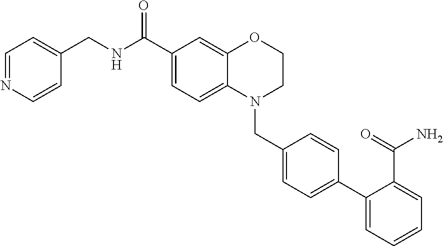 [SMILES: NC(=O)c1ccccc1-c1ccc(CN2CCOc3cc(C(=O)NCc4ccncc4)ccc32)cc1]

The target compound was obtained by the same manner as described in Example 59 except that 4′-((7-((pyridine-4-ylmethyl)carbamoyl)-2H-benzo[b][1,4]oxazine-4(3H)-yl)methyl)-[1,1′-biphenyl]-2-carboxylic acid obtained in Example 90 was used instead of (S)-4′-((7-((1-phenylethyl)carbamoyl)-2H-benzo[b][1,4]oxazine-4(3H)-yl)methyl)-[1,1′-biphenyl]-2-carboxylic acid (50 mg, 0.1 mmol).

¹H NMR (400 MHz, DMSO-d₆) δ 8.76-8.73 (m, 1H), 8.49-8.47 (m, 2H), 7.65 (m, 1H), 7.49-7.43 (m, 1H), 7.41-7.35 (m, 6H), 7.32-7.25 (m, 6H), 6.76-6.74 (m, 1H), 4.63 (s, 2H), 4.44-4.42 (m, 2H), 4.27-4.25 (m, 2H), 3.54-3.52 (m, 2H).

### Example 99

### (S)-4-((2′-carbamoyl-[1,1′-biphenyl]-4-yl)methyl)-N-(1-(4-((trifluoromethyl)thio)phenyl)ethyl)-3, 4-dihydro-2H-benzo[b][1,4]oxazine-7-carboxamide

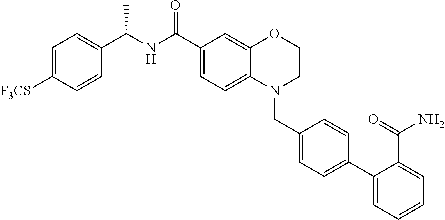

The target compound was obtained by the same manner as described in Example 59 except that (S)-4′-((7-((1-(4-((trifluoromethyl)thio)phenyl)ethyl)carbamoyl)-2H-benzo[b][1,4]oxazine-4(3H)-yl)methyl)-[1,1′-biphenyl]-2-carboxylic acid obtained in Example 77 was used instead of (S)-4′-((7-((1-phenylethyl)carbamoyl)-2H-benzo[b][1,4]oxazine-4(3H)-yl)methyl)-[1,1′-biphenyl]-2-carboxylic acid (50 mg, 0.1 mmol).

¹H NMR (400 MHz, DMSO-d₆) δ 8.50-8.48 (m, 1H), 7.67-7.65 (m, 3H), 7.51-7.27 (m, 13H), 6.74-6.72 (m, 1H), 5.16-5.13 (m, 1H), 4.62 (s, 2H), 4.26-4.25 (m, 2H), 3.52-3.51 (m, 2H), 1.46-4.11 (m, 3H).

### Example 100

### N-benzyl-4-((2′-carbamoyl-[1,1′-biphenyl]-4-yl)methyl)-3,4-dihydro-2H-benzo[b][1,4]oxazine-7-carboxamide

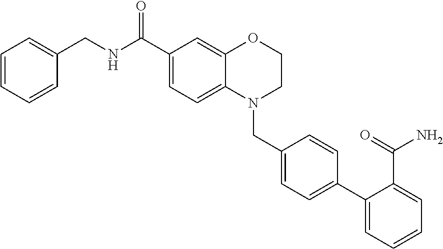 [SMILES: NC(=O)c1ccccc1-c1ccc(CN2CCOc3cc(C(=O)NCc4ccccc4)ccc32)cc1]

The target compound was obtained by the same manner as described in Example 59 except that 4′-((7-(benzylcarbamoyl)-2H-benzo[b][1,4]oxazine-4(3H)-yl)methyl)-[1,1′-biphenyl]-2-carboxylic acid obtained in Example 91 was used instead of (S)-4′-((7-((1-phenylethyl)carbamoyl)-2H-benzo[b][1,4]oxazine-4(3H)-yl)methyl)-[1,1′-biphenyl]-2-carboxylic acid (50 mg, 0.1 mmol).

¹H NMR (400 MHz, CDCl₃) δ 7.77-7.74 (m, 1H), 7.54-7.50 (m, 1H), 7.49-7.42 (m, 3H), 7.38-7.28 (m, 10H), 6.66-6.64 (m, 1H), 6.24-6.21 (m, NH, 1H), 5.59 (br, NH, 1H), 5.34 (br, NH, 1H), 4.64-4.63 (m, 2H), 4.59 (s, 2H), 4.32-4.30 (m, 2H), 3.53-3.51 (m, 2H).

### Example 101

### N-(2-bromobenzyl)-4-((2′-carbamoyl-[1,1′-biphenyl]-4-yl)methyl)-3,4-dihydro-2H-benzo[b][1,4]oxazine-7-carboxamide

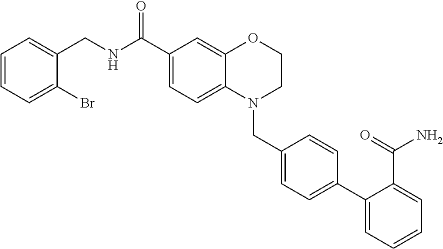 [SMILES: NC(=O)c1ccccc1-c1ccc(CN2CCOc3cc(C(=O)NCc4ccccc4Br)ccc32)cc1]

The target compound was obtained by the same manner as described in Example 59 except that 4′-((7-((2-bromobenzyl)carbamoyl)-2H-benzo[b][1,4]oxazine-4(3H)-yl)methyl)-[1,1′-biphenyl]-2-carboxylic acid obtained in Example 92 was used instead of (S)-4′-((7-((1-phenylethyl)carbamoyl)-2H-benzo[b][1,4]oxazine-4(3H)-yl)methyl)-[1,1′-biphenyl]-2-carboxylic acid (50 mg, 0.1 mmol).

¹H NMR (400 MHz, CDCl₃) δ 7.70-7.75 (m, 1H), 7.58-7.56 (m, 1H), 7.54-7.48 (m, 2H), 7.46-7.42 (m, 4H), 7.38-7.36 (m, 1H), 7.33-7.28 (m, 4H), 7.18-7.14 (m, 1H), 6.66-6.64 (m, 1H), 6.49-6.46 (m, NH, 1H), 5.48 (br, NH, 1H), 5.32 (br, NH, 1H), 4.70-4.69 (m, 2H), 4.59 (s, 2H), 4.33-4.30 (m, 2H), 3.54-3.51 (m, 2H).

### Example 102

### 4-((2′-carbamoyl-[1,1′-biphenyl]-4-yl)methyl)-N-((2,3-dihydrobenzo[b][1,4]dioxin-6-yl)methyl)-3,4-dihydro-2H-benzo[b][1,4]oxazine-7-carboxamide

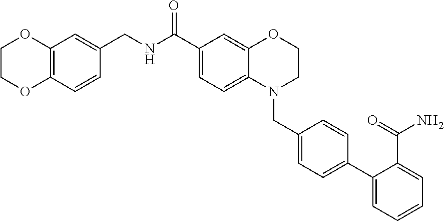 [SMILES: NC(=O)c1ccccc1-c1ccc(CN2CCOc3cc(C(=O)NCc4ccc5c(c4)OCCO5)ccc32)cc1]

The target compound was obtained by the same manner as described in Example 59 except that 4′-((7-(((2,3-dihydrobenzo[b][1,4]dioxin-6-yl)methyl)carbamoyl)-2H-benzo[b][1,4]oxazine-4(3H)-yl)methyl)-[1,1′-biphenyl]-2-carboxylic acid obtained in Example 93 was used instead of (S)-4′-((7-((1-phenylethyl)carbamoyl)-2H-benzo[b][1,4]oxazine-4(3H)-yl)methyl)-[1,1′-biphenyl]-2-carboxylic acid (50 mg, 0.1 mmol).

¹H NMR (400 MHz, CDCl₃) δ 7.77-7.75 (m, 1H), 7.53-7.50 (m, 1H), 7.48-7.42 (m, 3H), 7.38-7.36 (m, 1H), 7.33-7.31 (m, 2H), 7.28-7.27 (m, 2H), 6.86-6.81 (m, 3H), 6.65-6.63 (m, 1H), 6.16-6.14 (m, NH, 1H), 5.50 (br, NH, 1H), 5.34 (br, NH, 1H), 4.58 (s, 2H), 4.51-4.50 (m, 2H), 4.32-4.30 (m, 2H), 4.26 (s, 4H), 3.53-3.51 (m, 2H).

### Example 103

### sodium (S)-4′-((7-((1-phenylethyl)carbamoyl)-2H-benzo[b][1,4]oxazine-4(3H)-yl)methyl)-[1,1′-biphenyl]-2-carboxylate

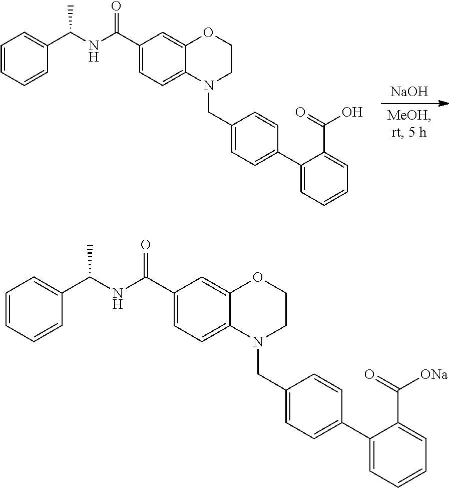

(S)-4′-((7-((1-phenylethyl)carbamoyl)-2H-benzo[b][1,4]oxazine-4(3H)-yl)methyl)-[1,1′-biphenyl]-2-carboxylic acid (300 mg, 0.61 mmol) obtained in Example 58 was dissolved in MeOH (10 ml) in a 25 mL flask, to which NaOH (24.4 mg, 0.61 mmol) was added, followed by stirring at room temperature for 5 hours.

Upon completion of the reaction, the reaction mixture was concentrated under reduced pressure. As a result, sodium (S)-4′-((7-((1-phenylethyl)carbamoyl)-2H-benzo[b][1,4]oxazine-4(3H)-yl)methyl)-[1,1′-biphenyl]-2-carboxylate was obtained (300 mg).

¹H NMR (400 MHz, DMSO-d₆) δ 8.41 (d, J=8.0 Hz, 1H), 7.48 (m, 2H), 7.39-7.25 (m, 7H), 7.23-7.15 (m, 6H), 6.74 (d, J=8.4 Hz, 1H), 5.12 (m, 1H), 4.58 (s, 2H), 4.24 (t, J=4.0 Hz, 2H), 3.51 (t, J=4.0 Hz, 2H), 1.44 (d, J=7.2 Hz, 3H).

### Example 104

### sodium (S)-4′-((7-((1-(4-cyanophenyl)ethyl)carbamoyl)-2H-benzo[b][1,4]oxazine-4(3H)-yl)methyl)-[1,1′-biphenyl]-2-carboxylate

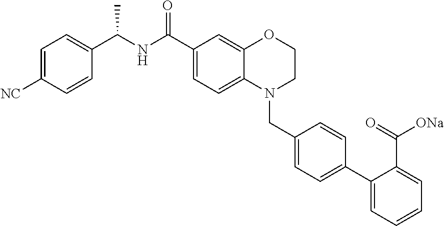

The target compound was obtained by the same manner as described in Example 103 except that (S)-4′-((7-((1-(4-cyanophenyl)ethyl)carbamoyl)-2H-benzo[b][1,4]oxazine-4(3H)-yl)methyl)-[1,1′-biphenyl]-2-carboxylic acid obtained in Example 74 was used instead of (S)-4′-((7-((1-phenylethyl)carbamoyl)-2H-benzo[b][1,4]oxazine-4(3H)-yl)methyl)-[1,1′-biphenyl]-2-carboxylic acid.

¹H NMR (400 MHz, DMSO-d₆) δ 8.55 (d, J=7.6 Hz, 1H), 7.76 (d, J=8.4 Hz, 2H), 7.54, (d, J=8.4 Hz, 2H), 7.49-7.42 (m, 3H), 7.39-7.30 (m, 2H), 7.28-7.15 (m, 5H), 6.73 (d, J=8.4 Hz, 1H), 5.14 (m, 1H), 4.56 (s, 2H), 4.22 (t, J=4.0 Hz, 2H), 3.48 (t, J=4.0 Hz, 2H), 1.44 (d, J=7.2 Hz, 3H).

### Example 105

### sodium (S)-4′-((7-((1-(4-nitrophenyl)ethyl)carbamoyl)-2-benzo[b][1,4]oxazine-4(3H)-yl)methyl)-[1,1′-biphenyl]-2-carboxylate

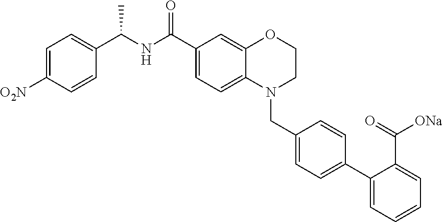

The target compound was obtained by the same manner as described in Example 103 except that (S)-4′-((7-((1-(4-nitrophenyl)ethyl)carbamoyl)-2H-benzo[b][1,4]oxazine-4(3H)-yl)methyl)-[1,1′-biphenyl]-2-carboxylic acid obtained in Example 60 was used instead of (S)-4′-((7-((1-phenylethyl)carbamoyl)-2H-benzo[b][1,4]oxazine-4(3H)-yl)methyl)-[1,1′-biphenyl]-2-carboxylic acid.

¹H NMR (400 MHz, DMSO-d₆) δ 8.61 (d, J=7.6 Hz, 1H), 8.16 (d, J=8.4 Hz, 2H), 7.61 (d, J=8.8 Hz, 2H), 7.46 (d, J=8.4 Hz, 2H), 7.37-7.16 (m, 8H), 6.75 (d, J=8.8 Hz, 1H), 5.18 (m, 1H), 4.59 (s, 2H), 4.25 (t, J=4.0 Hz, 2H), 3.52 (t, J=4.0 Hz, 2H), 1.46 (d, J=7.2 Hz, 3H).

### Example 106

### (S)-1-((2′-carbamoyl-3-fluoro-[1,1′-biphenyl]-4-yl)methyl)-N-(1-phenylethyl)-1,2,3,4-tetrahydroquinoline-6-carboxylic acid

### Step 1 (S)-tert-butyl 3′-fluoro-4′-((6-((1-phenylethyl)carbamoyl)-3,4-dihydroquinoline-1(2H)-yl)methyl)-[1,1′-biphenyl]-2-carboxylate

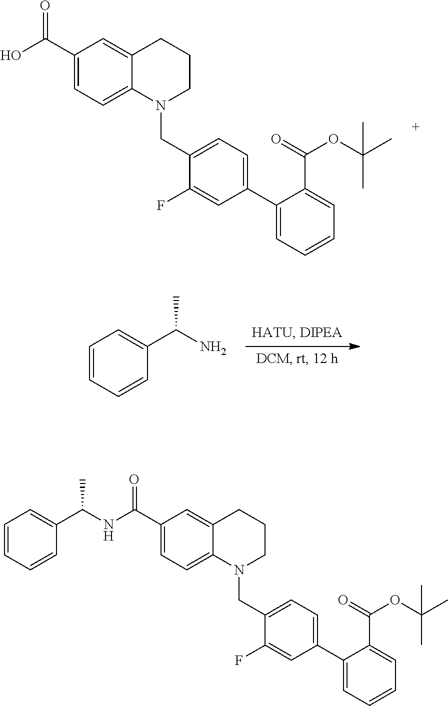

1-((2′-(tert-butoxycarbonyl)-3-fluoro-[1,1′-biphenyl]-4-yl)methyl)-1,2,3,4-tetrahydroquinoline-6-carboxylic acid (300 mg, 0.67 mmol) obtained in Preparative Example 3 was dissolved in CH₂Cl₂ (10 ml) in a 100 mL flask, to which HATU (386 mg, 1.01 mmol) and DIPEA (353 ul, 2.02 mmol) were added, followed by stirring at room temperature for 12 hours. Upon completion of the reaction, the organic layer was separated by using CH₂Cl₂ and H₂O. The separated organic layer was dried over MgSO₄ and filtered. The mixture was separated by column chromatography (EA/n-Hex=1:1) to give (S)-tert-butyl 3′-fluoro-4′-((6-((1-phenylethyl)carbamoyl)-3,4-dihydroquinoline-1(2H)-yl)methyl)-[1,1′-biphenyl]-2-carboxylate (169 mg, 0.295 mmol, 44%).

### Step 2 (S)-1-((2′-carbamoyl-3-fluoro-[1,1′-biphenyl]-4-yl)methyl)-N-(1-phenylethyl)-1,2,3,4-tetrahydroquinoline-6-carboxylic acid

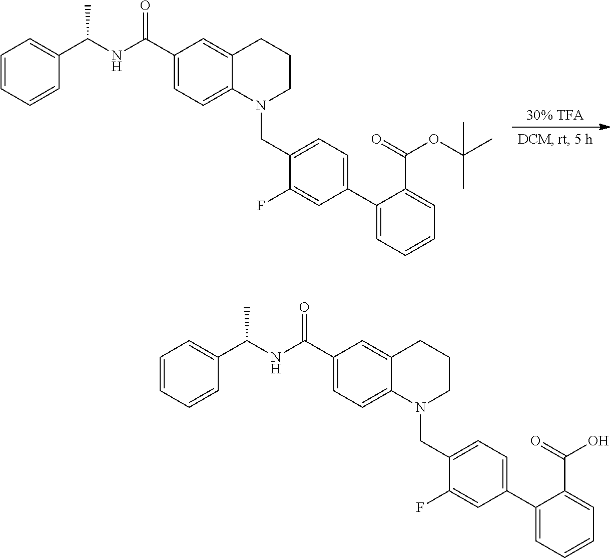 [SMILES: C[C@H](NC(=O)c1ccc2c(c1)CCCN2Cc1ccc(-c2ccccc2C(=O)OC(C)(C)C)cc1F)c1ccccc1 ; "30% TFA" ; "DCM, rt, 5 h" ; C[C@H](NC(=O)c1ccc2c(c1)CCCN2Cc1ccc(-c2ccccc2C(=O)O)cc1F)c1ccccc1]

(S)-tert-butyl 3′-fluoro-4′-((6-((1-phenylethyl)carbamoyl)-3,4-dihydroquinoline-1(2H)-yl)methyl)-[1,1′-biphenyl]-2-carboxylate (168 mg, 0.3 mmol) obtained in step 1 was dissolved in CH₂Cl₂ (10 ml) in a 50 mL flask, to which 30% TFA (3 ml) was added, followed by stirring at room temperature for 5 hours. Upon completion of the reaction the reaction mixture was concentrated under reduced pressure. The mixture was separated by column chromatography (EA) to give (S)-1-((2′-carbamoyl-3-fluoro-[1,1′-biphenyl]-4-yl)methyl)-N-(1-phenylethyl)-1,2,3,4-tetrahydroquinoline-6-carboxylic acid (83 mg, 0.16 mmol, 54%).

¹H NMR (400 MHz, CDCl₃) δ 7.93 (d, J=7.2 Hz, 1H), 7.56-7.25 (m, 10H), 7.10-7.07 (m, 2H), 7.02-7.00 (m, 1H), 6.45 (d, J=7.6 Hz, 1H), 6.16 (d, J=7.6 Hz, 1H), 5.38-5.32 (m, 1H), 4.60 (s, 2H), 3.47 (t, J=4.0 Hz, 2H), 2.85 (t, J=4.0 Hz, 2H), 2.03 (t, J=4.0 Hz, 2H), 1.57 (d, J=6.8 Hz, 3H).

### Example 107

### (S)-1-((2′-carbamoyl-3-fluoro-[1,1′-biphenyl]-4-yl)methyl)-N-(1-phenylethyl)-1,2,3,4-tetrahydroquinoline-6-carboxamide

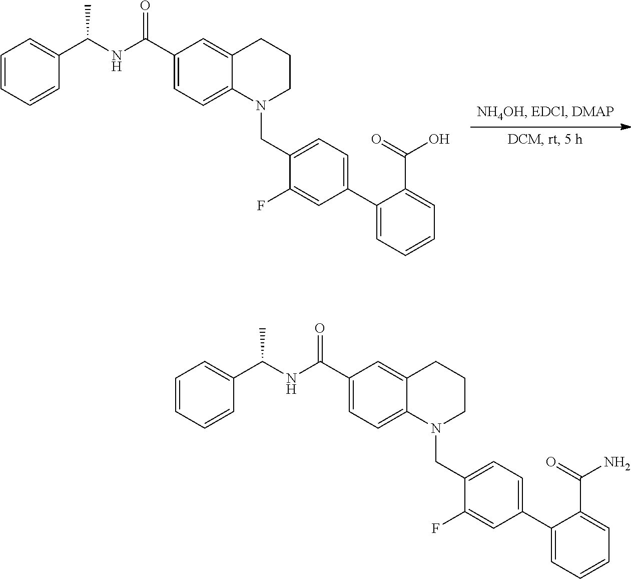 [SMILES: C[C@H](NC(=O)c1ccc2c(c1)CCCN2Cc1ccc(-c2ccccc2C(=O)O)cc1F)c1ccccc1 ; "DCM, rt, 5 h" ; "NH4OH, EDCl, DMAP" ; C[C@H](NC(=O)c1ccc2c(c1)CCCN2Cc1ccc(-c2ccccc2C(N)=O)cc1F)c1ccccc1]

(S)-1-((2′-carbamoyl-3-fluoro-[1,1′-biphenyl]-4-yl)methyl)-N-(1-phenylethyl)-1,2,3,4-tetrahydroquinoline-6-carboxylic acid (78 mg, 0.15 mmol) obtained in Example 106 was dissolved in CH₂Cl₂ (2 ml) in a 25 mL flask, to which EDCI (45 mg, 0.23 mmol) and HOBt (24 mg, 0.18 mmol) were added, followed by stirring. Ammonium hydroxide solution (3 ml) was added thereto, followed by stirring at room temperature for 5 hours. Upon completion of the reaction, the organic layer was separated by using CH₂Cl₂ and H₂O. The separated organic layer was dried over MgSO₄ and filtered. The mixture was separated by column chromatography (MeOH/n-Hex/CH₂Cl₂=0.5:0.5:9) to give (S)-1-((2′-carbamoyl-3-fluoro-[1,1′-biphenyl]-4-yl)methyl)-N-(1-phenylethyl)-1,2,3,4-tetrahydroquinoline-6-carboxamide (76 mg, 0.14 mmol, 97.6%).

¹H NMR (400 MHz, CDCl₃) δ 7.73-7.71 (m, 1H), 7.54-7.25 (m, 10H), 7.23-7.14 (m, 3H), 6.46 (d, J=8.8 Hz, 1H), 6.14 (d, J=8.0 Hz, 1H), 5.51 (s, 1H), 5.40 (s, 1H), 5.38-5.31 (m, 1H), 4.62 (s, 2H), 3.49 (t, J=5.6 Hz, 2H), 2.87 (t, J=6.4 Hz, 2H), 2.10-2.04 (m, 2H), 1.59 (d, J=6.8 Hz, 3H).

### Example 108

### (S)-3′-fluoro-4′-((6-((1-(naphthalene-2-yl)ethyl)carbamoyl)-3,4-dihydroquinoline-1(28)-yl)methyl)-[1,1′-biphenyl]-2-carboxylic acid

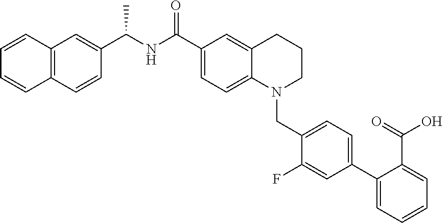 [SMILES: C[C@H](NC(=O)c1ccc2c(c1)CCCN2Cc1ccc(-c2ccccc2C(=O)O)cc1F)c1ccc2ccccc2c1]

The target compound was obtained by the same manner as described in Example 106 except that (S)-1-(naphthalene-2-yl)ethaneamine was used instead of (S)-1-phenylethylamine.

¹H NMR (400 MHz, CDCl₃) δ 7.91 (s, 1H), 7.82-7.80 (m, 4H), 7.54-7.36 (m, 8H), 7.32-7.30 (m, 1H), 7.09-7.07 (m, 2H), 7.02-6.98 (m, 1H), 6.43 (d, J=8.4 Hz, 1H), 6.26 (d, J=7.6 Hz, 1H), 5.51-5.47 (m, 1H), 4.58 (s, 2H), 3.44 (t, J=4.0 Hz, 2H), 2.83 (t, J=4.0 Hz, 2H), 2.01 (t, J=4.0 Hz, 2H), 1.65 (d, J=6.8 Hz, 3H).

### Example 109

### (S)-3′-fluoro-4′-((6-((1-(4-nitrophenyl)ethyl)carbamoyl)-3,4-dihydroquinoline-1(2H)-yl)methyl)-[1,1′-biphenyl]-2-caboxylic acid

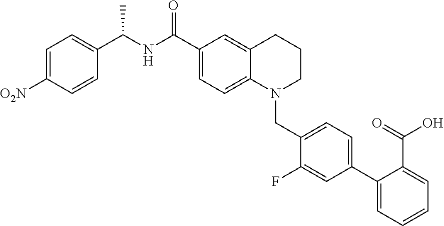 [SMILES: C[C@H](NC(=O)c1ccc2c(c1)CCCN2Cc1ccc(-c2ccccc2C(=O)O)cc1F)c1ccc([N+](=O)[O-])cc1]

The target compound was obtained by the same manner as described in Example 106 except that (S)-1-(4-nitrophenyl)ethaneamine was used instead of (S)-1-phenylethylamine.

¹H NMR (400 MHz, CDCl₃) δ 8.17 (d, J=8.8 Hz, 2H), 7.97 (d, J=7.6 Hz, 1H), 7.61-7.57 (m, 1H), 7.53-7.42 (m, 5H), 7.35 (d, J=7.6 Hz, 1H), 7.16-7.09 (m, 2H), 7.03 (d, J=8.0 Hz, 1H), 6.48 (d, J=8.4 Hz, 1H), 6.27 (d, J=7.2 Hz, 1H), 5.38-5.31 (m, 1H), 4.63 (s, 2H), 3.53-3.48 (m, 2H), 2.86 (t, J=6.0 Hz, 2H), 2.07-2.04 (m, 2H), 1.58 (d, J=6.8 Hz, 3H).

### Example 110

### (S)-4′-((6-((1-(4-cyanophenyl)ethyl)carbamoyl)-3,4-dihydroquinoline-1(2H)-yl)methyl)-3′-fluoro-[1,1′-biphenyl]-2-carboxylic acid

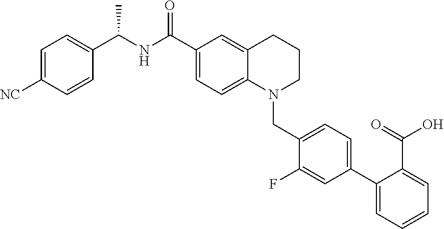 [SMILES: C[C@H](NC(=O)c1ccc2c(c1)CCCN2Cc1ccc(-c2ccccc2C(=O)O)cc1F)c1ccc(C#N)cc1]

The target compound was obtained by the same manner as described in Example 106 except that (S)-1-(4-cyanophenyl)ethaneamine was used instead of (S)-1-phenylethylamine.

¹H NMR (400 MHz, CDCl₃) δ 7.98 (d, J=6.4 Hz, 1H), 7.68-7.58 (m, 3H), 7.49-7.42 (m, 6H), 7.36-7.33 (m, 2H), 7.16-7.09 (m, 2H), 7.05-7.02 (m, 1H), 6.49 (d, J=8.8 Hz, 1H), 6.19 (d, J=8.0 Hz, 1H), 5.35-5.30 (m, 1H), 4.64 (s, 2H), 3.51-3.48 (m, 2H), 2.87 (t, J=6.8 Hz, 2H), 2.07-2.00 (m, 2H), 1.57 (d, J=6.8 Hz, 3H).

### Example 111

### (S)-3′-fluoro-4′-((6-((1-(3-fluoro-4-methylphenyl)ethyl)carbamoyl)-3,4-dihydroquinoline-1(2H)-yl)methyl)-[1,1′-biphenyl]-2-caboxylic acid

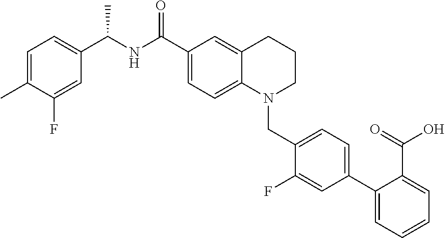 [SMILES: Cc1ccc([C@H](C)NC(=O)c2ccc3c(c2)CCCN3Cc2ccc(-c3ccccc3C(=O)O)cc2F)cc1F]

The target compound was obtained by the same manner as described in Example 106 except that (S)-1-(3-fluoro-4-methylphenyl)ethaneamine was used instead of (S)-1-phenylethylamine.

¹H NMR (400 MHz, CDCl₃) δ 7.97 (d, J=8.0 Hz, 1H), 7.61-7.57 (m, 1H), 7.49-7.35 (m, 5H), 7.16-7.00 (m, 6H), 6.48 (d, J=8.8 Hz, 1H), 6.13 (d, J=7.2 Hz, 1H), 5.32-5.26 (m, 1H), 4.63 (s, 2H), 3.50 (t, J=6.4 Hz, 2H), 2.86 (t, J=5.6 Hz, 2H), 2.26 (s, 3H) 2.12-2.06 (m, 2H), 1.55 (d, J=6.8 Hz, 3H).

### Example 112

### (S)-1-((2′-carbamoyl-3-fluoro-[1,1′-biphenyl]-4-yl)methyl)-N-(1-(4-cyanophenyl)ethyl)-1,2,3,4-tetrahydroquinoline-6-carboxamide

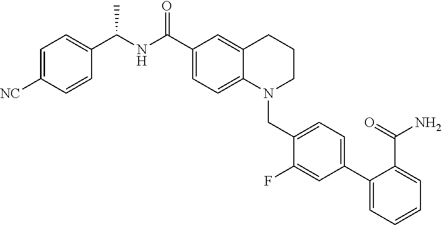 [SMILES: C[C@H](NC(=O)c1ccc2c(c1)CCCN2Cc1ccc(-c2ccccc2C(N)=O)cc1F)c1ccc(C#N)cc1]

The target compound was obtained by the same manner as described in Example 107 except that (S)-4′-((6-((1-(4-cyanophenyl)ethyl)carbamoyl)-3,4-dihydroquinoline-1(2H)-yl)methyl)-3′-fluoro-[1,1′-biphenyl]-2-carboxylic acid obtained in Example 110 was used instead of (S)-1-((2′-carbamoyl-3-fluoro-[1,1′-biphenyl]-4-yl)methyl)-N-(1-phenylethyl)-1,2,3,4-tetrahydroquinoline-6-carboxylic acid.

¹H NMR (400 MHz, CDCl₃) δ 7.72 (d, J=8.0 Hz, 1H), 7.63 (d, J=8.0 Hz, 1H), 7.54-7.43 (m, 6H), 7.36 (d, J=8.4 Hz, 1H), 7.23-7.15 (m, 3H), 6.48 (d, J=8.8 Hz, 1H), 6.17 (d, J=7.2 Hz, 1H), 5.48 (s, 1H), 5.41 (s, 1H), 5.36-5.29 (m, 1H), 4.64 (s, 2H), 3.51 (t, J=5.6 Hz, 2H), 2.88 (t, J=5.6 Hz, 2H), 2.09-2.05 (m, 2H), 1.58 (d, J=7.2 Hz, 3H).

### Example 113

### (S)-1-((2′-carbamoyl-3-fluoro-[1,1′-biphenyl]-4-yl)methyl)-N-(1-(4-nitrophenyl)ethyl)-1,2,3,4-tetrahydroquinoline-6-carboxamide

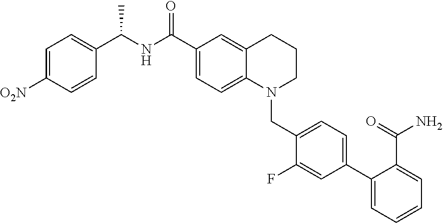 [SMILES: C[C@H](NC(=O)c1ccc2c(c1)CCCN2Cc1ccc(-c2ccccc2C(N)=O)cc1F)c1ccc([N+](=O)[O-])cc1]

The target compound was obtained by the same manner as described in Example 107 except that (S)-3′-fluoro-4′-((6-((1-(4-nitrophenyl)ethyl)carbamoyl)-3,4-dihydroquinoline-1(2H)-yl)methyl)-[1,1′-biphenyl]-2-carboxylic acid obtained in Example 109 was used instead of (S)-1-((2′-carbamoyl-3-fluoro-[1,1′-biphenyl]-4-yl)methyl)-N-(1-phenylethyl)-1,2,3,4-tetrahydroquinoline-6-carboxylic acid.

¹H NMR (400 MHz, CDCl₃) δ 8.20 (d, J=8.8 Hz, 2H), 7.72 (d, J=6.8 Hz, 1H), 7.55-7.44 (m, 6H), 7.36 (d, J=5.6 Hz, 1H), 7.23-7.15 (m, 3H), 6.49 (d, J=8.8 Hz, 1H), 6.20 (d, J=7.2 Hz, 1H), 5.47 (s, 1H), 5.41 (s, 1H), 5.38-5.35 (m, 1H), 4.64 (s, 2H), 3.51 (t, J=5.6 Hz, 2H), 2.89 (t, J=6.0 Hz, 2H), 2.09-2.07 (m, 2H), 1.61 (d, J=7.2 Hz, 3H).

### Example 114

### (S)-1-((2′-carbamoyl-3-fluoro-[1,1′-biphenyl]-4-yl)methyl)-N-(1-(3-fluoro-4-methylphenyl)ethyl)-1,2,3,4-tetrahydroquinoline-6-carboxamide

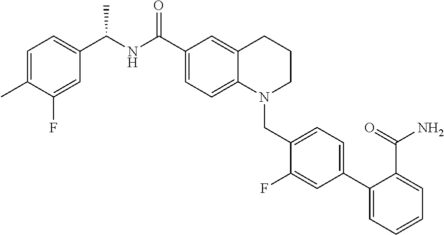 [SMILES: Cc1ccc([C@H](C)NC(=O)c2ccc3c(c2)CCCN3Cc2ccc(-c3ccccc3C(N)=O)cc2F)cc1F]

The target compound was obtained by the same manner as described in Example 107 except that (S)-3′-fluoro-4′-((6-((1-(3-fluoro-4-methylphenyl)ethyl)carbamoyl)-3,4-dihydroquinoline-1(2H)-yl)methyl)-[1,1′-biphenyl]-2-carboxylic acid obtained in Example 111 was used instead of (S)-1-((2′-carbamoyl-3-fluoro-[1,1′-biphenyl]-4-yl)methyl)-N-(1-phenylethyl)-1,2,3,4-tetrahydroquinoline-6-carboxylic acid.

¹H NMR (400 MHz, CDCl₃) δ 7.74-7.71 (m, 1H), 7.54-7.33 (m, 5H), 7.23-7.13 (m, 4H), 7.07-7.02 (m, 2H), 6.47 (d, J=8.4 Hz, 1H), 6.09 (d, J=7.6 Hz, 1H), 5.49 (s, 1H), 5.40 (s, 1H), 5.32-5.25 (m, 1H), 4.63 (s, 2H), 3.50 (t, J=5.2 Hz, 2H), 2.88 (t, J=6.0 Hz, 2H), 2.26 (s, 3H), 2.10-2.04 (m, 2H), 1.56 (d, J=6.8 Hz, 3H).

### Example 115

### (S)-1-((2′-carbamoyl-3-fluoro-[1,1′-biphenyl]-4-yl)methyl)-N-(1-(naphthalene-2-yl)ethyl)-1,2,3,4-tetrahydroquinoline-6-carboxamide

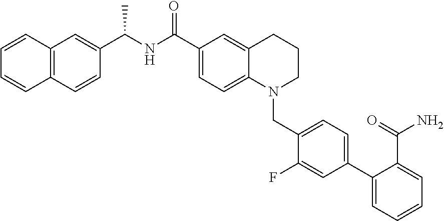 [SMILES: C[C@H](NC(=O)c1ccc2c(c1)CCCN2Cc1ccc(-c2ccccc2C(N)=O)cc1F)c1ccc2ccccc2c1]

The target compound was obtained by the same manner as described in Example 107 except that (S)-3′-fluoro-4′-((6-((1-(naphthalene-2-yl)ethyl)carbamoyl)-3,4-dihydroquinoline-1(2H)-yl)methyl)-[1,1′-biphenyl]-2-carboxylic acid obtained in Example 108 was used instead of (S)-1-((2′-carbamoyl-3-fluoro-[1,1′-biphenyl]-4-yl)methyl)-N-(1-phenylethyl)-1,2,3,4-tetrahydroquinoline-6-carboxylic acid.

¹H NMR (400 MHz, CDCl₃) δ 7.85-7.83 (m, 4H), 7.73-7.71 (m, 1H), 7.53-7.43 (m, 7H), 7.37-7.35 (m, 1H), 7.22-7.14 (m 3H), 6.47 (d, J=8.8 Hz, 1H), 6.22 (d, J=7.6 Hz, 1H), 5.55-5.48 (m, 2H), 5.40 (s, 1H), 4.63 (s, 2H), 3.50 (t, J=5.6 Hz, 2H), 2.88 (t, J=6.0 Hz, 2H), 2.09-2.04 (m, 2H), 1.69 (d, J=6.8 Hz, 3H).

### Example 116

### (S)-3′-((6-((1-phenylethyl)carbamoyl)-3,4-dihydroquinoline-1(2H)-yl)methyl)-[1,1′-biphenyl]-2-carboxylic acid

### Step 1 (S)-tert-butyl 3′-((6-((1-phenylethyl)carbamoyl)-3,4-dihydroquinoline-1(2H)-yl)methyl)-[1,1′-biphenyl]-2-carboxylate

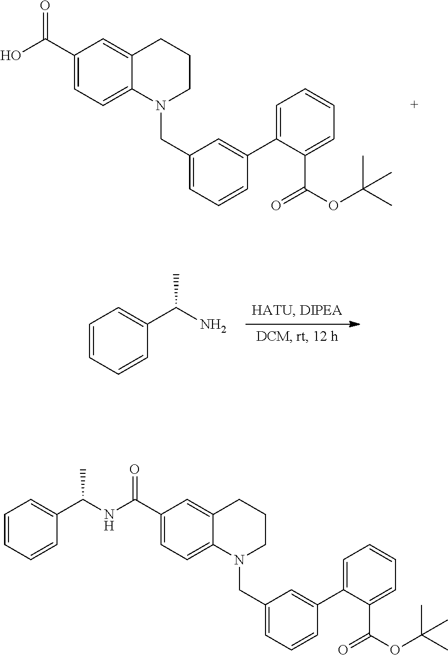

1-((2′-(tert-butoxycarbonyl)-[1,1′-biphenyl]-3-yl)methyl)-1,2,3,4-tetrahydroquinoline-6-carboxylic acid (300 mg, 0.67 mmol) obtained in Preparative Example 4 was dissolved in CH₂Cl₂ (10 ml) in a 100 mL flask, to which HATU (386 mg, 1.01 mmol) and DIPEA (353 ul, 2.02 mmol) were added, followed by stirring at room temperature for 12 hours. Upon completion of the reaction, the organic layer was separated by using CH₂Cl₂ and H₂O. The separated organic layer was dried over MgSO₄ and filtered. The mixture was separated by column chromatography (EA/n-Hex=1:1) to give (S)-tert-butyl 3′-((6-((1-phenylethyl)carbamoyl)-3,4-dihydroquinoline-1(2H)-yl)methyl)-[1,1′-biphenyl]-2-carboxylate (169 mg, 0.29 mmol, 44%).

### Step 2 (S)-3′-((6-((1-phenylethyl)carbamoyl)-3,4-dihydroquinoline-1(2H)-yl)methyl)-[1,1′-biphenyl]-2-carboxylic acid

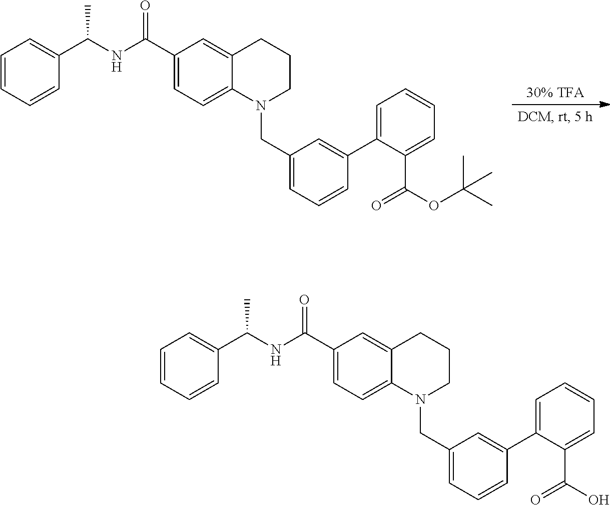 [SMILES: C[C@H](NC(=O)c1ccc2c(c1)CCCN2Cc1cccc(-c2ccccc2C(=O)OC(C)(C)C)c1)c1ccccc1 ; "30% TFA" ; "DCM, rt, 5 h" ; C[C@H](NC(=O)c1ccc2c(c1)CCCN2Cc1cccc(-c2ccccc2C(=O)O)c1)c1ccccc1]

(S)-tert-butyl 3′-((6-((1-phenylethyl)carbamoyl)-3,4-dihydroquinoline-1(2H)-yl)methyl)-[1,1′-biphenyl]-2-carboxylate (168 mg, 0.3 mmol) obtained in step 1 was dissolved in CH₂Cl₂ (10 ml) in a 50 mL flask, to which 30% TFA (3 ml) was added, followed by stirring at room temperature for 5 hours. Upon completion of the reaction the reaction mixture was concentrated under reduced pressure. The mixture was separated by column chromatography (EA) to give (S)-3′-((6-((1-phenylethyl)carbamoyl)-3,4-dihydroquinoline-1(2H)-yl)methyl)-[1,1′-biphenyl]-2-carboxylic acid (83 mg, 0.16 mmol, 54%).

¹H NMR (400 MHz, CDCl₃) δ 7.89 (d, J=8.0 Hz, 1H), 7.56-7.52 (m, 1H), 7.43-7.32 (m, 10H), 7.27-7.21 (m, 2H), 7.14 (s, 1H), 6.46 (d, J=9.2 Hz, 1H), 6.17 (d, J=8.0 Hz, 1H), 5.35-5.28 (m, 1H), 4.56 (s, 2H), 3.44 (t, J=5.6 Hz, 2H), 2.81 (t, J=6.0 Hz, 2H), 2.02-1.96 (m, 2H), 1.56 (d, J=6.4 Hz, 3H).

### Example 117

### (S)-1-((2′-carbamoyl-[1,1′-biphenyl]-3-yl)methyl)-N-(1-phenylethyl)-1,2,3,4-tetrahydroquinoline-6-carboxamide

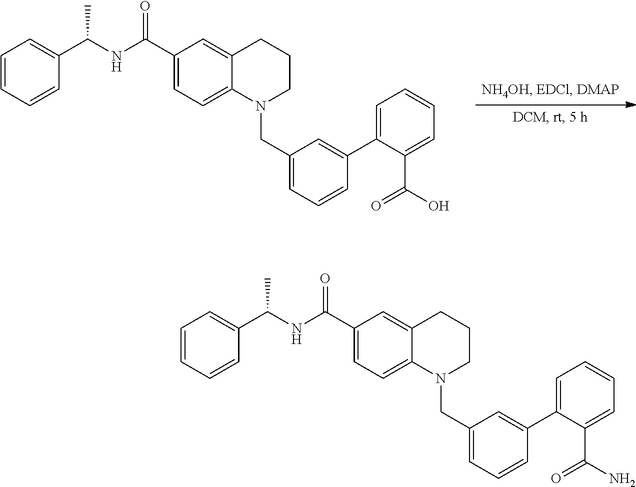 [SMILES: C[C@H](NC(=O)c1ccc2c(c1)CCCN2Cc1cccc(-c2ccccc2C(=O)O)c1)c1ccccc1 ; "DCM, rt, 5 h" ; "NH4OH, EDCl, DMAP" ; C[C@H](NC(=O)c1ccc2c(c1)CCCN2Cc1cccc(-c2ccccc2C(N)=O)c1)c1ccccc1]

(S)-3′-((6-((1-phenylethyl)carbamoyl)-3,4-dihydroquinoline-1(2H)-yl)methyl)-[1,1′-biphenyl]-2-carboxylic acid (78 mg, 0.15 mmol) obtained in Example 116 was dissolved in CH₂Cl₂ (2 ml) in a 25 mL flask, to which EDCI (45 mg, 0.23 mmol) and HOBt (24 mg, 0.18 mmol) were added, followed by stirring at room temperature for 5 hours. Upon completion of the reaction, the organic layer was separated by using CH₂Cl₂ and H₂O. The separated organic layer was dried over MgSO₄ and filtered. The mixture was separated by column chromatography (MeOH/n-Hex/CH₂Cl₂=0.5:0.5:9) to give (S)-1-((2′-carbamoyl-[1,1′-biphenyl]-3-yl)methyl)-N-(1-phenylethyl)-1,2,3,4-tetrahydroquinoline-6-carboxamide (76 mg, 0.14 mmol, 97.6%).

¹H NMR (400 MHz, CDCl₃) δ 7.75-7.73 (m, 1H), 7.54-7.02 (m, 14H), 6.45 (d, J=8.8 Hz, 1H), 6.17 (d, J=8.0 Hz, 1H), 5.37-5.30 (m, 1H), 5.13 (s, 2H), 4.59 (s, 2H), 3.53-3.48 (m, 2H), 2.87 (t, J=6.4 Hz, 2H), 2.07-2.03 (m, 2H), 1.59 (d, J=6.8 Hz, 3H).

### Example 118

### (S)-3′-((6-((1-(4-cyanophenyl)ethyl)carbamoyl)-3,4-dihydroquinoline-1(2H)-yl)methyl)-[1,1′-biphenyl]-2-carboxylic acid

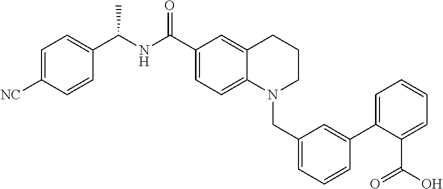 [SMILES: C[C@H](NC(=O)c1ccc2c(c1)CCCN2Cc1cccc(-c2ccccc2C(=O)O)c1)c1ccc(C#N)cc1]

The target compound was obtained by the same manner as described in Example 116 except that (S)-1-(4-cyanophenyl)ethaneamine was used instead of (S)-1-phenylethylamine.

¹H NMR (400 MHz, CDCl₃) δ 7.89 (d, J=7.6 Hz, 1H), 7.60-7.53 (m, 3H), 7.45-7.33 (m, 8H), 7.26-7.23 (m, 1H), 7.14 (s, 1H), 6.44 (d, J=8.8 Hz, 1H), 6.28 (d, J=7.6 Hz, 1H), 5.31-5.24 (m, 1H), 4.58 (s, 2H), 3.47 (t, J=5.2 Hz, 2H), 2.82 (t, J=6.0 Hz, 2H), 2.07-1.99 (m, 2H), 1.51 (d, J=6.8 Hz, 3H).

### Example 119

### (S)-3′-((6-((1-(4-nitrophenyl)ethyl)carbamoyl)-3,4-dihydroquinoline-1(2H)-yl)methyl)-[1,1′-biphenyl]-2-carboxylic acid

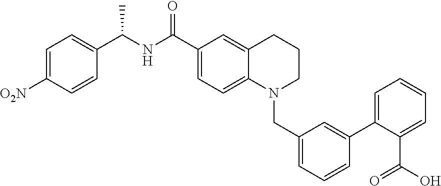 [SMILES: C[C@H](NC(=O)c1ccc2c(c1)CCCN2Cc1cccc(-c2ccccc2C(=O)O)c1)c1ccc([N+](=O)[O-])cc1]

The target compound was obtained by the same manner as described in Example 116 except that (S)-1-(4-nitrophenyl)ethaneamine was used instead of (S)-1-phenylethylamine.

¹H NMR (400 MHz, CDCl₃) δ 8.14 (d, J=8.8 Hz, 2H), 7.57-7.53 (m, 1H), 7.48-7.34 (m, 8H), 7.27-7.23 (m, 2H), 7.14 (s, 1H), 6.42 (d, J=8.8 Hz, 1H), 6.35 (d, J=7.2 Hz, 1H), 5.34-5.28 (m, 1H), 4.58 (s, 2H), 3.46 (t, J=6.0 Hz, 2H), 2.81 (t, J=6.0 Hz, 2H), 2.07-1.97 (m, 2H), 1.52 (d, J=7.2 Hz, 3H).

### Example 120

### (S)-3′-((6-((1-(naphthalene-2-yl)ethyl)carbamoyl)-3,4-dihydroquinoline-1(2H)-yl)methyl)-[1,1′-biphenyl]-2-carboxylic acid

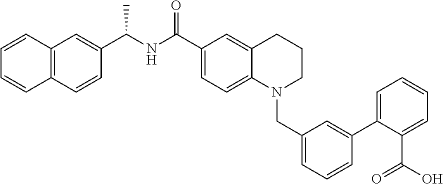 [SMILES: C[C@H](NC(=O)c1ccc2c(c1)CCCN2Cc1cccc(-c2ccccc2C(=O)O)c1)c1ccc2ccccc2c1]

The target compound was obtained by the same manner as described in Example 116 except that (S)-1-(naphthalene-2-yl)ethaneamine was used instead of (S)-1-phenylethylamine.

¹H NMR (400 MHz, CDCl₃) δ 7.89-7.81 (m, 5H), 7.55-7.33 (m, 9H), 7.28-7.21 (m, 2H), 7.14 (s, 1H), 6.46 (d, J=9.2 Hz, 1H), 6.27 (d, J=8.0 Hz, 1H), 5.52-5.45 (m, 1H), 4.56 (s, 2H), 3.43 (t, J=5.6 Hz, 2H), 2.80 (t, J=6.0 Hz, 2H), 2.07-1.95 (m, 2H), 1.54 (d, J=6.8 Hz, 3H).

### Example 121

### (S)-3′-((6-((1-(3-fluoro-4-methylphenyl)ethyl)carbamoyl)-3,4-dihydroquinoline-1(2H)-yl)methyl)-[1,1′-biphenyl]-2-carboxylic acid

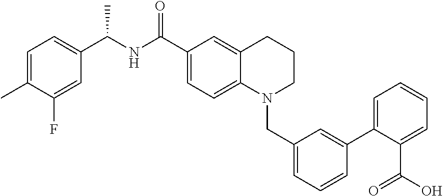 [SMILES: Cc1ccc([C@H](C)NC(=O)c2ccc3c(c2)CCCN3Cc2cccc(-c3ccccc3C(=O)O)c2)cc1F]

The target compound was obtained by the same manner as described in Example 116 except that (S)-1-(3-fluoro-4-methylphenyl)ethaneamine was used instead of (S)-1-phenylethylamine.

¹H NMR (400 MHz, CDCl₃) δ 7.88 (d, J=7.2 Hz, 1H), 7.56-7.52 (m, 1H), 7.42-7.34 (m, 5H), 7.25-7.21 (m, 2H), 7.14-7.11 (m, 2H), 7.04-6.99 (m, 2H), 6.46 (d, J=9.2 Hz, 1H), 6.15 (d, J=7.6 Hz, 1H), 5.29-5.21 (m, 1H), 4.57 (s, 2H), 3.44 (t, J=6.0 Hz, 2H), 2.81 (t, J=6.0 Hz, 2H), 2.25 (s, 3H), 2.01-1.98 (m. 2H), 1.52 (d, J=6.8 Hz, 3H).

### Example 122

### (S)-1-((2′-carbamoyl-[1,1′-biphenyl]-3-yl)methyl)-N-(1-(4-cyanophenyl)ethyl)-1,2,3,4-tetrahydroquinoline-6-carboxamide

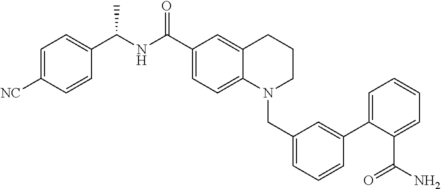 [SMILES: C[C@H](NC(=O)c1ccc2c(c1)CCCN2Cc1cccc(-c2ccccc2C(N)=O)c1)c1ccc(C#N)cc1]

The target compound was obtained by the same manner as described in Example 117 except that (S)-3′-((6-((1-(4-cyanophenyl)ethyl)carbamoyl)-3,4-dihydroquinoline-1(2H)-yl)methyl)-[1,1′-biphenyl]-2-carboxylic acid obtained in Example 118 was used instead of (S)-3′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.75-7.73 (m, 1H), 7.63 (d, J=8.4 Hz, 2H), 7.54-7.31 (m, 11H), 6.46 (d, J=8.8 Hz, 2H), 6.29 (d, J=6.8 Hz, 2H), 5.34-5.27 (m, 1H), 5.14 (s, 2H), 4.60 (s, 2H), 3.53-3.47 (m, 2H), 2.87 (t, J=6.8 Hz, 2H), 2.09-2.03 (m, 2H), 1.57 (d, J=3.6 Hz, 3H).

### Example 123

### (S)-1-((2′-carbamoyl-[1,1′-biphenyl]-3-yl)methyl)-N-(1-(4-nitrophenyl)ethyl)-1,2,3,4-tetrahydroquinoline-6-carboxamide

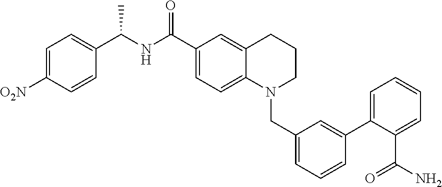 [SMILES: C[C@H](NC(=O)c1ccc2c(c1)CCCN2Cc1cccc(-c2ccccc2C(N)=O)c1)c1ccc([N+](=O)[O-])cc1]

The target compound was obtained by the same manner as described in Example 117 except that (S)-3′-((6-((1-(4-nitrophenyl)ethyl)carbamoyl)-3,4-dihydroquinoline-1(2H)-yl)methyl)-[1,1′-biphenyl]-2-carboxylic acid obtained in Example 119 was used instead of (S)-3′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 8.20-8.18 (m, 2H), 7.75-7.73 (m, 1H), 7.55-7.49 (m, 3H), 7.46-7.41 (m, 4H), 7.38-7.36 (m, 2H), 7.31-7.28 (m, 2H), 6.46 (d, J=8.8 Hz, 1H), 6.34 (d, J=7.2 Hz, 1H), 5.36-5.32 (m, 1H), 5.14 (s, 2H), 4.60 (s, 2H), 3.54-3.48 (m, 2H), 2.87 (t, J=6.0 Hz, 2H), 2.09-2.03 (m, 2H), 1.59 (d, J=7.2 Hz, 3H).

### Example 124

### (S)-1-((2′-carbamoyl-[1,1′-biphenyl]-3-yl)methyl)-N-(1-(naphthalene-2-yl)ethyl)-1,2,3,4-tetrahydroquinoline-6-carboxamide

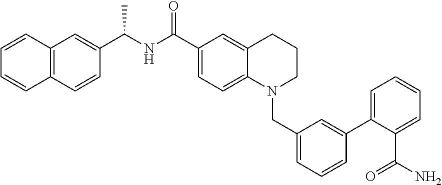 [SMILES: C[C@H](NC(=O)c1ccc2c(c1)CCCN2Cc1cccc(-c2ccccc2C(N)=O)c1)c1ccc2ccccc2c1]

The target compound was obtained by the same manner as described in Example 117 except that (S)-3′-((6-((1-(naphthalene-2-yl)ethyl)carbamoyl)-3,4-dihydroquinoline-1(2H)-yl)methyl)-[1,1′-biphenyl]-2-carboxylic acid obtained in Example 120 was used instead of (S)-3′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.98-7.81 (m, 4H), 7.74-7.73 (m, 1H), 7.54-7.28 (m, 12H), 6.46 (d, J=8.8 Hz, 1H), 6.27 (d, J=7.2 Hz, 1H), 5.52-5.49 (m, 1H), 5.13 (s, 2H), 4.59 (s, 2H), 3.51-3.48 (m, 2H), 2.87 (t, J=5.6 Hz, 2H), 2.09-2.05 (m, 2H), 1.67 (d, J=6.8 Hz, 3H).

### Example 125

### (S)-1-((2′-carbamoyl-[1,1′-biphenyl]-3-yl)methyl)-N-(1-(3-fluoro-4-methylphenyl)ethyl)-1,2,3,4-tetrahydroquinoline-6-carboxamide

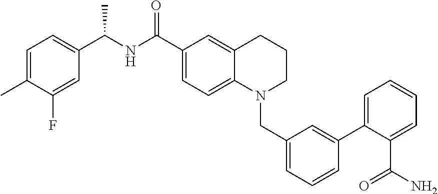 [SMILES: Cc1ccc([C@H](C)NC(=O)c2ccc3c(c2)CCCN3Cc2cccc(-c3ccccc3C(N)=O)c2)cc1F]

The target compound was obtained by the same manner as described in Example 117 except that (S)-3′-((6-((1-(3-fluoro-4-methylphenyl)ethyl)carbamoyl)-3,4-dihydroquinoline-1(2H)-yl)methyl)-[1,1′-biphenyl]-2-carboxylic acid obtained in Example 121 was used instead of (S)-3′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.75-7.30 (m, 1H), 7.54-7.23 (m, 10H), 7.16-7.13 (m, 1H), 7.07-7.01 (m, 2H), 6.46 (d, J=8.8 Hz, 1H), 6.15 (d, J=8.0 Hz, 1H), 5.30-5.23 (m, 1H), 5.09 (s, 2H), 4.59 (s, 2H), 3.53-3.48 (m, 2H), 2.87 (t, J=6.0 Hz, 2H), 2.26 (s, 2H), 2.09-2.04 (m, 3H), 1.55 (d, J=6.8 Hz, 3H).

### Example 126

### (S)-2′-fluoro-3′-((6-((1-phenylethyl)carbamoyl)-3,4-dihydroquinoline-1(2H)-yl)methyl)-[1,1′-biphenyl]-2-carboxylic acid

### Step 1 (S)-tert-butyl 2′-fluoro-3′-((6-((1-phenylethyl)carbamoyl)-3,4-dihydroquinoline-1(2H)-yl)methyl)-[1,1′-biphenyl]-2-carboxylate

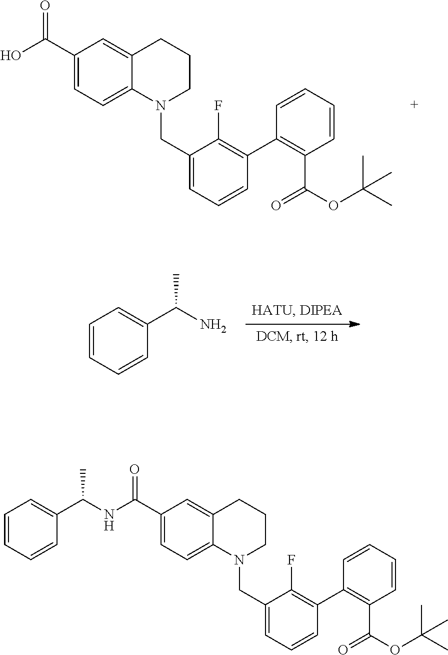

1-((2′-(tert-butoxycarbonyl)-2-fluoro-[1,1′-biphenyl]-3-yl)methyl)-1,2,3,4-tetrahydroquinoline-6-carboxylic acid (300 mg, 0.67 mmol) obtained in Preparative Example 5 was dissolved in CH₂Cl₂ (10 ml) in a 50 mL flask, to which HATU (386 mg, 1.01 mmol) and DIPEA (353 ul, 2.02 mmol) were added, followed by stirring. (S)-1-phenylethylamine (105 ul, 0.81 mmol) was added, followed by stirring at room temperature for 12 hours. Upon completion of the reaction, the organic layer was separated by using CH₂Cl₂ and H₂O. The separated organic layer was dried over MgSO₄ and filtered. The mixture was separated by column chromatography (EA/n-Hex=1:1) to give (S)-tert-butyl 2′-fluoro-3′-((6-((1-phenylethyl)carbamoyl)-3,4-dihydroquinoline-1(2H)-yl)methyl)-[1,1′-biphenyl]-2-carboxylate (169 mg, 0.29 mmol, 44%).

### Step 2 (S)-2′-fluoro-3′-((6-((1-phenylethyl)carbamoyl)-3,4-dihydroquinoline-1(2H)-yl)methyl)-[1,1′-biphenyl]-2-carboxylic acid

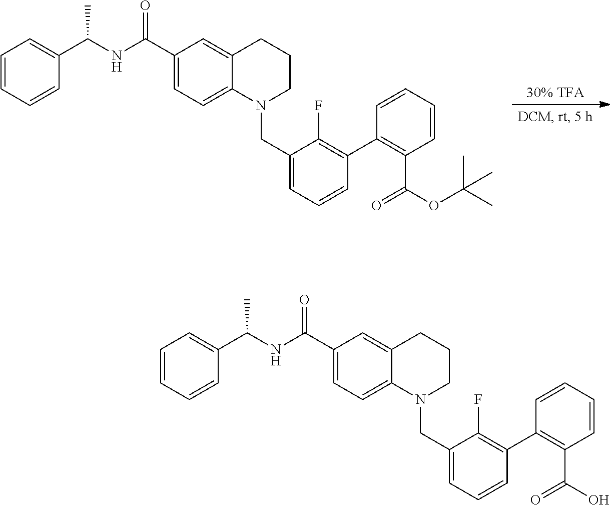 [SMILES: C[C@H](NC(=O)c1ccc2c(c1)CCCN2Cc1cccc(-c2ccccc2C(=O)OC(C)(C)C)c1F)c1ccccc1 ; "30% TFA" ; "DCM, rt, 5 h" ; C[C@H](NC(=O)c1ccc2c(c1)CCCN2Cc1cccc(-c2ccccc2C(=O)O)c1F)c1ccccc1]

(S)-tert-butyl 2′-fluoro-3′-((6-((1-phenylethyl)carbamoyl)-3,4-dihydroquinoline-1(2H)-yl)methyl)-[1,1′-biphenyl]-2-carboxylate (168 mg, 0.3 mmol) obtained in step 1 was dissolved in CH₂Cl₂ (10 ml) in a 50 mL flask, to which 30% TFA (3 ml) was added, followed by stirring at room temperature for 5 hours. Upon completion of the reaction the reaction mixture was concentrated under reduced pressure. The mixture was separated by column chromatography (EA) to give (S)-2′-fluoro-3′-((6-((1-phenylethyl)carbamoyl)-3,4-dihydroquinoline-1(2H)-yl)methyl)-[1,1′-biphenyl]-2-carboxylic acid (83 mg, 0.16 mmol, 54%).

¹H NMR (400 MHz, CDCl₃) δ 8.08 (d, J=7.2 Hz, 1H), 7.66-7.62 (m, 1H), 7.54-7.33 (m, 9H), 7.25-7.21 (m, 2H), 7.17-7.02 (m, 1H), 6.47 (d, J=8.4 Hz, 1H), 6.14 (d, J=7.6 Hz, 1H), 5.36-5.32 (m, 1H), 4.57 (s, 2H), 3.46 (d, J=5.6 Hz, 1H), 2.84 (t, J=6.0 Hz, 2H), 2.07-2.00 (m, 2H), 1.58 (d, J=6.8 Hz, 3H).

### Example 127

### (S)-1-((2′-carbamoyl-2-fluoro-[1,1′-biphenyl]-3-yl)methyl)-N-(1-phenylethyl)-1,2,3,4-tetrahydroquinoline-6-carboxamide

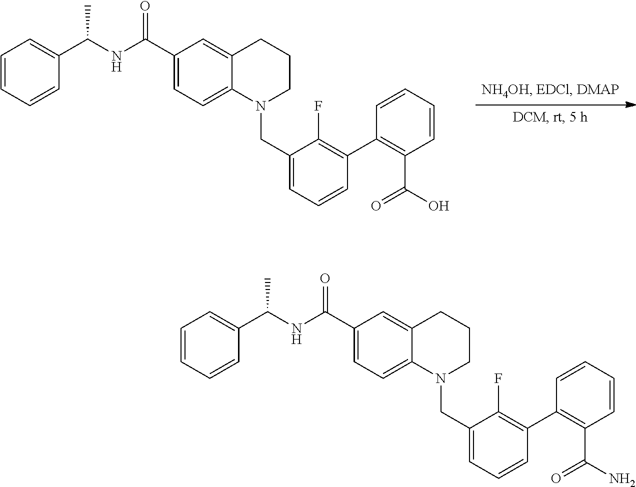 [SMILES: C[C@H](NC(=O)c1ccc2c(c1)CCCN2Cc1cccc(-c2ccccc2C(=O)O)c1F)c1ccccc1 ; "DCM, rt, 5 h" ; "NH4OH, EDCl, DMAP" ; C[C@H](NC(=O)c1ccc2c(c1)CCCN2Cc1cccc(-c2ccccc2C(N)=O)c1F)c1ccccc1]

(S)-2′-fluoro-3′-((6-((1-phenylethyl)carbamoyl)-3,4-dihydroquinoline-1(2H)-yl)methyl)-[1,1′-biphenyl]-2-carboxylic acid (78 mg, 0.15 mmol) obtained in Example 126 was dissolved in CH₂Cl₂ (2 ml) in a 25 mL flask, to which EDCI (45 mg, 0.23 mmol) and HOBt (24 mg, 0.18 mmol) were added, followed by stirring. Ammonium hydroxide solution (3 ml) was added thereto, followed by stirring at room temperature for 5 hours. Upon completion of the reaction, the organic layer was separated by using CH₂Cl₂ and H₂O. The separated organic layer was dried over MgSO₄ and filtered. The mixture was separated by column chromatography (MeOH/n-Hex/CH₂Cl₂=0.5:0.5:9) to give (S)-1-((2′-carbamoyl-2-fluoro-[1,1′-biphenyl]-3-yl)methyl)-N-(1-phenylethyl)-1,2,3,4-tetrahydroquinoline-6-carboxamide (76 mg, 0.14 mmol, 97.6%).

¹H NMR (400 MHz, CDCl₃) δ 7.83-7.81 (m, 1H), 7.59-7.54 (m, 1H), 7.52-7.48 (m, 2H), 7.45-7.26 (m, 8H), 7.21-7.13 (m, 2H), 7.31-7.28 (m, 2H), 6.46 (d, J=8.8 Hz, 1H), 6.15 (d, J=8.4 Hz, 1H), 5.45-5.32 (m, 3H), 4.62 (s, 2H), 3.49 (d, J=6.0 Hz, 1H), 2.87 (t, J=6.8 Hz, 2H), 2.09-2.03 (m, 2H), 1.59 (d, J=6.8 Hz, 3H).

### Example 128

### (S)-2′-fluoro-3′-((6-((1-(4-nitrophenyl)ethyl)carbamoyl)-3,4-dihydroquinoline-1(2H)-yl)methyl)-[1,1′-biphenyl]-2-caboxylic acid

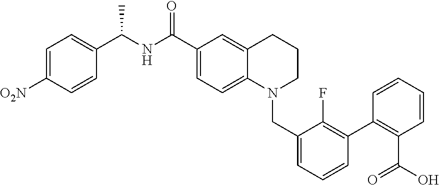 [SMILES: C[C@H](NC(=O)c1ccc2c(c1)CCCN2Cc1cccc(-c2ccccc2C(=O)O)c1F)c1ccc([N+](=O)[O-])cc1]

The target compound was obtained by the same manner as described in Example 126 except that (S)-1-(4-nitrophenyl)ethaneamine was used instead of (S)-1-phenylethylamine.

¹H NMR (400 MHz, CDCl₃) δ 8.19-8.17 (m, 2H), 7.10-7.08 (m, 1H), 7.78-7.63 (m, 1H), 7.53-7.39 (m, 6H), 7.26-7.24 (m, 1H), 7.16-7.13 (m, 2H), 6.49 (d, J=8.4 Hz, 1H), 6.22 (d, J=6.8 Hz, 1H), 5.37-5.32 (m, 1H), 4.59 (s, 2H), 3.49 (t, J=6.0 Hz, 2H), 2.85 (t, J=6.4 Hz, 2H), 2.07-2.03 (m, 2H), 1.57 (d, J=6.8 Hz, 3H).

### Example 129

### (S)-2′-fluoro-3′-((6-((1-(naphthalene-2-yl)ethyl)carbamoyl)-3,4-dihydroquinoline-1(28)-yl)methyl)-[1,1′-biphenyl]-2-carboxylic acid

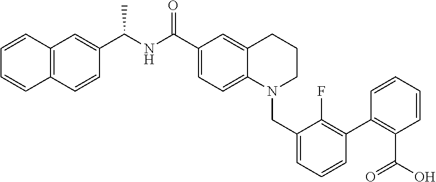 [SMILES: C[C@H](NC(=O)c1ccc2c(c1)CCCN2Cc1cccc(-c2ccccc2C(=O)O)c1F)c1ccc2ccccc2c1]

The target compound was obtained by the same manner as described in Example 126 except that (S)-1-(naphthalene-2-yl)ethaneamine was used instead of (S)-1-phenylethylamine.

¹H NMR (400 MHz, CDCl₃) δ 8.08-8.06 (m, 1H), 7.84-7.81 (m, 4H), 7.63-7.59 (m, 1H), 7.54-7.43 (m, 6H), 7.39-7.37 (m, 1H), 7.23-7.20 (m, 1H), 7.16-7.09 (m, 2H), 6.46 (d, J=8.4 Hz, 1H), 6.23 (d, J=8.4 Hz, 1H), 5.52-5.49 (m, 1H), 4.55 (s, 2H), 3.45 (t, J=6.0 Hz, 2H), 2.83 (t, J=6.0 Hz, 2H), 2.07-2.00 (m, 2H), 1.66 (d, J=6.8 Hz, 3H).

### Example 130

### (S)-2′-fluoro-3′-((6-((1-(3-fluoro-4-methylphenyl)ethyl) carbamoyl)-3, 4-dihydroquinoline-1(2H)-yl)methyl)-[1,1′-biphenyl]-2-caboxylic acid

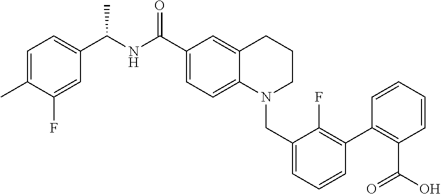 [SMILES: Cc1ccc([C@H](C)NC(=O)c2ccc3c(c2)CCCN3Cc2cccc(-c3ccccc3C(=O)O)c2F)cc1F]

The target compound was obtained by the same manner as described in Example 126 except that (S)-1-(3-fluoro-4-methylphenyl)ethaneamine was used instead of (S)-1-phenylethylamine.

¹H NMR (400 MHz, CDCl₃) δ 8.10-8.08 (m, 1H), 7.66-7.62 (m, 1H), 7.54-7.39 (m, 4H), 7.25-7.21 (m, 1H), 7.16-7.10 (m, 3H), 7.06-7.00 (m, 2H), 6.48 (d, J=8.8 Hz, 1H), 6.10 (d, J=7.2 Hz, 1H), 5.29-5.26 (m, 1H), 4.58 (s, 2H), 3.47 (t, J=6.0 Hz, 2H), 2.85 (t, J=5.6 Hz, 2H), 2.25 (s, 3H), 2.07-2.01 (m, 2H), 1.54 (d, J=6.8 Hz, 3H).

### Example 131

### (S)-1-((2′-carbamoyl-2-fluoro-[1,1′-biphenyl]-3-yl)methyl)-N-(1-(4-nitrophenyl)ethyl)-1,2,3,4-tetrahydroquinoline-6-carboxamide

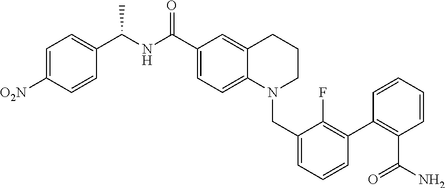 [SMILES: C[C@H](NC(=O)c1ccc2c(c1)CCCN2Cc1cccc(-c2ccccc2C(N)=O)c1F)c1ccc([N+](=O)[O-])cc1]

The target compound was obtained by the same manner as described in Example 127 except that (S)-2′-fluoro-3′-((6-((1-(4-nitrophenyl)ethyl)carbamoyl)-3,4-dihydroquinoline-1(2H)-yl)methyl)-[1,1′-biphenyl]-2-carboxylic acid obtained in Example 128 was used instead of (S)-2′-fluoro-3′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 8.21-8.19 (m, 2H), 7.82-7.80 (m, 1H), 7.60-7.49 (m, 5H), 7.46-7.45 (m, 1H), 7.44-7.43 (m, 1H), 7.41-7.39 (m, 1H), 7.21-7.14 (m, 2H), 6.49 (d, J=8.8 Hz, 1H), 6.26 (d, J=6.8 Hz, 1H), 5.46-5.32 (m, 3H), 4.63 (s, 2H), 3.51 (t, J=5.6 Hz, 2H), 2.87 (t, J=6.0 Hz, 2H), 2.10-2.04 (m, 2H), 1.60 (d, J=7.2 Hz, 3H).

### Example 132

### (S)-1-((2′-carbamoyl-2-fluoro-[1,1′-biphenyl]-3-yl)methyl)-N-(1-(naphthalene-2-yl)ethyl)-1,2,3,4-tetrahydroquinoline-6-carboxamide

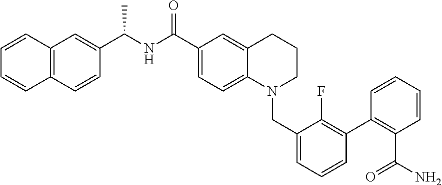 [SMILES: C[C@H](NC(=O)c1ccc2c(c1)CCCN2Cc1cccc(-c2ccccc2C(N)=O)c1F)c1ccc2ccccc2c1]

The target compound was obtained by the same manner as described in Example 127 except that (S)-2′-fluoro-3′-((6-((1-(naphthalene-2-yl)ethyl)carbamoyl)-3,4-dihydroquinoline-1(2H)-yl)methyl)-[1,1′-biphenyl]-2-carboxylic acid obtained in Example 129 was used instead of (S)-2′-fluoro-3′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.85-7.80 (m, 5H), 7.59-7.45 (m, 7H), 7.40-7.38 (m, 1H), 7.31-7.28 (m, 1H), 7.21-7.13 (m, 2H), 6.47 (d, J=8.8 Hz, 1H), 6.24 (d, J=8.0 Hz, 1H), 5.53-5.40 (m, 3H), 4.62 (s, 2H), 3.49 (t, J=5.6 Hz, 2H), 2.87 (t, J=6.0 Hz, 2H), 2.07-2.04 (m, 2H), 1.69 (d, J=6.8 Hz, 3H).

### Example 133

### (S)-1-((2′-carbamoyl-2-fluoro-[1,1′-biphenyl]-3-yl)methyl)-N-(1-(3-fluoro-4-methylphenyl)ethyl)-1,2,3,4-tetrahydroquinoline-6-carboxamide

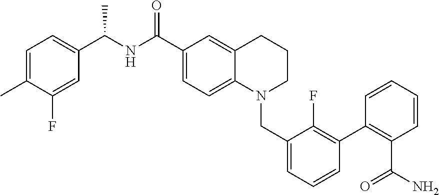 [SMILES: Cc1ccc([C@H](C)NC(=O)c2ccc3c(c2)CCCN3Cc2cccc(-c3ccccc3C(N)=O)c2F)cc1F]

The target compound was obtained by the same manner as described in Example 127 except that (S)-2′-fluoro-3′-((6-((1-(3-fluoro-4-methylphenyl)ethyl)carbamoyl)-3,4-dihydroquinoline-1(2H)-yl)methyl)-[1,1′-biphenyl]-2-carboxylic acid obtained in Example 130 was used instead of (S)-2′-fluoro-3′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.83-7.81 (m, 1H), 7.59-7.54 (m, 1H), 7.52-7.48 (m, 2H), 7.44-7.39 (m, 2H), 7.32-7.28 (m, 2H), 7.21-7.13 (m, 3H), 7.07-7.02 (m, 2H), 6.47 (d, J=8.4 Hz, 1H), 6.11 (d, J=7.6 Hz, 1H), 5.45-5.40 (m, 2H), 5.32-5.25 (m, 1H), 4.63 (s, 2H), 3.49 (t, J=5.2 Hz, 2H), 2.87 (t, J=6.4 Hz, 2H), 2.26 (s, 3H), 2.09-2.03 (m, 2H), 1.56 (d, J=8.4 Hz, 3H).

### Example 134

### (S)-4′-fluoro-3′-((6-((1-phenylethyl)carbamoyl)-3,4-dihydroquinoline-1(2H)-yl)methyl)-[1,1′-biphenyl]-2-carboxylic acid

### Step 1 (S)-tert-butyl 4′-fluoro-3′-((6-((1-phenylethyl)carbamoyl)-3,4-dihydroquinoline-1(2H)-yl)methyl)-[1,1′-biphenyl]-2-carboxylate

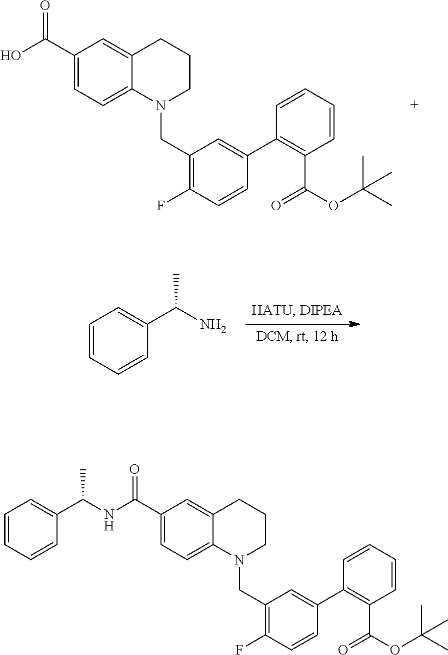

1-((2′-(tert-butoxycarbonyl)-4-fluoro-[1,1′-biphenyl]-3-yl)methyl)-1,2,3,4-tetrahydroquinoline-6-carboxylic acid (300 mg, 0.67 mmol) obtained in Preparative Example 6 was dissolved in CH₂Cl₂ (10 ml) in a 100 mL flask, to which HATU (386 mg, 1.01 mmol) and DIPEA (353 ul, 2.02 mmol) were added, followed by stirring at room temperature for 12 hours. Upon completion of the reaction, the organic layer was separated by using CH₂Cl₂ and H₂O. The separated organic layer was dried over MgSO₄ and filtered. The mixture was separated by column chromatography (EA/n-Hex=1:1) to give (S)-tert-butyl 4′-fluoro-3′-((6-((1-phenylethyl)carbamoyl)-3,4-dihydroquinoline-1(2H)-yl)methyl)-[1,1′-biphenyl]-2-carboxylate (169 mg, 0.29 mmol, 44%).

### Step 2 (S)-4′-fluoro-3′-((6-((1-phenylethyl)carbamoyl)-3,4-dihydroquinoline-1(2H)-yl)methyl)-[1,1′-biphenyl]-2-carboxylic acid

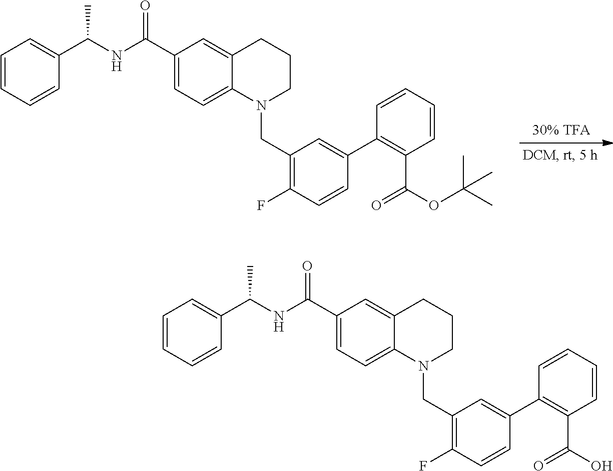 [SMILES: C[C@H](NC(=O)c1ccc2c(c1)CCCN2Cc1cc(-c2ccccc2C(=O)OC(C)(C)C)ccc1F)c1ccccc1 ; C[C@H](NC(=O)c1ccc2c(c1)CCCN2Cc1cc(-c2ccccc2C(=O)O)ccc1F)c1ccccc1]

(S)-tert-butyl 4′-fluoro-3′-((6-((1-phenylethyl)carbamoyl)-3,4-dihydroquinoline-1(2H)-yl)methyl)-[1,1′-biphenyl]-2-carboxylate (168 mg, 0.3 mmol) obtained in step 1 was dissolved in CH₂Cl₂ (10 ml) in a 50 mL flask, to which 30% TFA (3 ml) was added, followed by stirring at room temperature for 5 hours. Upon completion of the reaction the reaction mixture was concentrated under reduced pressure. The mixture was separated by column chromatography (EA) to give (S)-4′-fluoro-3′-((6-((1-phenylethyl)carbamoyl)-3,4-dihydroquinoline-1(2H)-yl)methyl)-[1,1′-biphenyl]-2-carboxylic acid (83 mg, 0.16 mmol, 54%).

¹H NMR (400 MHz, CDCl₃) δ 7.85-7.83 (d, J=7.2 Hz, 1H), 7.54-7.44 (m, 2H), 7.39-7.34 (m, 7H), 7.25-7.23 (m, 2H), 7.15-7.10 (m, 1H), 6.44 (d, J=8.8 Hz, 1H), 6.21 (d, J=8.0 Hz, 1H), 5.32-5.28 (m, 1H), 4.61 (s, 2H), 3.45 (d, J=5.6 Hz, 1H), 2.79 (t, J=6.4 Hz, 2H), 2.01-1.95 (m, 2H), 1.53 (d, J=5.6 Hz, 3H).

### Example 135

### (S)-1-((2′-carbamoyl-4-fluoro-[1,1′-biphenyl]-3-yl)methyl)-N-(1-phenylethyl)-1,2,3,4-tetrahydroquinoline-6-carboxamide

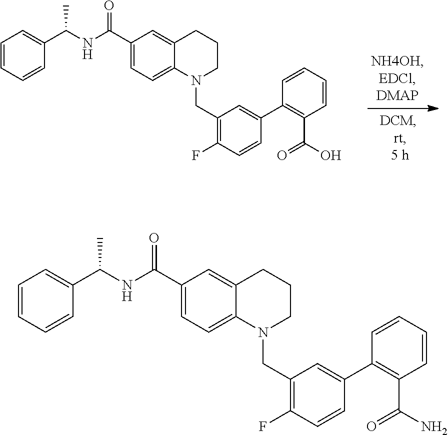 [SMILES: C[C@H](NC(=O)c1ccc2c(c1)CCCN2Cc1cc(-c2ccccc2C(=O)O)ccc1F)c1ccccc1 ; C[C@H](NC(=O)c1ccc2c(c1)CCCN2Cc1cc(-c2ccccc2C(N)=O)ccc1F)c1ccccc1]

(S)-4′-fluoro-3′-((6-((1-phenylethyl)carbamoyl)-3,4-dihydroquinoline-1(2H)-yl)methyl)-[1,1′-biphenyl]-2-carboxylic acid (78 mg, 0.15 mmol) obtained in Example 134 was dissolved in CH₂Cl₂ (2 ml) in a 25 mL flask, to which EDCI (45 mg, 0.23 mmol) and HOBt (24 mg, 0.18 mmol) were added, followed by stirring at room temperature for 5 hours. Upon completion of the reaction, the organic layer was separated by using CH₂Cl₂ and H₂O. The separated organic layer was dried over MgSO₄ and filtered. The mixture was separated by column chromatography (MeOH/n-Hex/CH₂Cl₂=0.5:0.5:9) to give (S)-1-((2′-carbamoyl-4-fluoro-[1,1′-biphenyl]-3-yl)methyl)-N-(1-phenylethyl)-1,2,3,4-tetrahydroquinoline-6-carboxamide (76 mg, 0.14 mmol, 97.6%).

¹H NMR (400 MHz, CDCl₃) δ 7.64-7.62 (m, 1H), 7.49-7.33 (m, 11H), 7.25-7.23 (m, 1H), 7.19-7.15 (m, 1H), 6.43 (d, J=7.2 Hz, 1H), 6.20 (d, J=7.6 Hz, 1H), 5.34-5.30 (m, 1H), 5.10 (s, 1H), 5.01 (s, 1H), 4.62 (s, 2H), 3.52 (d, J=6.0 Hz, 1H), 2.87 (t, J=5.6 Hz, 2H), 2.07-2.04 (m, 2H), 1.59 (d, J=6.8 Hz, 3H).

### Example 136

### (S)-4′-fluoro-3′-((6-((1-(4-nitrophenyl)ethyl) carbamoyl)-3, 4-dihydroquinoline-1(2H)-yl)methyl)-[1,1′-biphenyl]-2-caboxylic acid

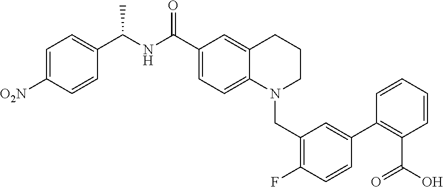 [SMILES: C[C@H](NC(=O)c1ccc2c(c1)CCCN2Cc1cc(-c2ccccc2C(=O)O)ccc1F)c1ccc([N+](=O)[O-])cc1]

The target compound was obtained by the same manner as described in Example 134 except that (S)-1-(4-nitrophenyl)ethaneamine was used instead of (S)-1-phenylethylamine.

¹H NMR (400 MHz, CDCl₃) δ 8.10-8.08 (m, 2H), 7.84-7.82 (m, 1H), 7.53-7.50 (m, 1H), 7.41-7.37 (m, 4H), 7.28-7.21 (m, 3H), 7.14-7.09 (m, 1H), 7.01-6.99 (m, 1H), 6.52 (d, J=7.6 Hz, 1H), 6.29 (d, J=8.4 Hz, 1H), 5.27-5.23 (m, 1H), 4.62 (s, 2H), 3.48 (t, J=5.2 Hz, 2H), 2.80 (t, J=6.4 Hz, 2H), 2.07-2.00 (m, 2H), 1.47 (d, J=6.8 Hz, 3H).

### Example 137

### (S)-4′-fluoro-3′-((6-((1-(naphthalene-2-yl)ethyl)carbamoyl)-3,4-dihydroquinoline-1(2)-yl)methyl)-[1,1′-biphenyl]-2-carboxylic acid

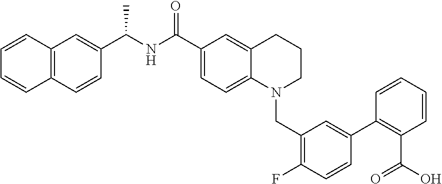 [SMILES: C[C@H](NC(=O)c1ccc2c(c1)CCCN2Cc1cc(-c2ccccc2C(=O)O)ccc1F)c1ccc2ccccc2c1]

The target compound was obtained by the same manner as described in Example 134 except that (S)-1-(naphthalene-2-yl)ethaneamine was used instead of (S)-1-phenylethylamine.

¹H NMR (400 MHz, CDCl₃) δ 7.84-7.80 (m, 5H), 7.54-7.44 (m, 5H), 7.38-7.35 (m, 2H), 7.27-7.22 (m, 2H), 7.15-7.10 (m, 1H), 7.00-6.98 (m, 1H), 6.43 (d, J=8.8 Hz, 1H), 6.33 (d, J=8.0 Hz, 1H), 5.48-5.44 (m, 1H), 4.60 (s, 2H), 3.44 (t, J=6.0 Hz, 2H), 2.77 (t, J=6.0 Hz, 2H), 2.00-1.94 (m, 2H), 1.63 (d, J=7.2 Hz, 3H).

### Example 138

### (S)-4′-fluoro-3′-((6-((1-(3-fluoro-4-methylphenyl)ethyl) carbamoyl)-3, 4-dihydroquinoline-1(2H)-yl)methyl)-[1,1′-biphenyl]-2-caboxylic acid

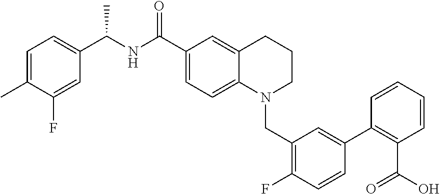 [SMILES: Cc1ccc([C@H](C)NC(=O)c2ccc3c(c2)CCCN3Cc2cc(-c3ccccc3C(=O)O)ccc2F)cc1F]

The target compound was obtained by the same manner as described in Example 134 except that (S)-1-(3-fluoro-4-methylphenyl)ethaneamine was used instead of (S)-1-phenylethylamine.

¹H NMR (400 MHz, CDCl₃) δ 7.84 (t, J=7.6 Hz, 2H), 7.54-7.49 (m, 1H), 7.44-7.34 (m, 4H), 7.26-7.23 (m, 2H), 7.15-7.11 (m, 2H), 7.04-6.99 (m, 3H), 6.43 (d, J=8.8 Hz, 1H), 6.20 (d, J=8.0 Hz, 1H), 5.25-5.22 (m, 1H), 4.62 (s, 2H), 3.46 (t, J=5.6 Hz, 2H), 2.80 (t, J=6.0 Hz, 2H), 2.25 (s, 3H), 2.07-1.98 (m, 2H), 1.51 (d, J=7.2 Hz, 3H).

### Example 139

### (S)-1-((2′-carbamoyl-4-fluoro-[1,1′-biphenyl]-3-yl)methyl)-N-(1-(4-nitrophenyl)ethyl)-1,2,3,4-tetrahydroquinoline-6-carboxamide

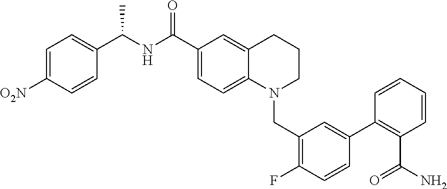 [SMILES: C[C@H](NC(=O)c1ccc2c(c1)CCCN2Cc1cc(-c2ccccc2C(N)=O)ccc1F)c1ccc([N+](=O)[O-])cc1]

The target compound was obtained by the same manner as described in Example 135 except that (S)-4′-fluoro-3′-((6-((1-(4-nitrophenyl)ethyl)carbamoyl)-3,4-dihydroquinoline-1(2H)-yl)methyl)-[1,1′-biphenyl]-2-carboxylic acid obtained in Example 136 was used instead of (S)-4′-fluoro-3′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 8.18-8.16 (m, 2H), 7.65-7.62 (m, 1H), 7.54-7.28 (m, 8H), 7.23-7.15 (m, 2H), 6.49 (d, J=7.2 Hz, 1H), 6.40 (d, J=8.4 Hz, 1H), 5.34-5.28 (m, 1H), 5.21 (s, 1H), 5.11 (s, 1H), 4.64 (s, 2H), 3.53 (t, J=5.6 Hz, 2H), 2.86 (t, J=6.0 Hz, 2H), 2.08-2.03 (m, 2H), 1.58 (d, J=6.8 Hz, 3H).

### Example 140

### (S)-1-((2′-carbamoyl-4-fluoro-[1,1′-biphenyl]-3-yl)methyl)-N-(1-(naphthalene-2-yl)ethyl)-1,2,3,4-tetrahydroquinoline-6-carboxamide

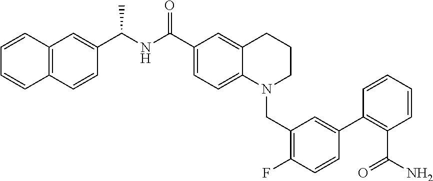 [SMILES: C[C@H](NC(=O)c1ccc2c(c1)CCCN2Cc1cc(-c2ccccc2C(N)=O)ccc1F)c1ccc2ccccc2c1]

The target compound was obtained by the same manner as described in Example 135 except that (S)-4′-fluoro-3′-((6-((1-(naphthalene-2-yl)ethyl)carbamoyl)-3,4-dihydroquinoline-1(2H)-yl)methyl)-[1,1′-biphenyl]-2-carboxylic acid obtained in Example 137 was used instead of (S)-4′-fluoro-3′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.84-7.82 (m, 4H), 7.63-7.61 (m, 1H), 7.54-7.45 (m, 7H), 7.41-7.34 (m, 2H), 7.25-7.23 (m, 1H), 7.17-7.14 (m, 1H), 6.43 (d, J=9.2 Hz, 1H), 6.31 (d, J=7.6 Hz, 1H), 5.51-5.47 (m, 1H), 5.10 (s, 1H), 5.03 (s, 1H), 4.62 (s, 2H), 3.52 (t, J=6.0 Hz, 2H), 2.86 (t, J=6.4 Hz, 2H), 2.07-2.04 (m, 2H), 1.68 (d, J=6.8 Hz, 3H).

### Example 141

### (S)-1-((2′-carbamoyl-4-fluoro-[1,1′-biphenyl]-3-yl)methyl)-N-(1-(3-fluoro-4-methylphenyl)ethyl)-1,2,3,4-tetrahydroquinoline-6-carboxamide

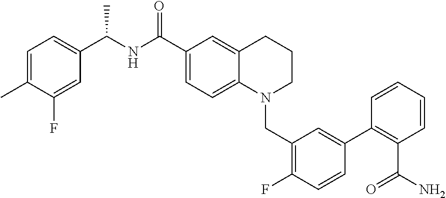 [SMILES: Cc1ccc([C@H](C)NC(=O)c2ccc3c(c2)CCCN3Cc2cc(-c3ccccc3C(N)=O)ccc2F)cc1F]

The target compound was obtained by the same manner as described in Example 135 except that (S)-4′-fluoro-3′-((6-((1-(3-fluoro-4-methylphenyl)ethyl)carbamoyl)-3,4-dihydroquinoline-1(2H)-yl)methyl)-[1,1′-biphenyl]-2-carboxylic acid obtained in Example 138 was used instead of (S)-4′-fluoro-3′-((6-((1-phenylethyl)carbamoyl)-3,4-dihydroquinoline-1(2H)-yl)methyl)-[1,1′-biphenyl]-2-carboxylic acid.

¹H NMR (400 MHz, CDCl₃) δ 7.64-7.62 (m, 1H), 7.54-7.34 (m, 7H), 7.30-7.28 (m, 1H), 7.25-7.22 (m, 1H), 7.19-7.12 (m, 2H), 7.07-7.01 (m, 2H), 6.43 (d, J=9.2 Hz, 1H), 6.21 (d, J=7.2 Hz, 1H), 5.27-5.24 (m, 1H), 5.11 (s, 1H), 5.03 (s, 1H), 4.63 (s, 2H), 3.52 (t, J=6.0 Hz, 2H), 2.86 (t, J=6.4 Hz, 2H), 2.25 (s, 3H), 2.07-2.03 (m, 2H), 1.55 (d, J=6.8 Hz, 3H).

### Example 142

### (S)-2′-((7-((1-phenylethyl)carbamoyl)-2H-benzo[b][1,4]oxazine-4(3H)-yl)methyl)-[1,1′-biphenyl]-4-carboxylic acid

### Step 1 (S)—N-(1-phenylethyl)-3,4-dihydro-2H-benzo[b][1,4]oxazine-7-carboxamide

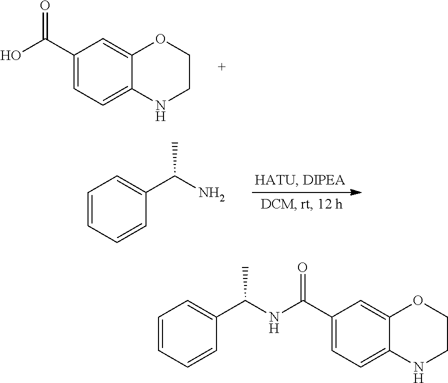

3,4-dihydro-2H-benzo[b][1,4]oxazine-7-carboxylic acid (314 mg, 1.69 mmol) obtained in Preparative Example 7 was dissolved in CH₂Cl₂ (10 ml) in a 100 mL flask, to which HATU (969 mg, 1.54 mmol) and DIPEA (888 ul, 5.09 mmol) were added, followed by stirring. (S)-1-phenylethylamine (547 ul, 4.24 mmol) was added, followed by stirring at room temperature for 12 hours. Upon completion of the reaction, the organic layer was separated by using CH₂Cl₂ and H₂O. The separated organic layer was dried over MgSO₄ and filtered. The mixture was separated by column chromatography (EA/n-Hex=1:1) to give (S)—N-(1-phenylethyl)-3,4-dihydro-2H-benzo[b][1,4]oxazine-7-carboxamide (210 mg, 1.34 mmol, 79%).

### Step 2 (S)-methyl 2′-((7-((1-phenylethyl)carbamoyl)-2H-benzo[b][1,4]oxazine-4(3H)-yl)methyl)-[1,1′-biphenyl]-4-carboxylate

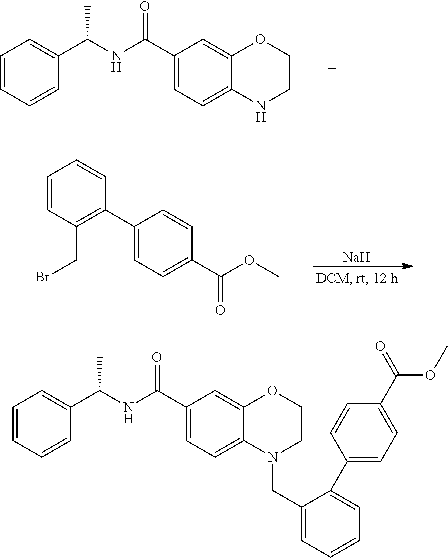

(S)—N-(1-phenylethyl)-3,4-dihydro-2H-benzo[b][1,4]oxazine-7-carboxamide (210 mg, 0.83 mmol) was dissolved in DMF (3 ml) in a 25 mL flask, to which NaH (33 mg, 0.83 mmol) and methyl 2′-(bromomethyl)-[1,1′-biphenyl]-4-carboxylate (254 mg, 0.83 mmol) were added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give (S)-methyl 2′-((7-((1-phenylethyl)carbamoyl)-2H-benzo[b][1,4]oxazine-4(3H)-yl)methyl)-[1,1′-biphenyl]-4-carboxylate (160 mg).

### Step 3 (S)-2′-((7-((1-phenylethyl)carbamoyl)-2H-benzo[b][1,4]oxazine-4(3H)-yl)methyl)-[1,1′-biphenyl]-4-carboxylic acid

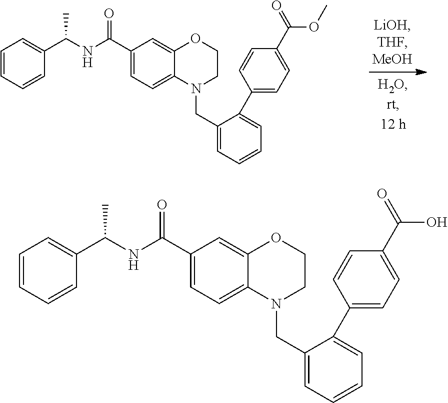 [SMILES: COC(=O)c1ccc(-c2ccccc2CN2CCOc3cc(C(=O)N[C@@H](C)c4ccccc4)ccc32)cc1 ; C[C@H](NC(=O)c1ccc2c(c1)OCCN2Cc1ccccc1-c1ccc(C(=O)O)cc1)c1ccccc1]

(S)-methyl 2′-((7-((1-phenylethyl)carbamoyl)-2H-benzo[b][1,4]oxazine-4(3H)-yl)methyl)-[1,1′-biphenyl]-4-carboxylate (160 mg, 0.31 mmol) was dissolved in MeOH (5 ml) and THF (10 ml) in a 25 mL flask, to which LiOH (2 g) dissolved in H₂O (5 ml) was added, followed by stirring at room temperature for 12 hours. Upon completion of the reaction, the solvent was concentrated. PH of the reaction mixture was adjusted to 5 by using 2N-HCl. The mixture was extracted with EA. The organic layer was dried over MgSO₄ and filtered. As a result, (S)-2′-((7-((1-phenylethyl)carbamoyl)-2H-benzo[b][1,4]oxazine-4(3H)-yl)methyl)-[1,1′-biphenyl]-4-carboxylic acid (180 mg) was obtained (3 g).

¹H NMR (400 MHz, CDCl₃) δ 8.18 (d, J=8.4 Hz, 2H), 7.46 (d, J=8.4 Hz, 2H), 7.40-7.20 (m, 11H), 6.43 (d, J=8.8 Hz, 1H), 6.13 (d, J=7.6 Hz, 1H), 5.34-5.30 (m, 1H), 4.43 (s, 2H), 4.19 (t, J=4.0 Hz, 2H), 3.35 (t, J=4.4 Hz, 2H), 1.58 (d, J=6.8 Hz, 3H).

### Example 143

### (S)-4-((4′-carbamoyl-[1,1′-biphenyl]-2-yl)methyl)-N-(1-phenylethyl)-3,4-dihydro-2H-benzo[b][1,4]oxazine-7-carboxamide

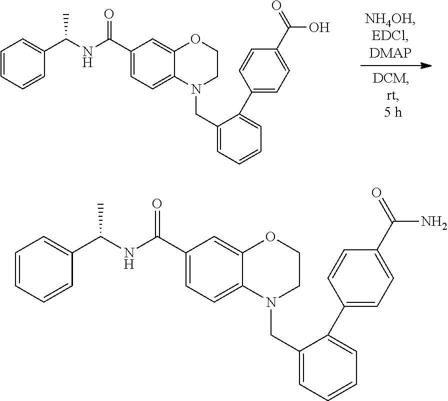 [SMILES: C[C@H](NC(=O)c1ccc2c(c1)OCCN2Cc1ccccc1-c1ccc(C(=O)O)cc1)c1ccccc1 ; C[C@H](NC(=O)c1ccc2c(c1)OCCN2Cc1ccccc1-c1ccc(C(N)=O)cc1)c1ccccc1]

(S)-2′-((7-((1-phenylethyl)carbamoyl)-2H-benzo[b][1,4]oxazine-4(3H)-yl)methyl)-[1,1′-biphenyl]-4-carboxylic acid (70 mg, 0.14 mmol) obtained in Example 142 was dissolved in CH₂Cl₂ (2 ml) in a 25 mL flask, to which EDCI (41 mg, 0.21 mmol) and HOBt (23 mg, 0.17 mmol) were added, followed by stirring at room temperature for 5 hours. Upon completion of the reaction, the organic layer was separated by using CH₂Cl₂ and H₂O. The separated organic layer was dried over MgSO₄ and filtered. The mixture was separated by column chromatography (MeOH/n-Hex/CH₂Cl₂=0.5:0.5:9) to give (S)-4-((4′-carbamoyl-[1,1′-biphenyl]-2-yl)methyl)-N-(1-phenylethyl)-3,4-dihydro-2H-benzo[b][1,4]oxazine-7-carboxamide (30 mg, 0.06 mmol, 42.9%).

¹H NMR (400 MHz, CDCl₃) δ 7.85 (d, J=8.4 Hz, 2H), 7.50-7.28 (m, 11H), 7.20-7.15 (m, 2H), 6.37 (d, J=8.4 Hz, 1H), 6.16-6.14 (m, 2H), 5.55 (s, 1H), 5.35-5.27 (m, 1H), 4.42 (s, 2H), 4.16 (t, J=4.0 Hz, 2H), 3.32 (t, J=4.4 Hz, 2H), 1.59 (d, J=7.2 Hz, 3H).

### Example 144

### (S)-2′-((7-((1-(4-cyanophenyl)ethyl)carbamoyl)-2H-benzo[b][1,4]oxazine-4(3H)-yl)methyl)-[1,1′-biphenyl]-4-carboxylic acid

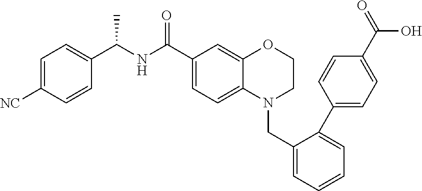 [SMILES: C[C@H](NC(=O)c1ccc2c(c1)OCCN2Cc1ccccc1-c1ccc(C(=O)O)cc1)c1ccc(C#N)cc1]

The target compound was obtained by the same manner as described in Example 142 except that (S)-1-(4-cyanophenyl)ethaneamine was used instead of (S)-1-phenylethylamine.

¹H NMR (400 MHz, CDCl₃) δ 8.17 (d, J=8.4 Hz, 2H), 7.62 (d, J=8.4 Hz, 2H), 7.48-7.45 (m, 4H), 7.42-7.24 (m, 4H), 7.22-7.02 (m, 2H), 6.42 (d, J=9.2 Hz, 1H), 6.16 (d, J=7.2 Hz, 1H), 5.34-5.27 (m, 1H), 4.44 (s, 2H), 4.19 (t, J=4.4 Hz, 2H), 3.35 (t, J=4.4 Hz, 2H), 1.57 (d, J=7.2 Hz, 3H).

### Example 145

### (S)-2′-((7-((1-(3-fluoro-4-methylphenyl)ethyl)carbamoyl)-2H-benzo[b][1,4]oxazine-4(3H)-yl)methyl)-[1,1′-biphenyl]-4-carboxylic acid

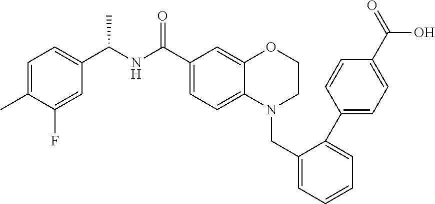 [SMILES: Cc1ccc([C@H](C)NC(=O)c2ccc3c(c2)OCCN3Cc2ccccc2-c2ccc(C(=O)O)cc2)cc1F]

The target compound was obtained by the same manner as described in Example 142 except that (S)-1-(3-fluoro-4-methylphenyl)ethaneamine was used instead of (S)-1-phenylethylamine.

¹H NMR (400 MHz, CDCl₃) δ 8.18 (d, J=8.4 Hz, 2H), 7.45 (d, J=8.0 Hz, 2H), 7.41-7.34 (m, 3H), 7.32-7.31 (m, 1H), 7.22-7.20 (m, 2H), 7.16-7.12 (m, 1H), 7.05-7.00 (m, 2H), 6.42 (d, J=9.2 Hz, 1H), 6.09 (d, J=7.6 Hz, 1H), 5.28-5.24 (m, 1H), 4.43 (s, 2H), 4.19 (t, J=4.0 Hz, 2H), 3.34 (t, J=4.0 Hz, 2H), 2.25 (s, 3H), 1.54 (d, J=6.8 Hz, 3H).

### Example 146

### (S)-2′-((7-((1-(4-nitrophenyl)ethyl)carbamoyl)-2H-benzo[b][1,4]oxazine-4 (3H)-yl)methyl)-[1,1′-biphenyl]-4-carboxylic acid

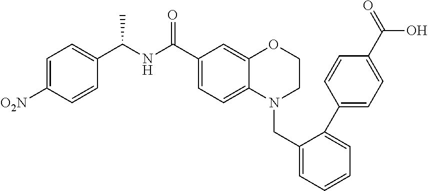 [SMILES: C[C@H](NC(=O)c1ccc2c(c1)OCCN2Cc1ccccc1-c1ccc(C(=O)O)cc1)c1ccc([N+](=O)[O-])cc1]

The target compound was obtained by the same manner as described in Example 142 except that (S)-1-(4-nitrophenyl)ethaneamine was used instead of (S)-1-phenylethylamine.

¹H NMR (400 MHz, CDCl₃) δ 8.21-8.15 (m, 4H), 7.54 (d, J=1.6 Hz, 2H), 7.45 (d, J=8.0 Hz, 2H), 7.40-7.30 (m, 4H), 7.23-7.21 (m, 2H), 6.42 (d, J=9.2 Hz, 1H), 6.19 (d, J=6.8 Hz, 1H), 5.34-5.32 (m, 1H), 4.44 (s, 2H), 4.18 (t, J=4.0 Hz, 2H), 3.35 (t, J=4.0 Hz, 2H), 1.59 (d, J=6.8 Hz, 3H).

### Example 147

### (S)-2′-((7-((1-(naphthalene-2-yl)ethyl)carbamoyl)-2H-benzo[b][1,4]oxazine-4(3H)-yl)methyl)-[1,1′-biphenyl]-4-carboxylic acid

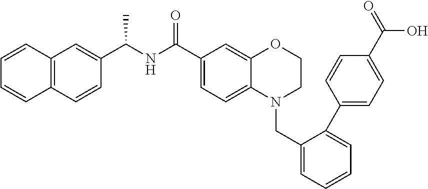 [SMILES: C[C@H](NC(=O)c1ccc2c(c1)OCCN2Cc1ccccc1-c1ccc(C(=O)O)cc1)c1ccc2ccccc2c1]

The target compound was obtained by the same manner as described in Example 142 except that (S)-1-(naphthalene-2-yl)ethaneamine was used instead of (S)-1-phenylethylamine.

¹H NMR (400 MHz, CDCl₃) δ 8.17 (d, J=8.4 Hz, 2H), 7.84-7.82 (m, 4H), 7.54-7.46 (m, 6H), 7.38-7.34 (m, 3H), 7.31-7.02 (m, 7H), 6.42 (d, J=8.0 Hz, 1H), 6.21 (d, J=7.2 Hz, 1H), 5.51-5.47 (m, 1H), 4.42 (s, 2H), 4.19 (t, J=4.0 Hz, 2H), 3.34 (t, J=4.8 Hz, 2H), 1.67 (d, J=6.8 Hz, 3H).

### Example 148

### (S)-4-((4′-carbamoyl-[1,1′-biphenyl]-2-yl)methyl)-N-(1-(3-fluoro-4-methylphenyl)ethyl)-3,4-dihydro-2H-benzo[b][1,4]oxazine-7-carboxamide

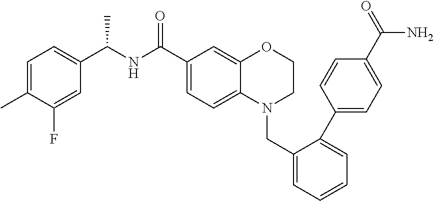 [SMILES: Cc1ccc([C@H](C)NC(=O)c2ccc3c(c2)OCCN3Cc2ccccc2-c2ccc(C(N)=O)cc2)cc1F]

The target compound was obtained by the same manner as described in Example 143 except that (S)-2′-((7-((1-(3-fluoro-4-methylphenyl)ethyl)carbamoyl)-2H-benzo[b][1,4]oxazine-4(3H)-yl)methyl)-[1,1′-biphenyl]-4-carboxylic acid obtained in Example 145 was used instead of (S)-2′-((7-((1-phenylethyl)carbamoyl)-2H-benzo[b][1,4]oxazine-4(3H)-yl)methyl)-[1,1′-biphenyl]-4-carboxylic acid.

¹H NMR (400 MHz, CDCl₃) δ 7.86 (d, J=7.6 Hz, 2H), 7.54-7.28 (m, 6H), 7.17-7.13 (m, 3H), 7.06-7.01 (m, 2H), 6.38 (d, J=8.4 Hz, 1H), 6.25-6.12 (m, 2H), 5.61 (s, 1H), 5.32-5.21 (m, 1H), 4.42 (s, 2H), 4.16 (t, J=4.0 Hz, 2H), 3.32 (t, J=4.0 Hz, 2H), 2.26 (s, 1H), 1.55 (d, J=6.4 Hz, 3H).

### Example 149

### (S)-4-((4′-carbamoyl-[1,1′-biphenyl]-2-yl)methyl)-N-(1-(4-cyanophenyl)ethyl)-3,4-dihydro-2H-benzo[b][1,4]oxazine-7-carboxamide

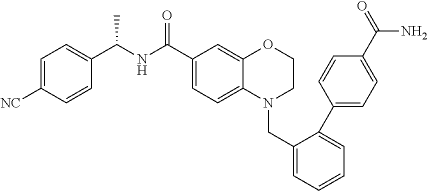 [SMILES: C[C@H](NC(=O)c1ccc2c(c1)OCCN2Cc1ccccc1-c1ccc(C(N)=O)cc1)c1ccc(C#N)cc1]

The target compound was obtained by the same manner as described in Example 143 except that (S)-2′-((7-((1-(4-cyanophenyl)ethyl)carbamoyl)-2H-benzo[b][1,4]oxazine-4(3H)-yl)methyl)-[1,1′-biphenyl]-4-carboxylic acid obtained in Example 144 was used instead of (S)-2′-((7-((1-phenylethyl)carbamoyl)-2H-benzo[b][1,4]oxazine-4(3H)-yl)methyl)-[1,1′-biphenyl]-4-carboxylic acid.

¹H NMR (400 MHz, CDCl₃) δ 7.86 (d, J=8.4 Hz, 2H), 7.64 (d, J=8.0 Hz, 2H), 7.52-7.47 (m, 2H), 7.40-7.30 (m, 6H), 7.21-7.16 (m, 2H), 6.38 (d, J=8.4 Hz, 1H), 6.23 (d, J=6.8 Hz, 1H), 6.12 (s, 1H), 5.64 (s, 1H), 5.32-5.26 (m, 1H), 4.43 (s, 2H), 4.16 (t, J=4.0 Hz, 2H), 3.33 (t, J=4.4 Hz, 2H), 1.57 (d, J=7.2 Hz, 3H).

### Example 150

### (S)-4-((4′-carbamoyl-[1,1′-biphenyl]-2-yl)methyl)-N-(1-(4-nitrophenyl)ethyl)-3,4-dihydro-2H-benzo[b][1,4]oxazine-7-carboxamide

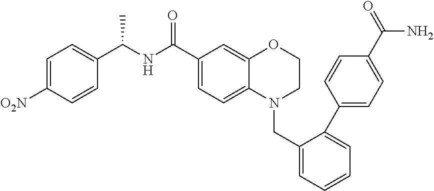 [SMILES: C[C@H](NC(=O)c1ccc2c(c1)OCCN2Cc1ccccc1-c1ccc(C(N)=O)cc1)c1ccc([N+](=O)[O-])cc1]

The target compound was obtained by the same manner as described in Example 143 except that (S)-2′-((7-((1-(4-nitrophenyl)ethyl)carbamoyl)-2H-benzo[b][1,4]oxazine-4(3H)-yl)methyl)-[1,1′-biphenyl]-4-carboxylic acid obtained in Example 146 was used instead of (S)-2′-((7-((1-phenylethyl)carbamoyl)-2H-benzo[b][1,4]oxazine-4(3H)-yl)methyl)-[1,1′-biphenyl]-4-carboxylic acid.

¹H NMR (400 MHz, CDCl₃) δ 8.20 (d, J=8.4 Hz, 2H), 7.85 (d, J=8.0 Hz, 2H), 7.54 (d, J=8.8 Hz, 2H), 7.42-7.30 (m, 6H), 7.21-7.16 (m, 2H), 6.38 (d, J=8.4 Hz, 1H), 6.27 (d, J=7.2 Hz, 1H), 6.11 (s, 1H), 5.60 (s, 1H), 5.37-5.28 (m, 1H), 4.43 (s, 2H), 4.17 (t, J=4.0 Hz, 2H), 3.33 (t, J=4.4 Hz, 2H), 1.62 (d, J=8.8 Hz, 3H).

### Example 151

### (S)-4-((4′-carbamoyl-[1,1′-biphenyl]-2-yl)methyl)-N-(1-(naphthalene-2-yl)ethyl)-3,4-dihydro-2H-benzo[b][1,4]oxazine-7-carboxamide

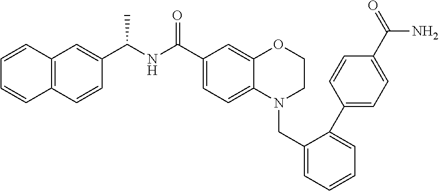 [SMILES: C[C@H](NC(=O)c1ccc2c(c1)OCCN2Cc1ccccc1-c1ccc(C(N)=O)cc1)c1ccc2ccccc2c1]

The target compound was obtained by the same manner as described in Example 143 except that (S)-2′-((7-((1-(naphthalene-2-yl)ethyl)carbamoyl)-2H-benzo[b][1,4]oxazine-4(3H)-yl)methyl)-[1,1′-biphenyl]-4-carboxylic acid obtained in Example 147 was used instead of (S)-2′-((7-((1-phenylethyl)carbamoyl)-2H-benzo[b][1,4]oxazine-4(3H)-yl)methyl)-[1,1′-biphenyl]-4-carboxylic acid.

¹H NMR (400 MHz, CDCl₃) δ 7.86-7.82 (m, 6H), 7.53-7.45 (m, 3H), 7.41-7.27 (m, 6H), 7.22-7.17 (m, 2H), 7.21-7.16 (m, 2H), 6.39 (d, J=8.8 Hz, 1H), 6.25 (d, J=8.0 Hz, 1H), 6.17 (s, 1H), 5.62 (s, 1H), 5.54-5.45 (m, 1H), 4.41 (s, 2H), 4.16 (t, J=4.4 Hz, 2H), 3.31 (t, J=4.4 Hz, 2H), 1.68 (d, J=6.8 Hz, 3H).

### Example 152

### (S)-2′-((6-((1-phenylethyl) carbamoyl)-3, 4-dihydroquinoline-1 (2H)-yl)methyl)-[1,1′-biphenyl]-4-carboxylic acid

### Step 1 (S)—N-(1-phenylethyl)-1,2,3,4-tetrahydroquinoline-6-carboxamide

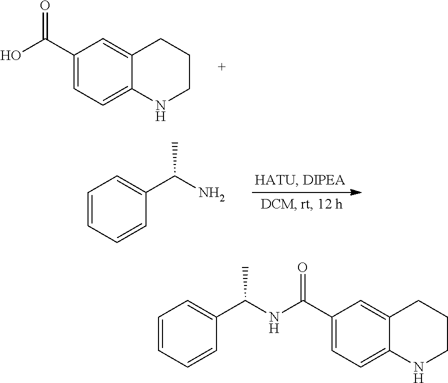

1,2,3,4-tetrahydroquinoline-6-carboxylic acid (300 mg, 1.63 mmol) obtained in Preparative Example 8 was dissolved in CH₂Cl₂ (10 ml) in a 100 mL flask, to which HATU (934 mg, 2.45 mmol) and DIPEA (856 ul, 4.91 mmol) were added, followed by stirring at room temperature for 12 hours. Upon completion of the reaction, the organic layer was separated by using CH₂Cl₂ and H₂O. The separated organic layer was dried over MgSO₄ and filtered. The mixture was separated by column chromatography (EA/n-Hex=1:1) to give (S)—N-(1-phenylethyl)-1,2,3,4-tetrahydroquinoline-6-carboxamide (210 mg).

### Step 2 (S)-methyl 2′-((6-((1-phenylethyl)carbamoyl)-3,4-dihydroquinoline-1(2H)-yl)methyl)-[1,1′-biphenyl]-4-carboxylate

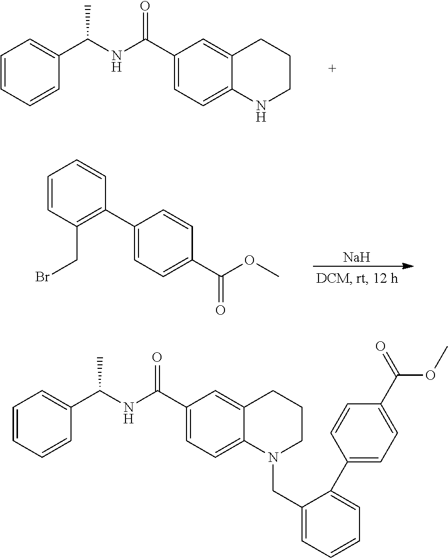

(S)—N-(1-phenylethyl)-1,2,3,4-tetrahydroquinoline-6-carboxamide (182 mg, 0.65 mmol) was dissolved in DMF (3 ml) in a 25 mL flask, to which NaH (26 mg, 0.65 mmol) and methyl 2′-(bromomethyl)-[1,1′-biphenyl]-4-carboxylate (200 mg, 0.65 mmol) were added, followed by stirring at room temperature for 12 hours. The organic layer was separated by using ethyl acetate and brine. The separated organic layer was dried over MgSO₄ and filtered. The solvent was eliminated from the mixture via vacuum distillation, followed by column chromatography to give (S)-methyl 2′-((6-((1-phenylethyl)carbamoyl)-3,4-dihydroquinoline-1(2H)-yl)methyl)-[1,1′-biphenyl]-4-carboxylate (140 mg).

### Step 3 (S)-2′-((6-((1-phenylethyl)carbamoyl)-3,4-dihydroquinoline-1(2H)-yl)methyl)-[1,1′-biphenyl]-4-carboxylic acid

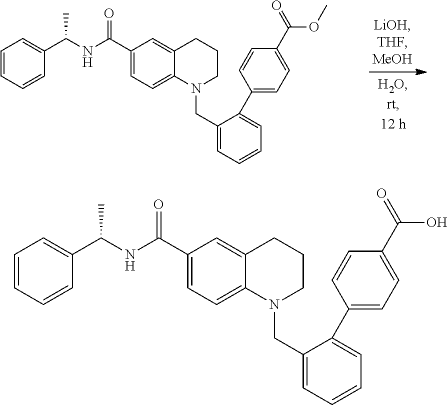 [SMILES: COC(=O)c1ccc(-c2ccccc2CN2CCCc3cc(C(=O)N[C@@H](C)c4ccccc4)ccc32)cc1 ; C[C@H](NC(=O)c1ccc2c(c1)CCCN2Cc1ccccc1-c1ccc(C(=O)O)cc1)c1ccccc1]

(S)-methyl 2′-((6-((1-phenylethyl)carbamoyl)-3,4-dihydroquinoline-1(2H)-yl)methyl)-[1,1′-biphenyl]-4-carboxylate (140 mg, 0.27 mmol) was dissolved in MeOH (5 ml) and THF (10 ml) in a 25 mL flask, to which LiOH (2 g) dissolved in H₂O (5 ml) was added, followed by stirring at room temperature for 12 hours. Upon completion of the reaction, the solvent was concentrated. PH of the reaction mixture was adjusted to 5 by using 2N-HCl. The mixture was extracted with EA. The organic layer was dried over MgSO₄ and filtered. As a result, (S)-2′-((6-((1-phenylethyl)carbamoyl)-3,4-dihydroquinoline-1(2H)-yl)methyl)-[1,1′-biphenyl]-4-carboxylic acid (99.4 mg) was obtained.

¹H NMR (400 MHz, CDCl₃) δ 8.19 (d, J=8.0 Hz, 2H), 7.48-7.44 (m, 4H), 7.41-7.25 (m, 9H), 7.58-7.27 (m, 8H), 6.28 (d, J=8.8 Hz, 1H), 6.12 (d, J=8.0 Hz, 1H), 5.36-5.32 (m, 1H), 4.42 (s, 2H), 3.34 (t, J=5.6 Hz, 2H), 2.80 (t, J=6.0 Hz, 2H), 2.01-1.95 (m, 2H), 1.59 (d, J=7.2 Hz, 3H).

### Example 153

### (S)-1-((4′-carbamoyl-[1,1′-biphenyl]-2-yl)methyl)-N-(1-phenylethyl)-1,2,3,4-tetrahydroquinolin-6-carboxamide

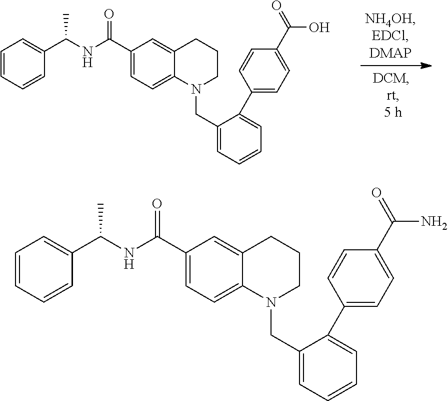 [SMILES: C[C@H](NC(=O)c1ccc2c(c1)CCCN2Cc1ccccc1-c1ccc(C(=O)O)cc1)c1ccccc1 ; C[C@H](NC(=O)c1ccc2c(c1)CCCN2Cc1ccccc1-c1ccc(C(N)=O)cc1)c1ccccc1]

(S)-2′-((6-((1-phenylethyl)carbamoyl)-3,4-dihydroquinoline-1(2H)-yl)methyl)-[1,1′-biphenyl]-4-carboxylic acid (66.4 mg, 0.14 mmol) obtained in Example 152 was dissolved in CH₂Cl₂ (2 ml) in a 25 mL flask, to which EDCI (41 mg, 0.21 mmol) and HOBt (23 mg, 0.17 mmol) were added, followed by stirring. Ammonium hydroxide solution (3 ml) was added thereto, followed by stirring at room temperature for 5 hours. Upon completion of the reaction, the organic layer was separated by using CH₂Cl₂ and H₂O. The separated organic layer was dried over MgSO₄ and filtered. The mixture was separated by column chromatography (MeOH/n-Hex/CH₂Cl₂=0.5:0.5:9) to give (S)-1-((4′-carbamoyl-[1,1′-biphenyl]-2-yl)methyl)-N-(1-phenylethyl)-1,2,3,4-tetrahydroquinoline-6-carboxamide (54.7 mg).

1H NMR (400 MHz, CDCl₃) δ 7.86 (d, J=8.0 Hz, 2H), 7.43-7.26 (m, 12H), 6.26 (d, J=8.8 Hz, 2H), 6.20-6.11 (m, 2H), 6.64 (s, 1H), 5.37-5.30 (m, 1H), 4.41 (s, 2H), 3.31 (t, J=5.6 Hz, 2H), 2.78 (t, J=6.0 Hz, 2H), 1.99-1.93 (m, 2H), 1.57 (d, J=6.4 Hz, 3H).

### Example 154

### (S)-2′-((6-((1-(3-fluoro-4-methylphenyl)ethyl)carbamoyl)-3,4-dihydroquinoline-1(2H)-yl)methyl)-[1,1′-biphenyl]-4-carboxylic acid

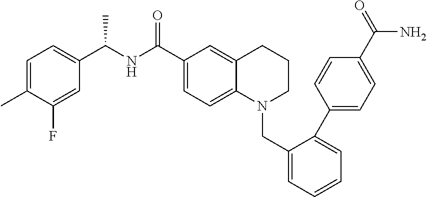 [SMILES: Cc1ccc([C@H](C)NC(=O)c2ccc3c(c2)CCCN3Cc2ccccc2-c2ccc(C(N)=O)cc2)cc1F]

The target compound was obtained by the same manner as described in Example 152 except that (S)-1-(3-fluoro-4-methylphenyl)ethaneamine was used instead of (S)-1-phenylethylamine.

¹H NMR (400 MHz, CDCl₃) δ 8.18 (d, J=8.0 Hz, 2H), 7.47-7.42 (m, 3H), 7.38-7.28 (m, 5H), 7.16-7.12 (m, 1H), 7.06-7.00 (m, 2H), 6.27 (d, J=8.8 Hz, 1H), 6.08 (d, J=8.0 Hz, 1H), 5.32-5.26 (m, 1H), 4.42 (s, 2H), 3.33 (t, J=4.0 Hz, 2H), 2.78 (t, J=4.0 Hz, 2H), 2.25 (s, 3H), 1.99-1.97 (m, 2H), 1.54 (d, J=6.8 Hz, 3H).

### Example 155

### (S)-2′-((6-((1-(4-nitrophenyl)ethyl)carbamoyl)-3,4-dihydroquinoline-1(2H)-yl)methyl)-[1,1′-biphenyl]-4-carboxylic acid

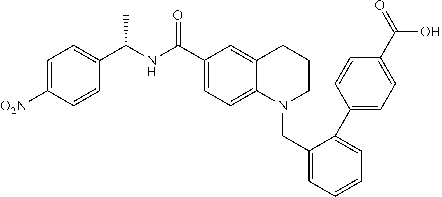 [SMILES: C[C@H](NC(=O)c1ccc2c(c1)CCCN2Cc1ccccc1-c1ccc(C(=O)O)cc1)c1ccc([N+](=O)[O-])cc1]

The target compound was obtained by the same manner as described in Example 152 except that (S)-1-(4-nitrophenyl)ethaneamine was used instead of (S)-1-phenylethylamine.

¹H NMR (400 MHz, CDCl₃) δ 8.18 (d, J=8.4 Hz, 4H), 7.54-7.28 (m, 10H), 6.28 (d, J=8.4 Hz, 1H), 6.20 (d, J=6.8 Hz, 1H), 5.38-5.32 (m, 1H), 4.43 (s, 2H), 3.35 (t, J=4.4 Hz, 2H), 2.80 (t, J=5.6 Hz, 2H), 1.98 (t, J=4.8 Hz, 2H), 1.59 (d, J=6.8 Hz, 3H).

### Example 156

### (S)-2′-((6-((1-(naphthalene-2-yl)ethyl)carbamoyl)-3,4-dihydroquinoline-1(2H)-yl)methyl)-[1,1′-biphenyl]-4-carboxylic acid

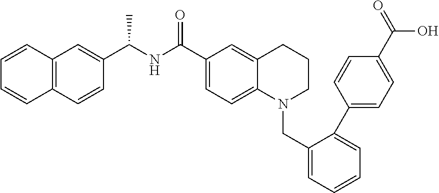 [SMILES: C[C@H](NC(=O)c1ccc2c(c1)CCCN2Cc1ccccc1-c1ccc(C(=O)O)cc1)c1ccc2ccccc2c1]

The target compound was obtained by the same manner as described in Example 152 except that (S)-1-(naphthalene-2-yl)ethaneamine was used instead of (S)-1-phenylethylamine.

¹H NMR (400 MHz, CDCl₃) δ 8.18 (d, J=7.6 Hz, 2H), 7.84-7.81 (m, 4H), 7.53-7.28 (m, 11H), 6.28 (d, J=8.8 Hz, 1H), 6.22 (d, J=7.6 Hz, 1H), 5.53-5.48 (m, 1H), 4.41 (s, 2H), 3.32 (t, J=4.4 Hz, 2H), 2.79 (t, J=5.6 Hz, 2H), 1.97 (t, J=4.8 Hz, 2H), 1.68 (d, J=6.8 Hz, 3H).

### Example 157

### (S)-2′-((6-((1-(4-cyanophenyl)ethyl)carbamoyl)-3,4-dihydroquinoline-1(2H)-yl)methyl)-[1,1′-biphenyl]-4-carboxylic acid

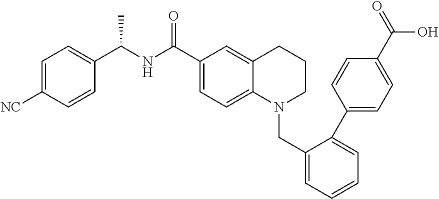 [SMILES: C[C@H](NC(=O)c1ccc2c(c1)CCCN2Cc1ccccc1-c1ccc(C(=O)O)cc1)c1ccc(C#N)cc1]

The target compound was obtained by the same manner as described in Example 152 except that (S)-1-(4-cyanophenyl)ethaneamine was used instead of (S)-1-phenylethylamine.

¹H NMR (400 MHz, CDCl₃) δ 8.18 (d, J=8.0 Hz, 2H), 7.62 (d, J=8.0 Hz, 2H), 7.48-7.28 (m, 10H), 6.27 (d, J=8.4 Hz, 1H), 6.16 (d, J=7.6 Hz, 1H), 5.34-5.30 (m, 1H), 4.43 (s, 2H), 3.34 (t, J=5.6 Hz, 2H), 2.80 (t, J=6.4 Hz, 2H), 2.00-1.97 (m, 2H), 1.57 (d, J=6.8 Hz, 3H).

### Example 158

### (S)-1-((4′-carbamoyl-[1,1′-biphenyl]-2-yl)methyl)-N-(1-(3-fluoro-4-methylphenyl)ethyl)-1,2,3,4-tetrahydroquinoline-6-carboxamide

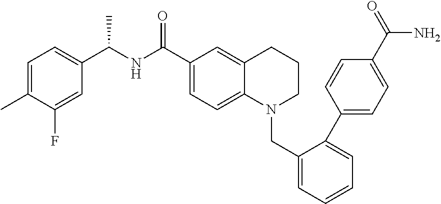 [SMILES: Cc1ccc([C@H](C)NC(=O)c2ccc3c(c2)CCCN3Cc2ccccc2-c2ccc(C(N)=O)cc2)cc1F]

The target compound was obtained by the same manner as described in Example 153 except that (S)-2′-((6-((1-(3-fluoro-4-methylphenyl)ethyl)carbamoyl)-3,4-dihydroquinoline-1(2H)-yl)methyl)-[1,1′-biphenyl]-4-carboxylic acid obtained in Example 154 was used instead of (S)-2′-((6-((1-phenylethyl)carbamoyl)-3,4-dihydroquinoline-1(2H)-yl)methyl)-[1,1′-biphenyl]-4-carboxylic acid.

¹H NMR (400 MHz, CDCl₃) δ 7.87 (d, J=7.6 Hz, 2H), 7.44-7.32 (m, 8H), 7.17-7.13 (m, 1H), 7.07-7.02 (m, 2H), 6.26 (d, J=8.4 Hz, 1H), 6.10-6.08 (m, 2H), 5.57 (s, 1H), 5.33-5.25 (m, 1H), 4.42 (s, 2H), 3.32 (t, J=5.2 Hz, 2H), 2.79 (t, J=6.0 Hz, 2H), 2.26 (s, 3H), 1.98-1.95 (m, 2H), 1.55 (d, J=6.8 Hz, 3H).

### Example 159

### (S)-1-((4′-carbamoyl-[1,1′-biphenyl]-2-yl)methyl)-N-(1-(4-nitrophenyl)ethyl)-1,2,3,4-tetrahydroquinoline-6-carboxamide

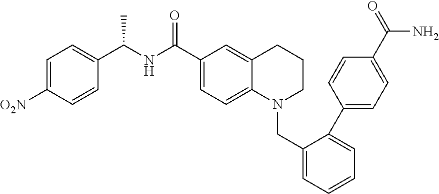 [SMILES: C[C@H](NC(=O)c1ccc2c(c1)CCCN2Cc1ccccc1-c1ccc(C(N)=O)cc1)c1ccc([N+](=O)[O-])cc1]

The target compound was obtained by the same manner as described in Example 153 except that (S)-2′-((6-((1-(4-nitrophenyl)ethyl)carbamoyl)-3,4-dihydroquinoline-1(2H)-yl)methyl)-[1,1′-biphenyl]-4-carboxylic acid obtained in Example 155 was used instead of (S)-2′-((6-((1-phenylethyl)carbamoyl)-3,4-dihydroquinoline-1(2H)-yl)methyl)-[1,1′-biphenyl]-4-carboxylic acid.

¹H NMR (400 MHz, CDCl₃) δ 8.20 (d, J=8.8 Hz, 2H), 7.87 (d, J=8.0 Hz, 2H), 7.54 (d, J=8.8 Hz, 2H), 7.50-7.33 (m, 8H), 6.26 (d, J=8.4 Hz, 1H), 6.21 (d, J=7.2 Hz, 1H), 6.01 (s, 1H), 5.58 (s, 1H), 5.38-5.32 (m, 1H), 4.42 (s, 2H), 3.33 (t, J=5.6 Hz, 2H), 2.79 (t, J=5.6 Hz, 2H), 1.99-1.96 (m, 2H), 1.60 (d, J=6.8 Hz, 3H).

### Example 160

### (S)-1-((4′-carbamoyl-[1,1′-biphenyl]-2-yl)methyl)-N-(1-(naphthalene-2-yl)ethyl)-1,2,3,4-tetrahydroquinoline-6-carboxamide

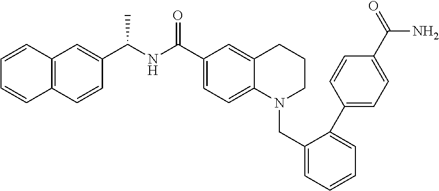 [SMILES: C[C@H](NC(=O)c1ccc2c(c1)CCCN2Cc1ccccc1-c1ccc(C(N)=O)cc1)c1ccc2ccccc2c1]

The target compound was obtained by the same manner as described in Example 153 except that (S)-2′-((6-((1-(naphthalene-2-yl)ethyl)carbamoyl)-3,4-dihydroquinoline-1(2H)-yl)methyl)-[1,1′-biphenyl]-4-carboxylic acid obtained in Example 156 was used instead of (S)-2′-((6-((1-phenylethyl)carbamoyl)-3,4-dihydroquinoline-1(2H)-yl)methyl)-[1,1′-biphenyl]-4-carboxylic acid.

¹H NMR (400 MHz, CDCl₃) δ 7.87-7.83 (m, 6H), 7.54-7.26 (d, 11H), 6.27 (d, J=8.4 Hz, 1H), 6.21 (d, J=8.0 Hz, 1H), 6.13 (s, 1H), 5.55-5.48 (m, 2H), 4.41 (s, 2H), 3.31 (t, J=5.2 Hz, 2H), 2.78 (t, J=5.6 Hz, 2H), 2.01-1.96 (m, 2H), 1.68 (d, J=6.8 Hz, 3H).

### Example 161

### (S)-1-((4′-carbamoyl-[1,1′-biphenyl]-2-yl)methyl)-N-(1-(4-cyanophenyl)ethyl)-1,2,3,4-tetrahydroquinoline-6-carboxamide

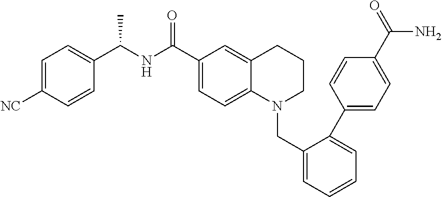 [SMILES: C[C@H](NC(=O)c1ccc2c(c1)CCCN2Cc1ccccc1-c1ccc(C(N)=O)cc1)c1ccc(C#N)cc1]

The target compound was obtained by the same manner as described in Example 153 except that (S)-2′-((6-((1-(4-cyanophenyl)ethyl)carbamoyl)-3,4-dihydroquinoline-1(2H)-yl)methyl)-[1,1′-biphenyl]-4-carboxylic acid obtained in Example 157 was used instead of (S)-2′-((6-((1-phenylethyl)carbamoyl)-3,4-dihydroquinoline-1(2H)-yl)methyl)-[1,1′-biphenyl]-4-carboxylic acid.

¹H NMR (400 MHz, CDCl₃) δ 7.97 (d, J=8.8 Hz, 2H), 7.87 (d, J=8.0 Hz, 2H), 7.63 (d, J=8.0 Hz, 2H), 7.58-7.27 (m, 8H), 6.26 (d, J=8.8 Hz, 1H), 6.19-6.04 (m, 2H), 5.57 (s, 1H), 5.34-5.27 (m, 1H), 4.42 (s, 2H), 3.33 (t, J=5.6 Hz, 2H), 2.79 (t, J=6.0 Hz, 2H), 2.07-1.95 (m, 2H), 1.57 (d, J=6.4 Hz, 3H).

### Example 162

### (S)-2′-fluoro-4′-((7-((1-phenylethyl)carbamoyl)-2H-benzo[b][1,4]oxazine-4(3H)-yl)methyl)-[1,1′-biphenyl]-2-carboxylic acid

### Step 1: (S)-tert-butyl 2′-fluoro-4′-((7-((1-phenylethyl)carbamoyl)-2H-benzo[b][1,4]oxazine-4(3H)-yl)methyl)-[1,1′-biphenyl]-2-carboxylate

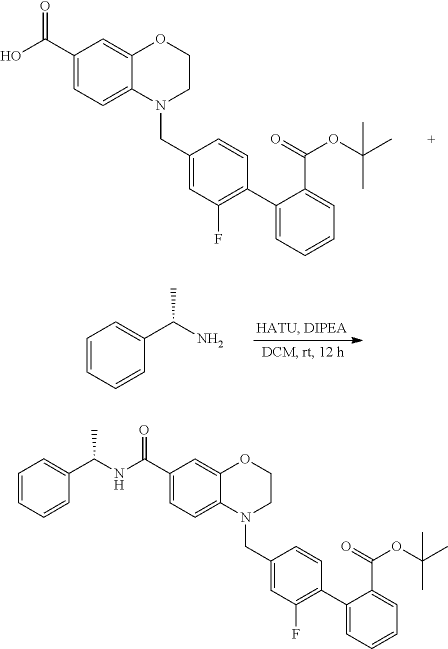

4-((2′-(tert-butoxycarbonyl)-2-fluoro-[1,1′-biphenyl]-4-yl)methyl)-3, 4-dihydro-2H-benzo[b][1,4]oxazine-7-carboxylic acid (300 mg, 0.65 mmol) obtained in Preparative Example 9 was dissolved in CH₂Cl₂ (10 ml) in a 100 mL flask, to which HATU (386 mg, 1.01 mmol) and DIPEA (353 ul, 2.02 mmol) were added, followed by stirring. (S)-1-phenylethylamine (105 ul, 0.81 mmol) was added thereto, followed by stirring at room temperature for 12 hours. Upon completion of the reaction, the organic layer was separated by using CH₂Cl₂ and H₂O. The separated organic layer was dried over MgSO₄ and filtered. The mixture was separated by column chromatography (EA/n-Hex=1:1) to give (S)-tert-butyl 2′-fluoro-4′-((7-((1-phenylethyl)carbamoyl)-2H-benzo[b][1,4]oxazine-4(3H)-yl)methyl)-[1,1′-biphenyl]-2-carboxylate (180 mg, 0.31 mmol, 47%).

### Step 2 (S)-2′-fluoro-4′-((7-((1-phenylethyl)carbamoyl)-2H-benzo[b][1,4]oxazine-4(3H)-yl)methyl)-[1,1′-biphenyl]-2-carboxylic acid

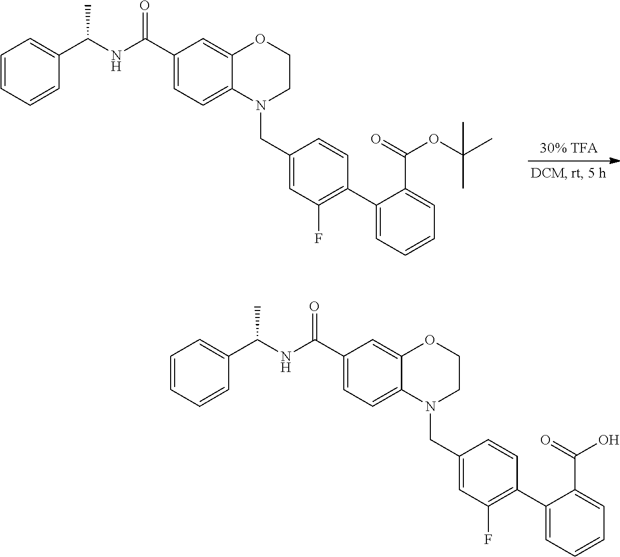 [SMILES: C[C@H](NC(=O)c1ccc2c(c1)OCCN2Cc1ccc(-c2ccccc2C(=O)OC(C)(C)C)c(F)c1)c1ccccc1 ; C[C@H](NC(=O)c1ccc2c(c1)OCCN2Cc1ccc(-c2ccccc2C(=O)O)c(F)c1)c1ccccc1]

(S)-tert-butyl 2′-fluoro-4′-((7-((1-phenylethyl)carbamoyl)-2H-benzo[b][1,4]oxazine-4(3H)-yl)methyl)-[1,1′-biphenyl]-2-carboxylate (180 mg, 0.31 mmol) obtained in step 1 was dissolved in CH₂Cl₂ (10 ml) in a 50 mL flask, to which 30% TFA (3 ml) was added, followed by stirring at room temperature for 5 hours. Upon completion of the reaction the reaction mixture was concentrated under reduced pressure. The mixture was separated by column chromatography (EA) to give (S)-2′-fluoro-4′-((7-((1-phenylethyl)carbamoyl)-2H-benzo[b][1,4]oxazine-4(3H)-yl)methyl)-[1,1′-biphenyl]-2-carboxylic acid (100 mg, 0.19 mmol, 61%).

¹H NMR (400 MHz, CDCl₃) δ 8.07-8.05 (m, 1H), 7.65-7.61 (m, 1H), 7.51-7.47 (m, 1H), 7.40-7.33 (m, 6H), 7.30-7.11 (m, 3H), 7.11-7.09 (m, 1H), 7.05-6.96 (m, 3H), 6.62 (d, J=8.4 Hz, 1H), 6.19 (d, J=7.6 Hz, 1H), 5.29-5.22 (m, 1H), 4.55 (s, 2H), 4.29 (t, J=4.4 Hz, 2H), 3.50 (t, J=4.4 Hz, 2H), 2.25 (s, 3H), 1.53 (d, J=7.2 Hz, 3H).

### Example 163

### (S)-2′-fluoro-4′-((7-((1-(naphthalene-2-yl)ethyl)carbamoyl)-2H-benzo[b][1,4]oxazine-4(3H)-yl)methyl)-[1,1′-biphenyl]-2-carboxylic acid

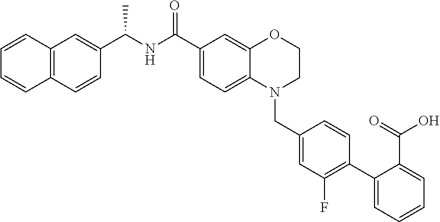 [SMILES: C[C@H](NC(=O)c1ccc2c(c1)OCCN2Cc1ccc(-c2ccccc2C(=O)O)c(F)c1)c1ccc2ccccc2c1]

The target compound was obtained by the same manner as described in Example 162 except that (S)-1-(naphthalene-2-yl)ethaneamine was used instead of (S)-1-phenylethylamine.

¹H NMR (400 MHz, DMSO-d₆) δ 12.67 (br, OH, 1H), 8.56-8.53 (m, 1H), 7.87-7.81 (m, 5H), 7.63-7.48 (m, 5H), 7.38-7.35 (m, 4H), 7.17-7.08 (m, 2H), 6.75-7.71 (m, 1H), 5.30-5.28 (m, 1H), 4.65 (s, 2H), 4.27-4.21 (m, 2H), 3.54 (s, 2H), 1.56-1.47 (m, 3H).

### Example 164

### (S)-2′-fluoro-4′-((7-(((3-fluoro-4-methylphenyl)ethyl)carbamoyl)-2H-benzo[b][1,4]oxazine-4(3)-yl)methyl)-[1,1′-biphenyl]-2-carboxylic acid

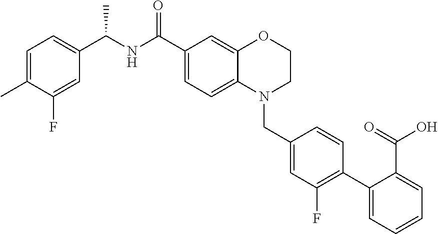 [SMILES: Cc1ccc([C@H](C)NC(=O)c2ccc3c(c2)OCCN3Cc2ccc(-c3ccccc3C(=O)O)c(F)c2)cc1F]

The target compound was obtained by the same manner as described in Example 162 except that (S)-1-(3-fluoro-4-methylphenyl)ethaneamine was used instead of (S)-1-phenylethylamine.

¹H NMR (400 MHz, CDCl₃) δ 8.07-8.05 (m, 1H), 7.64-7.60 (m, 1H), 7.51-7.47 (m, 1H), 7.40-7.36 (m, 1H), 7.30-7.26 (m, 3H), 7.15-7.08 (m, 2H), 7.02-6.79 (m, 1H), 6.63 (d, J=8.4 Hz, 1H), 6.18 (d, J=7.6 Hz, 1H), 5.36-5.28 (m, 1H), 4.55 (s, 2H), 4.29 (t, J=4.4 Hz, 2H), 3.50 (t, J=4.4 Hz, 2H), 1.58 (d, J=7.8 Hz, 3H).

### Comparative Example 1

### SR1664

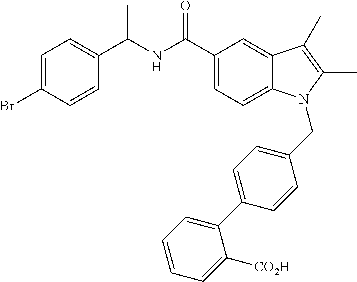 [SMILES: Cc1c(C)n(Cc2ccc(-c3ccccc3C(=O)O)cc2)c2ccc(C(=O)NC(C)c3ccc(Br)cc3)cc12]

### Comparative Example 2

### SR1824

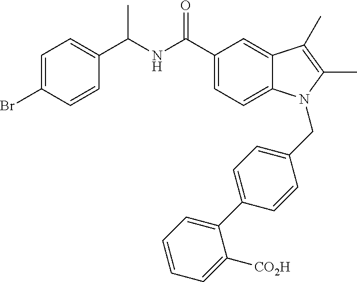 [SMILES: Cc1c(C)n(Cc2ccc(-c3ccccc3C(=O)O)cc2)c2ccc(C(=O)NC(C)c3ccc(Br)cc3)cc12]

The chemical formulae of the compounds prepared in Examples 1-164 are shown in Table 1.

TABLE 1

| Example | Formula |
|---|---|
| 1 | 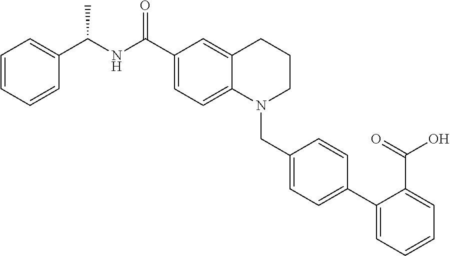 [SMILES: C[C@H](NC(=O)c1ccc2c(c1)CCCN2Cc1ccc(-c2ccccc2C(=O)O)cc1)c1ccccc1] |
| 2 | 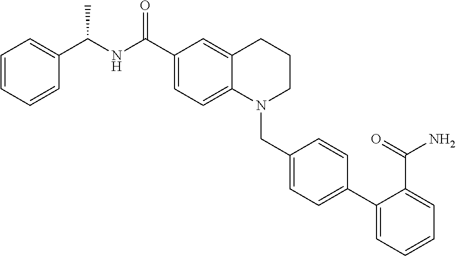 [SMILES: C[C@H](NC(=O)c1ccc2c(c1)CCCN2Cc1ccc(-c2ccccc2C(N)=O)cc1)c1ccccc1] |
| 3 | 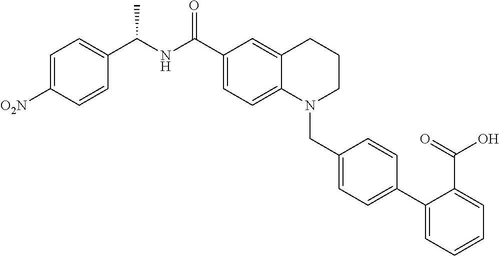 [SMILES: C[C@H](NC(=O)c1ccc2c(c1)CCCN2Cc1ccc(-c2ccccc2C(=O)O)cc1)c1ccc([N+](=O)[O-])cc1] |
| 4 | 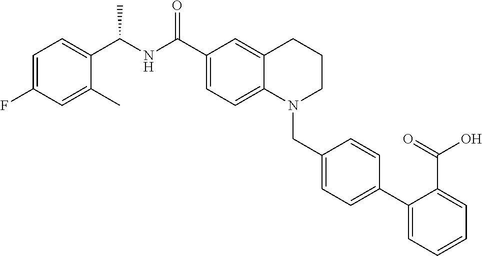 [SMILES: Cc1cc(F)ccc1[C@H](C)NC(=O)c1ccc2c(c1)CCCN2Cc1ccc(-c2ccccc2C(=O)O)cc1] |
| 5 | 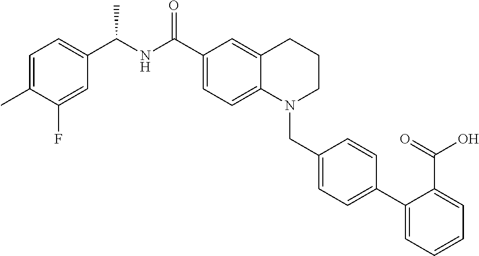 [SMILES: Cc1ccc([C@H](C)NC(=O)c2ccc3c(c2)CCCN3Cc2ccc(-c3ccccc3C(=O)O)cc2)cc1F] |
| 6 | 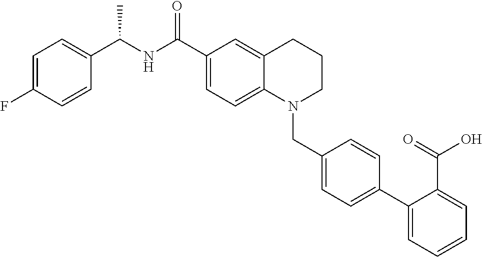 [SMILES: C[C@H](NC(=O)c1ccc2c(c1)CCCN2Cc1ccc(-c2ccccc2C(=O)O)cc1)c1ccc(F)cc1] |
| 7 | 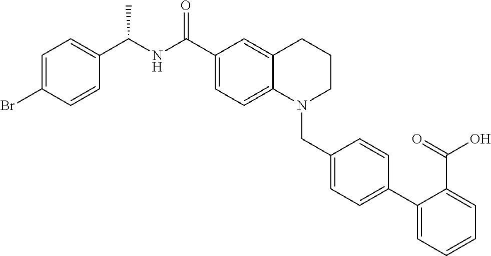 [SMILES: C[C@H](NC(=O)c1ccc2c(c1)CCCN2Cc1ccc(-c2ccccc2C(=O)O)cc1)c1ccc(Br)cc1] |
| 8 | 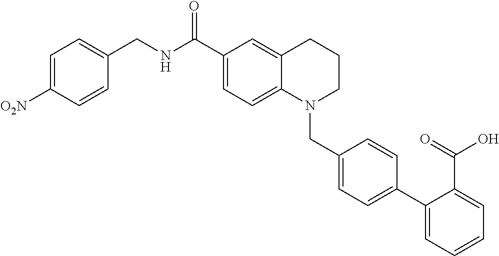 [SMILES: O=C(NCc1ccc([N+](=O)[O-])cc1)c1ccc2c(c1)CCCN2Cc1ccc(-c2ccccc2C(=O)O)cc1] |
| 9 | 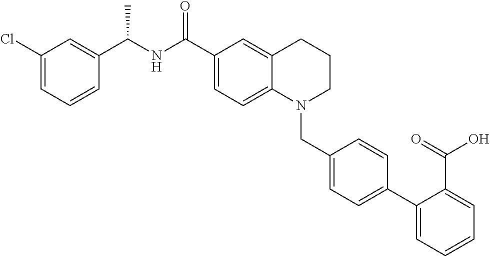 [SMILES: C[C@H](NC(=O)c1ccc2c(c1)CCCN2Cc1ccc(-c2ccccc2C(=O)O)cc1)c1cccc(Cl)c1] |
| 10 | 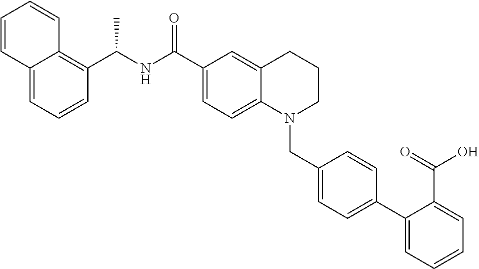 [SMILES: C[C@H](NC(=O)c1ccc2c(c1)CCCN2Cc1ccc(-c2ccccc2C(=O)O)cc1)c1cccc2ccccc12] |
| 11 | 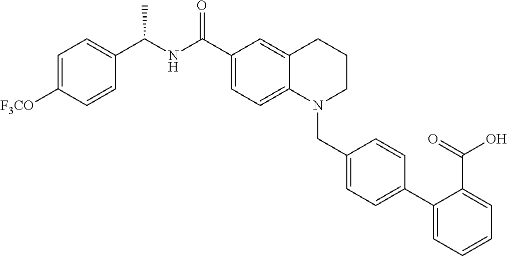 [SMILES: C[C@H](NC(=O)c1ccc2c(c1)CCCN2Cc1ccc(-c2ccccc2C(=O)O)cc1)c1ccc(OC(F)(F)F)cc1] |
| 12 | 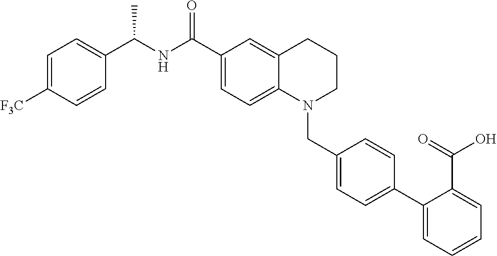 [SMILES: C[C@H](NC(=O)c1ccc2c(c1)CCCN2Cc1ccc(-c2ccccc2C(=O)O)cc1)c1ccc(C(F)(F)F)cc1] |
| 13 | 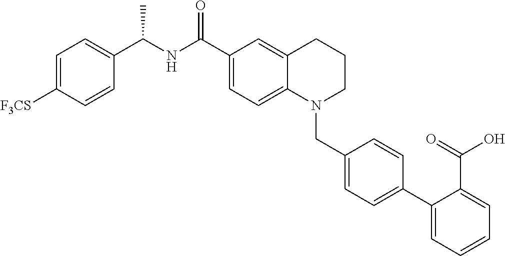 |
| 14 | 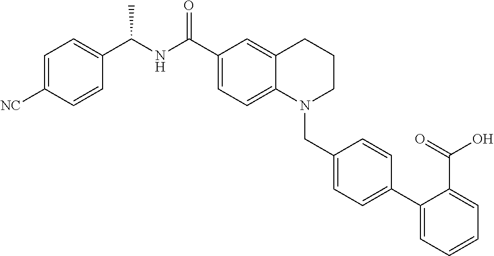 [SMILES: C[C@H](NC(=O)c1ccc2c(c1)CCCN2Cc1ccc(-c2ccccc2C(=O)O)cc1)c1ccc(C#N)cc1] |
| 15 | 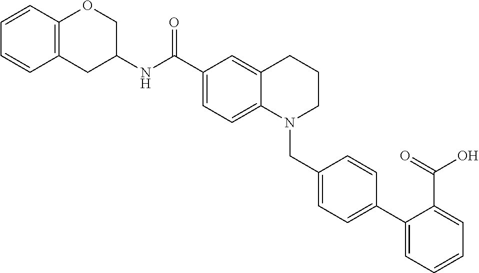 [SMILES: O=C(NC1COc2ccccc2C1)c1ccc2c(c1)CCCN2Cc1ccc(-c2ccccc2C(=O)O)cc1] |
| 16 | 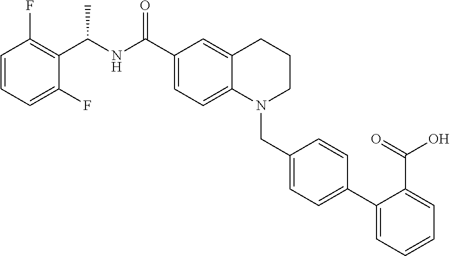 [SMILES: C[C@H](NC(=O)c1ccc2c(c1)CCCN2Cc1ccc(-c2ccccc2C(=O)O)cc1)c1c(F)cccc1F] |
| 17 | 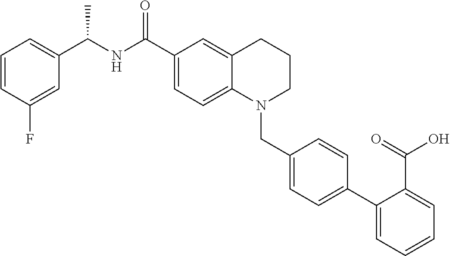 [SMILES: C[C@H](NC(=O)c1ccc2c(c1)CCCN2Cc1ccc(-c2ccccc2C(=O)O)cc1)c1cccc(F)c1] |
| 18 | 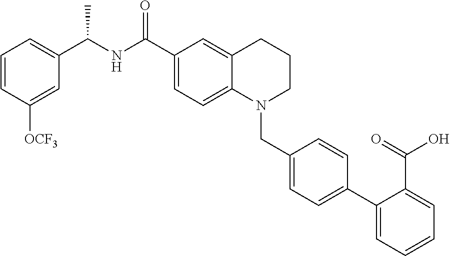 [SMILES: C[C@H](NC(=O)c1ccc2c(c1)CCCN2Cc1ccc(-c2ccccc2C(=O)O)cc1)c1cccc(OC(F)(F)F)c1] |
| 19 | 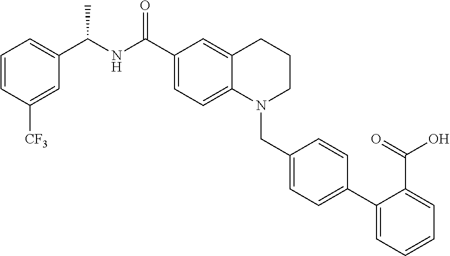 [SMILES: C[C@H](NC(=O)c1ccc2c(c1)CCCN2Cc1ccc(-c2ccccc2C(=O)O)cc1)c1cccc(C(F)(F)F)c1] |
| 20 | 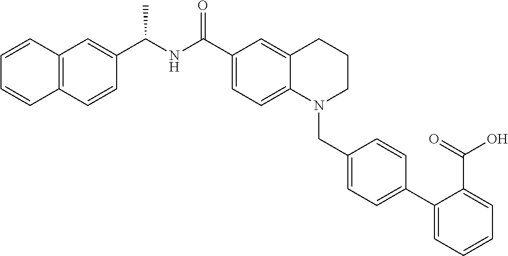 [SMILES: C[C@H](NC(=O)c1ccc2c(c1)CCCN2Cc1ccc(-c2ccccc2C(=O)O)cc1)c1ccc2ccccc2c1] |
| 21 | 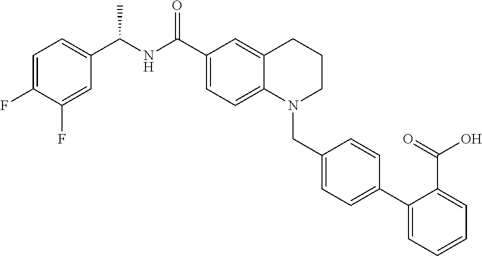 [SMILES: C[C@H](NC(=O)c1ccc2c(c1)CCCN2Cc1ccc(-c2ccccc2C(=O)O)cc1)c1ccc(F)c(F)c1] |
| 22 | 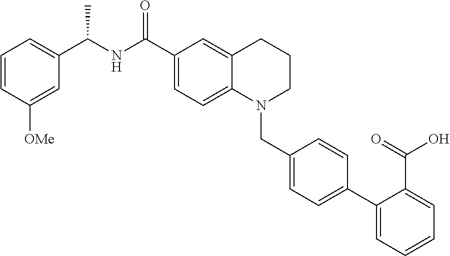 [SMILES: COc1cccc([C@H](C)NC(=O)c2ccc3c(c2)CCCN3Cc2ccc(-c3ccccc3C(=O)O)cc2)c1] |
| 23 | 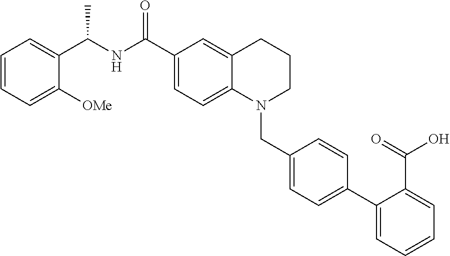 [SMILES: COc1ccccc1[C@H](C)NC(=O)c1ccc2c(c1)CCCN2Cc1ccc(-c2ccccc2C(=O)O)cc1] |
| 24 | 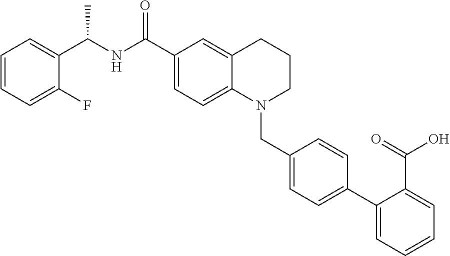 [SMILES: C[C@H](NC(=O)c1ccc2c(c1)CCCN2Cc1ccc(-c2ccccc2C(=O)O)cc1)c1ccccc1F] |
| 25 | 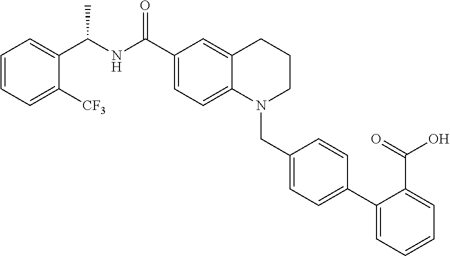 [SMILES: C[C@H](NC(=O)c1ccc2c(c1)CCCN2Cc1ccc(-c2ccccc2C(=O)O)cc1)c1ccccc1C(F)(F)F] |
| 26 | 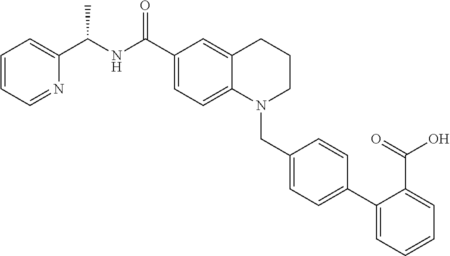 [SMILES: C[C@H](NC(=O)c1ccc2c(c1)CCCN2Cc1ccc(-c2ccccc2C(=O)O)cc1)c1ccccn1] |
| 27 | 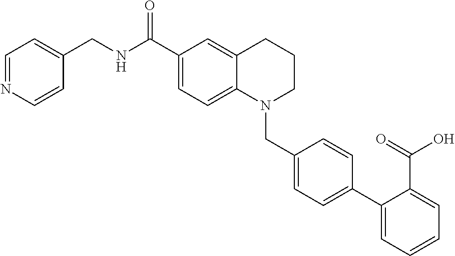 [SMILES: O=C(NCc1ccncc1)c1ccc2c(c1)CCCN2Cc1ccc(-c2ccccc2C(=O)O)cc1] |
| 28 | 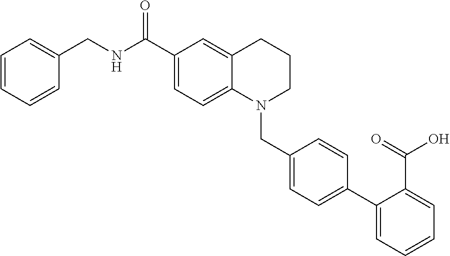 [SMILES: O=C(NCc1ccccc1)c1ccc2c(c1)CCCN2Cc1ccc(-c2ccccc2C(=O)O)cc1] |
| 29 | 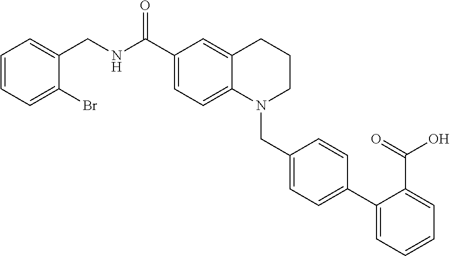 [SMILES: O=C(NCc1ccccc1Br)c1ccc2c(c1)CCCN2Cc1ccc(-c2ccccc2C(=O)O)cc1] |
| 30 | 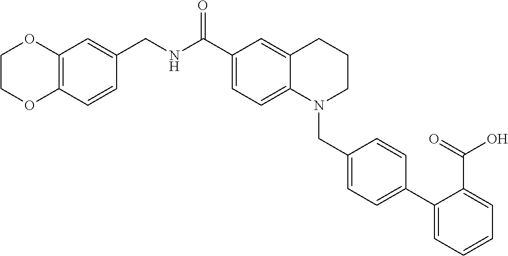 [SMILES: O=C(NCc1ccc2c(c1)OCCO2)c1ccc2c(c1)CCCN2Cc1ccc(-c2ccccc2C(=O)O)cc1] |
| 31 | 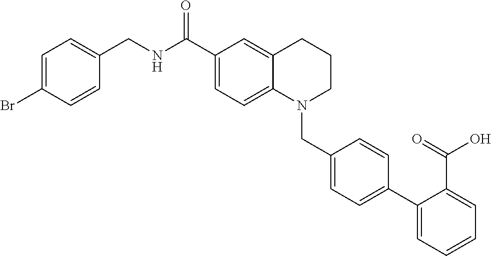 [SMILES: O=C(NCc1ccc(Br)cc1)c1ccc2c(c1)CCCN2Cc1ccc(-c2ccccc2C(=O)O)cc1] |
| 32 | 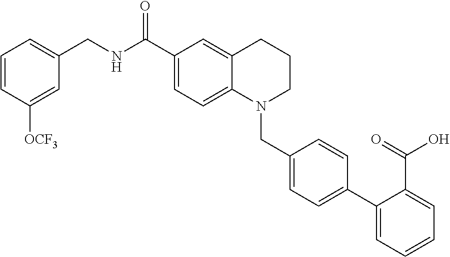 [SMILES: O=C(NCc1cccc(OC(F)(F)F)c1)c1ccc2c(c1)CCCN2Cc1ccc(-c2ccccc2C(=O)O)cc1] |
| 33 | 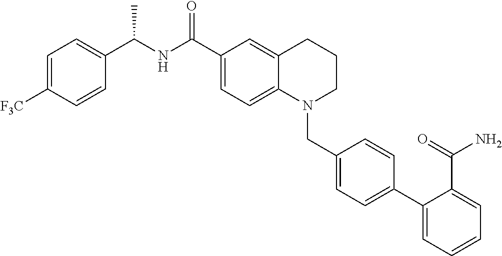 [SMILES: C[C@H](NC(=O)c1ccc2c(c1)CCCN2Cc1ccc(-c2ccccc2C(N)=O)cc1)c1ccc(C(F)(F)F)cc1] |
| 34 | 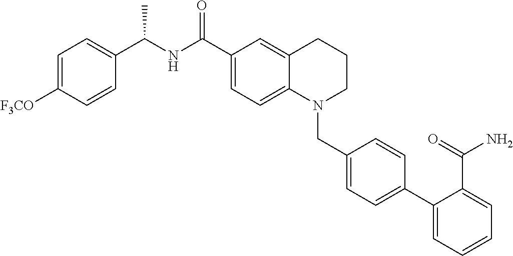 [SMILES: C[C@H](NC(=O)c1ccc2c(c1)CCCN2Cc1ccc(-c2ccccc2C(N)=O)cc1)c1ccc(OC(F)(F)F)cc1] |
| 35 | 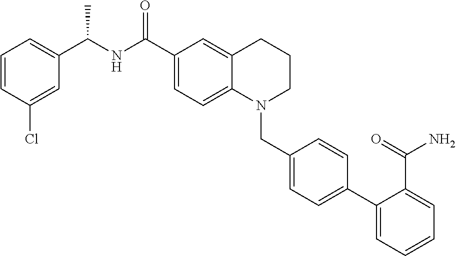 [SMILES: C[C@H](NC(=O)c1ccc2c(c1)CCCN2Cc1ccc(-c2ccccc2C(N)=O)cc1)c1cccc(Cl)c1] |
| 36 | 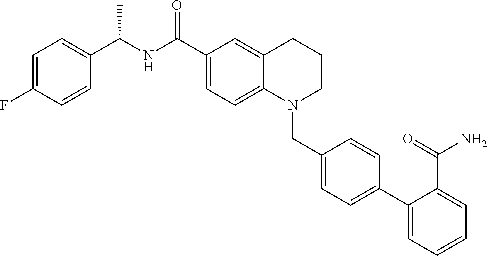 [SMILES: C[C@H](NC(=O)c1ccc2c(c1)CCCN2Cc1ccc(-c2ccccc2C(N)=O)cc1)c1ccc(F)cc1] |
| 37 | 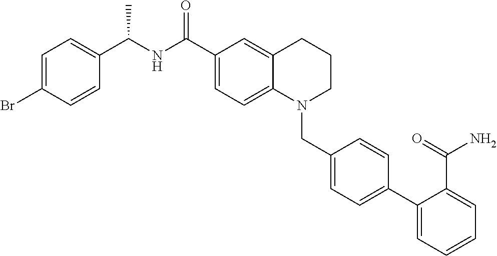 [SMILES: C[C@H](NC(=O)c1ccc2c(c1)CCCN2Cc1ccc(-c2ccccc2C(N)=O)cc1)c1ccc(Br)cc1] |
| 38 | 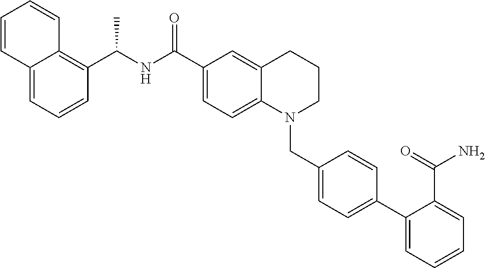 [SMILES: C[C@H](NC(=O)c1ccc2c(c1)CCCN2Cc1ccc(-c2ccccc2C(N)=O)cc1)c1cccc2ccccc12] |
| 39 | 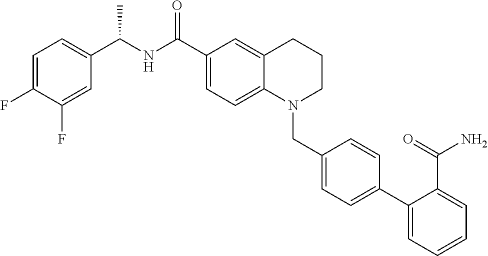 [SMILES: C[C@H](NC(=O)c1ccc2c(c1)CCCN2Cc1ccc(-c2ccccc2C(N)=O)cc1)c1ccc(F)c(F)c1] |
| 40 | 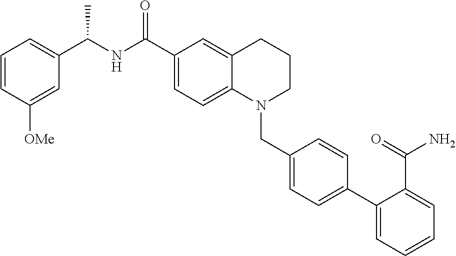 [SMILES: COc1cccc([C@H](C)NC(=O)c2ccc3c(c2)CCCN3Cc2ccc(-c3ccccc3C(N)=O)cc2)c1] |
| 41 | 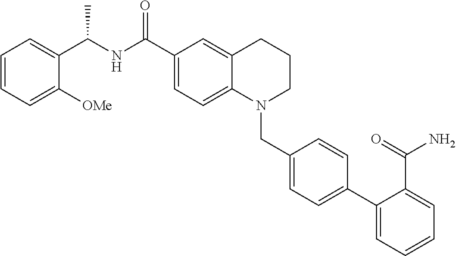 [SMILES: COc1ccccc1[C@H](C)NC(=O)c1ccc2c(c1)CCCN2Cc1ccc(-c2ccccc2C(N)=O)cc1] |
| 42 | 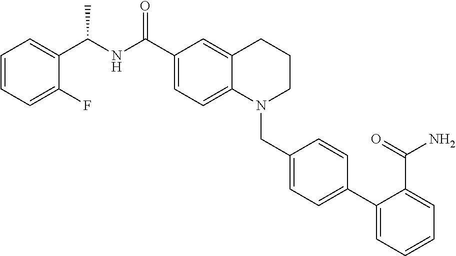 [SMILES: C[C@H](NC(=O)c1ccc2c(c1)CCCN2Cc1ccc(-c2ccccc2C(N)=O)cc1)c1ccccc1F] |
| 43 | 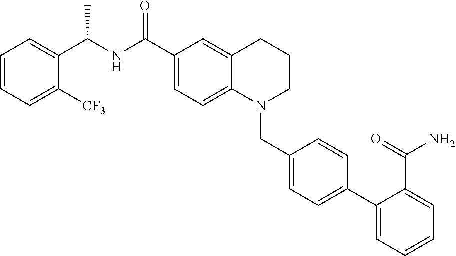 [SMILES: C[C@H](NC(=O)c1ccc2c(c1)CCCN2Cc1ccc(-c2ccccc2C(N)=O)cc1)c1ccccc1C(F)(F)F] |
| 44 | 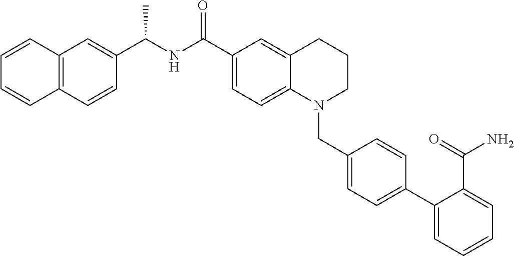 [SMILES: C[C@H](NC(=O)c1ccc2c(c1)CCCN2Cc1ccc(-c2ccccc2C(N)=O)cc1)c1ccc2ccccc2c1] |
| 45 | 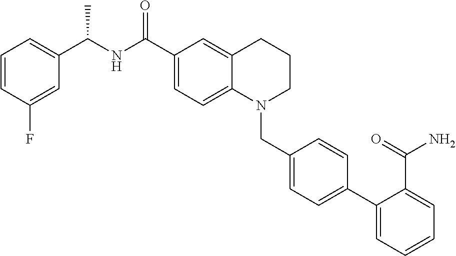 [SMILES: C[C@H](NC(=O)c1ccc2c(c1)CCCN2Cc1ccc(-c2ccccc2C(N)=O)cc1)c1cccc(F)c1] |
| 46 | 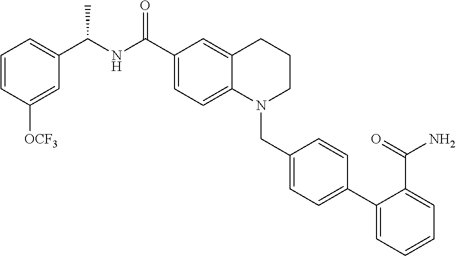 [SMILES: C[C@H](NC(=O)c1ccc2c(c1)CCCN2Cc1ccc(-c2ccccc2C(N)=O)cc1)c1cccc(OC(F)(F)F)c1] |
| 47 | 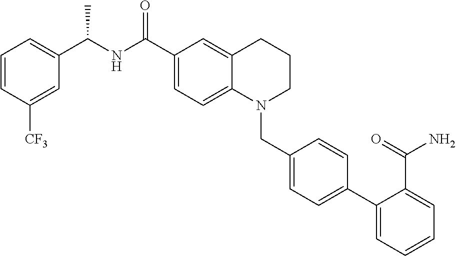 [SMILES: C[C@H](NC(=O)c1ccc2c(c1)CCCN2Cc1ccc(-c2ccccc2C(N)=O)cc1)c1cccc(C(F)(F)F)c1] |
| 48 | 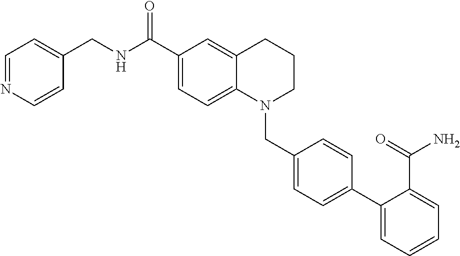 [SMILES: NC(=O)c1ccccc1-c1ccc(CN2CCCc3cc(C(=O)NCc4ccncc4)ccc32)cc1] |
| 49 | 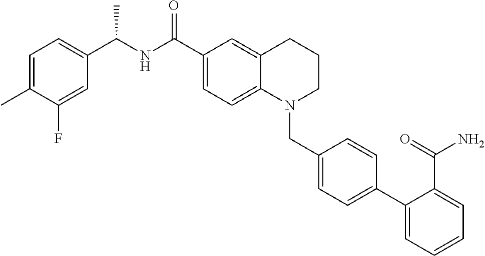 [SMILES: Cc1ccc([C@H](C)NC(=O)c2ccc3c(c2)CCCN3Cc2ccc(-c3ccccc3C(N)=O)cc2)cc1F] |
| 50 | 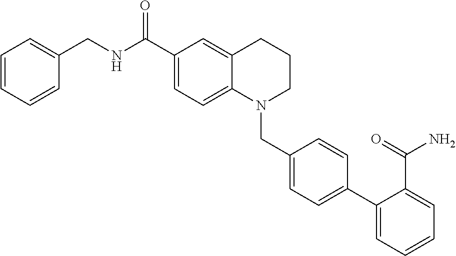 [SMILES: NC(=O)c1ccccc1-c1ccc(CN2CCCc3cc(C(=O)NCc4ccccc4)ccc32)cc1] |
| 51 | 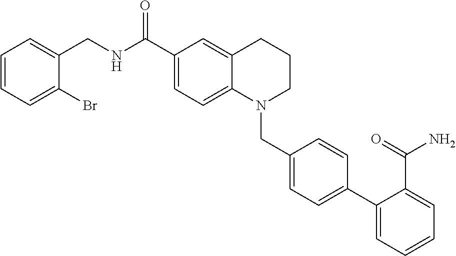 [SMILES: NC(=O)c1ccccc1-c1ccc(CN2CCCc3cc(C(=O)NCc4ccccc4Br)ccc32)cc1] |
| 52 | 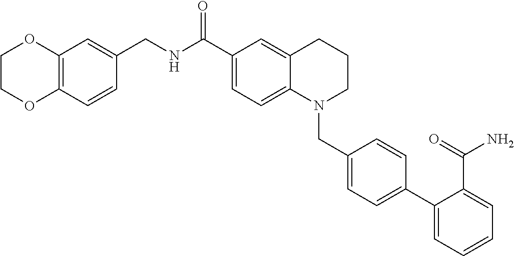 [SMILES: NC(=O)c1ccccc1-c1ccc(CN2CCCc3cc(C(=O)NCc4ccc5c(c4)OCCO5)ccc32)cc1] |
| 53 | 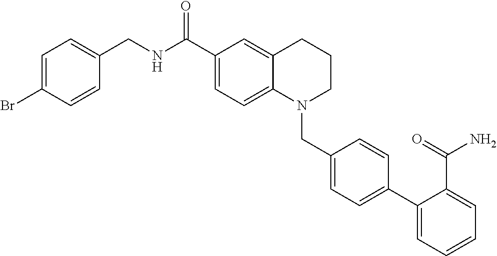 [SMILES: NC(=O)c1ccccc1-c1ccc(CN2CCCc3cc(C(=O)NCc4ccc(Br)cc4)ccc32)cc1] |
| 54 | 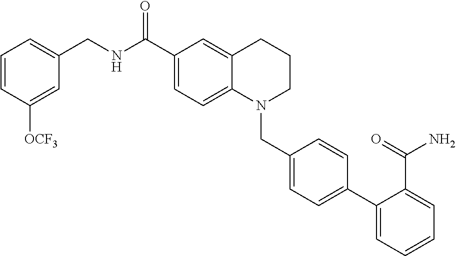 [SMILES: NC(=O)c1ccccc1-c1ccc(CN2CCCc3cc(C(=O)NCc4cccc(OC(F)(F)F)c4)ccc32)cc1] |
| 55 | 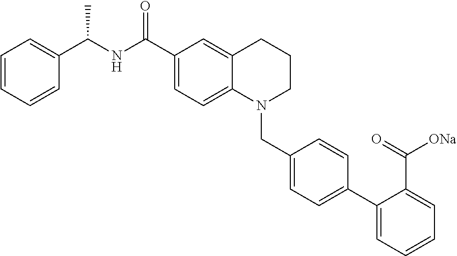 |
| 56 | 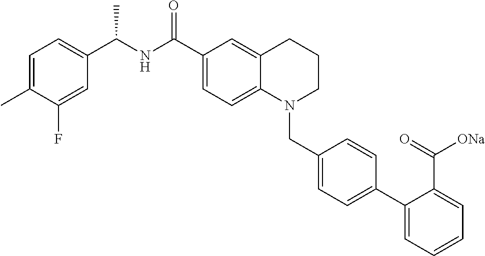 |
| 57 | 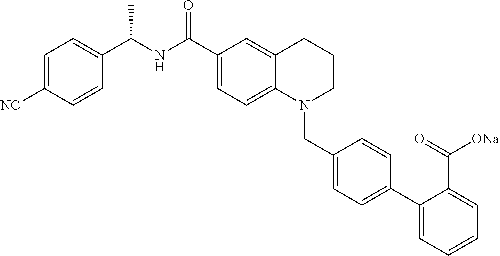 |
| 58 | 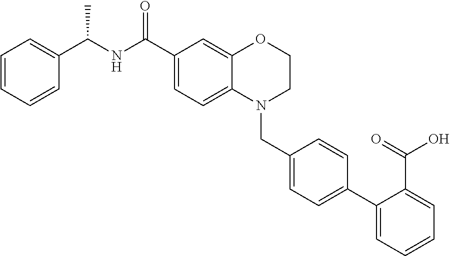 [SMILES: C[C@H](NC(=O)c1ccc2c(c1)OCCN2Cc1ccc(-c2ccccc2C(=O)O)cc1)c1ccccc1] |
| 59 | 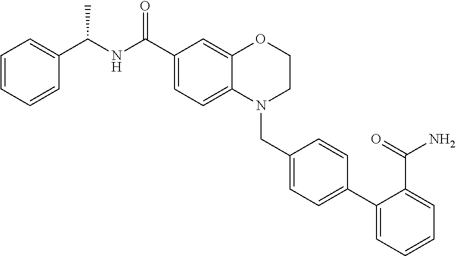 [SMILES: C[C@H](NC(=O)c1ccc2c(c1)OCCN2Cc1ccc(-c2ccccc2C(N)=O)cc1)c1ccccc1] |
| 60 | 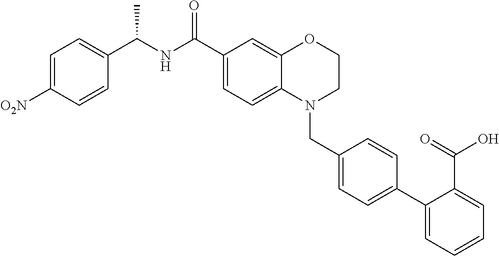 [SMILES: C[C@H](NC(=O)c1ccc2c(c1)OCCN2Cc1ccc(-c2ccccc2C(=O)O)cc1)c1ccc([N+](=O)[O-])cc1] |
| 61 | 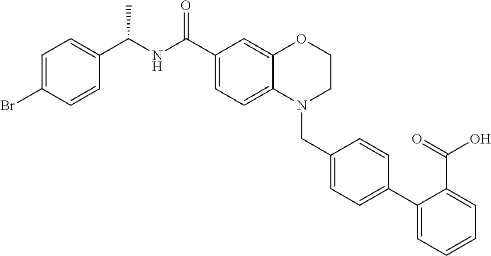 [SMILES: C[C@H](NC(=O)c1ccc2c(c1)OCCN2Cc1ccc(-c2ccccc2C(=O)O)cc1)c1ccc(Br)cc1] |
| 62 | 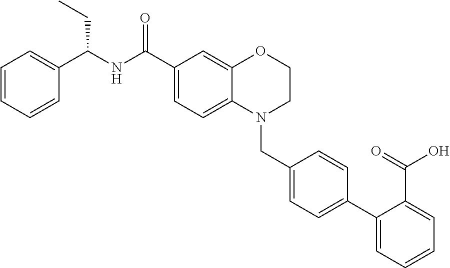 [SMILES: CC[C@H](NC(=O)c1ccc2c(c1)OCCN2Cc1ccc(-c2ccccc2C(=O)O)cc1)c1ccccc1] |
| 63 | 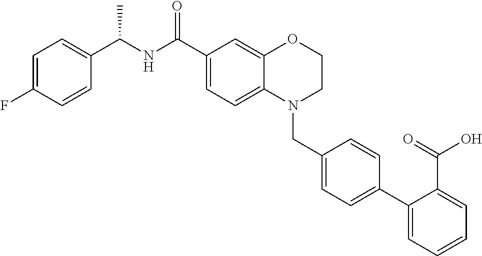 [SMILES: C[C@H](NC(=O)c1ccc2c(c1)OCCN2Cc1ccc(-c2ccccc2C(=O)O)cc1)c1ccc(F)cc1] |
| 64 | 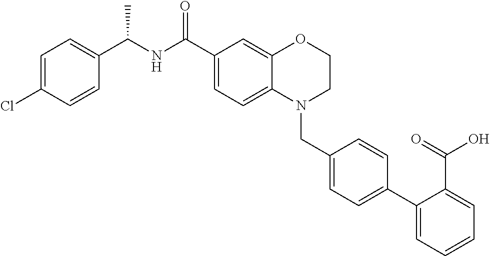 [SMILES: C[C@H](NC(=O)c1ccc2c(c1)OCCN2Cc1ccc(-c2ccccc2C(=O)O)cc1)c1ccc(Cl)cc1] |
| 65 | 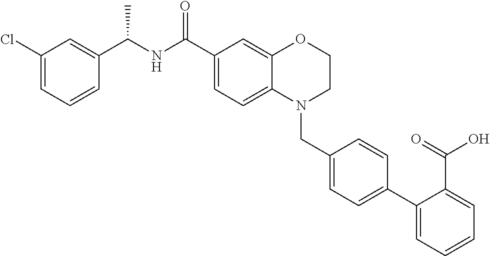 [SMILES: C[C@H](NC(=O)c1ccc2c(c1)OCCN2Cc1ccc(-c2ccccc2C(=O)O)cc1)c1cccc(Cl)c1] |
| 66 | 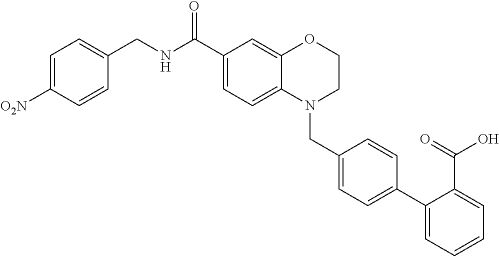 [SMILES: O=C(NCc1ccc([N+](=O)[O-])cc1)c1ccc2c(c1)OCCN2Cc1ccc(-c2ccccc2C(=O)O)cc1] |
| 67 | 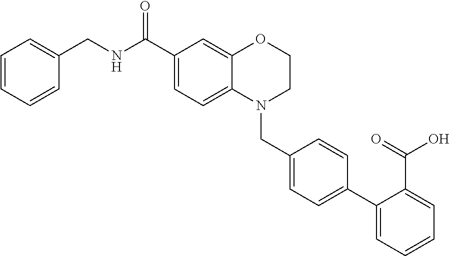 [SMILES: O=C(NCc1ccccc1)c1ccc2c(c1)OCCN2Cc1ccc(-c2ccccc2C(=O)O)cc1] |
| 68 | 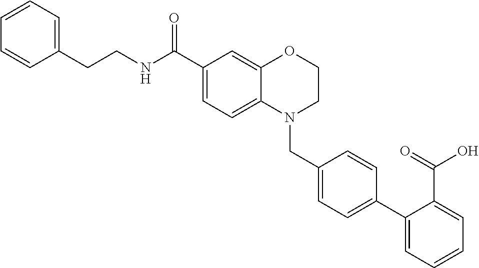 [SMILES: O=C(NCCc1ccccc1)c1ccc2c(c1)OCCN2Cc1ccc(-c2ccccc2C(=O)O)cc1] |
| 69 | 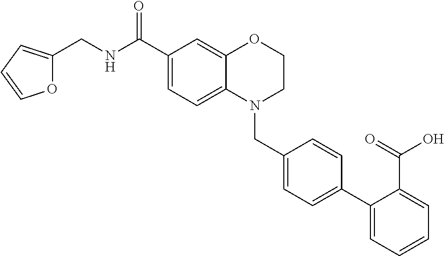 [SMILES: O=C(NCc1ccco1)c1ccc2c(c1)OCCN2Cc1ccc(-c2ccccc2C(=O)O)cc1] |
| 70 | 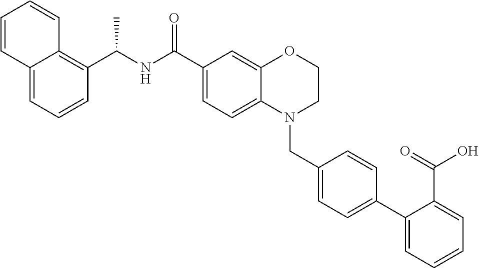 [SMILES: C[C@H](NC(=O)c1ccc2c(c1)OCCN2Cc1ccc(-c2ccccc2C(=O)O)cc1)c1cccc2ccccc12] |
| 71 | 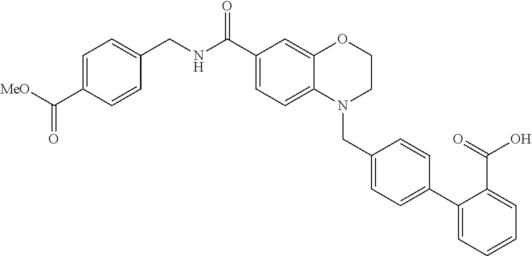 [SMILES: COC(=O)c1ccc(CNC(=O)c2ccc3c(c2)OCCN3Cc2ccc(-c3ccccc3C(=O)O)cc2)cc1] |
| 72 | 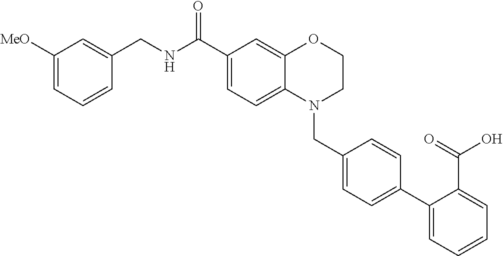 [SMILES: COc1cccc(CNC(=O)c2ccc3c(c2)OCCN3Cc2ccc(-c3ccccc3C(=O)O)cc2)c1] |
| 73 | 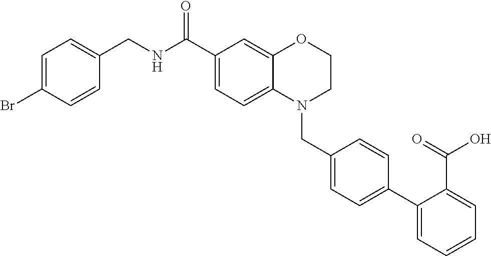 [SMILES: O=C(NCc1ccc(Br)cc1)c1ccc2c(c1)OCCN2Cc1ccc(-c2ccccc2C(=O)O)cc1] |
| 74 | 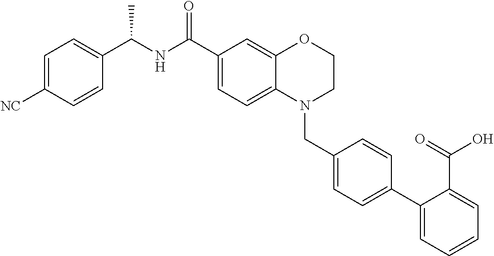 [SMILES: C[C@H](NC(=O)c1ccc2c(c1)OCCN2Cc1ccc(-c2ccccc2C(=O)O)cc1)c1ccc(C#N)cc1] |
| 75 | 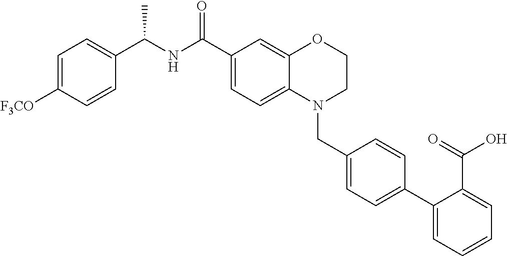 [SMILES: C[C@H](NC(=O)c1ccc2c(c1)OCCN2Cc1ccc(-c2ccccc2C(=O)O)cc1)c1ccc(OC(F)(F)F)cc1] |
| 76 | 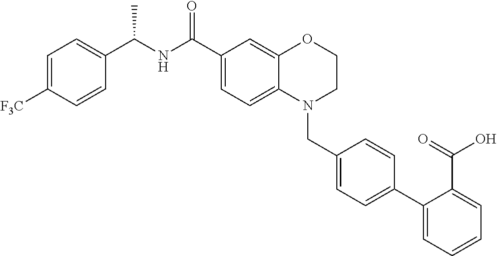 [SMILES: C[C@H](NC(=O)c1ccc2c(c1)OCCN2Cc1ccc(-c2ccccc2C(=O)O)cc1)c1ccc(C(F)(F)F)cc1] |
| 77 | 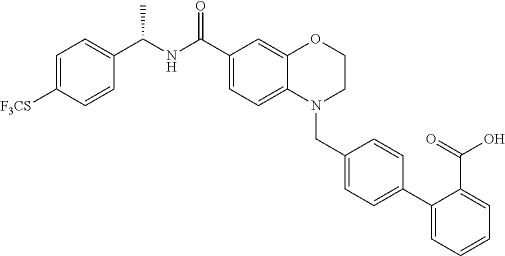 |
| 78 | 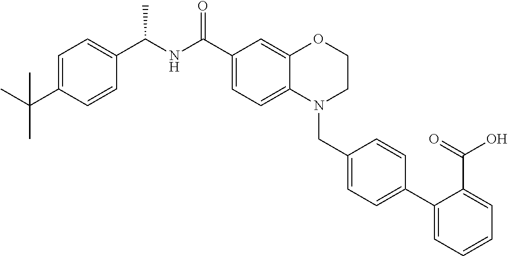 [SMILES: C[C@H](NC(=O)c1ccc2c(c1)OCCN2Cc1ccc(-c2ccccc2C(=O)O)cc1)c1ccc(C(C)(C)C)cc1] |
| 79 | 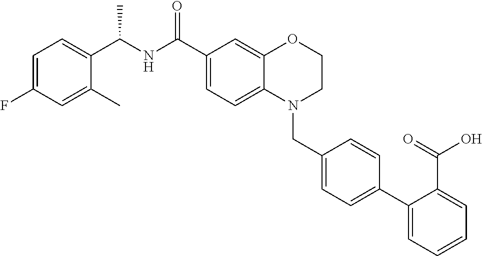 [SMILES: Cc1cc(F)ccc1[C@H](C)NC(=O)c1ccc2c(c1)OCCN2Cc1ccc(-c2ccccc2C(=O)O)cc1] |
| 80 | 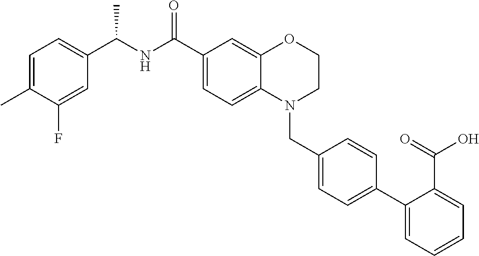 [SMILES: Cc1ccc([C@H](C)NC(=O)c2ccc3c(c2)OCCN3Cc2ccc(-c3ccccc3C(=O)O)cc2)cc1F] |
| 81 | 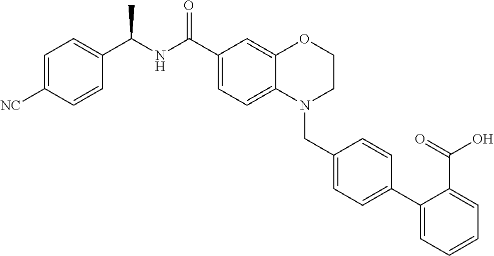 [SMILES: C[C@@H](NC(=O)c1ccc2c(c1)OCCN2Cc1ccc(-c2ccccc2C(=O)O)cc1)c1ccc(C#N)cc1] |
| 82 | 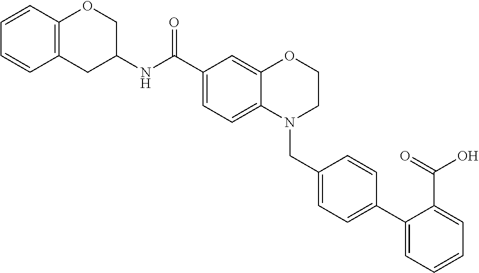 [SMILES: O=C(NC1COc2ccccc2C1)c1ccc2c(c1)OCCN2Cc1ccc(-c2ccccc2C(=O)O)cc1] |
| 83 | 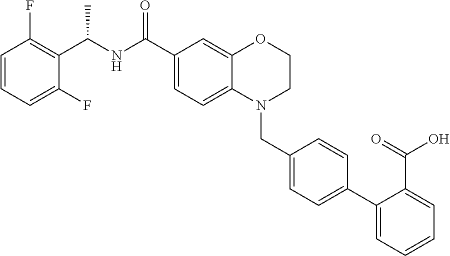 [SMILES: C[C@H](NC(=O)c1ccc2c(c1)OCCN2Cc1ccc(-c2ccccc2C(=O)O)cc1)c1c(F)cccc1F] |
| 84 | 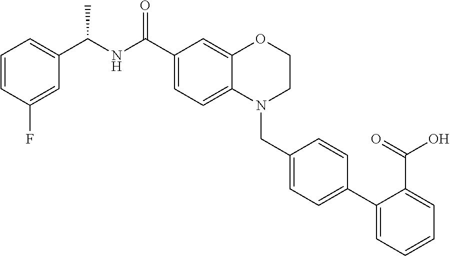 [SMILES: C[C@H](NC(=O)c1ccc2c(c1)OCCN2Cc1ccc(-c2ccccc2C(=O)O)cc1)c1cccc(F)c1] |
| 85 | 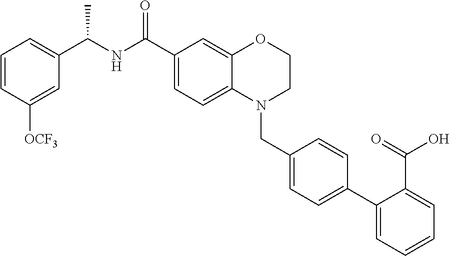 [SMILES: C[C@H](NC(=O)c1ccc2c(c1)OCCN2Cc1ccc(-c2ccccc2C(=O)O)cc1)c1cccc(OC(F)(F)F)c1] |
| 86 | 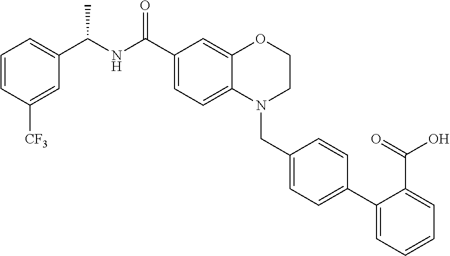 [SMILES: C[C@H](NC(=O)c1ccc2c(c1)OCCN2Cc1ccc(-c2ccccc2C(=O)O)cc1)c1cccc(C(F)(F)F)c1] |
| 87 | 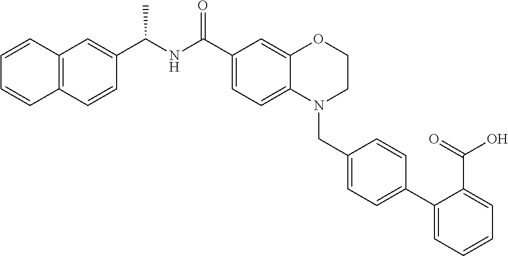 [SMILES: C[C@H](NC(=O)c1ccc2c(c1)OCCN2Cc1ccc(-c2ccccc2C(=O)O)cc1)c1ccc2ccccc2c1] |
| 88 | 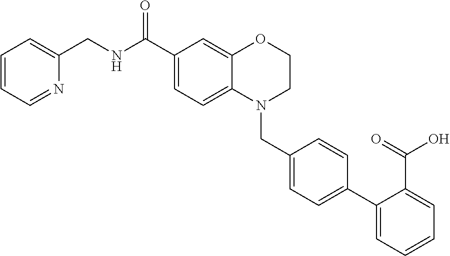 [SMILES: O=C(NCc1ccccn1)c1ccc2c(c1)OCCN2Cc1ccc(-c2ccccc2C(=O)O)cc1] |
| 89 | 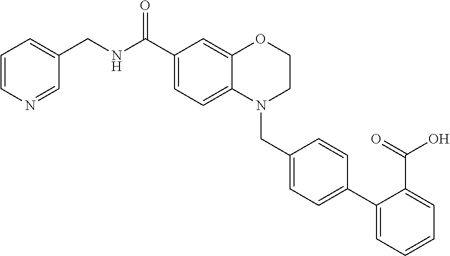 [SMILES: O=C(NCc1cccnc1)c1ccc2c(c1)OCCN2Cc1ccc(-c2ccccc2C(=O)O)cc1] |
| 90 | 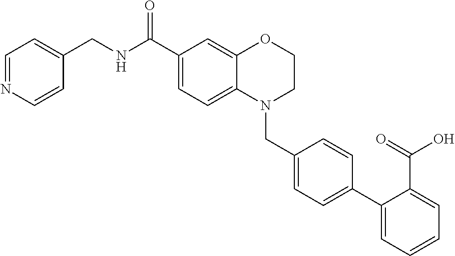 [SMILES: O=C(NCc1ccncc1)c1ccc2c(c1)OCCN2Cc1ccc(-c2ccccc2C(=O)O)cc1] |
| 91 | 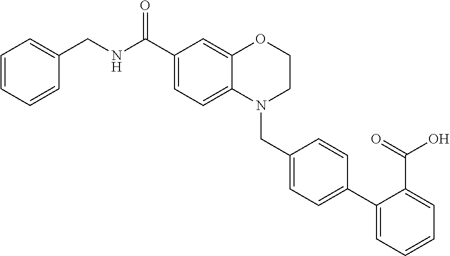 [SMILES: O=C(NCc1ccccc1)c1ccc2c(c1)OCCN2Cc1ccc(-c2ccccc2C(=O)O)cc1] |
| 92 | 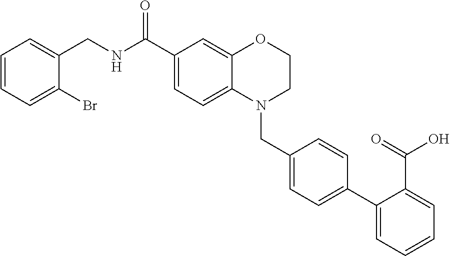 [SMILES: O=C(NCc1ccccc1Br)c1ccc2c(c1)OCCN2Cc1ccc(-c2ccccc2C(=O)O)cc1] |
| 93 | 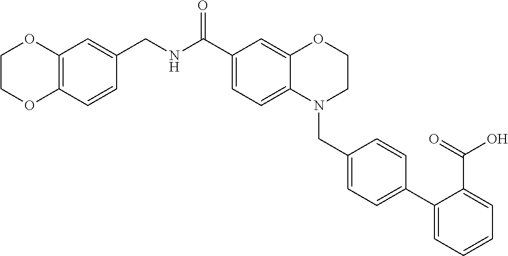 [SMILES: O=C(NCc1ccc2c(c1)OCCO2)c1ccc2c(c1)OCCN2Cc1ccc(-c2ccccc2C(=O)O)cc1] |
| 94 | 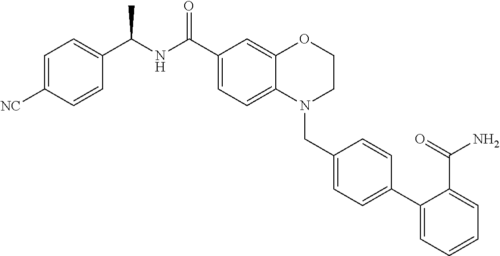 [SMILES: C[C@@H](NC(=O)c1ccc2c(c1)OCCN2Cc1ccc(-c2ccccc2C(N)=O)cc1)c1ccc(C#N)cc1] |
| 95 | 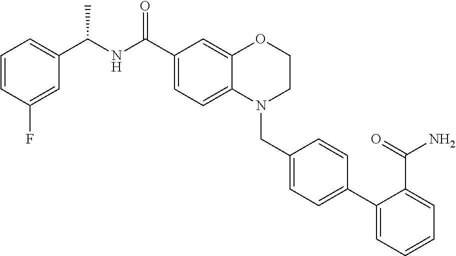 [SMILES: C[C@H](NC(=O)c1ccc2c(c1)OCCN2Cc1ccc(-c2ccccc2C(N)=O)cc1)c1cccc(F)c1] |
| 96 | 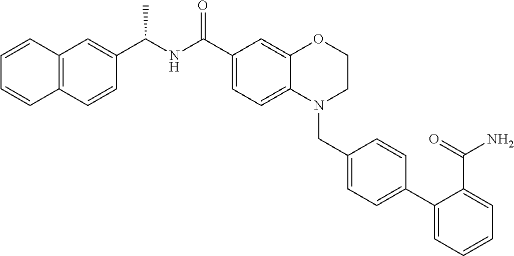 [SMILES: C[C@H](NC(=O)c1ccc2c(c1)OCCN2Cc1ccc(-c2ccccc2C(N)=O)cc1)c1ccc2ccccc2c1] |
| 97 | 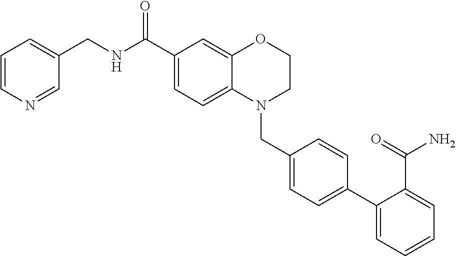 [SMILES: NC(=O)c1ccccc1-c1ccc(CN2CCOc3cc(C(=O)NCc4cccnc4)ccc32)cc1] |
| 98 | 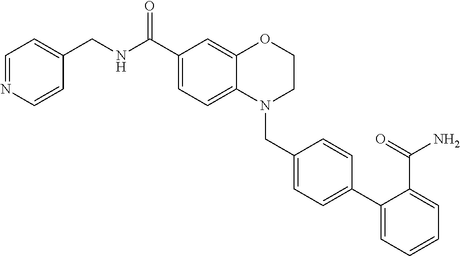 [SMILES: NC(=O)c1ccccc1-c1ccc(CN2CCOc3cc(C(=O)NCc4ccncc4)ccc32)cc1] |
| 99 | 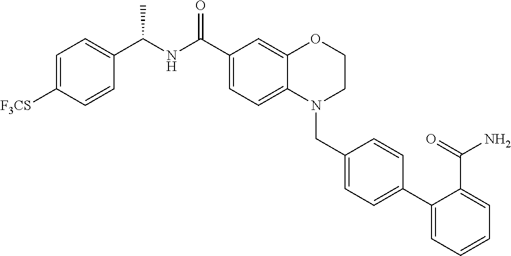 |
| 100 | 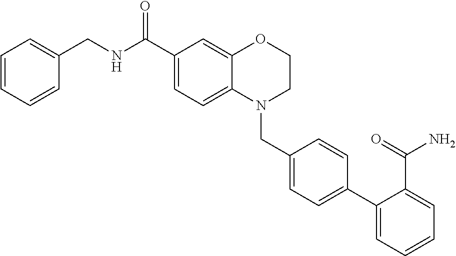 [SMILES: NC(=O)c1ccccc1-c1ccc(CN2CCOc3cc(C(=O)NCc4ccccc4)ccc32)cc1] |
| 101 | 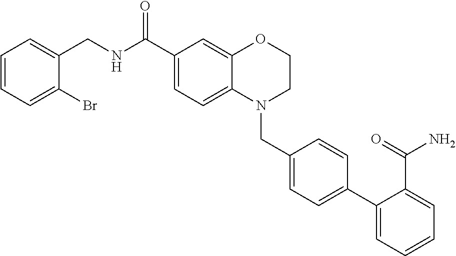 [SMILES: NC(=O)c1ccccc1-c1ccc(CN2CCOc3cc(C(=O)NCc4ccccc4Br)ccc32)cc1] |
| 102 | 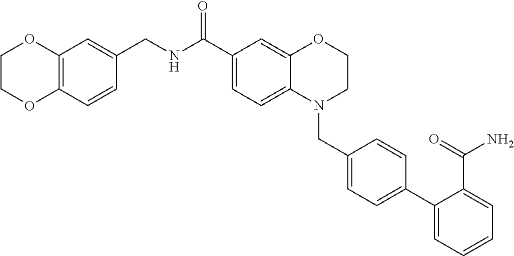 [SMILES: NC(=O)c1ccccc1-c1ccc(CN2CCOc3cc(C(=O)NCc4ccc5c(c4)OCCO5)ccc32)cc1] |
| 103 | 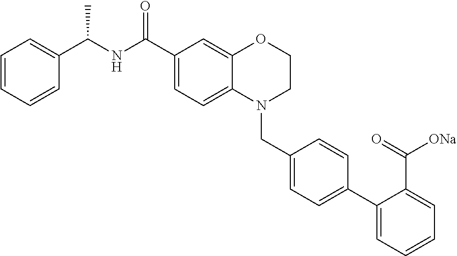 |
| 104 | 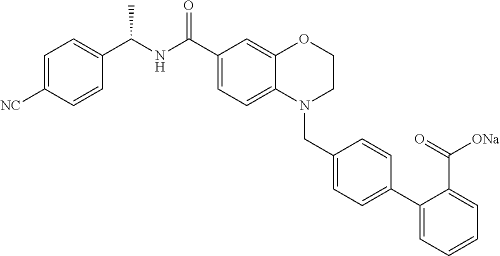 |
| 105 | 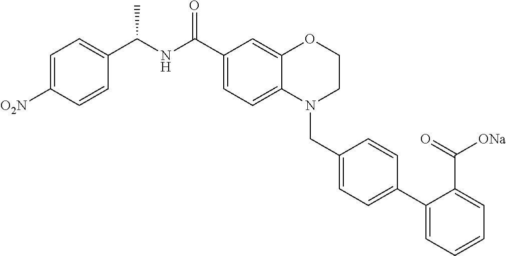 |
| 106 | 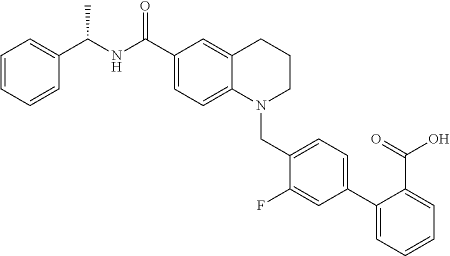 [SMILES: C[C@H](NC(=O)c1ccc2c(c1)CCCN2Cc1ccc(-c2ccccc2C(=O)O)cc1F)c1ccccc1] |
| 107 | 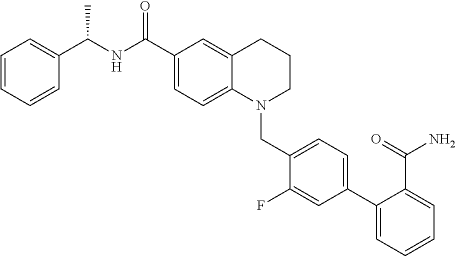 [SMILES: C[C@H](NC(=O)c1ccc2c(c1)CCCN2Cc1ccc(-c2ccccc2C(N)=O)cc1F)c1ccccc1] |
| 108 | 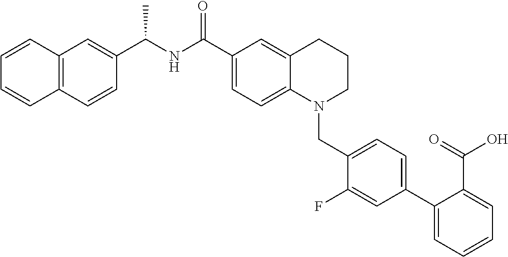 [SMILES: C[C@H](NC(=O)c1ccc2c(c1)CCCN2Cc1ccc(-c2ccccc2C(=O)O)cc1F)c1ccc2ccccc2c1] |
| 109 | 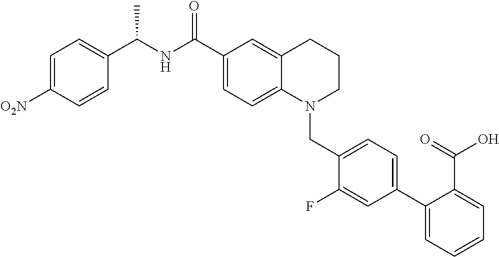 [SMILES: C[C@H](NC(=O)c1ccc2c(c1)CCCN2Cc1ccc(-c2ccccc2C(=O)O)cc1F)c1ccc([N+](=O)[O-])cc1] |
| 110 | 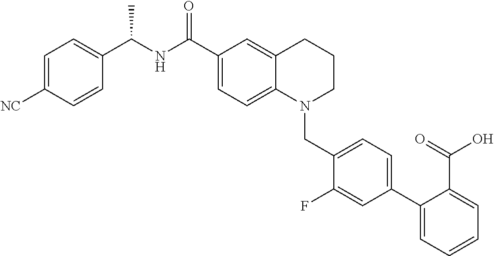 [SMILES: C[C@H](NC(=O)c1ccc2c(c1)CCCN2Cc1ccc(-c2ccccc2C(=O)O)cc1F)c1ccc(C#N)cc1] |
| 111 | 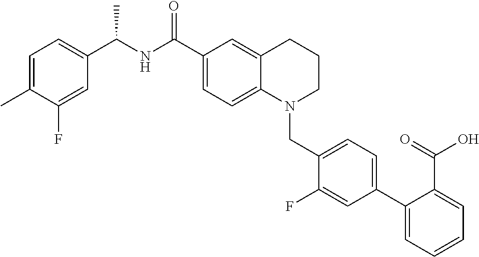 [SMILES: Cc1ccc([C@H](C)NC(=O)c2ccc3c(c2)CCCN3Cc2ccc(-c3ccccc3C(=O)O)cc2F)cc1F] |
| 112 | 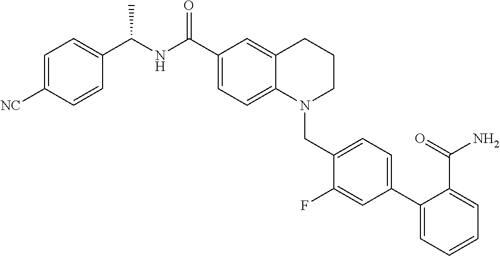 [SMILES: C[C@H](NC(=O)c1ccc2c(c1)CCCN2Cc1ccc(-c2ccccc2C(N)=O)cc1F)c1ccc(C#N)cc1] |
| 113 | 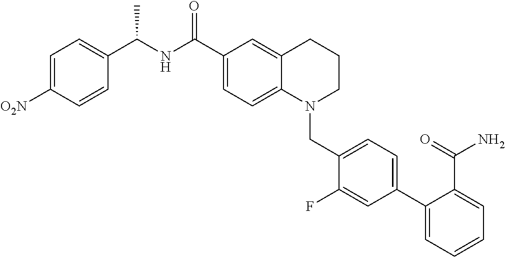 [SMILES: C[C@H](NC(=O)c1ccc2c(c1)CCCN2Cc1ccc(-c2ccccc2C(N)=O)cc1F)c1ccc([N+](=O)[O-])cc1] |
| 114 | 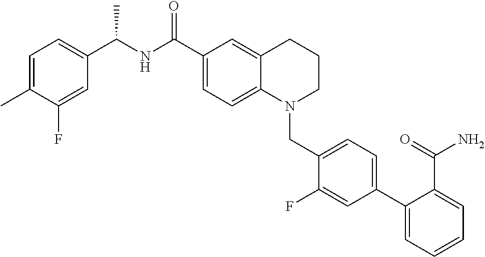 [SMILES: Cc1ccc([C@H](C)NC(=O)c2ccc3c(c2)CCCN3Cc2ccc(-c3ccccc3C(N)=O)cc2F)cc1F] |
| 115 | 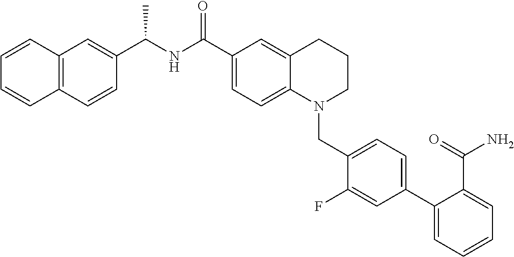 [SMILES: C[C@H](NC(=O)c1ccc2c(c1)CCCN2Cc1ccc(-c2ccccc2C(N)=O)cc1F)c1ccc2ccccc2c1] |
| 116 | 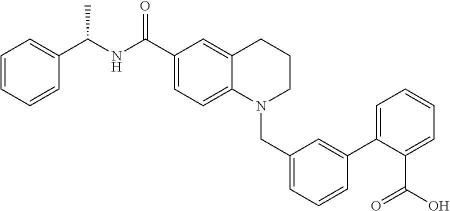 [SMILES: C[C@H](NC(=O)c1ccc2c(c1)CCCN2Cc1cccc(-c2ccccc2C(=O)O)c1)c1ccccc1] |
| 117 | 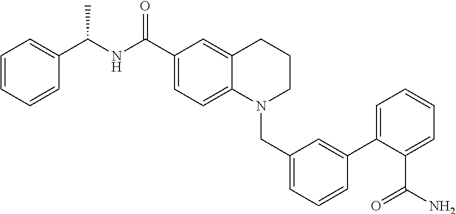 [SMILES: C[C@H](NC(=O)c1ccc2c(c1)CCCN2Cc1cccc(-c2ccccc2C(N)=O)c1)c1ccccc1] |
| 118 | 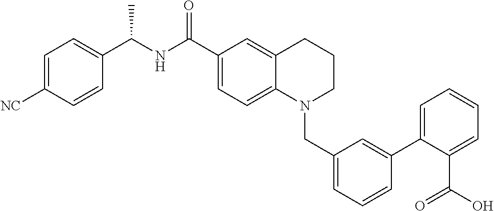 [SMILES: C[C@H](NC(=O)c1ccc2c(c1)CCCN2Cc1cccc(-c2ccccc2C(=O)O)c1)c1ccc(C#N)cc1] |
| 119 | 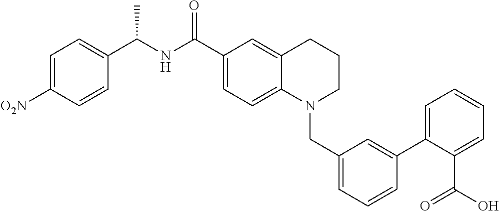 [SMILES: C[C@H](NC(=O)c1ccc2c(c1)CCCN2Cc1cccc(-c2ccccc2C(=O)O)c1)c1ccc([N+](=O)[O-])cc1] |
| 120 | 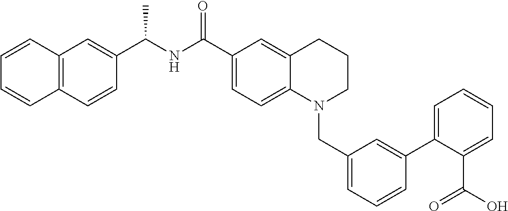 [SMILES: C[C@H](NC(=O)c1ccc2c(c1)CCCN2Cc1cccc(-c2ccccc2C(=O)O)c1)c1ccc2ccccc2c1] |
| 121 | 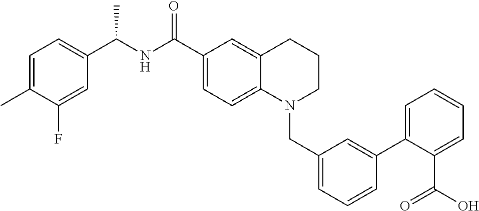 [SMILES: Cc1ccc([C@H](C)NC(=O)c2ccc3c(c2)CCCN3Cc2cccc(-c3ccccc3C(=O)O)c2)cc1F] |
| 122 | 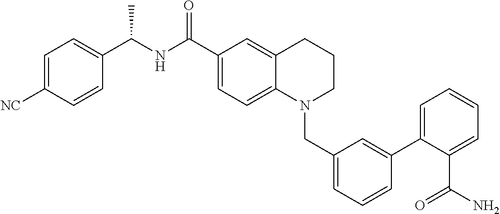 [SMILES: C[C@H](NC(=O)c1ccc2c(c1)CCCN2Cc1cccc(-c2ccccc2C(N)=O)c1)c1ccc(C#N)cc1] |
| 123 | 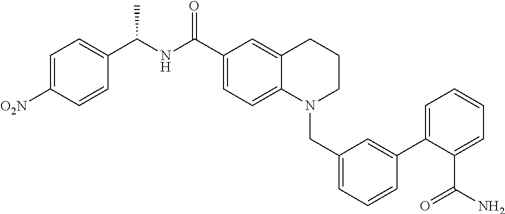 [SMILES: C[C@H](NC(=O)c1ccc2c(c1)CCCN2Cc1cccc(-c2ccccc2C(N)=O)c1)c1ccc([N+](=O)[O-])cc1] |
| 124 | 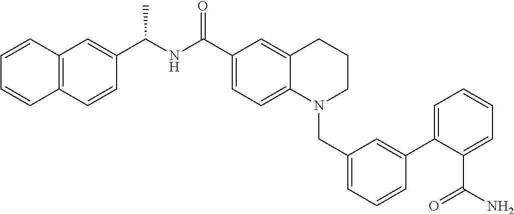 [SMILES: C[C@H](NC(=O)c1ccc2c(c1)CCCN2Cc1cccc(-c2ccccc2C(N)=O)c1)c1ccc2ccccc2c1] |
| 125 | 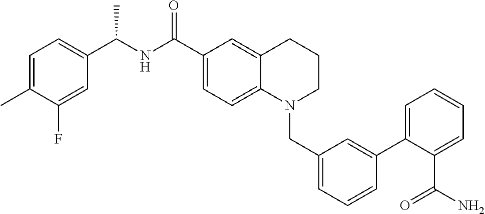 [SMILES: Cc1ccc([C@H](C)NC(=O)c2ccc3c(c2)CCCN3Cc2cccc(-c3ccccc3C(N)=O)c2)cc1F] |
| 126 | 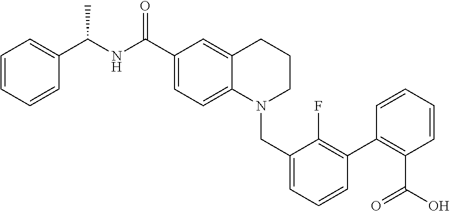 [SMILES: C[C@H](NC(=O)c1ccc2c(c1)CCCN2Cc1cccc(-c2ccccc2C(=O)O)c1F)c1ccccc1] |
| 127 | 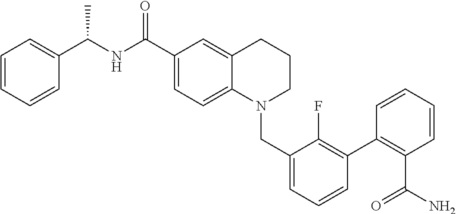 [SMILES: C[C@H](NC(=O)c1ccc2c(c1)CCCN2Cc1cccc(-c2ccccc2C(N)=O)c1F)c1ccccc1] |
| 128 | 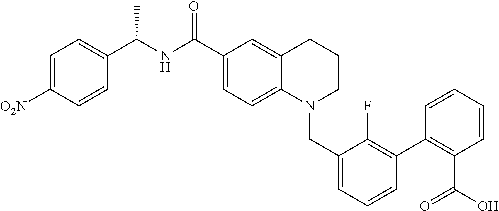 [SMILES: C[C@H](NC(=O)c1ccc2c(c1)CCCN2Cc1cccc(-c2ccccc2C(=O)O)c1F)c1ccc([N+](=O)[O-])cc1] |
| 129 | 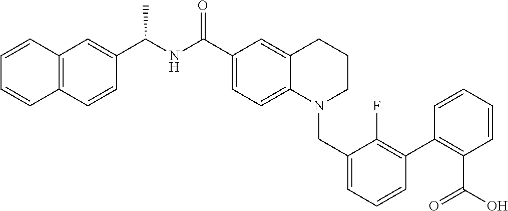 [SMILES: C[C@H](NC(=O)c1ccc2c(c1)CCCN2Cc1cccc(-c2ccccc2C(=O)O)c1F)c1ccc2ccccc2c1] |
| 130 | 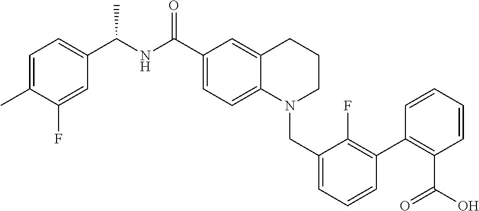 [SMILES: Cc1ccc([C@H](C)NC(=O)c2ccc3c(c2)CCCN3Cc2cccc(-c3ccccc3C(=O)O)c2F)cc1F] |
| 131 | 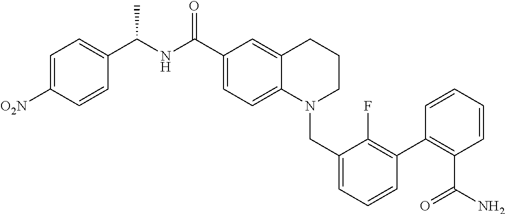 [SMILES: C[C@H](NC(=O)c1ccc2c(c1)CCCN2Cc1cccc(-c2ccccc2C(N)=O)c1F)c1ccc([N+](=O)[O-])cc1] |
| 132 | 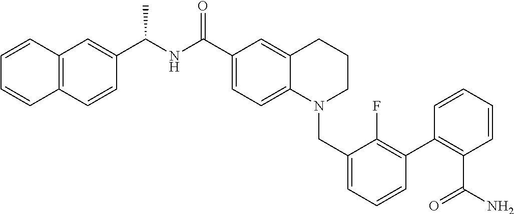 [SMILES: C[C@H](NC(=O)c1ccc2c(c1)CCCN2Cc1cccc(-c2ccccc2C(N)=O)c1F)c1ccc2ccccc2c1] |
| 133 | 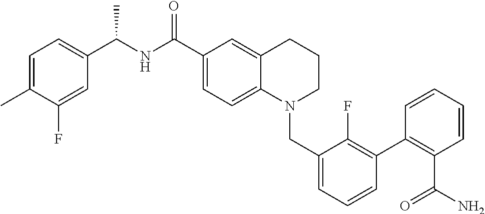 [SMILES: Cc1ccc([C@H](C)NC(=O)c2ccc3c(c2)CCCN3Cc2cccc(-c3ccccc3C(N)=O)c2F)cc1F] |
| 134 | 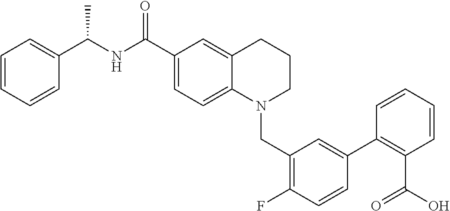 [SMILES: C[C@H](NC(=O)c1ccc2c(c1)CCCN2Cc1cc(-c2ccccc2C(=O)O)ccc1F)c1ccccc1] |
| 135 | 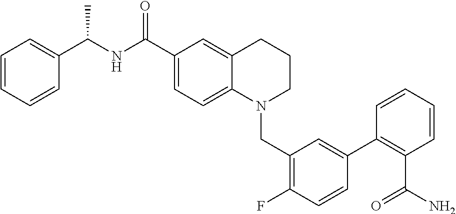 [SMILES: C[C@H](NC(=O)c1ccc2c(c1)CCCN2Cc1cc(-c2ccccc2C(N)=O)ccc1F)c1ccccc1] |
| 136 | 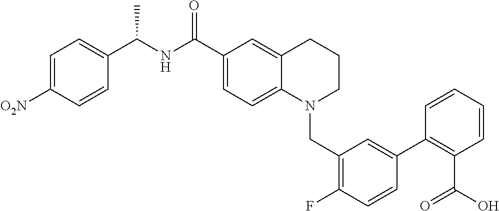 [SMILES: C[C@H](NC(=O)c1ccc2c(c1)CCCN2Cc1cc(-c2ccccc2C(=O)O)ccc1F)c1ccc([N+](=O)[O-])cc1] |
| 137 | 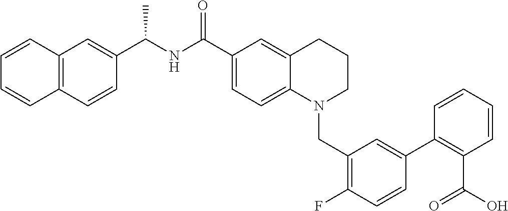 [SMILES: C[C@H](NC(=O)c1ccc2c(c1)CCCN2Cc1cc(-c2ccccc2C(=O)O)ccc1F)c1ccc2ccccc2c1] |
| 138 | 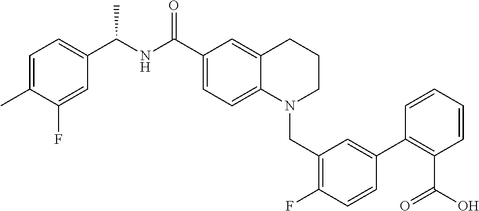 [SMILES: Cc1ccc([C@H](C)NC(=O)c2ccc3c(c2)CCCN3Cc2cc(-c3ccccc3C(=O)O)ccc2F)cc1F] |
| 139 | 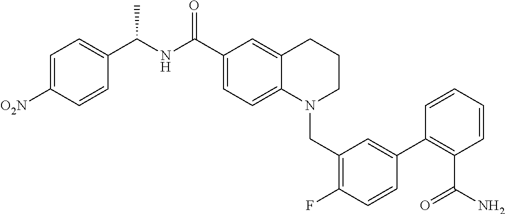 [SMILES: C[C@H](NC(=O)c1ccc2c(c1)CCCN2Cc1cc(-c2ccccc2C(N)=O)ccc1F)c1ccc([N+](=O)[O-])cc1] |
| 140 | 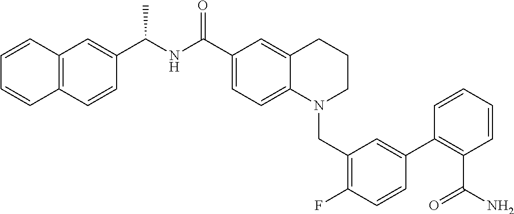 [SMILES: C[C@H](NC(=O)c1ccc2c(c1)CCCN2Cc1cc(-c2ccccc2C(N)=O)ccc1F)c1ccc2ccccc2c1] |
| 141 | 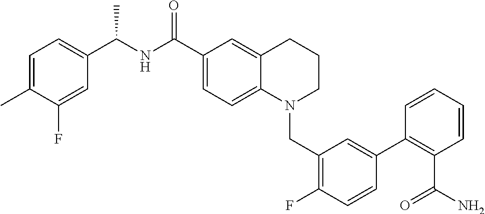 [SMILES: Cc1ccc([C@H](C)NC(=O)c2ccc3c(c2)CCCN3Cc2cc(-c3ccccc3C(N)=O)ccc2F)cc1F] |
| 142 | 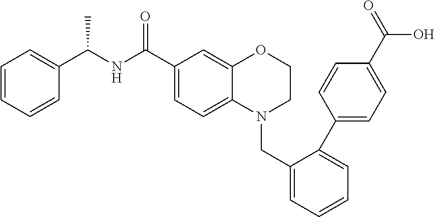 [SMILES: C[C@H](NC(=O)c1ccc2c(c1)OCCN2Cc1ccccc1-c1ccc(C(=O)O)cc1)c1ccccc1] |
| 143 | 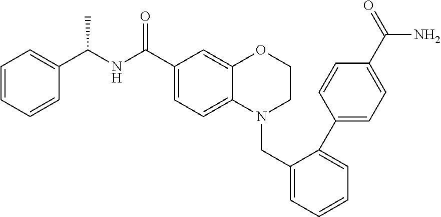 [SMILES: C[C@H](NC(=O)c1ccc2c(c1)OCCN2Cc1ccccc1-c1ccc(C(N)=O)cc1)c1ccccc1] |
| 144 | 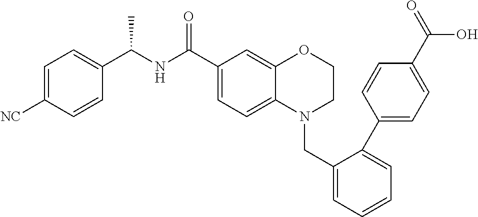 [SMILES: C[C@H](NC(=O)c1ccc2c(c1)OCCN2Cc1ccccc1-c1ccc(C(=O)O)cc1)c1ccc(C#N)cc1] |
| 145 | 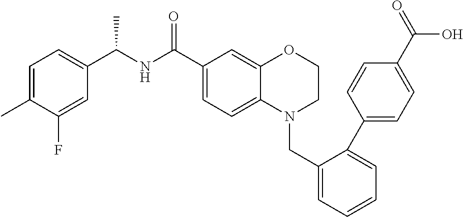 [SMILES: Cc1ccc([C@H](C)NC(=O)c2ccc3c(c2)OCCN3Cc2ccccc2-c2ccc(C(=O)O)cc2)cc1F] |
| 146 | 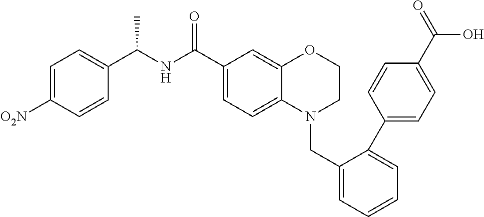 [SMILES: C[C@H](NC(=O)c1ccc2c(c1)OCCN2Cc1ccccc1-c1ccc(C(=O)O)cc1)c1ccc([N+](=O)[O-])cc1] |
| 147 | 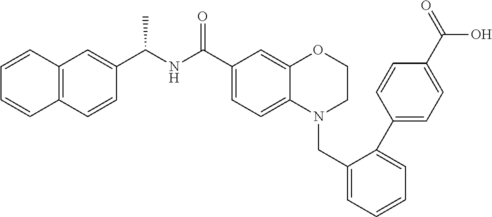 [SMILES: C[C@H](NC(=O)c1ccc2c(c1)OCCN2Cc1ccccc1-c1ccc(C(=O)O)cc1)c1ccc2ccccc2c1] |
| 148 | 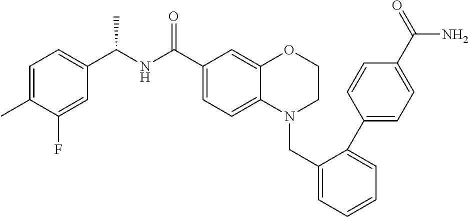 [SMILES: Cc1ccc([C@H](C)NC(=O)c2ccc3c(c2)OCCN3Cc2ccccc2-c2ccc(C(N)=O)cc2)cc1F] |
| 149 | 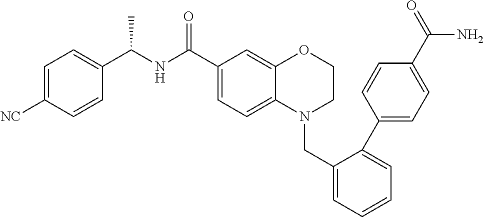 [SMILES: C[C@H](NC(=O)c1ccc2c(c1)OCCN2Cc1ccccc1-c1ccc(C(N)=O)cc1)c1ccc(C#N)cc1] |
| 150 | 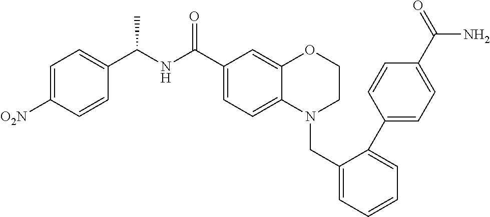 [SMILES: C[C@H](NC(=O)c1ccc2c(c1)OCCN2Cc1ccccc1-c1ccc(C(N)=O)cc1)c1ccc([N+](=O)[O-])cc1] |
| 151 | 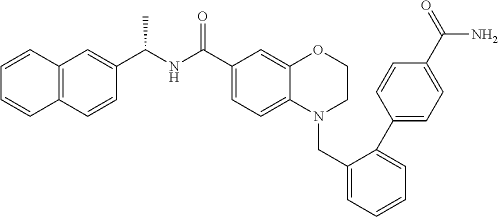 [SMILES: C[C@H](NC(=O)c1ccc2c(c1)OCCN2Cc1ccccc1-c1ccc(C(N)=O)cc1)c1ccc2ccccc2c1] |
| 152 | 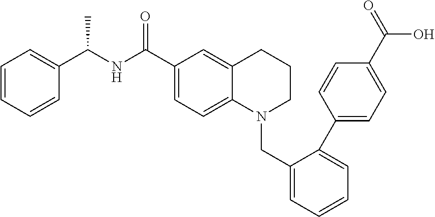 [SMILES: C[C@H](NC(=O)c1ccc2c(c1)CCCN2Cc1ccccc1-c1ccc(C(=O)O)cc1)c1ccccc1] |
| 153 | 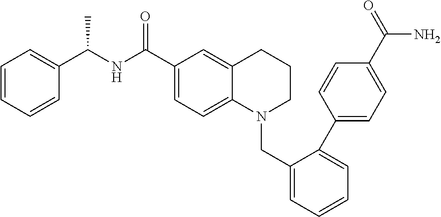 [SMILES: C[C@H](NC(=O)c1ccc2c(c1)CCCN2Cc1ccccc1-c1ccc(C(N)=O)cc1)c1ccccc1] |
| 154 | 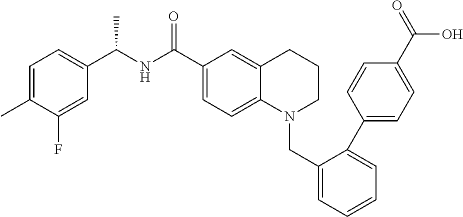 [SMILES: Cc1ccc([C@H](C)NC(=O)c2ccc3c(c2)CCCN3Cc2ccccc2-c2ccc(C(=O)O)cc2)cc1F] |
| 155 | 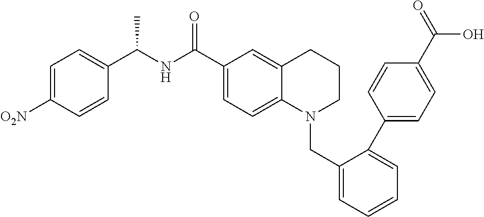 [SMILES: C[C@H](NC(=O)c1ccc2c(c1)CCCN2Cc1ccccc1-c1ccc(C(=O)O)cc1)c1ccc([N+](=O)[O-])cc1] |
| 156 | 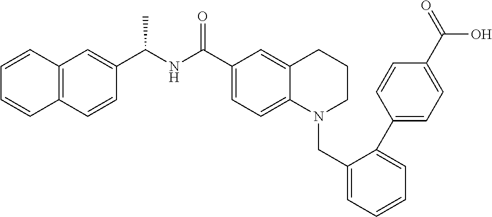 [SMILES: C[C@H](NC(=O)c1ccc2c(c1)CCCN2Cc1ccccc1-c1ccc(C(=O)O)cc1)c1ccc2ccccc2c1] |
| 157 | 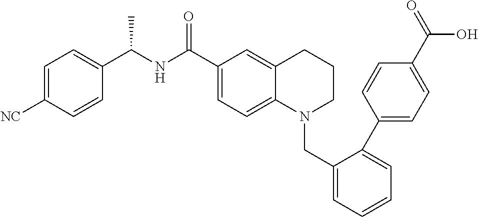 [SMILES: C[C@H](NC(=O)c1ccc2c(c1)CCCN2Cc1ccccc1-c1ccc(C(=O)O)cc1)c1ccc(C#N)cc1] |
| 158 | 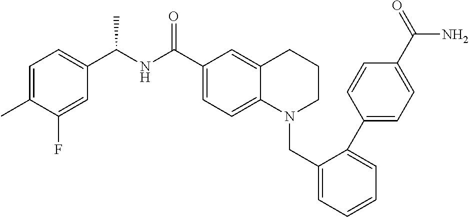 [SMILES: Cc1ccc([C@H](C)NC(=O)c2ccc3c(c2)CCCN3Cc2ccccc2-c2ccc(C(N)=O)cc2)cc1F] |
| 159 | 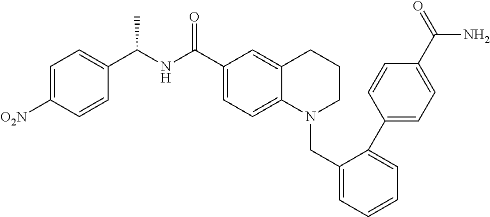 [SMILES: C[C@H](NC(=O)c1ccc2c(c1)CCCN2Cc1ccccc1-c1ccc(C(N)=O)cc1)c1ccc([N+](=O)[O-])cc1] |
| 160 | 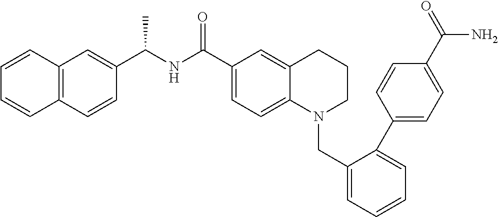 [SMILES: C[C@H](NC(=O)c1ccc2c(c1)CCCN2Cc1ccccc1-c1ccc(C(N)=O)cc1)c1ccc2ccccc2c1] |
| 161 | 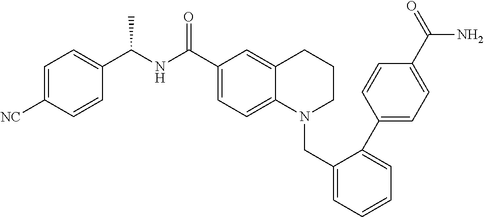 [SMILES: C[C@H](NC(=O)c1ccc2c(c1)CCCN2Cc1ccccc1-c1ccc(C(N)=O)cc1)c1ccc(C#N)cc1] |
| 162 | 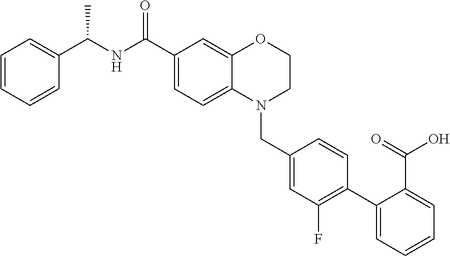 [SMILES: C[C@H](NC(=O)c1ccc2c(c1)OCCN2Cc1ccc(-c2ccccc2C(=O)O)c(F)c1)c1ccccc1] |
| 163 | 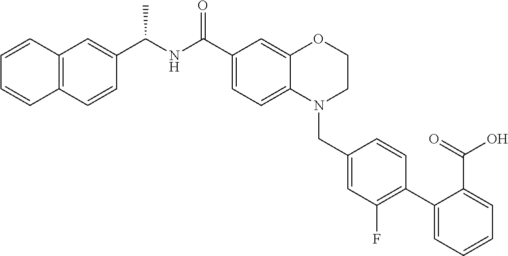 [SMILES: C[C@H](NC(=O)c1ccc2c(c1)OCCN2Cc1ccc(-c2ccccc2C(=O)O)c(F)c1)c1ccc2ccccc2c1] |
| 164 | 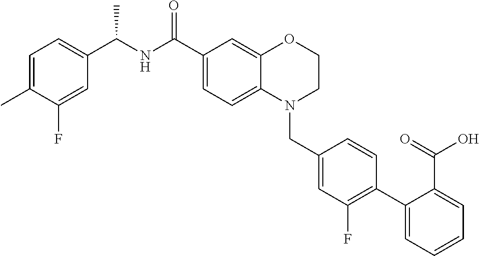 [SMILES: Cc1ccc([C@H](C)NC(=O)c2ccc3c(c2)OCCN3Cc2ccc(-c3ccccc3C(=O)O)c(F)c2)cc1F] |

### Experimental Example 1

### Evaluation of CDK5 (Cyclin-Dependent Kinase 5) Phosphorylation-Suppressing Activity

The following experiment was performed to investigate whether or not the compounds prepared in the examples of the invention could bind to PPARG but did not act as a CDK5 (cyclin-dependant kinase 5) promoter so that it did inhibit the phosphorylation of the 273^{rd} amino acid serine of PPARG.

The compounds of examples 1-164 and SR1664 and SR1824 of the comparative examples were diluted in DMSO (dimethyl sulfoxide) to meet the final test concentration 2000×. The 2000× compounds were diluted in 50% DMSO (DMSO:DW=1:1) at the ratio of 1:100. Premix in total volume of 36 μL was prepared by mixing 0.43 μg of PPARG-LBD (ligand binding domain) (human recombinant) (Cayman, 10007941), Cdk5/p35, 100 ng of active (Millipore, 14-477), 10× kinase buffer (Cell Signaling, 9802S), and DW (Data warehousing) (the premix was prepared on the ice and stored in the ice).

2 μL of the compound of the invention was mixed in 36 μL of the premix, followed by reaction in the ice for 10 minutes. 2 μL of 10 mM ATP (negative control: DW 2 μL) was added thereto, followed by reaction in a 37° C. water bath for 15 minutes. Upon completion of the reaction, the mixture was transferred onto the ice swiftly so that the mixture was cooled down for 1-2 minutes. 10 μL of 5× sample buffer was mixed with the reaction mixture, followed by heating at 95° C. in heat block for 8-10 minutes. Upon completion of the treatment in heat block, the mixture was taken out and cooled down for a while, followed by SDS-PAGE (sodium dodecyl sulfate-polyacryl amide gel electrophoresis) on 10% SDS-gel.

Then, the protein on SDS-gel was transferred onto nitrocellulose membrane (IB3010-32, Invitrogen) by using iBlot (IB1001, Invitrogen).

Upon completion of the transfer, the membrane proceeded to blocking in 5% BSA-TBST (bovine serum albumin-tris buffered saline and tween 20) for 30 minutes-1 hour, followed by reaction with the primary antibody phospho-PPARG-Ser273 (Hyundai Pharm. Co., Ltd.) for at least 18 hours in a 4° C. refrigerator. The membrane was washed with TBST (Tris-Buffered Saline and Tween 20), followed by reaction with the secondary antibody Anti-rabbit IgG (HRP-linked antibody, 7074S, Cell Signaling) at room temperature for 1 hour. Upon completion of the reaction, the membrane was washed with TBST, followed by reaction in ECL (enhanced chemiluminescence) solution (NCI34095KR, Thermo Scientific). The band signal for phospho-PPARG-Ser273 was measured by Las4000mini (Fujifilm corp). After measuring the phospho-Ser273, the membrane was reacted in stripping buffer (NCI1059KR, Thermo Scientific) for 30 minutes, and then was washed, which was the ready for the total PPARG measurement.

The membrane was reacted with PPARG (E-8) antibody (sc-7273, Santa Cruz), the primary antibody, at room temperature for 1 hour, followed by another reaction with anti-mouse IgG (HRP-linked antibody, sc-2005, Santa Cruz), the secondary antibody, for 1 hour.

Upon completion of the reaction, the membrane was washed and reacted in ECL solution (RPN2232, GE Healthcare). The total PPARG band signal was measured by Las4000mini (Fujifilm corp).

The compounds prepared in the examples of the present invention were confirmed to bind to PPARG (peroxisome proliferator activated receptor-gamma) but not to act as a CDK5 (cyclin-dependant kinase 5) promoter, indicating that they are excellent in suppressing the phosphorylation of serine, the 273^{rd} amino acid of PPARG.

Therefore, the compounds of examples of the invention bind to PPARG (peroxisome proliferator activated receptor-gamma) but do not activate CDK5 (cyclin-dependant kinase 5), the phosphorylation inducer, so that they can be effectively used as a therapeutic agent for PPARG-related disease by preventing side effects accompanied by the phosphorylation of serine, the 273^{rd} amino acid of PPARG.

### Experimental Example 2

### Evaluation of PPARG (Peroxisome Proliferator Activated Receptor-Gamma) Binding Capacity and Transcriptional Activity

The following experiment was performed to evaluate the PPARG (peroxisome proliferator activated receptor-gamma) binding capacity and transcriptional activity of the compounds prepared in the examples of the invention.

For the experiment, LanthaScreen™ TR-FRET PPARG Competitive Binding Assay Kit (PV4894, Invitrogen) was used according to the manufacturer's instruction.

30 μL of the compound or the control substance (SR-1664, Rosiglitazone) diluted in assay buffer (50×) in a 96-well plate (SPL, 34096) was mixed with 15 μL of 5 nM Fluormone™ H Pan-PPAR Green, to which 15 μL of 5 nM GST-PPARG-LBD and 5 nM Tb-GST-antibody were added. The mixture was loaded in a 384-well plate (Greiner, 784075) by 20 μL per well, followed by reaction at room temperature for 1 hour.

Upon completion of the reaction, fluorescence value was measured by using Flexstation 3 (Molecular Devices) with time-resolved fluorescence (RFU) mode (excitation 1: 340 nm, emission 1: 518 nm, excitation 2: 340 nm, emission 2: 488 nm, integration delay 50 μs, integration 400 μs). The results were calculated by using 518 nm RFUs/488 nm RFUs ratio. The activity of the compounds of the invention was compared with that of the vehicle, which was calculated by ratio according to the formula [100%−compound of the example ratio/vehicle ratio]. At this time, the binding activity ratio to rosiglitazone was compared.

The following experiment was performed to evaluate PPARG (peroxisome proliferator activated receptor-gamma) transcriptional activity of the compounds of the invention.

HEK293 cells were plated in a 24-well plate (SPL, 30024) at the density of 2.5×10⁴ cells/well. The cells were transfected with PPRE, PPARG or PPARα, RXR, and Renilla DNA by using FuGENE HD (Promega, E2312). Upon completion of the gene transfection, the cells were treated with rosiglitazone, SR1664, and the compounds of the invention respectively for 24 hours. 24 hours later, the cells were collected, followed by reporter gene assay with Dual Reporter gene assay kit (Promega, E1980). Luciferase assay activity was calculated by normalization of renilla activity.

TABLE 2

| | Binding assay | PPRE |
|---|---|---|
| Example | (1 μLM) | EC50 (nM) |
| 1 | +++ | * |
| 2 | +++ | *** |
| 3 | + | NA |
| 4 | +++ | *** |
| 5 | ++ | NA |
| 6 | +++ | * |
| 7 | ++++ | * |
| 8 | +++ | * |
| 9 | ++++ | *** |
| 10 | +++ | NA |
| 11 | ++++ | *** |
| 12 | ++++ | *** |
| 13 | ++++ | NA |
| 14 | ++ | NA |
| 15 | + | * |
| 16 | +++ | ** |
| 17 | +++ | ** |
| 18 | +++ | *** |
| 19 | +++ | *** |
| 20 | ++++ | NA |
| 21 | +++ | * |
| 22 | +++ | * |
| 23 | +++ | * |
| 24 | +++ | ** |
| 25 | +++ | NA |
| 26 | ++ | * |
| 27 | + | NA |
| 28 | ++++ | ** |
| 29 | ++++ | NA |
| 30 | +++ | * |
| 31 | ++++ | *** |
| 32 | +++ | *** |
| 33 | +++ | NA |
| 34 | ++++ | NA |
| 35 | +++ | *** |
| 36 | +++ | NA |
| 37 | +++ | NA |
| 38 | ++ | NA |
| 39 | +++ | *** |
| 40 | +++ | *** |
| 41 | +++ | NA |
| 42 | +++ | NA |
| 43 | ++ | NA |
| 44 | +++ | NA |
| 45 | +++ | *** |
| 46 | +++ | NA |
| 47 | +++ | NA |
| 48 | + | NA |
| 49 | +++ | NA |
| 50 | +++ | *** |
| 51 | +++ | NA |
| 52 | +++ | NA |
| 53 | +++ | *** |
| 54 | +++ | *** |
| 55 | NT | NT |
| 56 | ++++ | NA |
| 57 | +++ | NA |
| 58 | + | NA |
| 59 | ++ | *** |
| 60 | ++ | NA |
| 61 | ++ | NA |
| 62 | +++ | * |
| 63 | + | NA |
| 64 | + | NA |
| 65 | ++ | NA |
| 66 | + | NA |
| 67 | ++ | * |
| 68 | ++ | * |
| 69 | + | NA |
| 70 | ++ | NA |
| 71 | + | * |
| 72 | + | NA |
| 73 | + | * |
| 74 | + | NA |
| 75 | +++ | * |
| 76 | +++ | NA |
| 77 | +++ | * |
| 78 | ++ | NA |
| 79 | ++ | * |
| 80 | ++ | NA |
| 81 | +++ | * |
| 82 | +++ | * |
| 83 | ++ | * |
| 84 | ++ | * |
| 85 | +++ | *** |
| 86 | +++ | NA |
| 87 | ++++ | NA |
| 88 | + | * |
| 89 | + | NA |
| 90 | + | NA |
| 91 | +++ | * |
| 92 | +++ | NA |
| 93 | ++ | * |
| 94 | ++ | *** |
| 95 | +++ | *** |
| 96 | +++ | NA |
| 97 | + | NA |
| 98 | + | NA |
| 99 | +++ | NA |
| 100 | ++ | *** |
| 101 | ++ | * |
| 102 | + | NA |
| 103 | ++ | NA |
| 104 | NT | NT |
| 105 | + | NA |
| 106 | ++++ | *** |
| 107 | +++ | *** |
| 108 | ++++ | NA |
| 109 | +++ | * |
| 110 | +++ | NA |
| 111 | ++++ | *** |
| 112 | +++ | NA |
| 113 | ++ | NA |
| 114 | +++ | NA |
| 115 | +++ | NA |
| 116 | +++ | * |
| 117 | +++ | *** |
| 118 | + | NA |
| 119 | ++ | * |
| 120 | ++++ | * |
| 121 | +++ | ** |
| 122 | ++ | NA |
| 123 | +++ | NA |
| 124 | ++++ | ** |
| 125 | +++ | NA |
| 126 | +++ | * |
| 127 | +++ | * |
| 128 | ++ | * |
| 129 | ++++ | ** |
| 130 | +++ | *** |
| 131 | +++ | NA |
| 132 | ++++ | * |
| 133 | +++ | NA |
| 134 | ++++ | * |
| 135 | +++ | *** |
| 136 | +++ | * |
| 137 | ++++ | *** |
| 138 | ++++ | *** |
| 139 | +++ | NA |
| 140 | ++++ | *** |
| 141 | +++ | NA |
| 142 | ++ | * |
| 143 | ++ | NA |
| 144 | + | *** |
| 145 | ++ | NA |
| 146 | + | * |
| 147 | +++ | * |
| 148 | ++ | NA |
| 149 | + | NA |
| 150 | ++ | NA |
| 151 | +++ | *** |
| 152 | ++ | ** |
| 153 | ++ | *** |
| 154 | + | NA |
| 155 | + | NA |
| 156 | + | NA |
| 157 | ++ | NA |
| 158 | + | NA |
| 159 | + | NA |
| 160 | ++ | *** |
| 161 | ++ | NA |
| 162 | +++ | NT |
| 163 | +++ | NT |
| 164 | +++ | NT |

In Table 2, NT (No Tested) means that the experiment was not performed; “+” indicates the degree of the binding activity, and as the number of “+” increases, the binding activity is stronger; “*” indicates the degree of the transcriptional activity, and as the number of “*” increases, the transcription activity is stronger; and “NA” indicates that no transcriptional activity is detected because the compound is not acting as an agonist.

As shown in Table 2, the compounds of the examples of present invention displayed excellent binding activity to PPARG (peroxisome proliferator activated receptor-gamma). At this time, the binding activity number indicates the binding or no binding and does not relate directly to the pharmacological activity.

The compounds of the examples of present invention were confirmed not to induce the transcription of PPARG (peroxisome proliferator activated receptor-gamma).

Therefore, the compounds of the examples of the present invention bind specifically to PPARG (peroxisome proliferator activated receptor-gamma) but do not induce the transcription of PPARG gene and thereby prevent the side effects due to PPARG phosphorylation, suggesting that the compounds can be effectively used as a therapeutic agent for PPARG-related diseases.

### Experimental Example 3

### Evaluation of CYP (Cytochrome P450)-Suppressing Effects

To evaluate the CYP (cytochrome P450)-suppressing activity of the compounds prepared in the examples of the invention, the following experiment was performed.

The analysis of CYP1A2, 2C19, and 3A4 suppressing activity was performed by using a BD GENTEST (459100, 459400, and 459500) kit, and the analysis of CYP 2C9- and 2D6-suppressing activity was performed by using an Invitrogen (P2861 and P2862) kit.

When the Invitrogen kit was used, the compounds of the present invention were diluted in Vivid CYP450 reaction buffer (1×) at the final concentration of 2.5×. The P450 BACULOSOMES sample and Regeneration system (100×) were diluted in Vivid CYP450 reaction buffer (1×) at different proper concentrations. 80 μL of the compound of the invention (2.5×) and 100 μL of the diluted P450 BACULOSOMES sample mixture were mixed in a U-bottom 96-well plate, followed by pre-incubation for 10 minutes. Vivid CYP450 Substrate and NADP+ (100×) were diluted in Vivid CYP450 reaction buffer (1×) at a desired concentration, respectively, according to their corresponding types of CYP450 and substrates. 20 μL of Substrate-NADP+ mix was loaded in the plate when the pre-incubation had been completed, followed by reaction for 1 hour. Upon completion of the reaction, the reaction mixture was transferred in a white plate and fluorescent wavelength was measured with a microplate reader (2C9 excitation 415 nm, emission 460 nm, 2D6 excitation 400 nm, emission 502 nm).

When the BD GENTEST kit was used, the compounds of the present invention were diluted in acetonitrile at the final concentration of 50×. The NADPH-Cofactor mix was prepared by diluting the Cofactor, G6PDH, and control protein provided by the kit in DW according to the instructed concentration. 4 μL of the compound of the example (50×) was mixed with 96 μL of NADPH-Cofactor mix in a 96-well plate, followed by pre-incubation in a 37° C. incubator for 10 minutes.

The enzyme/substrate mixture was diluted in the buffer (0.5 M potassium phosphate, pH 7.4) provided by the kit according to the instructed concentration. Each CYP450 enzyme substrate was diluted in DW at a desired concentration respectively according to the corresponding types of CYP450. 100 μL of the enzyme/substrate mixture was loaded in the plate when the pre-incubation had been completed, followed by reaction in a 37° C. incubator for 15 minutes (1A2) and 30 minutes (3A4 and 2C19). Upon completion of the reaction, the mixture was transferred to a white plate, and the fluorescent wavelength was measured with a microplate reader (1A2, 2C19 excitation 410 nm, emission 460 nm; 3A4, excitation 409 nm, emission 530 nm).

The test results were calculated by the following formula:

(1−(fluorescence value of the test material/fluorescence value of the vehicle))×100%.

TABLE 3

| | CYP(10 μL, %) | | | | |
|---|---|---|---|---|---|
| Example | 1A2 | 2C9 | 2C19 | 2D6 | 3A4 |
| 14 | 97.99% | 64.28% | 79.96% | 86.13% | 71.24% |
| 36 | 98.84% | 51.03% | 52.68% | 75.73% | 58.18% |
| 44 | 94.56% | 56.40% | 59.55% | 97.07% | 70.42% |
| 56 | 100.73% | 55.36% | 85.71% | 94.15% | 54.62% |
| 57 | 100.32% | 58.87% | 75.69% | 82.59% | 59.17% |
| 58 | 100.93% | 57.47% | 70.53% | 55.23% | 56.92% |
| 60 | 100.63% | 80.25% | 69.60% | 62.27% | 70.64% |
| 74 | 102.89% | 80.68% | 91.36% | 83.97% | 81.98% |
| 96 | 92.41% | 57.63% | 52.35% | 98.66% | 98.04% |
| 110 | 97.28% | 55.18% | 69.53% | 88.44% | 53.03% |
| 113 | 97.27% | 67.73% | 54.70% | 78.65% | 85.01% |
| 115 | 97.62% | 63.87% | 59.33% | 95.56% | 77.73% |
| 118 | 101.50% | 52.02% | 74.03% | 94.83% | 71.21% |
| 123 | 102.45% | 59.36% | 58.85% | 82.48% | 52.65% |
| 131 | 94.17% | 52.84% | 54.84% | 86.92% | 55.88% |
| 139 | 94.00% | 63.24% | 53.50% | 80.63% | 60.95% |
| Comparative | 60.95% | 3.52% | 33.88% | 50.18% | 58.56% |
| Example 1 | | | | | |
| (SR1664) | | | | | |
| Comparative | 64.12% | 6.43% | 32.45% | 68.34% | 71.88% |
| Example 2 | | | | | |
| (SR1824) | | | | | |

As shown in Table 3, the compounds of the examples of 14, 36, 44, 56, 57, 58, 60, 74, 96, 110, 113, 115, 118, 123, 131, and 139 of the invention suppressed the CYP isozymes 1A2, 2C9, 2C19, and 2D6 less than the compounds of the comparative examples (SR1664 and SR1824).

Particularly, SR1664 suppressed the CYP isozyme 2C9 activity significantly, so that approximately 96% of the drug remained without being decomposed. Therefore, when SR1664 was used as a drug, the amount that reached a target was more than the proper dose as a drug, which could cause toxicity.

However, the compounds of the present invention suppressed the CYP activity moderately compared with SR1664, so the drug decomposition rate was at least 10 times higher than SR1664, suggesting that a proper amount of the drug would reach a target. Therefore, the compounds of the present invention were confirmed to be more effective as a drug than the conventional SR1664 and SR1824.

### Experimental Example 4

### Evaluation of Cardiotoxicity

To evaluate the cardiotoxicity of the compounds prepared in the examples of the present invention, the following experiment was performed.

HEK293 cells expressing HERG (Human Ether-a-go-go-Related Gene) channel safely were cultured in MEM (Minimum Essential Medium) supplemented with 10% fetal bovine serum (FBS), 1 mM sodium pyruvate, 0.1 mM non-essential amino acid solution, 100 units/ml penicillin-streptomycin, 100 μg/ml streptomycin sulfate and 100 μg/ml zeocin under the condition of air:CO₂=95:5. When the cells were grown to 60-80% confluency, the cells were loaded in the medium supplemented with 0.25% trypsin and 0.02% EDTA (ethylenediaminetertraacetic acid) for 3 minutes. The cells were then washed with a fresh medium and then dispensed on a new plastic culture dish. For the electrophysiological record, the cells were seeded on a glass cover slip of 5 mm in diameter, followed by incubation in a 24-well plate for 5-24 hours. Then, the cells were transferred into the recording chamber.

The current value of all the cells was measured by the conventional patch-clamp technique or Port-a-Patch (Nanion, Germany) semiautomatic technique. The pH of the external bath solution containing 136 mM NaCl, 5.4 mM KCl, 1.8 mM CaCl₂, 1 mM MgCl₂, 10 mM glucose, and 10 mM HEPES (4-(2-hydroxyethyl)-1-piperazineethanesulfonic acid) was adjusted to 7.4 using NaOH. The pH of the intracellular pipette solution containing 130 mM KCl, 1 mM MgCl₂, 10 mM EGTA (ethylene glycol tetraacetic acid), 5 mM Mg-ATP, and mM HEPES was adjusted to 7.2 using KOH.

In the conventional patch-clamp technique, the patch pipette was pulled by a PP-83 pipette puller (Narishige, Tokyo, Japan), so that it had 2-4 MΩ of resistance in the external bath solution. The ionic current was recorded by using EPC7 pulse amplifier (HEKA electronic, Germany). The voltage-clamp amplification was regulated using the pClamp computer program (Axon Instruments, USA), and the obtained data were analyzed. During the recording, the solution above was continually spread on HEK293 cells in the chamber. For the recording, the Port-a-Patch (Nanion, Germany) semiautomatic patch system was used, and the amplifier used herein was EPC10.

The membrane potential was maintained at −80 mV, which was changed to +20 mV for 4 seconds, then changed to −50 mV for 6 seconds and then maintained again at −80 mV. The changes were made every 25 seconds.

To obtain the concentration-reaction curve in the presence of the drug, dose-dependent inhibition was entered into the Hill formula represented by mathematical formula 1 below.

$$\frac{{I}_{drug}}{{I}_{control}} = \frac{1}{1 + {( \frac{[ D ]}{{IC}_{50}} )}^{h}} \qquad [ Mathematical Formula 1 ]$$

In mathematical formula 1, I_{drug} indicates the peak tail current in the presence of a drug, I_{control} indicates the maximum peak tail current in the absence of a drug, D indicates the concentration of a drug, h indicates Hill constant, and IC₅₀ indicates the concentration when the half-maximum peak tail current was suppressed.

TABLE 4

| Example | IC₅₀ (uM) |
|---|---|
| 157 | 42.67 |
| 156 | 55.21 |
| 155 | 73.60 |
| 56 | 72.60 |
| Comparative | 2.7 |
| Example 1 | |
| (SR-1664) | |

As shown in Table 4, SR-1664 displayed a significantly lower IC₅₀ (2.7 uM) than an IC₅₀ that can induce cardiotoxicity (10 uM), suggesting that, when it is used as a drug, there is a high chance of cardiotoxicity.

However, the compounds of the invention displayed a much higher IC₅₀ than the IC₅₀ that can induce cardiotoxicity (10 uM), suggesting that there is a low chance of cardiotoxicity when they are used as a drug.

Therefore, the compounds of the examples of the invention can be effectively used as a therapeutic agent for PPARG-related disease with significantly low chance of causing cardiotoxicity.

### Experimental Example 5

### Evaluation of Blood Glucose-Lowering Effects and Body Weight-Reducing Effects Through a DIO (Diet-Induced Obesity) Model

To evaluate the blood glucose-lowering effects and body weight-reducing effects of the compounds prepared in the examples of the invention, the following experiment was performed.

The male C57BL/6 mice at 4 seeks were given a high fat diet (Lab. Diet Co.) to construct a high fat diet-induced obesity (DIO) mouse model.

Among those mice that gained more weight by a high fat diet, those that weighed more than 40 g were selected. Groups were divided with the randomly selected mice (n=8). Each group was treated separately with a vehicle, the positive control drug, and the test compounds for 4 weeks at the different concentrations. Weight measurements were performed twice a week during the 4-week treatment period, and the weight changes were recorded.

Then, an oral glucose tolerance test (OGTT) was performed as follows.

30 minutes after the treatment of hte vehicle or the compounds of the example, the mice were orally administered 2 g/kg of glucose. Blood glucose was measured using an Accu-chek active strip (Roche Diagnostic Co.). The time point for the measurements were −30, 0, 20, 40, 60, and 120 minutes from the glucose administration via tail vein puncture. The obtained AUC results and the weight changes are shown in Table 5.

TABLE 5

| | Comparative | | | | |
|---|---|---|---|---|---|
| | Example 1 | Example | | | |
| | SR-1664 | 56 | 155 | 156 | 157 |
| | [20 mpk] | [10 mpk] | [10 mpk] | [10 mpk] | [10 mpk] |
| Weight | 1.6% ↓ | 10.6% ↓ | 5.24% ↓ | 6.26% ↓ | 8.76% ↓ |
| change | | | | | |
| Blood | 14% ↓ | 37.5% ↓ | 25.0% ↓ | 34.2% ↓ | 24.6% ↓ |
| glucose | | | | | |
| (oGTT) | | | | | |

As shown in Table 5, the comparative example compound SR-1664 (20 mpk) reduced the weight of the mice by 1.6%, while the compounds of the examples of 56, 155, 156, and 157 (10 mpk each) reduced the mouse weight at least 5% even at a low dose.

Further, the comparative example compound SR-1664 (20 mpk) reduced blood glucose by 14%, while the compounds of the examples 56, 155, 156, and 157 (10 mpk each) reduced blood glucose at least 24% even at a low dose.

Therefore, the compounds of the examples of the present invention had a significant effect of lowering body weight and blood glucose even at a low dose, so that they can be effectively used as a therapeutic agent for PPARG-related disease.

### Manufacturing Example 1

### Preparation of Pharmaceutical Formulations

### 1-1. Preparation of Powders

| | |
|---|---|
| The compound represented by formula 1 | 500 mg |
| Lactose | 100 mg |
| Talc | 10 mg |

Powders were prepared by mixing all the above components, which were filled in airtight packs according to the conventional method for preparing powders.

### 1-2. Preparation of Tablets

| | |
|---|---|
| The compound represented by formula 1 | 500 mg |
| Corn starch | 100 mg |
| Lactose | 100 mg |
| Magnesium stearate | 2 mg |

Tablets were prepared by mixing all the above components by the conventional method for preparing tablets.

### 1-3. Preparation of Capsules

| | |
|---|---|
| The compound represented by formula 1 | 500 mg |
| Corn starch | 100 mg |
| Lactose | 100 mg |
| Magnesium stearate | 2 mg |

Capsules were prepared by mixing all the above components, which were filled in gelatin capsules according to the conventional method for preparing capsules.

### 1-4. Preparation of Injectable Solution

| | |
|---|---|
| The compound represented by formula 1 | 500 mg |
| Sterilized distilled water | proper amount |
| pH regulator | proper amount |

Injectable solutions were prepared by mixing all the above components, putting the mixture into 2 mL ampoules and sterilization thereof by the conventional method for preparing injectable solutions.

### 1-5. Preparation of Liquid Formulations

| | | |
|---|---|---|
| The compound represented by formula 1 | 100 | mg |
| Isomerized sugar | 10 | g |
| Mannitol | 5 | g |

| | |
|---|---|
| Purified water | proper amount |

All the above components were dissolved in purified water. After adding lemon flavor, total volume was adjusted to be 100 mL by adding purified water. Liquid formulations were prepared by putting the mixture into brown bottles and sterilization thereof by the conventional method for preparing liquid formulations.

### INDUSTRIAL APPLICABILITY

The compound represented by formula 1 or the optical isomer thereof of the present invention binds to PPARG at a high affinity but does not act as an agonist and thereby does not induce gene transcription. The compound also blocks CDK5, which is the cause of PPARG phosporylation, so that the compound can suppress the side effects of conventional anti-diabetic agents and at the same time can sufficiently improve pharmacophysical properties to produce a PPARG-related disease-treating effect. Therefore, the compound of the invention can be effectively used as a pharmaceutical composition for treating PPARG-related disease.

What is claimed is:

1. A compound represented by formula 1 below, an optical isomer thereof, or a pharmaceutically acceptable salt thereof: 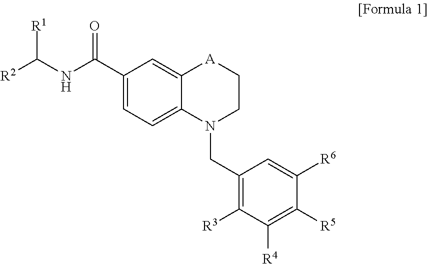
wherein,
R¹ is H, unsubstituted or substituted C₁₋₁₀ straight or branched alkyl wherein one or more halogens are substituted, or unsubstituted or substituted C₁₋₁₀ straight or branched alkoxy wherein one or more halogens are substituted;
R² is unsubstituted or substituted C₆₋₁₀ aryl, unsubstituted or substituted C₆₋₁₀ aryl C₁₋₁₀ straight or branched alkyl, or unsubstituted or substituted 5-membered heteroaryl containing one or more hetero atoms selected from the group consisting of N, O, and S;
for the substituted C₆₋₁₀ aryl, the substituted C₆₋₁₀ aryl C₁₋₁₀ straight or branched alkyl, and the substituted 5-10 membered heteroaryl, one or more substituents selected from the group consisting of C₁₋₁₀ straight or branched alkyl unsubstituted or substituted with one or more halogens, C₁₋₁₀ straight or branched alkoxy unsubstituted or substituted with one or more halogens, C₁₋₁₀ straight or branched alkylsulfonyl unsubstituted or substituted with one or more halogens, C₁₋₁₀ straight or branched alkoxycarbonyl, halogen, nitrile, and nitro can be substituted;
for the said unsubstituted or substituted C₆₋₁₀ aryl, phenyl or 5-8 membered heterocycloalkyl containing one or more hetero atoms selected from the group consisting of N, O, and S can be fused;
R¹ and R² can form C₅₋₁₀ cycloalkyl, 5-10 membered heterocycloalkyl containing one or more hetero atoms selected from the group consisting of N, O, and S, or 5-10 membered heteroaryl containing one or more hetero atoms selected from the group consisting of N, O, and S along with the carbon atoms which are conjugated to the same;
for the said C₅₋₁₀ cycloalkyl, the 5-10 membered heterocycloalkyl, or the 5-10 membered heteroaryl, phenyl can be fused;
R³, R⁴, R⁵, and R⁶ are independently H, halogen, unsubstituted or substituted C₁₋₁₀ straight or branched alkyl wherein one or more halogens are substituted, unsubstituted or substituted C₁₋₁₀ straight or branched alkoxy wherein one or more halogens are substituted, or unsubstituted or substituted C₆₋₁₀ aryl;
for the said substituted C₆₋₁₀ aryl, one or more substituents selected from the group consisting of halogen, unsubstituted or substituted C₁₋₁₀ straight or branched alkyl wherein one or more halogens are substituted, hydroxycarbonyl, aminocarbonyl, and sodiumoxycarbonyl can be substituted; and
A is —CH₂— or —O—.

2. The compound represented by formula 1, the optical isomer thereof, or the pharmaceutically acceptable salt thereof according to claim 1, wherein:
R¹ is H, unsubstituted or substituted C₁₋₅ straight or branched alkyl wherein one or more halogens are substituted, or unsubstituted or substituted C₁₋₅ straight or branched alkoxy wherein one or more halogens are substituted;
R² is unsubstituted or substituted C₆₋₈ aryl, unsubstituted or substituted C₆₋₈ aryl C₁₋₅ straight or branched alkyl, or unsubstituted or substituted 5-8 membered heteroaryl containing one or more hetero atoms selected from the group consisting of N, O, and S;
for the said substituted C₆₋₈ aryl, the substituted C₆₋₈ aryl C₁₋₅ straight or branched alkyl, and the substituted 5-8 membered heteroaryl, one or more substituents selected from the group consisting of C₁₋₅ straight or branched alkyl unsubstituted or substituted with one or more halogens, C₁₋₅ straight or branched alkoxy unsubstituted or substituted with one or more halogens, C₁₋₅ straight or branched alkylsulfonyl unsubstituted or substituted with one or more halogens, C₁₋₅ straight or branched alkoxycarbonyl, halogen, nitrile, and nitro can be substituted;
for the said unsubstituted or substituted C₆₋₈ aryl, phenyl or 5-8 membered heterocycloalkyl containing one or more hetero atoms selected from the group consisting of N, O, and S can be fused;
R¹ and R² can form C₅₋₈ cycloalkyl, 5-8 membered heterocycloalkyl containing one or more hetero atoms selected from the group consisting of N, O, and S, or 5-8 membered heteroaryl containing one or more hetero atoms selected from the group consisting of N, O, and S along with the carbon atoms which are conjugated to the same;
for the said C₅₋₈ cycloalkyl, the 5-8 membered heterocycloalkyl, or the 5-8 membered heteroaryl, phenyl can be fused;
R³, R⁴, R⁵, and R⁶ are independently H, halogen, unsubstituted or substituted C₁₋₅ straight or branched alkyl wherein one or more halogens are substituted, unsubstituted or substituted C₁₋₅ straight or branched alkoxy wherein one or more halogens are substituted, or unsubstituted or substituted C₆₋₈ aryl;
for the said substituted C₆₋₈ aryl, one or more substituents selected from the group consisting of halogen, unsubstituted or substituted C₁₋₅ straight or branched alkyl wherein one or more halogens are substituted, hydroxycarbonyl, aminocarbonyl, and sodiumoxycarbonyl can be substituted; and
A is —CH₂— or —O—.

3. The compound represented by formula 1, the optical isomer thereof, or the pharmaceutically acceptable salt thereof according to claim 1, wherein:
R¹ is H, methyl, or ethyl;
R² is unsubstituted or substituted phenyl, unsubstituted or substituted benzyl, or unsubstituted or substituted 5-6 membered heteroaryl containing one or more hetero atoms selected from the group consisting of N and O;
for the said substituted phenyl, the substituted benzyl, and the substituted 5-6 membered heteroaryl, one or more substituents selected from the group consisting of C₁₋₅ straight or branched alkyl unsubstituted or substituted with one or more halogens, C₁₋₅ straight or branched alkoxy unsubstituted or substituted with one or more halogens, C₁₋₅ straight or branched alkylsulfonyl unsubstituted or substituted with one or more halogens, C₁₋₅ straight or branched alkoxycarbonyl, halogen, nitrile, and nitro can be substituted;
for the said unsubstituted or substituted phenyl, phenyl or 6 membered heterocycloalkyl containing one or more hetero atoms selected from the group consisting of N and O can be fused;
R¹ and R² can form C₅₋₈ cycloalkyl, 5-8 membered heterocycloalkyl containing one or more hetero atoms selected from the group consisting of N, O, and S, or 5-8 membered heteroaryl containing one or more hetero atoms selected from the group consisting of N, O, and S along with the carbon atoms which are conjugated to the same;
for the said C₅₋₈ cycloalkyl, the 5-8 membered heterocycloalkyl, or the 5-8 membered heteroaryl, phenyl can be fused;
R³, R⁴, R⁵, and R⁶ are independently H, fluoro, or unsubstituted or substituted phenyl;
for the said substituted phenyl, one or more substituents selected from the group consisting of hydroxycarbonyl, aminocarbonyl, and sodiumoxycarbonyl can be substituted; and
A is —CH₂— or —O—.

4. The compound represented by formula 1, the optical isomer thereof, or the pharmaceutically acceptable salt thereof according to claim 1, wherein:
R¹ is H, methyl, or ethyl; 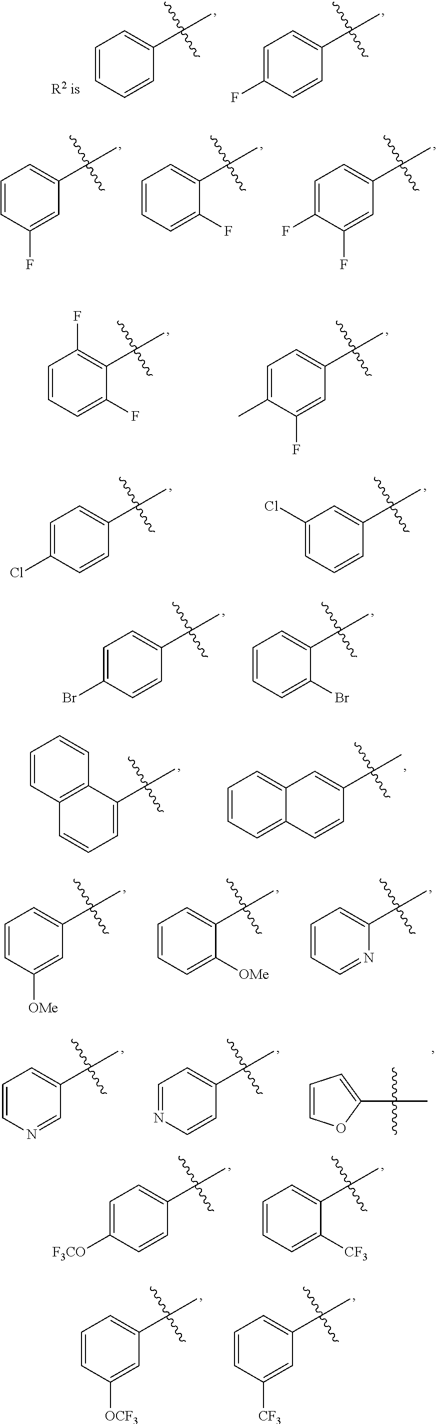 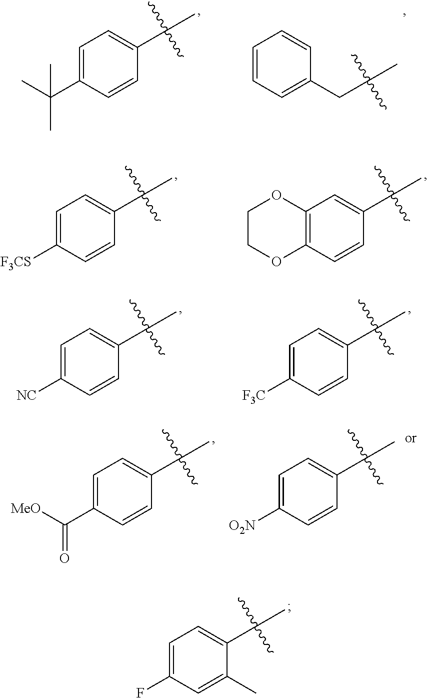
R³ is H, F, 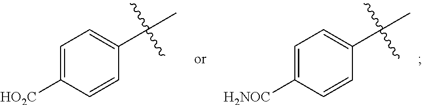 [SMILES: *c1ccc(C(=O)O)cc1 ; *c1ccc(C(N)=O)cc1]
R⁴ is H, F, or 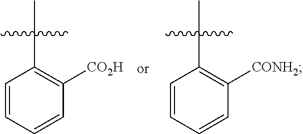 [SMILES: *c1ccccc1C(=O)O ; "or" ; *c1ccccc1C(N)=O]
R⁵ is H, 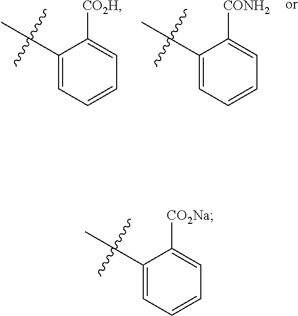
R⁶ is H, 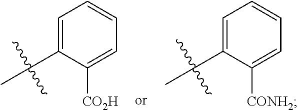 [SMILES: *c1ccccc1C(=O)O ; "or" ; *c1ccccc1C(N)=O]
and
A is —CH₂— or —O—.

5. The compound represented by formula 1, the optical isomer thereof, or the pharmaceutically acceptable salt thereof according to claim 1, wherein the compound represented by formula 1 is selected from the group consisting of the following compounds:
(1) (S)-4′-((6-((1-phenylethyl)carbamoyl)-3,4-dihydroquinoline-1(2H)-yl)methyl)-[1,1′-biphenyl]-2-carboxylic acid;
(2) (S)-1-((2′-carbamoyl-[1,1′-biphenyl]-4-yl)methyl)-N-(1-phenylethyl)-1,2,3,4-tetrahydroquinoline-6-carboxamide;
(3) (S)-4′-((6-((1-(4-nitrophenyl)ethyl)carbamoyl)-3,4-dihydroquinoline-1(2H)-yl)methyl)-[1,1′-biphenyl]-2-carboxylic acid;
(4) (S)-4′-((6-((1-(4-fluoro-2-methylphenyl)ethyl)carbamoyl)-3,4-dihydroquinoline-1(2H)-yl)methyl)-[1,1′-biphenyl]-2-carboxylic acid;
(5) (S)-4′-((6-((1-(3-fluoro-4-methylphenyl)ethyl)carbamoyl)-3,4-dihydroquinoline-1(2H)-yl)methyl)-[1,1′-biphenyl]-2-carboxylic acid;
(6) (S) 4′-((6-((1-(4 fluorophenyl)ethyl)carbamoyl)-3,4-dihydroquinoline-1(2H)-yl)methyl)-[1,1′-biphenyl]-2-carboxylic acid;
(7) (S) 4′-((6-((1-(4-bromophenyl)ethyl)carbamoyl)-3, 4-dihydroquinoline-1(2H)-yl)methyl)-[1,1′-biphenyl]-2-carboxylic acid;
(8) 4′-((6-((4-nitrobenzyl)carbamoyl)-3,4-dihydroquinoline-1(2H)-yl)methyl)-[1,1′-biphenyl]-2-carboxylic acid;
(9) (S)-4′-((6-((1-(3-chlorophenyl)ethyl)carbamoyl)-3, 4-dihydroquinoline-1(2H)-yl)methyl)-[1,1′-biphenyl]-2-carboxylic acid;
(10) (S)-4′-((6-((1-(naphthalene-1-yl)ethyl)carbamoyl)-3,4-dihydroquinoline-1(2H)-yl)methyl)-[1,1′-biphenyl]-2-carboxylic acid;
(11) (S)-4′-((6-((1-(4-(trifluoromethoxy)phenyl)ethyl)carbamoyl)-3,4-dihydroquinoline-1(2H)-yl)methyl)-[1,1′-biphenyl]-2-carboxylic acid;
(12) (S)-4′-((6-((1-(4-(trifluoromethyl)phenyl)ethyl)carbamoyl)-3,4-dihydroquinoline-1(2H)-yl)methyl)-[1,1′-biphenyl]-2-carboxylic acid;
(13) (S)-4′-((6-((1-(4-((trifluoromethyl)thio)phenyl)ethyl)carbamoyl)-3,4-dihydroquinoline-1 (2H)-yl)methyl)-[1,1′-biphenyl]-2-carboxylic acid;
(14) (S)-4′-((6-((1-(4-cyanophenyl)ethyl)carbamoyl)-3,4-dihydroquinoline-1(2H)-yl)methyl)-[1,1′-biphenyl]-2-carboxylic acid;
(15) 4′-((6-(cromene-3-ylcarbamoyl)-3,4-dihydroquinoline-1(2H)-yl)methyl)-[1,1′-biphenyl]-2-carboxylic acid;
(16) (S)-4′-((6-((1-(2,6- [symbol] fluorophenyl)ethyl)carbamoyl)-3, 4-dihydroquinoline-1(2H)-yl)methyl)-[1,1′-biphenyl]-2-carboxylic acid;
(17) (S)-4′-((6-((1-(3-fluorophenyl)ethyl)carbamoyl)-3,4-dihydroquinoline-1(2H)-yl)methyl)-[1,1′-biphenyl]-2-carboxylic acid;
(18) (S)-4′-((6-((1-(3-(trifluoromethoxy)phenyl)ethyl)carbamoyl)-3,4-dihydroquinoline-1(2H)-yl)methyl)-[1,1′-biphenyl]-2-carboxylic acid;
(19) (S)-4′-((6-((1-(3-(trifluoromethyl)phenyl)ethyl)carbamoyl)-3,4-dihydroquinoline-1(2H)-yl)methyl)-[1,1′-biphenyl]-2-carboxylic acid;
(20) (S)-4′-((6-((1-(naphthalene-2-yl)ethyl)carbamoyl)-3,4-dihydroquinoline-1(2H)-yl)methyl)-[1,1′-biphenyl]-2-carboxylic acid;
(21) (S)-4′-((6-((1-(3,4- [symbol] fluorophenyl)ethyl)carbamoyl)-3,4-dihydroquinoline-1(2H)-yl)methyl)-[1,1′-biphenyl]-2-carboxylic acid;
(22) (S)-4′-((6-((1-(3-methoxyphenyl)ethyl)carbamoyl)-3,4-dihydroquinoline-1(2H)-yl)methyl)-[1,1′-biphenyl]-2-carboxylic acid;
(23) (S)-4′-((6-((1-(2-methoxyphenyl)ethyl)carbamoyl)-3,4-dihydroquinoline-1(2H)-yl)methyl)-[1,1′-biphenyl]-2-carboxylic acid;
(24) (S)-4′-((6-((1-(2-fluorophenyl)ethyl)carbamoyl)-3, 4-dihydroquinoline-1(2H)-yl)methyl)-[1,1′-biphenyl]-2-carboxylic acid;
(25) (S)-4′-((6-((1-(2-(trifluoromethyl)phenyl)ethyl)carbamoyl)-3,4-dihydroquinoline-1(2H)-yl)methyl)-[1,1′-biphenyl]-2-carboxylic acid;
(26) 4′-((6-((pyridine-2-ylmethyl)carbamoyl)-3,4-dihydroquinoline-1(2H)-yl)methyl)-[1,1′-biphenyl]-2-carboxylic acid;
(27) 4′-((6-((pyridine-4-ylmethyl)carbamoyl)-3,4-dihydroquinoline-1(2H)-yl)methyl)-[1,1′-biphenyl]-2-carboxylic acid;
(28) 4′-((6-(benzylcarbamoyl)-3,4-dihydroquinoline-1(2H)-yl)methyl)-[1,1′-biphenyl]-2-carboxylic acid;
(29) 4′-((6-((2-bromobenzyl)carbamoyl)-3,4-dihydroquinoline-1(2H)-yl)methyl)-[1,1′-biphenyl]-2-carboxylic acid;
(30) 4′-((6-(((2,3-dihydrobenzo[b][1,4]dioxin-6-yl)methyl)carbamoyl)-3,4-dihydroquinoline-1(2H)-yl)methyl)-[1,1′-biphenyl]-2-carboxylic acid;
(31) 4′-((6-((4-bromobenzyl)carbamoyl)-3,4-dihydroquinoline-1(2H)-yl)methyl)-[1,1′-biphenyl]-2-carboxylic acid;
(32) 4′-((6-((3-(trifluoromethoxy)benzyl)carbamoyl)-3,4-dihydroquinoline-1(2H)-yl)methyl)-[1,1′-biphenyl]-2-carboxylic acid;
(33) (S)-1-((2′-carbamoyl-[1,1′-biphenyl]-4-yl)methyl)-N-(1-(4-(trifluoromethyl)phenyl)ethyl)-1,2,3,4-tetrahydroquinoline-6-carboxamide;
(34) (S)-1-((2′-carbamoyl-[1,1′-biphenyl]-4-yl)methyl)-N-(1-(4-(trifluoromethoxy)phenyl)ethyl)-1,2,3,4-tetrahydroquinoline-6-carboxamide;
(35) (S)-1-((2′-carbamoyl-[1,1′-biphenyl]-4-yl)methyl)-N-(1-(3-chlorophenyl)ethyl)-1,2,3,4-tetrahydroquinoline-6-carboxamide;
(36) (S)-1-((2′-carbamoyl-[1,1′-biphenyl]-4-yl)methyl)-N-(1-(4-fluoro phenyl)ethyl)-1,2,3,4-tetrahydroquinoline-6-carboxamide;
(37) (S)—N-(1-(4-bromophenyl)ethyl)-1-((2′-carbamoyl-[1,1′-biphenyl]-4-yl)methyl)-1,2,3,4-tetrahydroquinoline-6-carboxamide;
(38) (S)-1-((2′-carbamoyl-[1,1′-biphenyl]-4-yl)methyl)-N-(1-(naphthalene-1-yl)ethyl)-1,2,3,4-tetrahydroquinoline-6-carboxamide;
(39) (S)-1-((2′-carbamoyl-[1,1′-biphenyl]-4-yl)methyl)-N-(1-(3,4-difluorophenyl)ethyl)-1,2,3,4-tetrahydroquinoline-6-carboxamide;
(40) (S)-1-((2′-carbamoyl-[1,1′-biphenyl]-4-yl)methyl)-N-(1-(3-methoxyphenyl)ethyl)-1,2,3,4-tetrahydroquinoline-6-carboxamide;
(41) (S)-1-((2′-carbamoyl-[1,1′-biphenyl]-4-yl)methyl)-N-(1-(2-methoxyphenyl)ethyl)-1,2,3,4-tetrahydroquinoline-6-carboxamide;
(42) (S)-1-((2′-carbamoyl-[1,1′-biphenyl]-4-yl)methyl)-N-(1-(2-fluorophenyl)ethyl)-1,2,3,4-tetrahydroquinoline-6-carboxamide;
(43) (S)-1-((2′-carbamoyl-[1,1′-biphenyl]-4-yl)methyl)-N-(1-(2-(trifluoromethyl)phenyl)ethyl)-1,2,3,4-tetrahydroquinoline-6-carboxamide;
(44) (S)-1-((2′-carbamoyl-[1,1′-biphenyl]-4-yl)methyl)-N-(1-(naphthalene-2-yl)ethyl)-1,2,3,4-tetrahydroquinoline-6-carboxamide;
(45) (S)-1-((2′-carbamoyl-[1,1′-biphenyl]-4-yl)methyl)-N-(1-(3-fluorophenyl)ethyl)-1,2,3,4-tetrahydroquinoline-6-carboxamide;
(46) (S)-1-((2′-carbamoyl-[1,1′-biphenyl]-4-yl)methyl)-N-(1-(3-(trifluoromethoxy)phenyl)ethyl)-1,2,3,4-tetrahydroquinoline-6-carboxamide;
(47) (S)-1-((2′-carbamoyl-[1,1′-biphenyl]-4-yl)methyl)-N-(1-(3-(trifluoromethyl)phenyl)ethyl)-1,2,3,4-tetrahydroquinoline-6-carboxamide;
(48) 1-((2′-carbamoyl-[1,1′-biphenyl]-4-yl)methyl)-N-(pyridine-4-ylmethyl)-1,2,3,4-tetrahydroquinoline-6-carboxamide;
(49) (S)-1-((2′-carbamoyl-[1,1′-biphenyl]-4-yl)methyl)-N-(1-(3-fluoro-4-methylphenyl)ethyl)-1,2,3,4-tetrahydroquinoline-6-carboxamide;
(50) N-benzyl-1-((2′-carbamoyl-[1,1′-biphenyl]-4-yl)methyl)-1,2,3,4-tetrahydroquinoline-6-carboxamide;
(51) N-(2-bromobenzyl)-1-((2′-carbamoyl-[1,1′-biphenyl]-4-yl)methyl)-1,2,3,4-tetrahydroquinoline-6-carboxamide;
(52) 1-((2′-carbamoyl-[1,1′-biphenyl]-4-yl)methyl)-N-((2,3-dihydrobenzo[b][1,4]dioxin-6-yl)methyl)-1,2,3,4-tetrahydroquinoline-6-carboxamide;
(53) N-(4-bromobenzyl)-1-((2′-carbamoyl-[1,1′-biphenyl]-4-yl)methyl)-1,2,3,4-tetrahydroquinoline-6-carboxamide;
(54) 1-((2′-carbamoyl-[1,1′-biphenyl]-4-yl)methyl)-N-(3-(trifluoromethoxy)benzyl)-1,2,3,4-tetrahydroquinoline-6-carboxamide;
(55) sodium (S)-4′-((6-((1-phenylethyl)carbamoyl)-3,4-dihydroquinoline-1(2H)-yl)methyl)-[1,1′-biphenyl]-2-carboxylate;
(56) sodium (S)-4′-((6-((1-(3-fluoro-4-methylphenyl)ethyl)carbamoyl)-3,4-dihydroquinoline-1(2H)-yl)methyl)-[1,1′-biphenyl]-2-carboxylate;
(57) sodium (S)-4′-((6-((1-(4-cyanophenyl)ethyl)carbamoyl)-3, 4-dihydroquinoline-1(2H)-yl)methyl)-[1,1′-biphenyl]-2-carboxylate;
(58) (S)-4′-((7-((1-phenylethyl)carbamoyl)-2H-benzo[b][1,4]oxazine-4(3H)-yl)methyl)-[1,1′-biphenyl]2-carboxylic acid;
(59) (S)-4-((2′-carbamoyl-[1,1′-biphenyl]-4-yl)methyl)-N-(1-phenylethyl)-3,4-dihydro-2H-benzo[b][1,4]oxazine-7-carboxamide;
(60) (S)-4′-((7-((1-(4-nitrophenyl)ethyl)carbamoyl)-2H-benzo[b][1,4]oxazine-4(3H)-yl)methyl)-[1,1′-biphenyl]-2-carboxylic acid;
(61) (S)-4′-((7-((1-(4-bromophenyl)ethyl)carbamoyl)-2H-benzo[b][1,4]oxazine-4(3H)-yl)methyl)-[1,1′-biphenyl]-2-carboxylic acid;
(62) (S)-4′-((7-((1-phenylpropyl)carbamoyl)-2H-benzo[b][1,4]oxazine-4(3H)-yl)methyl)-[1,1′-biphenyl]-2-carboxylic acid;
(63) (S)-4′-((7-((1-(4-fluorophenyl)ethyl)carbamoyl)-2H-benzo[b][1,4]oxazine-4(3H)-yl)methyl)-[1,1′-biphenyl]-2-carboxylic acid;
(64) (S)-4′-((7-((1-(4-chlorophenyl)ethyl)carbamoyl)-2H-benzo[b][1,4]oxazine-4(3H)-yl)methyl)-[1,1′-biphenyl]-2-carboxylic acid;
(65) (S)-4′-((7-((1-(3-chlorophenyl)ethyl)carbamoyl)-2H-benzo[b][1,4]oxazine-4(3H)-yl)methyl)-[1,1′-biphenyl]-2-carboxylic acid;
(66) 4′-((7-((4-nitrobenzyl)carbamoyl)-2H-benzo[b][1,4]oxazine-4(3H)-yl)methyl)-[1,1′-biphenyl]-2-carboxylic acid;
(67) 4′-((7-(benzylcarbamoyl)-2H-benzo[b][1,4]oxazine-4(3H)-yl)methyl)-[1,1′-biphenyl]-2-carboxylic acid;
(68) 4′-((7-(phenethylcarbamoyl)-2H-benzo[b][1,4]oxazine-4(3H)-yl)methyl)-[1,1′-biphenyl]-2-carboxylic acid;
(69) 4′-((7-((furan-2-ylmethyl)carbamoyl)-2H-benzo[b][1,4]oxazine-4(3H)-yl)methyl)-[1,1′-biphenyl]-2-carboxylic acid;
(70) (S)-4′-((7-((1-(naphthalene-1-yl)ethyl)carbamoyl)-2H-benzo[b][1,4]oxazine-4(3H)-yl)methyl)-[1,1′-biphenyl]-2-carboxylic acid;
(71) 4′-((7-((4-(methoxycarbonyl)benzyl)carbamoyl)-2H-benzo[b][1,4]oxazine-4(3H)-yl)methyl)-[1,1′-biphenyl]-2-carboxylic acid;
(72) 4′-((7-((3-methoxybenzyl)carbamoyl)-2H-benzo[b][1,4]oxazine-4(3H)-yl)methyl)-[1,1′-biphenyl]-2-carboxylic acid;
(73) 4′-((7-((4-bromobenzyl)carbamoyl)-2H-benzo[b][1, 4]oxazine-4(3H)-yl)methyl)-[1,1′-biphenyl]-2-carboxylic acid;
(74) (S)-4′-((7-((1-(4-cyanophenyl)ethyl)carbamoyl)-2H-benzo[b][1,4]oxazine-4(3H)-yl)methyl)-[1,1′-biphenyl]-2-carboxylic acid;
(75) (S)-4′-((7-((1-(4-(trifluoromethoxy)phenyl)ethyl)carbamoyl)-2H-benzo[b][1,4]oxazine-4(3H)-yl)methyl)-[1,1′-biphenyl]-2-carboxylic acid;
(76) (S)-4′-((7-((1-(4-(trifluoromethyl)phenyl)ethyl)carbamoyl)-2H-benzo[b][1,4]oxazine-4(3H)-yl)methyl)-[1,1′-biphenyl]-2-carboxylic acid;
(77) (S)-4′-((7-((1-(4-((trifluoromethyl)thio)phenyl)ethyl)carbamoyl)-2H-benzo[b][1,4]oxazine-4(3H)-yl)methyl)-[1,1′-biphenyl]-2-carboxylic acid;
(78) (S)-4′-((7-((1-(4-(tert-butyl)phenyl)ethyl)carbamoyl)-2H-benzo[b][1,4]oxazine-4(3H)-yl)methyl)-[1,1′-biphenyl]-2-carboxylic acid;
(79) (S)-4′-((7-((1-(4-fluoro-2-methylphenyl)ethyl)carbamoyl)-2H-benzo[b][1,4]oxazine-4(3H)-yl)methyl)-[1,1′-biphenyl]-2-carboxylic acid;
(80) (S)-4′-((7-((1-(3-fluoro-4-methylphenyl)ethyl)carbamoyl)-2H-benzo[b][1,4]oxazine-4(3H)-yl)methyl)-[1,1′-biphenyl]-2-carboxylic acid;
(81) (R)-4′-((7-((1-(4-cyanophenyl)ethyl)carbamoyl)-2H-benzo[b][1,4]oxazine-4(3H)-yl)methyl)-[1,1′-biphenyl]-2-carboxylic acid;
(82) 4′-((7-(cromene-3-ylcarbamoyl)-2H-benzo[b][1,4]oxazine-4(3H)-yl)methyl)-[1,1′-biphenyl]-2-carboxylic acid;
(83) (S)-4′-((7-((1-(2,6- [symbol] fluorophenyl)ethyl)carbamoyl)-2H-benzo[b][1,4]oxazine-4(3H)-yl)methyl)-[1,1′-biphenyl]-2-carboxylic acid;
(84) (S)-4′-((7-((1-(3-fluorophenyl)ethyl)carbamoyl)-2H-benzo[b][1,4]oxazine-4(3H)-yl)methyl)-[1,1′-biphenyl]-2-carboxylic acid;
(85) (S)-4′-((7-((1-(3-(trifluoromethoxy)phenyl)ethyl)carbamoyl)-2H-benzo[b][1,4]oxazine-4(3H)-yl)methyl)-[1,1′-biphenyl]-2-carboxylic acid;
(86) (S)-4′-((7-((1-(3-(trifluoromethyl)phenyl)ethyl)carbamoyl)-2H-benzo[b][1,4]oxazine-4(3H)-yl)methyl)-[1,1′-biphenyl]-2-carboxylic acid;
(87) (S)-4′-((7-((1-(naphthalene-2-yl)ethyl)carbamoyl)-2H-benzo[b][1,4]oxazine-4(3H)-yl)methyl)-[1,1′-biphenyl]-2-carboxylic acid;
(88) 4′-((7-((pyridine-2-ylmethyl)carbamoyl)-2H-benzo[b][1,4]oxazine-4(3H)-yl)methyl)-[1,1′-biphenyl]-2-carboxylic acid;
(89) 4′-((7-((pyridine-3-ylmethyl)carbamoyl)-2H-benzo[b][1,4]oxazine-4(3H)-yl)methyl)-[1,1′-biphenyl]-2-carboxylic acid;
(90) 4′-((7-((pyridine-4-ylmethyl)carbamoyl)-2H-benzo[b][1,4]oxazine-4(3H)-yl)methyl)-[1,1′-biphenyl]-2-carboxylic acid;
(91) 4′-((7-(benzylcarbamoyl)-2H-benzo[b][1,4]oxazine-4(3H)-yl)methyl)-[1,1′-biphenyl]-2-carboxylic acid;
(92) 4′-((7-((2-bromobenzyl)carbamoyl)-2H-benzo[b][1,4]oxazine-4(3H)-yl)methyl)-[1,1′-biphenyl]-2-carboxylic acid;
(93) 4′-((7-(((2,3-dihydrobenzo[b][1,4]dioxin-6-yl)methyl)carbamoyl)-2H-benzo[b][1,4]oxazine-4(3H)-yl)methyl)-[1,1′-biphenyl]-2-carboxylic acid;
(94) (R)-4-((2′-carbamoyl-[1,1′-biphenyl]-4-yl)methyl)-N-(1-(4-cyanophenyl)ethyl)-3,4-dihydro-2H-benzo[b][1,4]oxazine-7-carboxamide;
(95) (S)-4-((2′-carbamoyl-[1,1′-biphenyl]-4-yl)methyl)-N-(1-(3-fluorophenyl)ethyl)-3,4-dihydro-2H-benzo[b][1,4]oxazine-7-carboxamide;
(96) (S)-4-((2′-carbamoyl-[1,1′-biphenyl]-4-yl)methyl)-N-(1-(naphthalene-2-yl)ethyl)-3,4-dihydro-2H-benzo[b][1,4]oxazine-7-carboxamide;
(97) 4-((2′-carbamoyl-[1,1′-biphenyl]-4-yl)methyl)-N-(pyridine-3-ylmethyl)-3,4-dihydro-2H-benzo[b][1,4]oxazine-7-carboxamide;
(98) 4-((2′-carbamoyl-[1,1′-biphenyl]-4-yl)methyl)-N-(pyridine-4-ylmethyl)-3,4-dihydro-2H-benzo[b][1,4]oxazine-7-carboxamide;
(99) (S)-4-((2′-carbamoyl-[1,1′-biphenyl]-4-yl)methyl)-N-(1-(4-((trifluoromethyl)thio)phenyl)ethyl)-3,4-dihydro-2H-benzo[b][1,4]oxazine-7-carboxamide;
(100) N-benzyl-4-((2′-carbamoyl-[1,1′-biphenyl]-4-yl)methyl)-3,4-dihydro-2H-benzo[b][1,4]oxazine-7-carboxamide;
(101) N-(2-bromobenzyl)-4-((2′-carbamoyl-[1,1′-biphenyl]-4-yl)methyl)-3, 4-dihydro-2H-benzo[b][1,4]oxazine-7-carboxamide;
(102) 4-((2′-carbamoyl-[1,1′-biphenyl]-4-yl)methyl)-N-((2,3-dihydrobenzo[b][1,4]dioxin-6-yl)methyl)-3,4-dihydro-2H-benzo[b][1,4]oxazine-7-carboxamide;
(103) sodium (S)-4′-((7-((1-phenylethyl)carbamoyl)-2H-benzo[b][1,4]oxazine-4(3H)-yl)methyl)-[1,1′-biphenyl]-2-carboxylate;
(104) sodium (S)-4′-((7-((1-(4-cyanophenyl)ethyl)carbamoyl)-2H-benzo[b][1,4]oxazine-4(3H)-yl)methyl)-[1,1′-biphenyl]-2-carboxylate;
(105) sodium (S)-4′-((7-((1-(4-nitrophenyl)ethyl)carbamoyl)-2H-benzo[b][1,4]oxazine-4(3H)-yl)methyl)-[1,1′-biphenyl]-2-carboxylate;
(106) (S)-1-((2′-carbamoyl-3-fluoro-[1,1′-biphenyl]-4-yl)methyl)-N-(1-phenylethyl)-1,2,3,4-tetrahydroquinoline-6-carboxylic acid;
(107) (S)-1-((2′-carbamoyl-3-fluoro-[1,1′-biphenyl]-4-yl)methyl)-N-(1-phenylethyl)-1,2,3,4-tetrahydroquinoline-6-carboxamide;
(108) (S)-3′-fluoro-4′-((6-((1-(naphthalene-2-yl)ethyl)carbamoyl)-3,4-dihydro quinoline-1(2H)-yl)methyl)-[1,1′-biphenyl]-2-carboxylic acid;
(109) (S)-3′-fluoro-4′-((6-((1-(4-nitrophenyl)ethyl)carbamoyl)-3,4-dihydroquinoline-1(2H)-yl)methyl)-[1,1′-biphenyl]-2-carboxylic acid;
(110) (S)-4′-((6-((1-(4-cyanophenyl)ethyl)carbamoyl)-3, 4-dihydroquinoline-1(2H)-yl)methyl)-3′-fluoro-[1,1′-biphenyl]-2-carboxylic acid;
(111) (S)-3′-fluoro-4′-((6-((1-(3-fluoro-4-methylphenyl)ethyl)carbamoyl)-3,4-dihydroquinoline-1(2H)-yl)methyl)-[1,1′-biphenyl]-2-carboxylic acid;
(112) (S)-1-((2′-carbamoyl-3-fluoro-[1,1′-biphenyl]-4-yl)methyl)-N-(1-(4-cyanophenyl)ethyl)-1,2,3,4-tetrahydroquinoline-6-carboxamide;
(113) (S)-1-((2′-carbamoyl-3-fluoro-[1,1′-biphenyl]-4-yl)methyl)-N-(1-(4-nitrophenyl)ethyl)-1,2,3,4-tetrahydroquinoline-6-carboxamide;
(114) (S)-1-((2′-carbamoyl-3-fluoro-[1,1′-biphenyl]-4-yl)methyl)-N-(1-(3-fluoro-4-methylphenyl)ethyl)-1,2,3,4-tetrahydroquinoline-6-carboxamide;
(115) (S)-1-((2′-carbamoyl-3-fluoro-[1,1′-biphenyl]-4-yl)methyl)-N-(1-(naphthalene-2-yl)ethyl)-1,2,3,4-tetrahydroquinoline-6-carboxamide;
(116) (S)-3′-((6-((1-phenylethyl)carbamoyl)-3,4-dihydroquinoline-1(2H)-yl)methyl)-[1,1′-biphenyl]-2-carboxylic acid;
(117) (S)-1-((2′-carbamoyl-[1,1′-biphenyl]-3-yl)methyl)-N-(1-phenylethyl)-1,2,3,4-tetrahydroquinoline-6-carboxamide;
(118) (S)-3′-((6-((1-(4-cyanophenyl)ethyl)carbamoyl)-3,4-dihydroquinoline-1(2H)-yl)methyl)-[1,1′-biphenyl]-2-carboxylic acid;
(119) (S)-3′-((6-((1-(4-nitrophenyl)ethyl)carbamoyl)-3,4-dihydroquinoline-1(2H)-yl)methyl)-[1,1′-biphenyl]-2-carboxylic acid;
(120) (S)-3′-((6-((1-(naphthalene-2-yl)ethyl)carbamoyl)-3,4-dihydroquinoline-1(2H)-yl)methyl)-[1,1′-biphenyl]-2-carboxylic acid;
(121) (S)-3′-((6-((1-(3-fluoro-4-methylphenyl)ethyl)carbamoyl)-3,4-dihydroquinoline-1(2H)-yl)methyl)-[1,1′-biphenyl]-2-carboxylic acid;
(122) (S)-1-((2′-carbamoyl-[1,1′-biphenyl]-3-yl)methyl)-N-(1-(4-cyanophenyl)ethyl)-1,2,3,4-tetrahydroquinoline-6-carboxamide;
(123) (S)-1-((2′-carbamoyl-[1,1′-biphenyl]-3-yl)methyl)-N-(1-(4-nitrophenyl)ethyl)-1,2,3,4-tetrahydroquinoline-6-carboxamide;
(124) (S)-1-((2′-carbamoyl-[1,1′-biphenyl]-3-yl)methyl)-N-(1-(naphthalene-2-yl)ethyl)-1,2,3,4-tetrahydroquinoline-6-carboxamide;
(125) (S)-1-((2′-carbamoyl-[1,1′-biphenyl]-3-yl)methyl)-N-(1-(3-fluoro-4-methylphenyl)ethyl)-1,2,3,4-tetrahydroquinoline-6-carboxamide;
(126) (S)-2′-fluoro-3′-((6-((1-phenylethyl)carbamoyl)-3,4-dihydroquinoline-1(2H)-yl)methyl)-[1,1′-biphenyl]-2-carboxylic acid;
(127) (S)-1-((2′-carbamoyl-2-fluoro-[1,1′-biphenyl]-3-yl)methyl)-N-(1-phenylethyl)-1,2,3,4-tetrahydroquinoline-6-carboxamide compound;
(128) (S)-2′-fluoro-3′-((6-((1-(4-nitrophenyl)ethyl)carbamoyl)-3,4-dihydroquinoline-1(2H)-yl)methyl)-[1,1′-biphenyl]-2-carboxylic acid;
(129) (S)-2′-fluoro-3′-((6-((1-(naphthalene-2-yl)ethyl)carbamoyl)-3,4-dihydroquinoline-1(2H)-yl)methyl)-[1,1′-biphenyl]-2-carboxylic acid;
(130) (S)-2′-fluoro-3′-((6-((1-(3-fluoro-4-methylphenyl)ethyl)carbamoyl)-3,4-dihydroquinoline-1(2H)-yl)methyl)-[1,1′-biphenyl]-2-carboxylic acid;
(131) (S)-1-((2′-carbamoyl-2-fluoro-[1,1′-biphenyl]-3-yl)methyl)-N-(1-(4-nitrophenyl)ethyl)-1,2,3,4-tetrahydroquinoline-6-carboxamide;
(132) (S)-1-((2′-carbamoyl-2-fluoro-[1,1′-biphenyl]-3-yl)methyl)-N-(1-(naphthalene-2-yl)ethyl)-1,2,3,4-tetrahydroquinoline-6-carboxamide;
(133) (S)-1-((2′-carbamoyl-2-fluoro-[1,1′-biphenyl]-3-yl)methyl)-N-(1-(3-fluoro-4-methylphenyl)ethyl)-1,2,3,4-tetrahydroquinoline-6-carboxamide;
(134) (S)-4′-fluoro-3′-((6-((1-phenylethyl)carbamoyl)-3,4-dihydroquinoline-1(2H)-yl)methyl)-[1,1′-biphenyl]-2-carboxylic acid;
(135) (S)-1-((2′-carbamoyl-4-fluoro-[1,1′-biphenyl]-3-yl)methyl)-N-(1-phenylethyl)-1,2,3,4-tetrahydroquinoline-6-carboxamide;
(136) (S)-4′-fluoro-3′-((6-((1-(4-nitrophenyl)ethyl)carbamoyl)-3,4-dihydroquinoline-1(2H)-yl)methyl)-[1,1′-biphenyl]-2-carboxylic acid;
(137) (S)-4′-fluoro-3′-((6-((1-(naphthalene-2-yl)ethyl)carbamoyl)-3,4-dihydroquinoline-1(2H)-yl)methyl)-[1,1′-biphenyl]-2-carboxylic acid;
(138) (S)-4′-fluoro-3′-((6-((1-(3-fluoro-4-methylphenyl)ethyl)carbamoyl)-3,4-dihydroquinoline-1(2H)-yl)methyl)-[1,1′-biphenyl]-2-carboxylic acid;
(139) (S)-1-((2′-carbamoyl-4-fluoro-[1,1′-biphenyl]-3-yl)methyl)-N-(1-(4-nitrophenyl)ethyl)-1,2,3,4-tetrahydroquinoline-6-carboxamide;
(140) (S)-1-((2′-carbamoyl-4-fluoro-[1,1′-biphenyl]-3-yl)methyl)-N-(1-(naphthalene-2-yl)ethyl)-1,2,3,4-tetrahydroquinoline-6-carboxamide;
(141) (S)-1-((2′-carbamoyl-4-fluoro-[1,1′-biphenyl]-3-yl)methyl)-N-(1-(3-fluoro-4-methylphenyl)ethyl)-1,2,3,4-tetrahydroquinoline-6-carboxamide;
(142) (S)-2′-((7-((1-phenylethyl)carbamoyl)-2H-benzo[b][1,4]oxazine-4(3H)-yl)methyl)-[1,1′-biphenyl]-4-carboxylic acid;
(143) (S)-4-((4′-carbamoyl-[1,1′-biphenyl]-2-yl)methyl)-N-(1-phenylethyl)-3,4-dihydro-2H-benzo[b][1,4]oxazine-7-carboxamide;
(144) (S)-2′-((7-((1-(4-cyanophenyl)ethyl)carbamoyl)-2H-benzo[b][1,4]oxazine-4(3H)-yl)methyl)-[1,1′-biphenyl]-4-carboxylic acid;
(145) (S)-2′-((7-((1-(3-fluoro-4-methylphenyl)ethyl)carbamoyl)-2H-benzo[b][1,4]oxazine-4(3H)-yl)methyl)-[1,1′-biphenyl]-4-carboxylic acid;
(146) (S)-2′-((7-((1-(4-nitrophenyl)ethyl)carbamoyl)-2H-benzo[b][1,4]oxazine-4(3H)-yl)methyl)-[1,1′-biphenyl]-4-carboxylic acid;
(147) (S)-2′-((7-((1-(naphthalene-2-yl)ethyl)carbamoyl)-2H-benzo[b][1,4]oxazine-4(3H)-yl)methyl)-[1,1′-biphenyl]-4-carboxylic acid;
(148) (S)-4-((4′-carbamoyl-[1,1′-biphenyl]-2-yl)methyl)-N-(1-(3-fluoro-4-methylphenyl)ethyl)-3,4-dihydro-2H-benzo[b][1,4]oxazine-7-carboxamide;
(149) (S)-4-((4′-carbamoyl-[1,1′-biphenyl]-2-yl)methyl)-N-(1-(4-cyanophenyl)ethyl)-3,4-dihydro-2H-benzo[b][1,4]oxazine-7-carboxamide;
(150) (S)-4-((4′-carbamoyl-[1,1′-biphenyl]-2-yl)methyl)-N-(1-(4-nitrophenyl)ethyl)-3,4-dihydro-2H-benzo[b][1,4]oxazine-7-carboxamide;
(151) (S)-4-((4′-carbamoyl-[1,1′-biphenyl]-2-yl)methyl)-N-(1-(naphthalene-2-yl)ethyl)-3,4-dihydro-2H-benzo[b][1,4]oxazine-7-carboxamide;
(152) (S)-2′-((6-((1-phenylethyl)carbamoyl)-3,4-dihydroquinoline-1(2H)-yl)methyl)-[1,1′-biphenyl]-4-carboxylic acid;
(153) (S)-1-((4′-carbamoyl-[1,1′-biphenyl]-2-yl)methyl)-N-(1-phenylethyl)-1,2,3,4-tetrahydroquinoline-6-carboxamide;
(154) (S)-2′-((6-((1-(3-fluoro-4-methylphenyl)ethyl)carbamoyl)-3,4-dihydroquinoline-1(2H)-yl)methyl)-[1,1′-biphenyl]-4-carboxylic acid;
(155) (S)-2′-((6-((1-(4-nitrophenyl)ethyl)carbamoyl)-3,4-dihydroquinoline-1(2H)-yl)methyl)-[1,1′-biphenyl]-4-carboxylic acid;
(156) (S)-2′-((6-((1-(naphthalene-2-yl)ethyl)carbamoyl)-3,4-dihydroquinoline-1(2H)-yl)methyl)-[1,1′-biphenyl]-4-carboxylic acid;
(157) (S)-2′-((6-((1-(4-cyanophenyl)ethyl)carbamoyl)-3,4-dihydroquinoline-1(2H)-yl)methyl)-[1,1′-biphenyl]-4-carboxylic acid;
(158) (S)-1-((4′-carbamoyl-[1,1′-biphenyl]-2-yl)methyl)-N-(1-(3-fluoro-4-methylphenyl)ethyl)-1,2,3,4-tetrahydroquinoline-6-carboxamide;
(159) (S)-1-((4′-carbamoyl-[1,1′-biphenyl]-2-yl)methyl)-N-(1-(4-nitrophenyl)ethyl)-1,2,3,4-tetrahydroquinoline-6-carboxamide;
(160) (S)-1-((4′-carbamoyl-[1,1′-biphenyl]-2-yl)methyl)-N-(1-(naphthalene-2-yl)ethyl)-1,2,3,4-tetrahydroquinoline-6-carboxamide;
(161) (S)-1-((4′-carbamoyl-[1,1′-biphenyl]-2-yl)methyl)-N-(1-(4-cyanophenyl)ethyl)-1,2,3,4-tetrahydroquinoline-6-carboxamide;
(162) (S)-2′-fluoro-4′-((7-((1-phenylethyl)carbamoyl)-2H-benzo[b][1,4]oxazine-4(3H)-yl)methyl)-[1,1′-biphenyl]-2-carboxylic acid;
(163) (S)-2′-fluoro-4′-((7-((1-(naphthalene-2-yl)ethyl)carbamoyl)-2H-benzo[b][1,4]oxazine-4(3H)-yl)methyl)-[1,1′-biphenyl]-2-carboxylic acid; and
(164) (S)-2′-fluoro-4′-((7-(((3-fluoro-4-methylphenyl)ethyl)carbamoyl)-2H-benzo[b][1,4]oxazine-4(3H)-yl)methyl)-[1,1′-biphenyl]-2-carboxylic acid.

6. A method for preparing the compound represented by formula 1 of claim 1 comprising the following steps, as shown in reaction formula 1 below:
preparing the compound represented by formula 4 by reacting the compound represented by formula 2 with the compound represented by formula 3 (step 1);
preparing the compound represented by formula 5 by substituting methoxycarbonyl group of the compound represented by formula 4 prepared in step 1 with carboxy group (step 2);
preparing the compound represented by formula 7 by reacting the compound represented by formula 5 prepared in step 2 with the compound represented by formula 6 (step 3); and
substituting t-butoxy group of the compound represented by formula 7 prepared in step 3 with —OH group (step 4); 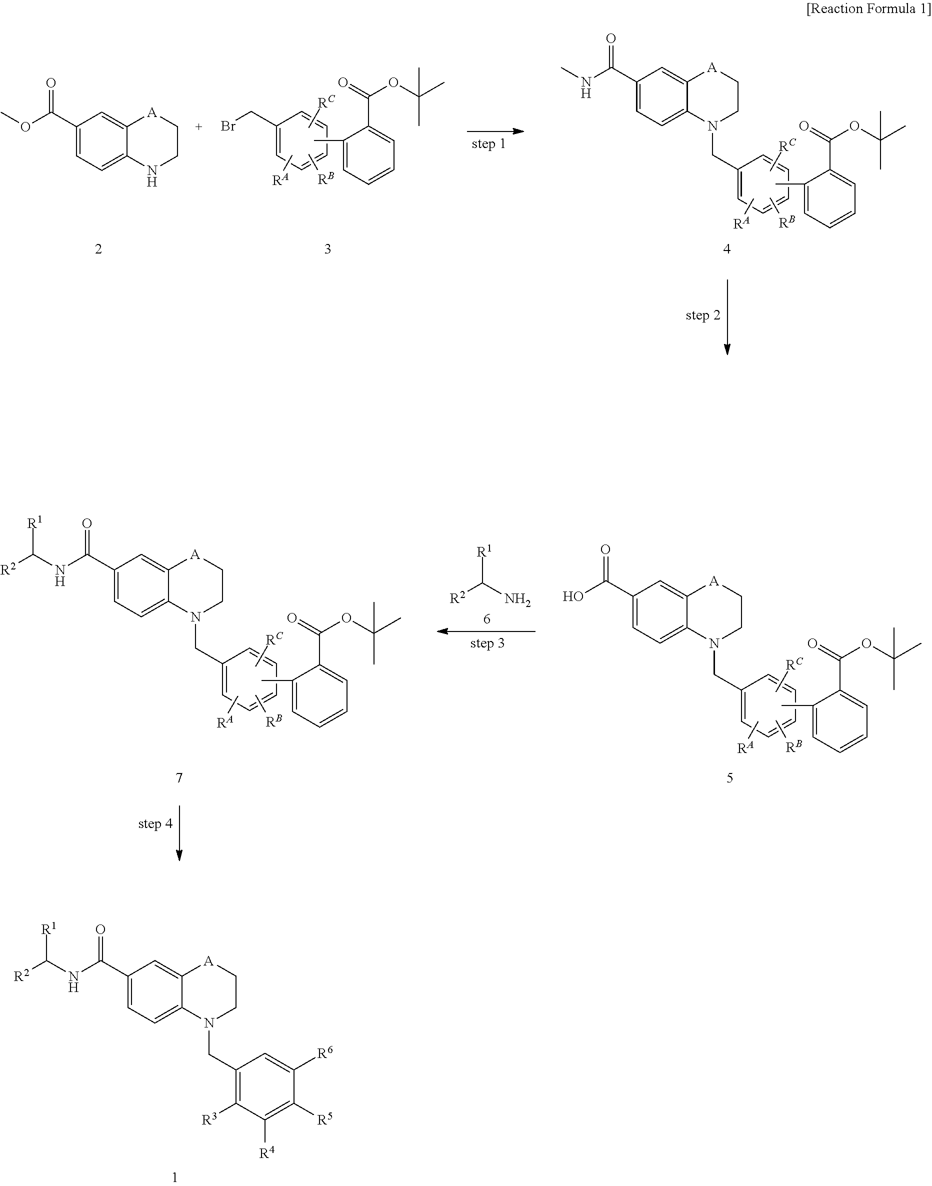
and in reaction formula 1,
R¹-R⁶ and A are as defined in formula 1; and
R^{A}, R^{B}, and R^{C} are independently as defined in R³-R⁶.

7. A method for preparing the compound represented by formula 1 of claim 1 comprising the following steps, as shown in reaction formula 2 below:
preparing the compound represented by formula 9 by reacting the compound represented by formula 8 with the compound represented by formula 6 (step 1);
preparing the compound represented by formula 7 by reacting the compound represented by formula 9 prepared in step 1 with the compound represented by formula 3 (step 2); and
substituting t-butoxy group of the compound represented by formula 7 prepared in step 2 with —OH group (step 3); 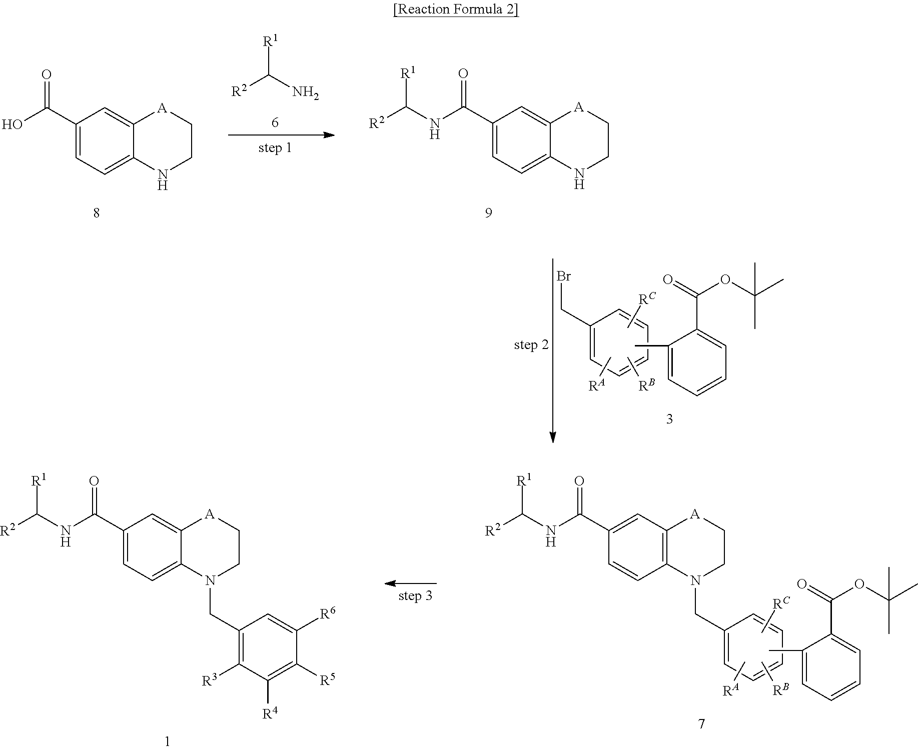
and in the reaction formula 2,
R¹-R⁶ and A are as defined in formula 1; and
R^{A}, R^{B}, and R^{C} are independently as defined in R³-R⁶.

8. A method for preparing the compound represented by formula 1 of claim 1, as shown in reaction formula 3 below, comprising the step of substituting carboxyl group of the compound represented by formula 1A with sodiumoxycarbonyl group or amide group (step 1); 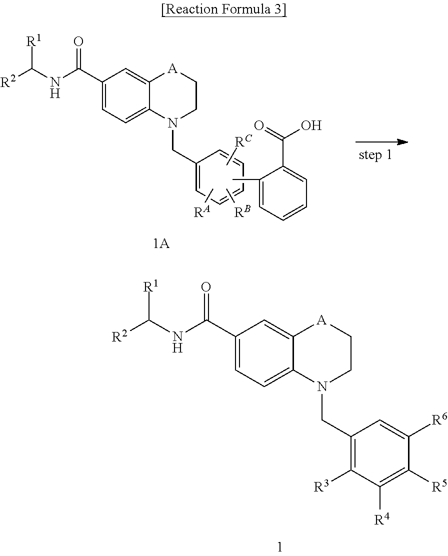
and in the reaction formula 3,
R¹-R⁶ and A are as defined in formula 1; and
R^{A}, R^{B}, and R^{C} are independently as defined in R³-R⁶.

9. A pharmaceutical composition for treating PPARG-related disease comprising the compound represented by formula 1, the optical isomer thereof, or the pharmaceutically acceptable salt of the same as an active ingredient.

10. The pharmaceutical composition for treating PPARG-related disease according to claim 9, wherein the PPARG-related disease is selected from the group consisting of diabetes, insulin resistance, impaired glucose tolerance, pre-diabetes, hyperglycemia, hyperinsulinemia, obesity, and inflammation.

11. The pharmaceutical composition for treating PPARG-related disease according to claim 9, wherein the pharmaceutical composition specifically binds to PPARG but does not act as an agonist thereof.

12. The pharmaceutical composition for treating PPARG-related disease according to claim 9, wherein the pharmaceutical composition specifically binds to PPARG but does not act as an agonist of CDK5 (cyclin dependent kinase 5).

13. The pharmaceutical composition for treating PPARG-related disease according to claim 12, wherein the pharmaceutical composition is characterized by not acting as an agonist of CDK5 that induces phosphorylation of serine, the 273^{rd} amino acid of PPARG.

14. The pharmaceutical composition for treating PPARG-related disease according to claim 13, wherein the pharmaceutical composition is characterized by not inducing side effect caused by the phosphorylation of serine, the 273^{rd} amino acid of PPARG.

15. The pharmaceutical composition for treating PPARG-related disease according to claim 14, wherein the side effect caused by the phosphorylation of serine, the 273^{rd} amino acid of PPARG is one or more side effects selected from the group consisting of weight gain, edema, impairment of bone growth or formation, and cardiac hypertrophy.

16. A method for treating PPARG-related disease in a subject, comprising administering to the subject an effective amount of a pharmaceutical composition comprising the compound of claim 1 as an active ingredient to treat the PPARG-related disease in the subject.

17. The method for treating PPARG-related disease according to claim 16, wherein the PPARG-related disease is diabetes, insulin resistance, impaired glucose tolerance, pre-diabetes, hyperglycemia, hyperinsulinemia, obesity, or inflammation.

18. The method for treating PPARG-related disease according to claim 16, wherein the compound specifically binds to PPARG but does not act as an agonist thereof.

19. The method for treating PPARG-related disease according to claim 16, wherein the compound specifically binds to PPARG but does not act as an agonist of CDK5 (cyclin dependent kinase 5).

20. The method for treating PPARG-related disease according to claim 19, wherein the compound does not act as an agonist of CDK5 that induces phosphorylation the 273^{rd} amino acid of PPARG, the 273^{rd} amino acid being a serine.
